(19) United States
(12) Patent Application Publication
(10) Pub. No.: US 2024/0180031 A1
(43) Pub. Date: May 30, 2024

(54) LIGHT-EMITTING DEVICE INCLUDING HETEROCYCLIC COMPOUND, ELECTRONIC APPARATUS INCLUDING THE SAME, AND THE HETEROCYCLIC COMPOUND

(71) Applicant: Samsung Display Co., LTD., Yongin-si (KR)

(72) Inventors: Hyoyoung Lee, Yongin-si (KR); Hyeongmin Kim, Yongin-si (KR); Juhui Yun, Yongin-si (KR); Yeseul Lee, Yongin-si (KR); Jiyong Choi, Yongin-si (KR); Moran Ha, Yongin-si (KR)

(21) Appl. No.: 18/322,402

(22) Filed: May 23, 2023

(30) Foreign Application Priority Data
Oct. 27, 2022 (KR) 10-2022-0140500

(51) Int. Cl. H10K 85/60; H10K 50/12

(52) U.S. Cl. CPC H10K 85/6572; H10K 50/12; H10K 85/654; H10K 85/6574; H10K 85/6576; H10K 2101/20

(57) ABSTRACT

Provided are a light-emitting device including a heterocyclic compound represented by Formula 1, an electronic apparatus including the light-emitting device, and the heterocyclic compound represented by Formula 1, wherein the description of Formula 1 is as described herein:

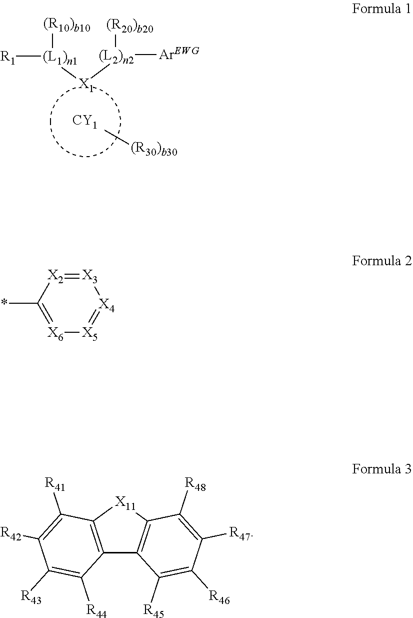

### CROSS-REFERENCE TO RELATED APPLICATION

This application claims priority to and the benefit of Korean Patent Application No. 10-2022-0140500, filed on Oct. 27, 2022, in the Korean Intellectual Property Office, the entire content of which is incorporated by reference herein in its entirety.

### BACKGROUND

### 1. Field

One or more embodiments of the present disclosure relate to a light-emitting device including a heterocyclic compound, an electronic apparatus including the light-emitting device, and the heterocyclic compound.

### 2. Description of the Related Art

Organic light-emitting devices among light-emitting devices are self-emissive devices that have wide viewing angles, high contrast ratios, short response times, and excellent characteristics in terms of luminance, driving voltage, and response speed, compared to other devices in the art.

Organic light-emitting devices may include a first electrode on a substrate, and a hole transport region, an emission layer, an electron transport region, and a second electrode sequentially stacked on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. The excitons may transition from an excited state to a ground state, thereby generating light.

### SUMMARY

One or more embodiments of the present disclosure include a light-emitting device including a heterocyclic compound, an electronic apparatus including the light-emitting device, and the heterocyclic compound.

Additional aspects of embodiments will be set forth in part in the description which follows and, in part, will be apparent from the description, or may be learned by practice of the presented embodiments of the disclosure.

According to one or more embodiments, a heterocyclic compound is represented by Formula 1 below.

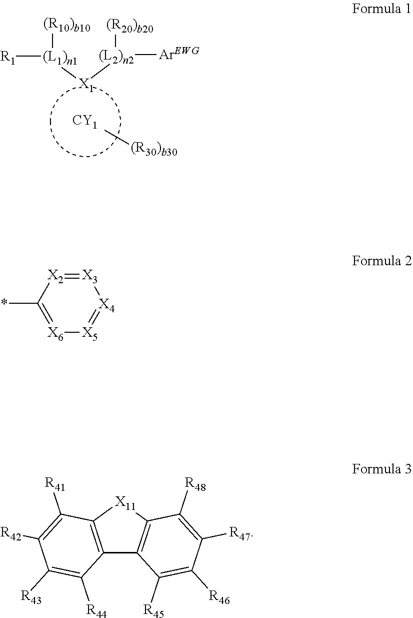

In Formulae 1 to 3,
X₁ may be an sp3 hybridized carbon atom,
CY₁ may be a C₁-C₆₀ carbocyclic group or a C₁-C₆₀ heterocyclic group,
Ar^{EWG} may be a group represented by Formula 2, X₂ may be N or C(R₂), X₃ may be N or C(R₃), X₄ may be N, C(R₄), or C(Ar₄), X₅ may be N, C(R₅), or C(Ar₅), and X₆ may be N or C(R₆),
at least one selected from X₂ to X₆ may be N,
R₁ to R₆ may each independently be a group represented by Formula 3, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one selected from R₁ to R₆ may be Ar^{EDG},
Ar^{EDG} may be a group represented by Formula 3,
L₁ and L₂ may each independently be a C₃-C₆₀ carbocyclic group or a C₁-C₆₀ heterocyclic group, n1 and n2 may each independently be an integer from 1 to 5, i) when n1 is an integer of 2 or more, a plurality of L₁(s) may be identical to or different from each other, and ii) when n2 is an integer of 2 or more, a plurality of L₂(s) may be identical to or different from each other,
R₁₀, R₂₀, and R₃₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b10, b20, and b30 may each independently be 0, 1, 2, 3, 4, 5, 6, 7, 8, 9, or 10,
X₁₁ may be O, S, Se, N(R₄₉), or P(R₄₉),
R₄₁ to R₄₉ may each independently be a binding site to a neighboring atom, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
any one selected from R₄₁ to R₄₉ may be a binding site to a neighboring atom,
R₁₀ₐ may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

According to one or more embodiments, an electronic apparatus includes a light-emitting device including the heterocyclic compound represented by Formula 1.

According to one or more embodiments, a heterocyclic compound represented by Formula 1 is provided.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic cross-sectional view of a light-emitting device according to one or more embodiments;

FIGS. 2 and 3 are each a cross-sectional view showing a light-emitting apparatus according to one or more embodiments;

FIG. 4 is a schematic perspective view of an electronic device including a light-emitting device according to one or more embodiments;

FIG. 5 is a perspective view of an exterior of a vehicle as an electronic device including a light-emitting device according to one or more embodiments; and

FIGS. 6A to 6C are each a schematic view of an interior of a vehicle according to one or more embodiments.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, example embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

A light-emitting device according to one or more embodiments may include: a first electrode; a second electrode facing the first electrode; an interlayer between the first electrode and the second electrode and including an emission layer; and a heterocyclic compound represented by Formula 1:

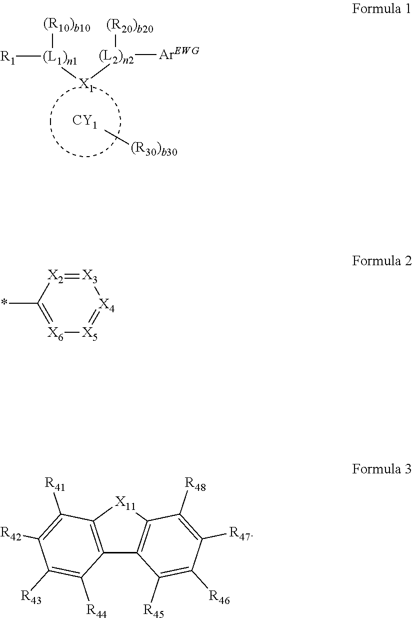

X₁ in Formulae 1 to 3 may be an sp3 hybridized carbon atom, CY₁ may be a C₁-C₆₀ carbocyclic group or a C₁-C₆₀ heterocyclic group, and R₁₀ₐ may be as described herein. The term “sp3 hybridized,” as used herein, may be given its ordinary and customary meaning, and may mean, for example, a carbon atom having four hybrid orbitals each of which combines the characteristics of one 2s orbital and three p orbitals.

For example, CY₁ may be represented by Formula 3A or 3B, and X₁ and R₃₁ to R₄₀ in Formula 3A or 3B may be as described for R₄₀:

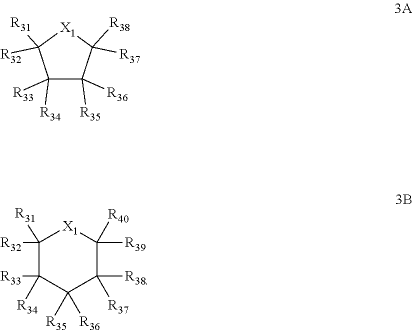

Ar^{EWG} may be a group represented by Formula 2, X₂ may be N or C(R₂), X₃ may be N or C(R₃), X₄ may be N, C(R₄), or C(Ar₄), X₅ may be N, C(R₅), or C(Ar₅), X₆ may be N or C(R₆), and at least one selected from X₂ to X₆ may be N.

In one or more embodiments, Ar^{EWG} may be a group represented by one selected from Formulae 2A to 2G, and R₂ to R₆ may be as described herein:

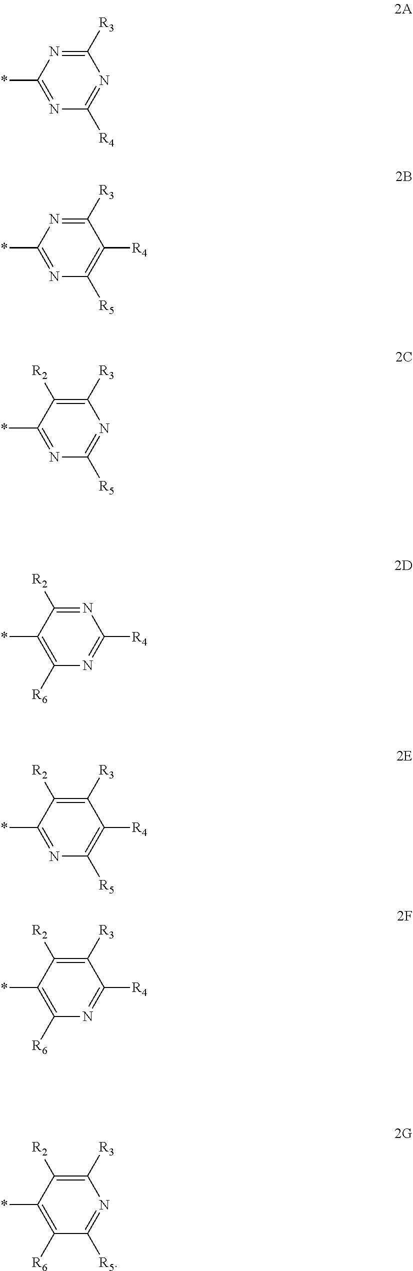

For example, Ar^{EWG} may be Formula 2A.

In one or more embodiments, at least two of X₂ to X₆ may be N.

R₁ to R₆ may each independently be a group represented by Formula 3, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
at least one selected from R₁ to R₆ may be Ar^{EDG}, Ar^{EDG} may be a group represented by Formula 3, and R₁₀ₐ and Q₁ to Q₃ may be as described herein.

In one or more embodiments, R₁ may be Ar^{E}DG. For example, Ar^{EDG} may be a heterocyclic compound represented by one selected from Formulae 3A to 3E:

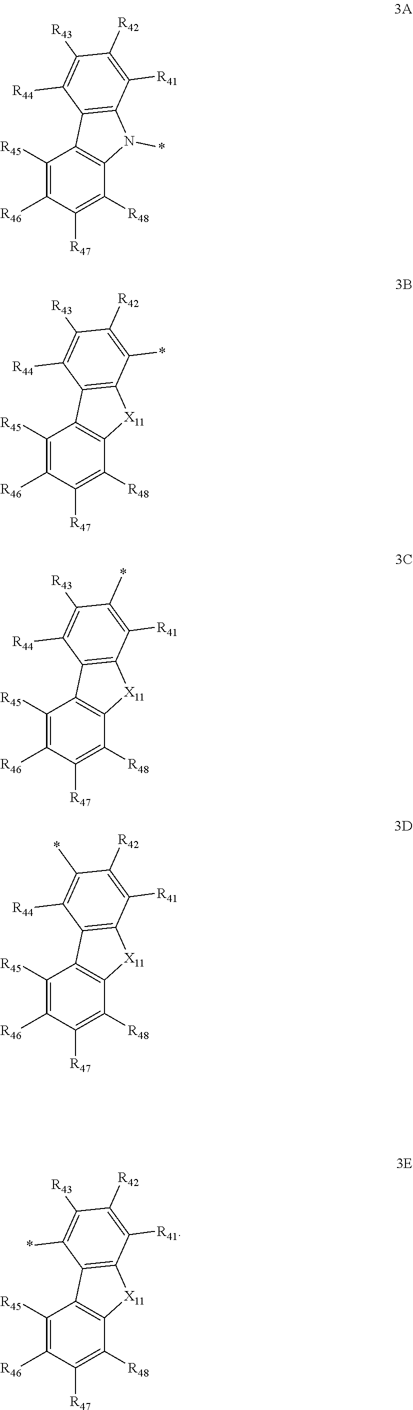

X₁₁ and R₄₁ to R₄₈ in Formulae 3A to 3E may be as described herein, and * may be a binding site to Formula 1.

In one or more embodiments, R₂ to R₆ may each independently be hydrogen, deuterium, —F, —Cl, —Br, —I, a cyano group, a nitro group, a C₁-C₂₀ alkyl group unsubstituted or substituted with at least one R₁₀ₐ, a C₂-C₂₀ alkenyl group unsubstituted or substituted with at least one R₁₀ₐ, a C₂-C₂₀ alkynyl group unsubstituted or substituted with at least one R₁₀ₐ, —C(═O)(Q₁), —S(O)₂(Q₁), —P(═O)(Q₁)(Q₂), a C₃-C₂₀ cycloalkyl group unsubstituted or substituted with at least one R₁₀ₐ, a C₃-C₂₀ cycloalkenyl group unsubstituted or substituted with at least one R₁₀ₐ, a C₃-C₂₀ cycloalkynyl group unsubstituted or substituted with at least one R₁₀ₐ, a C₆-C₂₀ aryl group unsubstituted or substituted with at least one R₁₀ₐ, or a C₁-C₂₀ heteroaryl group unsubstituted or substituted with at least one R₁₀ₐ, and R₁₀ₐ, Q₁, and Q₂ are as described herein.

In one or more embodiments, R₂ to R₆ may each independently be Ar^{EDG}; or a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, or a chrysenyl group, each unsubstituted or substituted with at least one R₁₀ₐ, and R₁₀ₐ may be as described herein.

In one or more embodiments, R₂ to R₆ may each independently be a phenyl group unsubstituted or substituted with deuterium or a carbazolyl group unsubstituted or substituted with deuterium.

L₁ and L₂ may each independently be a C₃-C₆₀ carbocyclic group or a C₁-C₆₀ heterocyclic group, n1 and n2 may each independently be an integer from 1 to 5, i) when n1 is an integer of 2 or more, a plurality of L₁(s) may be identical to or different from each other, and ii) when n2 is an integer of 2 or more, a plurality of L₂(s) may be identical to or different from each other.

In one or more embodiments, L₁ and L₂ may each independently be a benzene group, a naphthalene group, or a phenanthrene group.

In one or more embodiments, Formula 1 may be represented by one selected from Formula 1A to Formula 11, X₁, CY₁, R₃₀, b30, Ar^{EDG}, and Ar^{EWG} may be as described herein, R₁₁ to R₁₅ may be as described for R₁₀, and R₂₁ to R₂₅ may be as described for R₂₀:

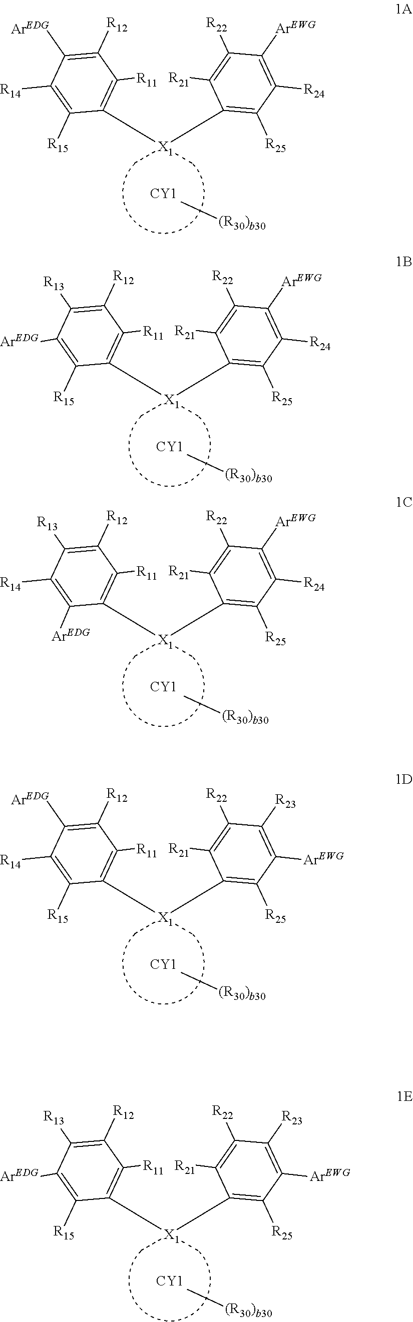

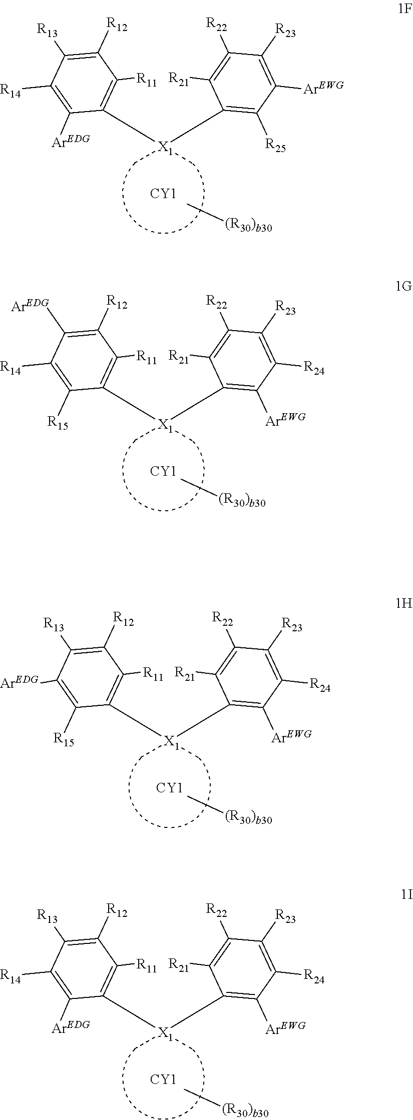

R₁₀, R₂₀, and R₃₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₁-C₆₀ arylthio group unsubstituted or substituted with at least one R₁₀ₐ, a C₇-C₆₀ arylalkyl group unsubstituted or substituted with at least one R₁₀ₐ, a substituted C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and b10, b20 and b30 may each independently be 0, 1, 2, 3, 4, 5, 6, 7, 8, 9, or 10.

In one or more embodiments, R₁₀ and R₂₀ may each independently be: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof; a C₃-C₆₀ carbocyclic group, a C₆-C₆₀ aryloxy group, a C₆-C₆₀ arylthio group, a C₇-C₆₀ arylalkyl group, or C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and Q₁₁ to Q₁₃, Q₂₁ to Q₂₃, and Q₃₁ to Q₃₃ are as described herein. For example, R₁₀ and R₂₀ may not each independently be a pyridinyl group, a pyrimidinyl group, a pyrazinyl group, a pyridazinyl group, and a carbazolyl group.

X₁₁ may be 0, S, Se, N(R₄₉), or P(R₄₉).

In one or more embodiments, X₁₁ may be O, S, or N(R₁).

R₄₁ to R₄₉ may each independently be a binding site to a neighboring atom, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and any one selected from R₄₁ to R₄₉ may be a binding site to a neighboring atom.

In one or more embodiments, R₄₁ to R₄₈ may each independently be a binding site to a neighboring atom, hydrogen, deuterium, a C₁-C₂₀ alkyl group unsubstituted or substituted with at least one R₁₀ₐ, a C₂-C₂₀ alkenyl group unsubstituted or substituted with at least one R₁₀ₐ, a C₂-C₂₀ alkynyl group unsubstituted or substituted with at least one R₁₀ₐ, a hydroxyl group, a C₁-C₆₀ alkoxy group unsubstituted or substituted with at least one R₁₀ₐ, —N(Q₁)(Q₂), a C₃-C₂₀ cycloalkyl group unsubstituted or substituted with at least one R₁₀ₐ, a C₃-C₂₀ cycloalkenyl group unsubstituted or substituted with at least one R₁₀ₐ, a C₃-C₂₀ cycloalkynyl group unsubstituted or substituted with at least one R₁₀ₐ, a C₆-C₂₀ aryl group unsubstituted or substituted with at least one R₁₀ₐ, or a C₁-C₂₀ heteroaryl group unsubstituted or substituted with at least one R₁₀ₐ, and R₁₀ₐ, Q₁, and Q₂ are as described herein.

In one or more embodiments, R₄₁ to R₄₈ may each independently be a binding site to a neighboring atom, hydrogen, or deuterium; or a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, an isoindolyl group, an indolyl group, an indazolyl group, a purinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or a combination thereof.

For example, R₄₁ to R₄₈ may each independently be a binding site to a neighboring atom, hydrogen, or deuterium; or a phenyl group unsubstituted or substituted with deuterium or a carbazolyl group unsubstituted or substituted with deuterium.

R₁₀ₐ may be deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

In one or more embodiments, the heterocyclic compound may be one selected from Compounds 1 to 165:

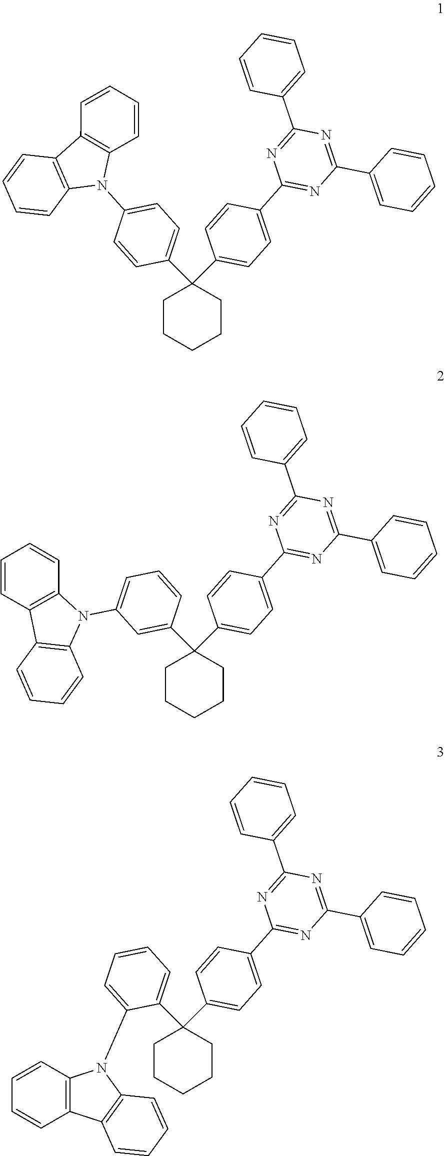 [SMILES: c1ccc(-c2nc(-c3ccccc3)nc(-c3ccc(C4(c5ccc(-n6c7ccccc7c7ccccc76)cc5)CCCCC4)cc3)n2)cc1 ; c1ccc(-c2nc(-c3ccccc3)nc(-c3ccc(C4(c5cccc(-n6c7ccccc7c7ccccc76)c5)CCCCC4)cc3)n2)cc1 ; c1ccc(-c2nc(-c3ccccc3)nc(-c3ccc(C4(c5ccccc5-n5c6ccccc6c6ccccc65)CCCCC4)cc3)n2)cc1]

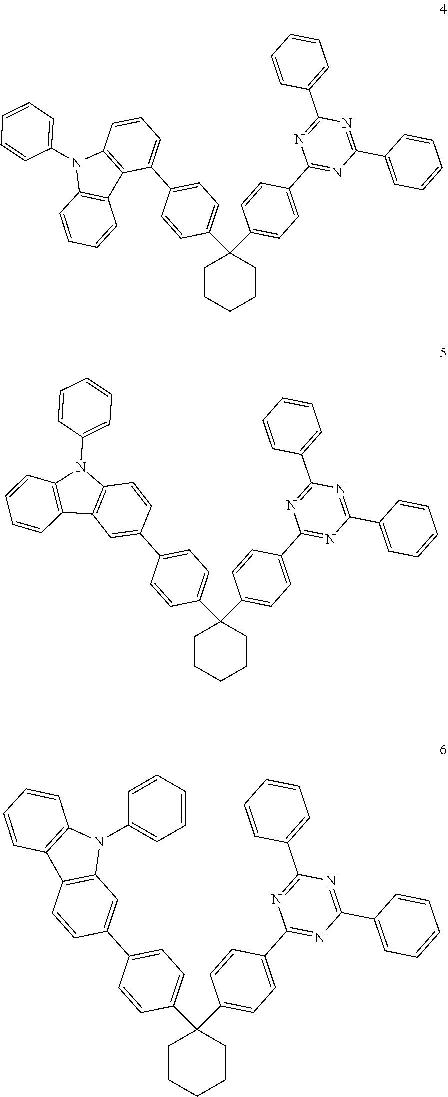 [SMILES: c1ccc(-c2nc(-c3ccccc3)nc(-c3ccc(C4(c5ccc(-c6cccc7c6c6ccccc6n7-c6ccccc6)cc5)CCCCC4)cc3)n2)cc1 ; c1ccc(-c2nc(-c3ccccc3)nc(-c3ccc(C4(c5ccc(-c6ccc7c(c6)c6ccccc6n7-c6ccccc6)cc5)CCCCC4)cc3)n2)cc1 ; c1ccc(-c2nc(-c3ccccc3)nc(-c3ccc(C4(c5ccc(-c6ccc7c8ccccc8n(-c8ccccc8)c7c6)cc5)CCCCC4)cc3)n2)cc1]

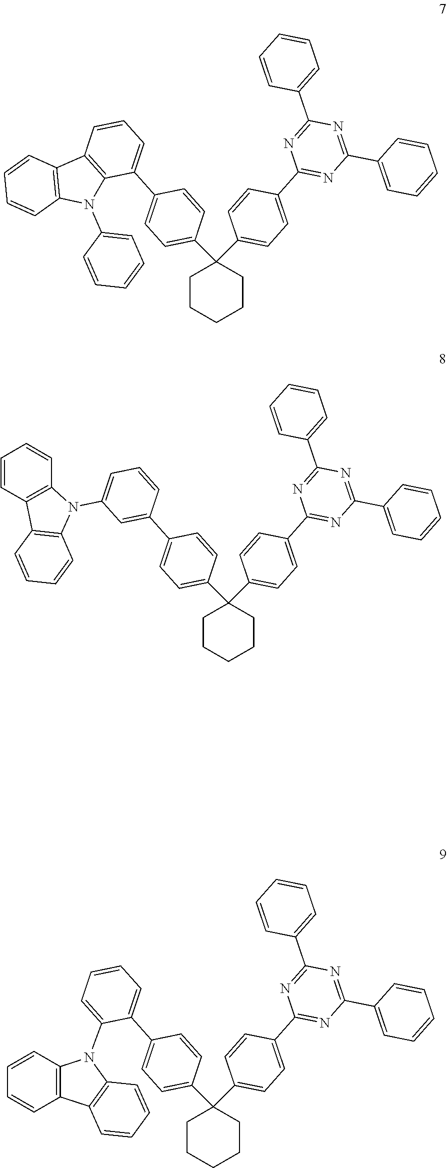 [SMILES: c1ccc(-c2nc(-c3ccccc3)nc(-c3ccc(C4(c5ccc(-c6cccc7c8ccccc8n(-c8ccccc8)c67)cc5)CCCCC4)cc3)n2)cc1 ; c1ccc(-c2nc(-c3ccccc3)nc(-c3ccc(C4(c5ccc(-c6cccc(-n7c8ccccc8c8ccccc87)c6)cc5)CCCCC4)cc3)n2)cc1 ; c1ccc(-c2nc(-c3ccccc3)nc(-c3ccc(C4(c5ccc(-c6ccccc6-n6c7ccccc7c7ccccc76)cc5)CCCCC4)cc3)n2)cc1]

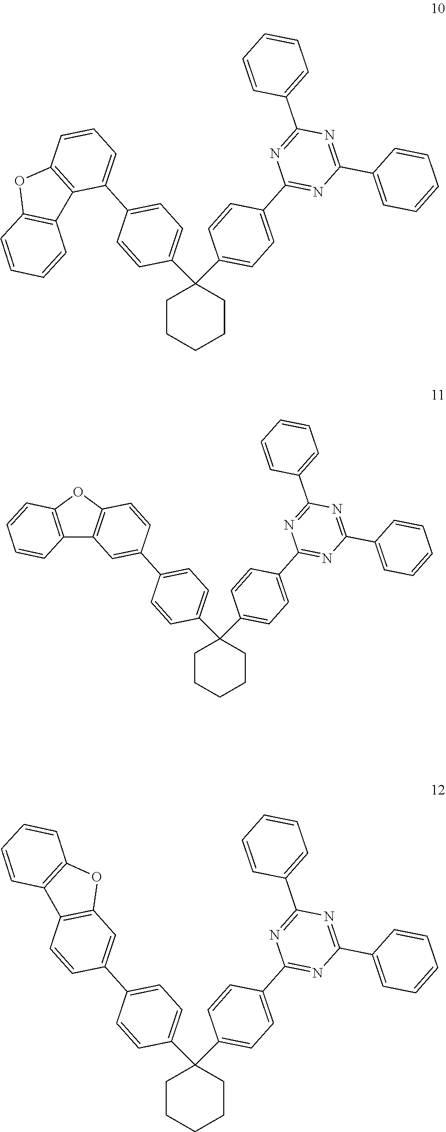 [SMILES: c1ccc(-c2nc(-c3ccccc3)nc(-c3ccc(C4(c5ccc(-c6cccc7oc8ccccc8c67)cc5)CCCCC4)cc3)n2)cc1 ; c1ccc(-c2nc(-c3ccccc3)nc(-c3ccc(C4(c5ccc(-c6ccc7oc8ccccc8c7c6)cc5)CCCCC4)cc3)n2)cc1 ; c1ccc(-c2nc(-c3ccccc3)nc(-c3ccc(C4(c5ccc(-c6ccc7c(c6)oc6ccccc67)cc5)CCCCC4)cc3)n2)cc1]

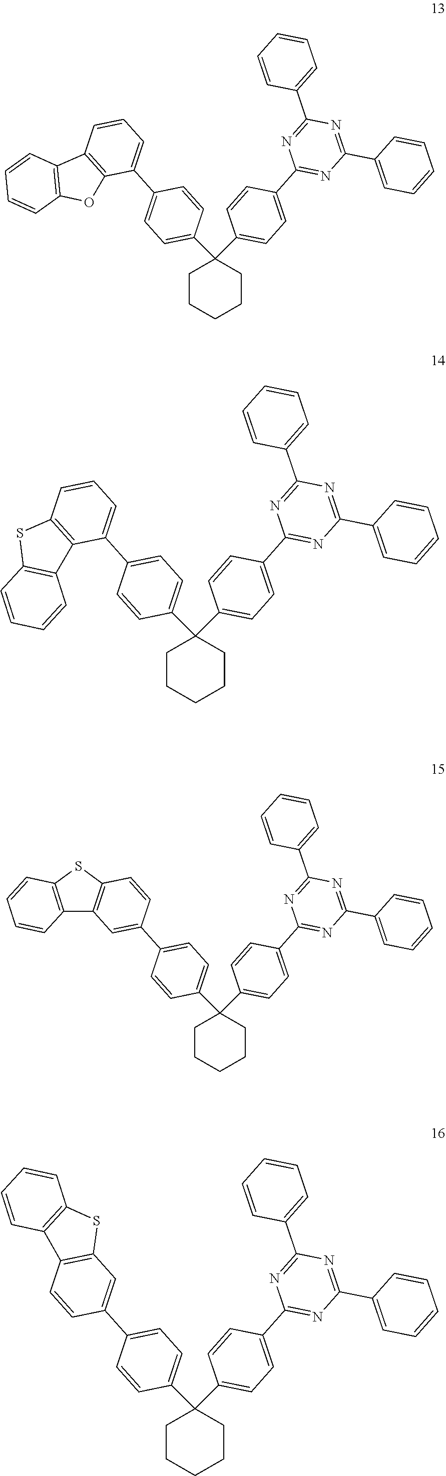 [SMILES: c1ccc(-c2nc(-c3ccccc3)nc(-c3ccc(C4(c5ccc(-c6cccc7c6oc6ccccc67)cc5)CCCCC4)cc3)n2)cc1 ; c1ccc(-c2nc(-c3ccccc3)nc(-c3ccc(C4(c5ccc(-c6cccc7sc8ccccc8c67)cc5)CCCCC4)cc3)n2)cc1 ; c1ccc(-c2nc(-c3ccccc3)nc(-c3ccc(C4(c5ccc(-c6ccc7sc8ccccc8c7c6)cc5)CCCCC4)cc3)n2)cc1 ; c1ccc(-c2nc(-c3ccccc3)nc(-c3ccc(C4(c5ccc(-c6ccc7c(c6)sc6ccccc67)cc5)CCCCC4)cc3)n2)cc1]

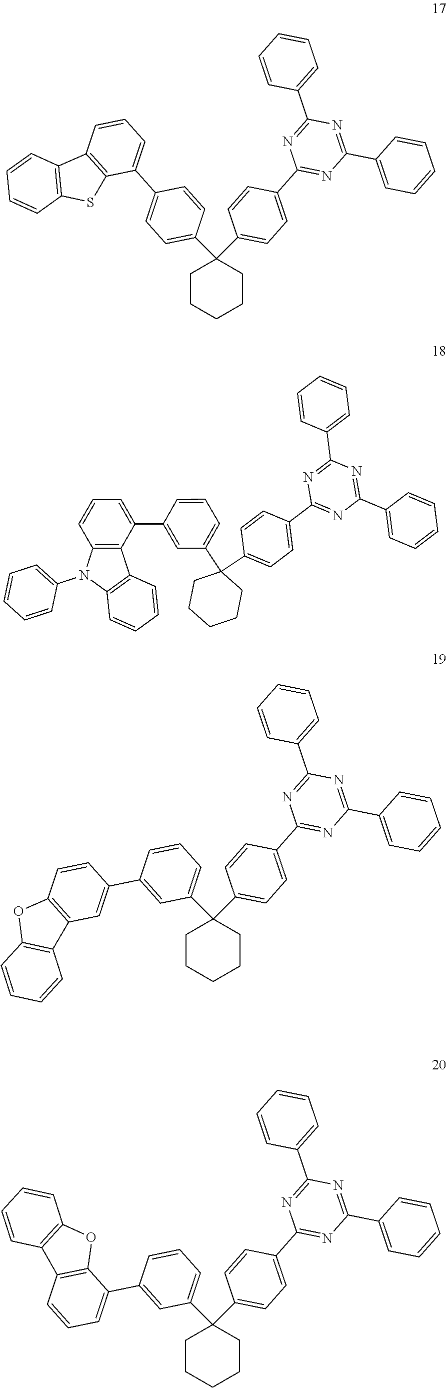 [SMILES: c1ccc(-c2nc(-c3ccccc3)nc(-c3ccc(C4(c5ccc(-c6cccc7c6sc6ccccc67)cc5)CCCCC4)cc3)n2)cc1 ; c1ccc(-c2nc(-c3ccccc3)nc(-c3ccc(C4(c5cccc(-c6cccc7c6c6ccccc6n7-c6ccccc6)c5)CCCCC4)cc3)n2)cc1 ; c1ccc(-c2nc(-c3ccccc3)nc(-c3ccc(C4(c5cccc(-c6ccc7oc8ccccc8c7c6)c5)CCCCC4)cc3)n2)cc1 ; c1ccc(-c2nc(-c3ccccc3)nc(-c3ccc(C4(c5cccc(-c6cccc7c6oc6ccccc67)c5)CCCCC4)cc3)n2)cc1]

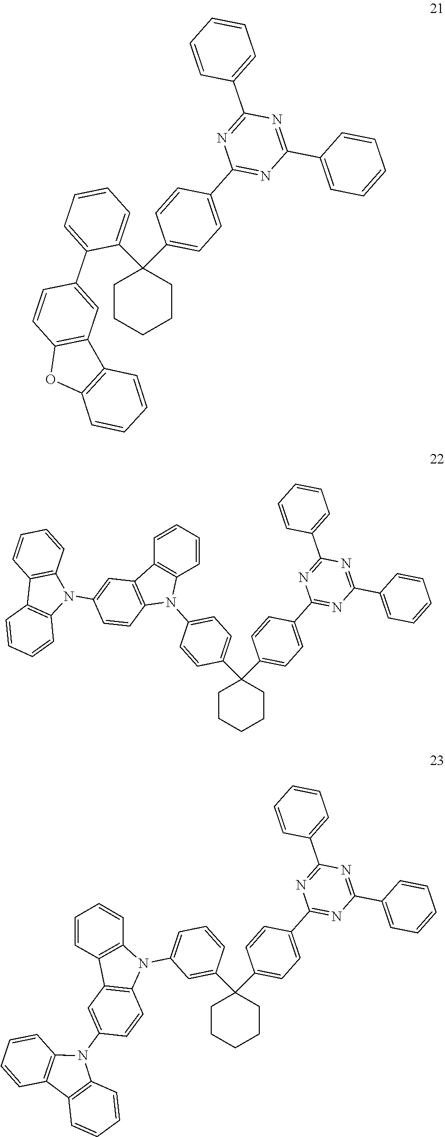 [SMILES: c1ccc(-c2nc(-c3ccccc3)nc(-c3ccc(C4(c5ccccc5-c5ccc6oc7ccccc7c6c5)CCCCC4)cc3)n2)cc1 ; c1ccc(-c2nc(-c3ccccc3)nc(-c3ccc(C4(c5ccc(-n6c7ccccc7c7cc(-n8c9ccccc9c9ccccc98)ccc76)cc5)CCCCC4)cc3)n2)cc1 ; c1ccc(-c2nc(-c3ccccc3)nc(-c3ccc(C4(c5cccc(-n6c7ccccc7c7cc(-n8c9ccccc9c9ccccc98)ccc76)c5)CCCCC4)cc3)n2)cc1]

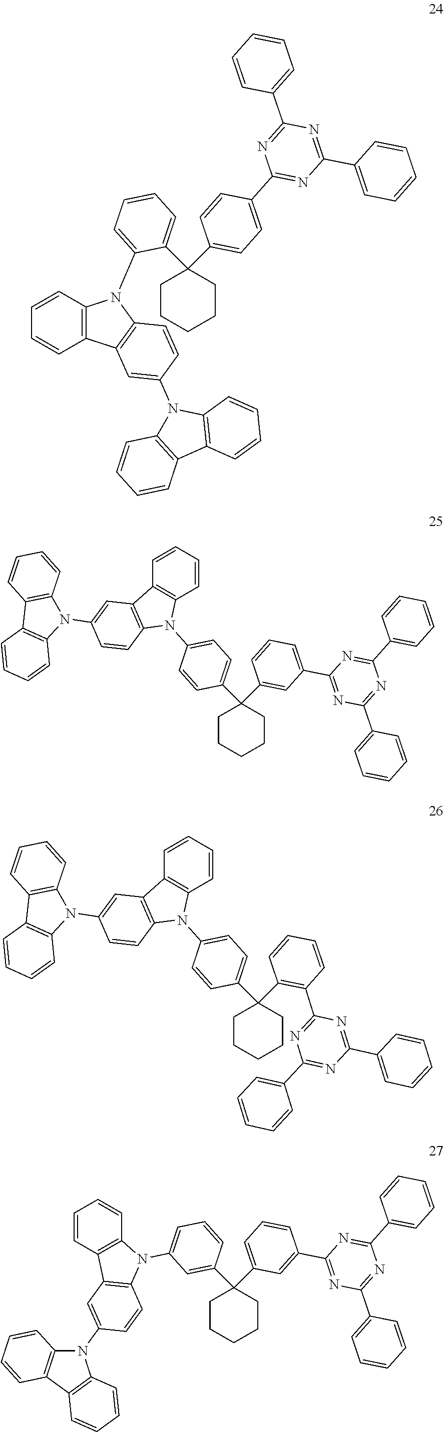 [SMILES: c1ccc(-c2nc(-c3ccccc3)nc(-c3ccc(C4(c5ccccc5-n5c6ccccc6c6cc(-n7c8ccccc8c8ccccc87)ccc65)CCCCC4)cc3)n2)cc1 ; c1ccc(-c2nc(-c3ccccc3)nc(-c3cccc(C4(c5ccc(-n6c7ccccc7c7cc(-n8c9ccccc9c9ccccc98)ccc76)cc5)CCCCC4)c3)n2)cc1 ; c1ccc(-c2nc(-c3ccccc3)nc(-c3ccccc3C3(c4ccc(-n5c6ccccc6c6cc(-n7c8ccccc8c8ccccc87)ccc65)cc4)CCCCC3)n2)cc1 ; c1ccc(-c2nc(-c3ccccc3)nc(-c3cccc(C4(c5cccc(-n6c7ccccc7c7cc(-n8c9ccccc9c9ccccc98)ccc76)c5)CCCCC4)c3)n2)cc1]

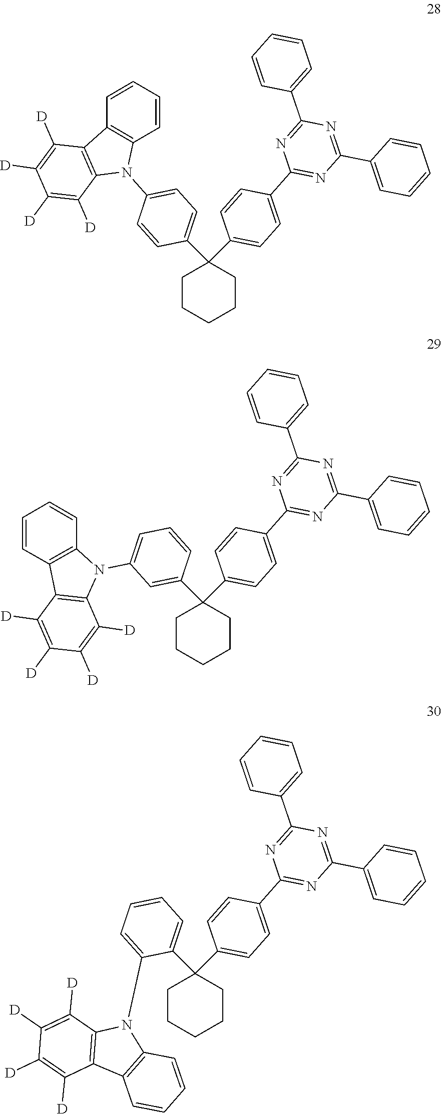 [SMILES: [2H]c1c([2H])c([2H])c2c(c1[2H])c1ccccc1n2-c1ccc(C2(c3ccc(-c4nc(-c5ccccc5)nc(-c5ccccc5)n4)cc3)CCCCC2)cc1 ; [2H]c1c([2H])c([2H])c2c(c1[2H])c1ccccc1n2-c1cccc(C2(c3ccc(-c4nc(-c5ccccc5)nc(-c5ccccc5)n4)cc3)CCCCC2)c1 ; [2H]c1c([2H])c([2H])c2c(c1[2H])c1ccccc1n2-c1ccccc1C1(c2ccc(-c3nc(-c4ccccc4)nc(-c4ccccc4)n3)cc2)CCCCC1]

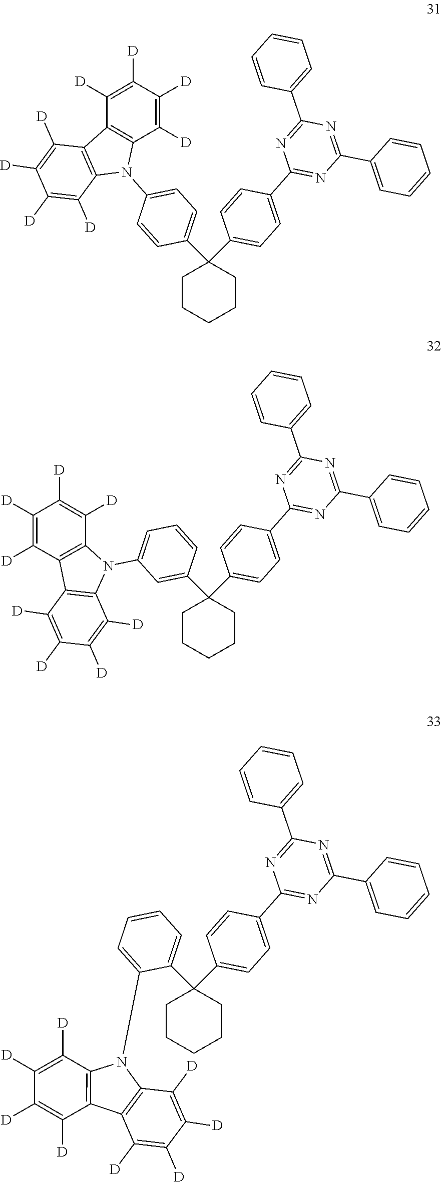 [SMILES: [2H]c1c([2H])c([2H])c2c(c1[2H])c1c([2H])c([2H])c([2H])c([2H])c1n2-c1ccc(C2(c3ccc(-c4nc(-c5ccccc5)nc(-c5ccccc5)n4)cc3)CCCCC2)cc1 ; [2H]c1c([2H])c([2H])c2c(c1[2H])c1c([2H])c([2H])c([2H])c([2H])c1n2-c1cccc(C2(c3ccc(-c4nc(-c5ccccc5)nc(-c5ccccc5)n4)cc3)CCCCC2)c1 ; [2H]c1c([2H])c([2H])c2c(c1[2H])c1c([2H])c([2H])c([2H])c([2H])c1n2-c1ccccc1C1(c2ccc(-c3nc(-c4ccccc4)nc(-c4ccccc4)n3)cc2)CCCCC1]

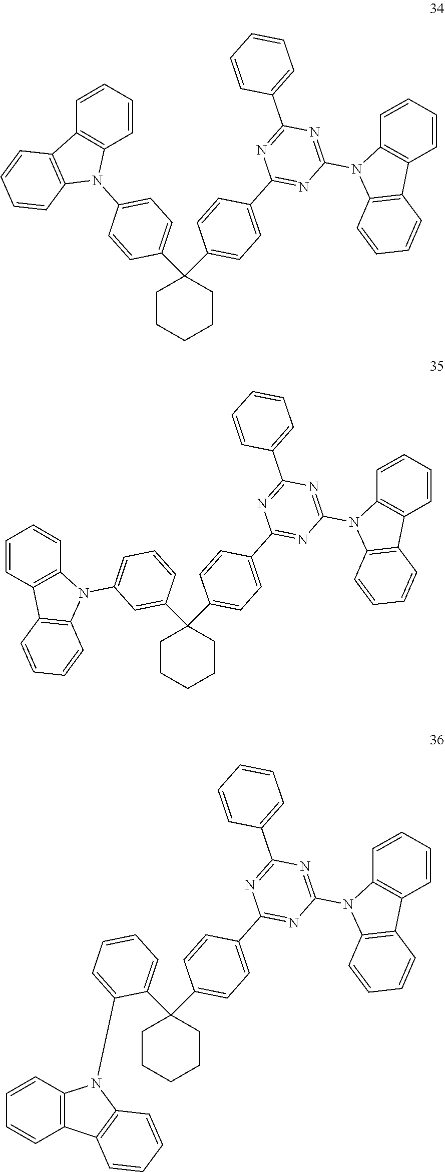 [SMILES: c1ccc(-c2nc(-c3ccc(C4(c5ccc(-n6c7ccccc7c7ccccc76)cc5)CCCCC4)cc3)nc(-n3c4ccccc4c4ccccc43)n2)cc1 ; c1ccc(-c2nc(-c3ccc(C4(c5cccc(-n6c7ccccc7c7ccccc76)c5)CCCCC4)cc3)nc(-n3c4ccccc4c4ccccc43)n2)cc1 ; c1ccc(-c2nc(-c3ccc(C4(c5ccccc5-n5c6ccccc6c6ccccc65)CCCCC4)cc3)nc(-n3c4ccccc4c4ccccc43)n2)cc1]

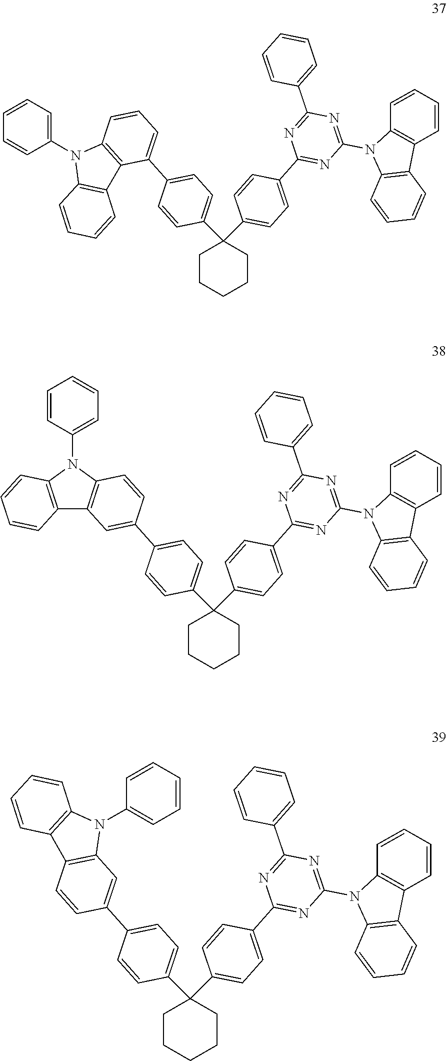 [SMILES: c1ccc(-c2nc(-c3ccc(C4(c5ccc(-c6cccc7c6c6ccccc6n7-c6ccccc6)cc5)CCCCC4)cc3)nc(-n3c4ccccc4c4ccccc43)n2)cc1 ; c1ccc(-c2nc(-c3ccc(C4(c5ccc(-c6ccc7c(c6)c6ccccc6n7-c6ccccc6)cc5)CCCCC4)cc3)nc(-n3c4ccccc4c4ccccc43)n2)cc1 ; c1ccc(-c2nc(-c3ccc(C4(c5ccc(-c6ccc7c8ccccc8n(-c8ccccc8)c7c6)cc5)CCCCC4)cc3)nc(-n3c4ccccc4c4ccccc43)n2)cc1]

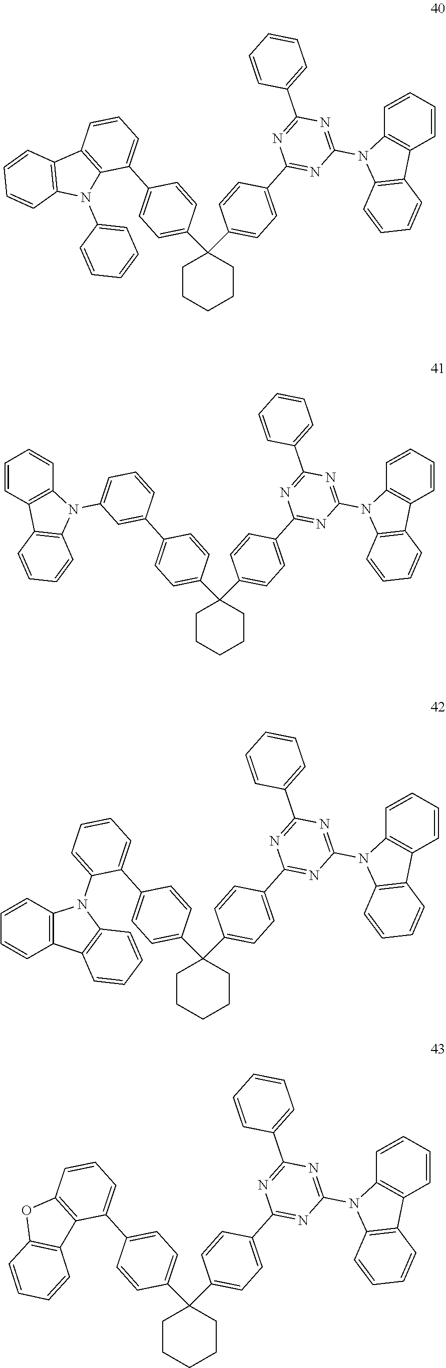 [SMILES: c1ccc(-c2nc(-c3ccc(C4(c5ccc(-c6cccc7c8ccccc8n(-c8ccccc8)c67)cc5)CCCCC4)cc3)nc(-n3c4ccccc4c4ccccc43)n2)cc1 ; c1ccc(-c2nc(-c3ccc(C4(c5ccc(-c6cccc(-n7c8ccccc8c8ccccc87)c6)cc5)CCCCC4)cc3)nc(-n3c4ccccc4c4ccccc43)n2)cc1 ; c1ccc(-c2nc(-c3ccc(C4(c5ccc(-c6ccccc6-n6c7ccccc7c7ccccc76)cc5)CCCCC4)cc3)nc(-n3c4ccccc4c4ccccc43)n2)cc1 ; c1ccc(-c2nc(-c3ccc(C4(c5ccc(-c6cccc7oc8ccccc8c67)cc5)CCCCC4)cc3)nc(-n3c4ccccc4c4ccccc43)n2)cc1]

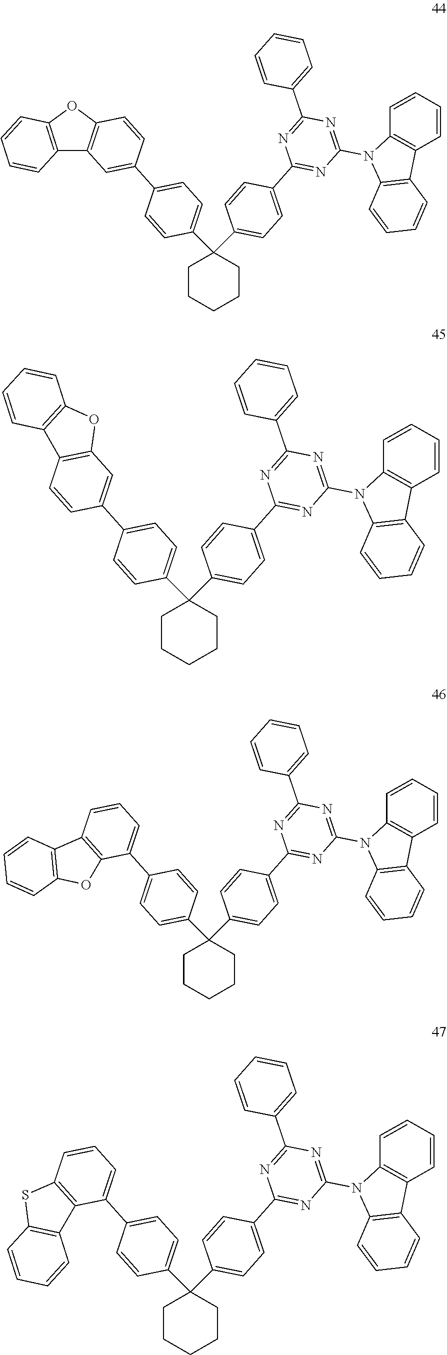 [SMILES: c1ccc(-c2nc(-c3ccc(C4(c5ccc(-c6ccc7oc8ccccc8c7c6)cc5)CCCCC4)cc3)nc(-n3c4ccccc4c4ccccc43)n2)cc1 ; c1ccc(-c2nc(-c3ccc(C4(c5ccc(-c6ccc7c(c6)oc6ccccc67)cc5)CCCCC4)cc3)nc(-n3c4ccccc4c4ccccc43)n2)cc1 ; c1ccc(-c2nc(-c3ccc(C4(c5ccc(-c6cccc7c6oc6ccccc67)cc5)CCCCC4)cc3)nc(-n3c4ccccc4c4ccccc43)n2)cc1 ; c1ccc(-c2nc(-c3ccc(C4(c5ccc(-c6cccc7sc8ccccc8c67)cc5)CCCCC4)cc3)nc(-n3c4ccccc4c4ccccc43)n2)cc1]

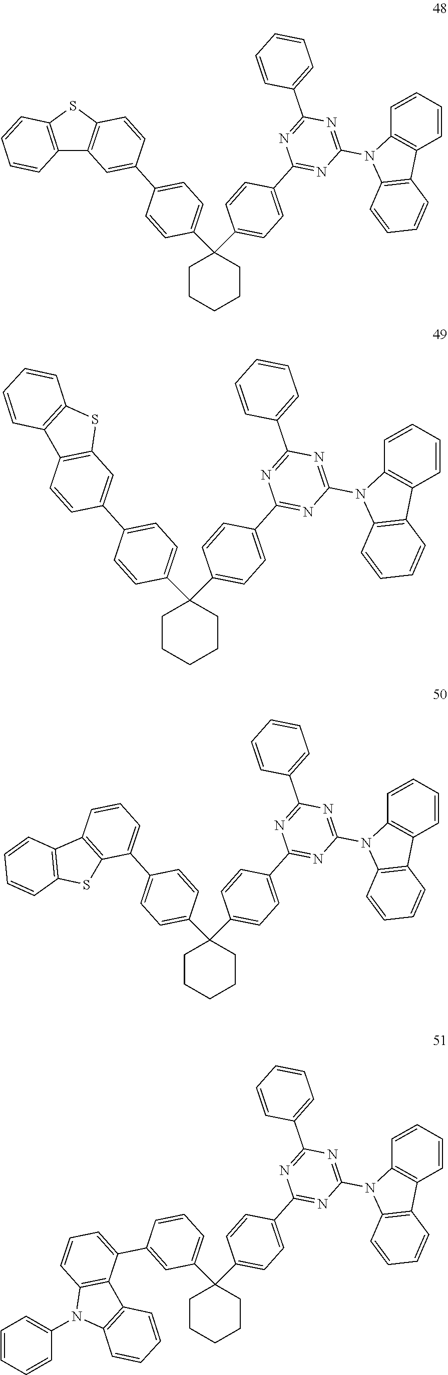 [SMILES: c1ccc(-c2nc(-c3ccc(C4(c5ccc(-c6ccc7sc8ccccc8c7c6)cc5)CCCCC4)cc3)nc(-n3c4ccccc4c4ccccc43)n2)cc1 ; c1ccc(-c2nc(-c3ccc(C4(c5ccc(-c6ccc7c(c6)sc6ccccc67)cc5)CCCCC4)cc3)nc(-n3c4ccccc4c4ccccc43)n2)cc1 ; c1ccc(-c2nc(-c3ccc(C4(c5ccc(-c6cccc7c6sc6ccccc67)cc5)CCCCC4)cc3)nc(-n3c4ccccc4c4ccccc43)n2)cc1 ; c1ccc(-c2nc(-c3ccc(C4(c5cccc(-c6cccc7c6c6ccccc6n7-c6ccccc6)c5)CCCCC4)cc3)nc(-n3c4ccccc4c4ccccc43)n2)cc1]

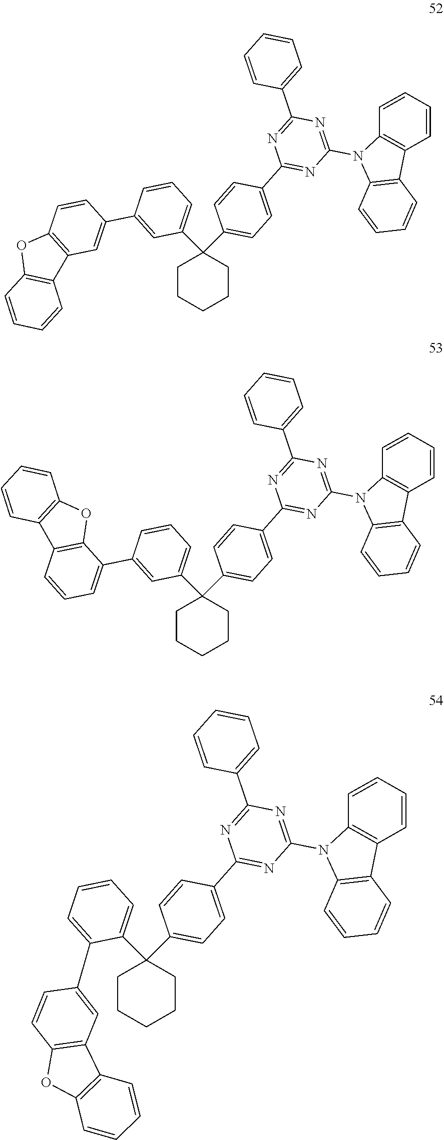 [SMILES: c1ccc(-c2nc(-c3ccc(C4(c5cccc(-c6ccc7oc8ccccc8c7c6)c5)CCCCC4)cc3)nc(-n3c4ccccc4c4ccccc43)n2)cc1 ; c1ccc(-c2nc(-c3ccc(C4(c5cccc(-c6cccc7c6oc6ccccc67)c5)CCCCC4)cc3)nc(-n3c4ccccc4c4ccccc43)n2)cc1 ; c1ccc(-c2nc(-c3ccc(C4(c5ccccc5-c5ccc6oc7ccccc7c6c5)CCCCC4)cc3)nc(-n3c4ccccc4c4ccccc43)n2)cc1]

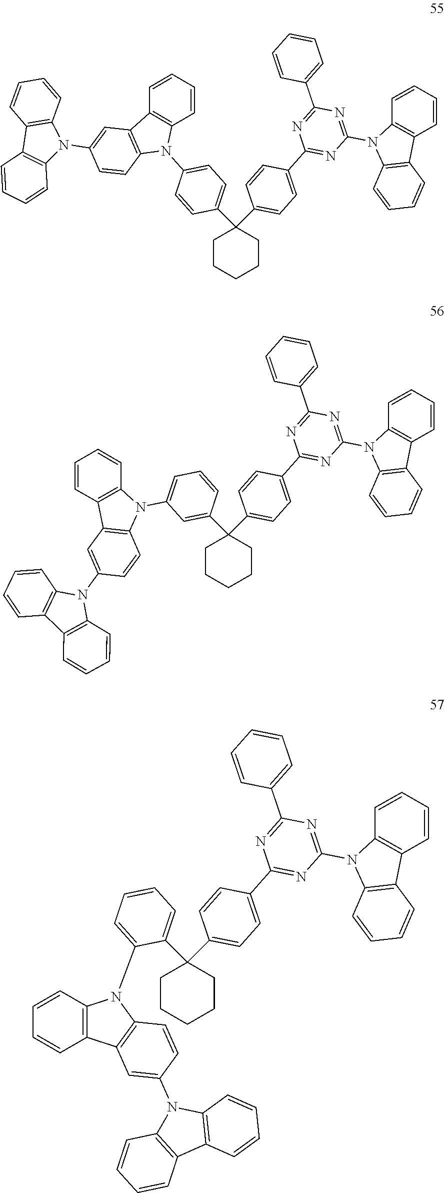 [SMILES: c1ccc(-c2nc(-c3ccc(C4(c5ccc(-n6c7ccccc7c7cc(-n8c9ccccc9c9ccccc98)ccc76)cc5)CCCCC4)cc3)nc(-n3c4ccccc4c4ccccc43)n2)cc1 ; c1ccc(-c2nc(-c3ccc(C4(c5cccc(-n6c7ccccc7c7cc(-n8c9ccccc9c9ccccc98)ccc76)c5)CCCCC4)cc3)nc(-n3c4ccccc4c4ccccc43)n2)cc1 ; c1ccc(-c2nc(-c3ccc(C4(c5ccccc5-n5c6ccccc6c6cc(-n7c8ccccc8c8ccccc87)ccc65)CCCCC4)cc3)nc(-n3c4ccccc4c4ccccc43)n2)cc1]

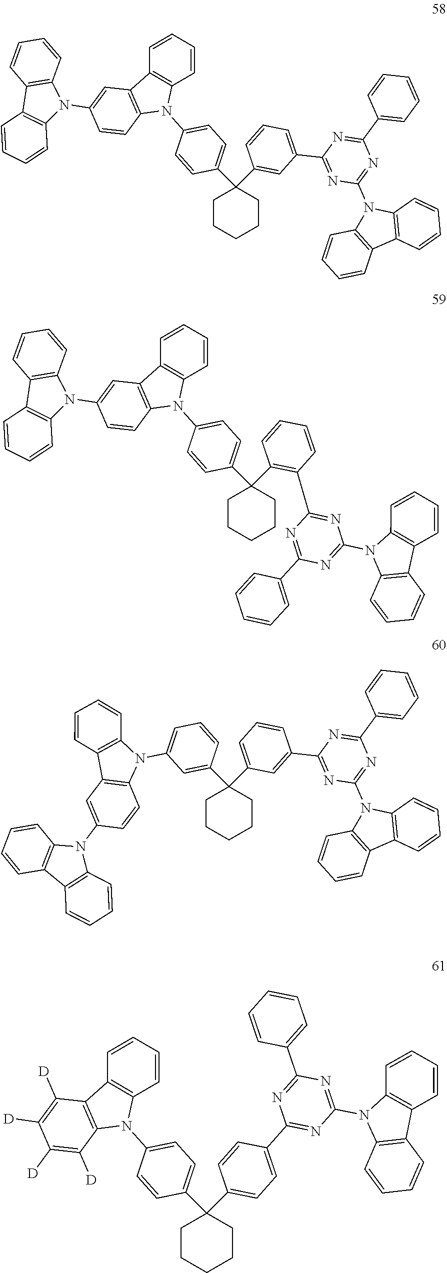 [SMILES: c1ccc(-c2nc(-c3cccc(C4(c5ccc(-n6c7ccccc7c7cc(-n8c9ccccc9c9ccccc98)ccc76)cc5)CCCCC4)c3)nc(-n3c4ccccc4c4ccccc43)n2)cc1 ; c1ccc(-c2nc(-c3ccccc3C3(c4ccc(-n5c6ccccc6c6cc(-n7c8ccccc8c8ccccc87)ccc65)cc4)CCCCC3)nc(-n3c4ccccc4c4ccccc43)n2)cc1 ; c1ccc(-c2nc(-c3cccc(C4(c5cccc(-n6c7ccccc7c7cc(-n8c9ccccc9c9ccccc98)ccc76)c5)CCCCC4)c3)nc(-n3c4ccccc4c4ccccc43)n2)cc1 ; [2H]c1c([2H])c([2H])c2c(c1[2H])c1ccccc1n2-c1ccc(C2(c3ccc(-c4nc(-c5ccccc5)nc(-n5c6ccccc6c6ccccc65)n4)cc3)CCCCC2)cc1]

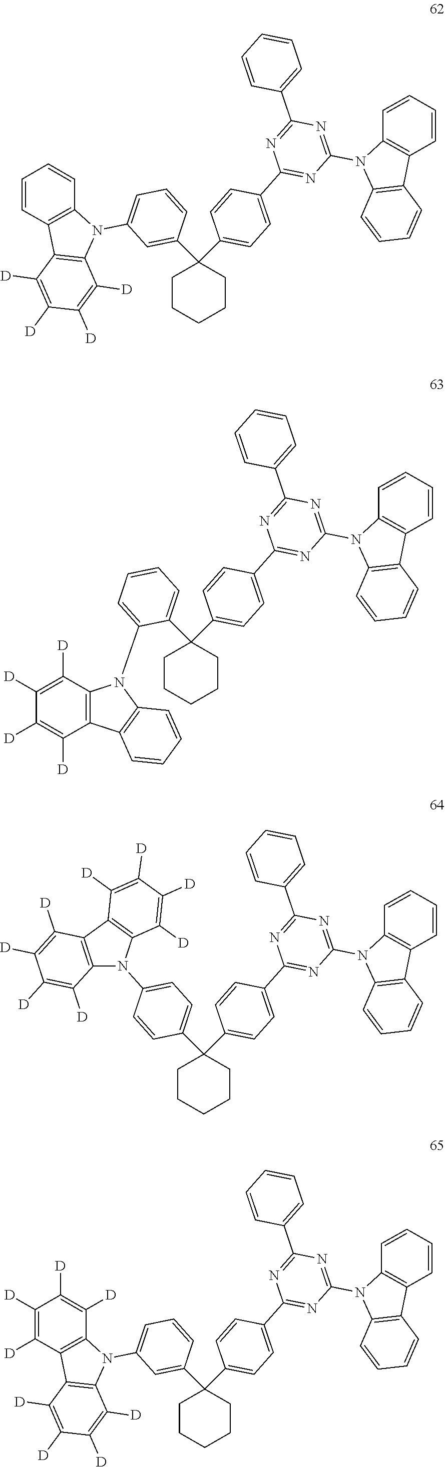

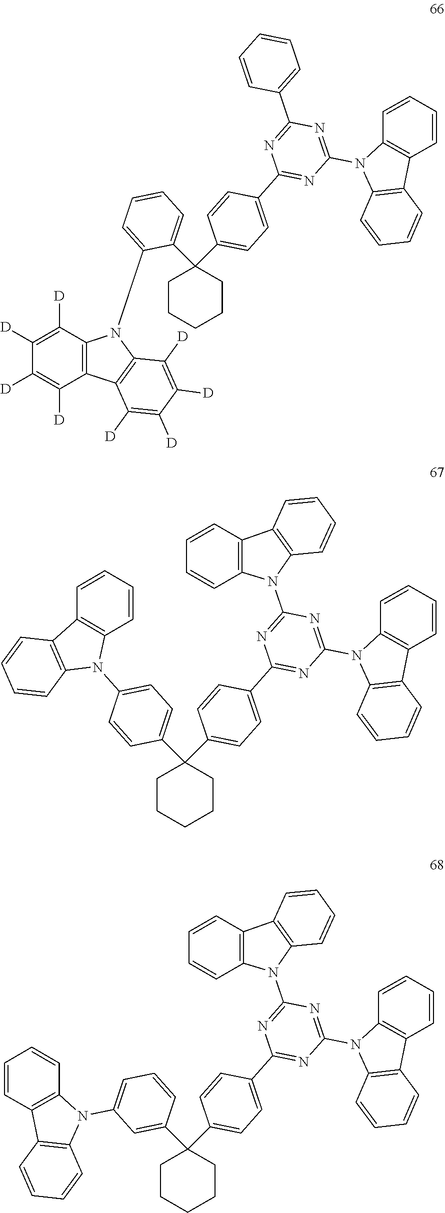 [SMILES: [2H]c1c([2H])c([2H])c2c(c1[2H])c1c([2H])c([2H])c([2H])c([2H])c1n2-c1ccccc1C1(c2ccc(-c3nc(-c4ccccc4)nc(-n4c5ccccc5c5ccccc54)n3)cc2)CCCCC1 ; c1ccc2c(c1)c1ccccc1n2-c1ccc(C2(c3ccc(-c4nc(-n5c6ccccc6c6ccccc65)nc(-n5c6ccccc6c6ccccc65)n4)cc3)CCCCC2)cc1 ; c1cc(-n2c3ccccc3c3ccccc32)cc(C2(c3ccc(-c4nc(-n5c6ccccc6c6ccccc65)nc(-n5c6ccccc6c6ccccc65)n4)cc3)CCCCC2)c1]

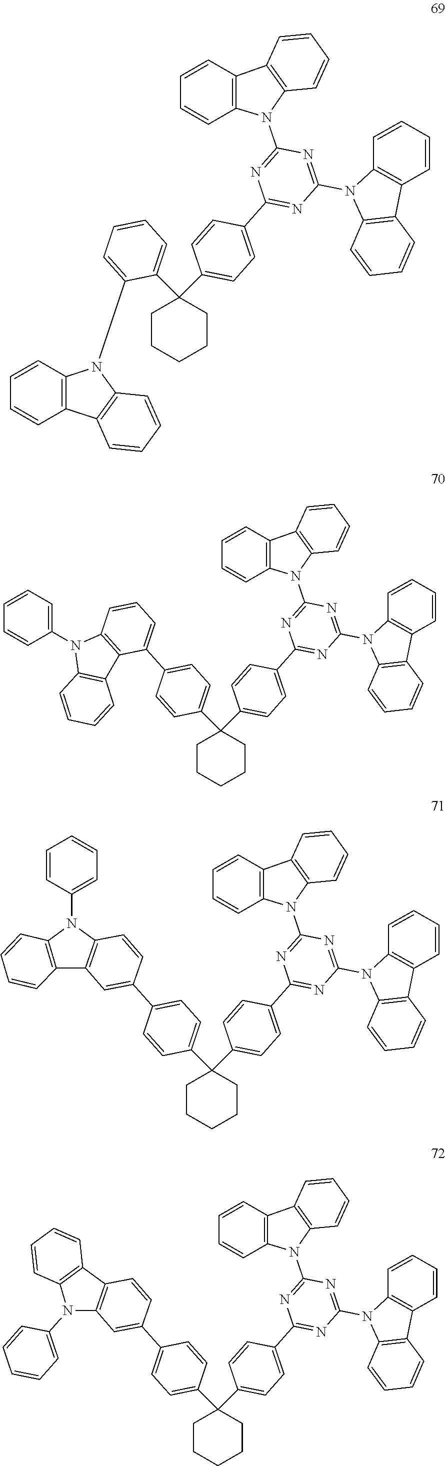 [SMILES: c1ccc(C2(c3ccc(-c4nc(-n5c6ccccc6c6ccccc65)nc(-n5c6ccccc6c6ccccc65)n4)cc3)CCCCC2)c(-n2c3ccccc3c3ccccc32)c1 ; c1ccc(-n2c3ccccc3c3c(-c4ccc(C5(c6ccc(-c7nc(-n8c9ccccc9c9ccccc98)nc(-n8c9ccccc9c9ccccc98)n7)cc6)CCCCC5)cc4)cccc32)cc1 ; c1ccc(-n2c3ccccc3c3cc(-c4ccc(C5(c6ccc(-c7nc(-n8c9ccccc9c9ccccc98)nc(-n8c9ccccc9c9ccccc98)n7)cc6)CCCCC5)cc4)ccc32)cc1 ; c1ccc(-n2c3ccccc3c3ccc(-c4ccc(C5(c6ccc(-c7nc(-n8c9ccccc9c9ccccc98)nc(-n8c9ccccc9c9ccccc98)n7)cc6)CCCCC5)cc4)cc32)cc1]

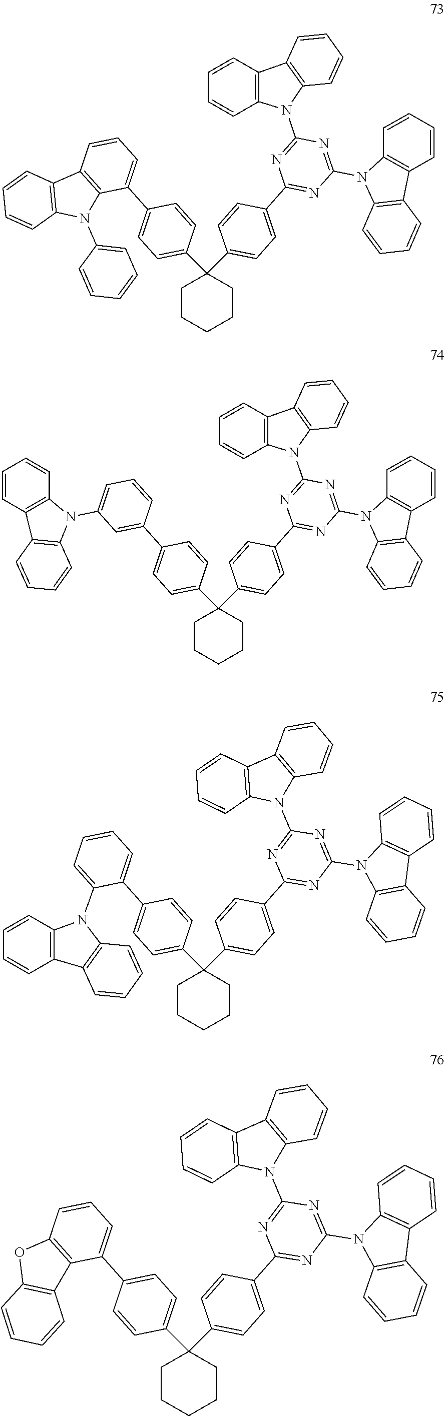 [SMILES: c1ccc(-n2c3ccccc3c3cccc(-c4ccc(C5(c6ccc(-c7nc(-n8c9ccccc9c9ccccc98)nc(-n8c9ccccc9c9ccccc98)n7)cc6)CCCCC5)cc4)c32)cc1 ; c1cc(-c2ccc(C3(c4ccc(-c5nc(-n6c7ccccc7c7ccccc76)nc(-n6c7ccccc7c7ccccc76)n5)cc4)CCCCC3)cc2)cc(-n2c3ccccc3c3ccccc32)c1 ; c1ccc(-n2c3ccccc3c3ccccc32)c(-c2ccc(C3(c4ccc(-c5nc(-n6c7ccccc7c7ccccc76)nc(-n6c7ccccc7c7ccccc76)n5)cc4)CCCCC3)cc2)c1 ; c1ccc2c(c1)oc1cccc(-c3ccc(C4(c5ccc(-c6nc(-n7c8ccccc8c8ccccc87)nc(-n7c8ccccc8c8ccccc87)n6)cc5)CCCCC4)cc3)c12]

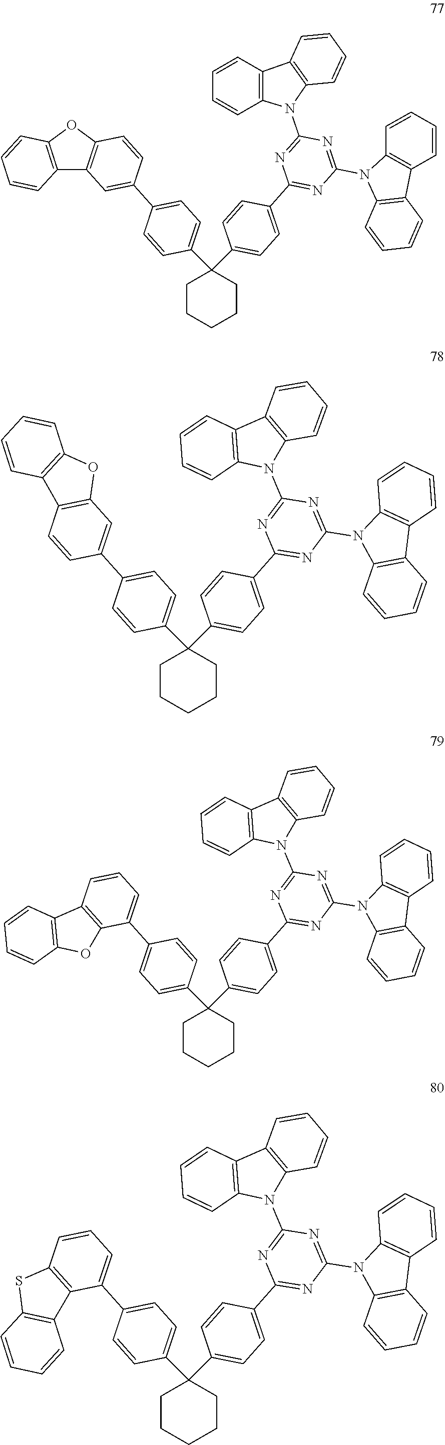 [SMILES: c1ccc2c(c1)oc1ccc(-c3ccc(C4(c5ccc(-c6nc(-n7c8ccccc8c8ccccc87)nc(-n7c8ccccc8c8ccccc87)n6)cc5)CCCCC4)cc3)cc12 ; c1ccc2c(c1)oc1cc(-c3ccc(C4(c5ccc(-c6nc(-n7c8ccccc8c8ccccc87)nc(-n7c8ccccc8c8ccccc87)n6)cc5)CCCCC4)cc3)ccc12 ; c1ccc2c(c1)oc1c(-c3ccc(C4(c5ccc(-c6nc(-n7c8ccccc8c8ccccc87)nc(-n7c8ccccc8c8ccccc87)n6)cc5)CCCCC4)cc3)cccc12 ; c1ccc2c(c1)sc1cccc(-c3ccc(C4(c5ccc(-c6nc(-n7c8ccccc8c8ccccc87)nc(-n7c8ccccc8c8ccccc87)n6)cc5)CCCCC4)cc3)c12]

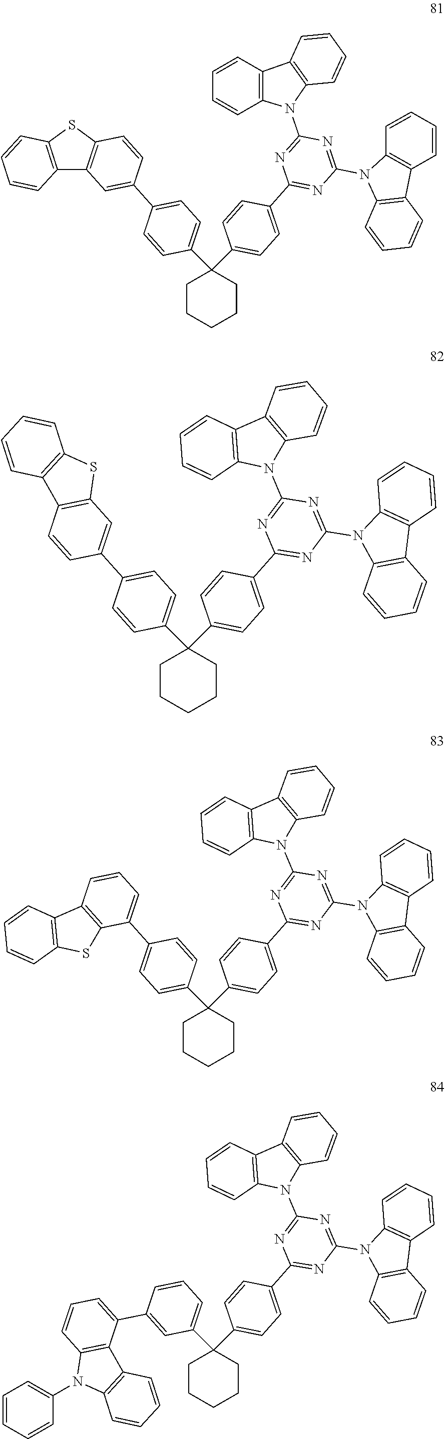 [SMILES: c1ccc2c(c1)sc1ccc(-c3ccc(C4(c5ccc(-c6nc(-n7c8ccccc8c8ccccc87)nc(-n7c8ccccc8c8ccccc87)n6)cc5)CCCCC4)cc3)cc12 ; c1ccc2c(c1)sc1cc(-c3ccc(C4(c5ccc(-c6nc(-n7c8ccccc8c8ccccc87)nc(-n7c8ccccc8c8ccccc87)n6)cc5)CCCCC4)cc3)ccc12 ; c1ccc2c(c1)sc1c(-c3ccc(C4(c5ccc(-c6nc(-n7c8ccccc8c8ccccc87)nc(-n7c8ccccc8c8ccccc87)n6)cc5)CCCCC4)cc3)cccc12 ; c1ccc(-n2c3ccccc3c3c(-c4cccc(C5(c6ccc(-c7nc(-n8c9ccccc9c9ccccc98)nc(-n8c9ccccc9c9ccccc98)n7)cc6)CCCCC5)c4)cccc32)cc1]

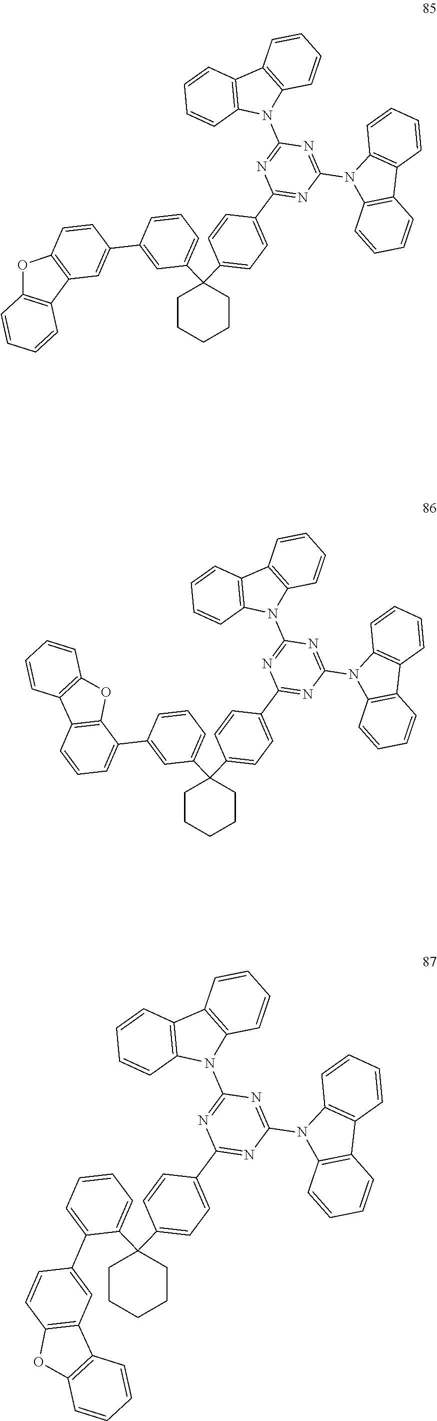 [SMILES: c1cc(-c2ccc3oc4ccccc4c3c2)cc(C2(c3ccc(-c4nc(-n5c6ccccc6c6ccccc65)nc(-n5c6ccccc6c6ccccc65)n4)cc3)CCCCC2)c1 ; c1cc(-c2cccc3c2oc2ccccc23)cc(C2(c3ccc(-c4nc(-n5c6ccccc6c6ccccc65)nc(-n5c6ccccc6c6ccccc65)n4)cc3)CCCCC2)c1 ; c1ccc(C2(c3ccc(-c4nc(-n5c6ccccc6c6ccccc65)nc(-n5c6ccccc6c6ccccc65)n4)cc3)CCCCC2)c(-c2ccc3oc4ccccc4c3c2)c1]

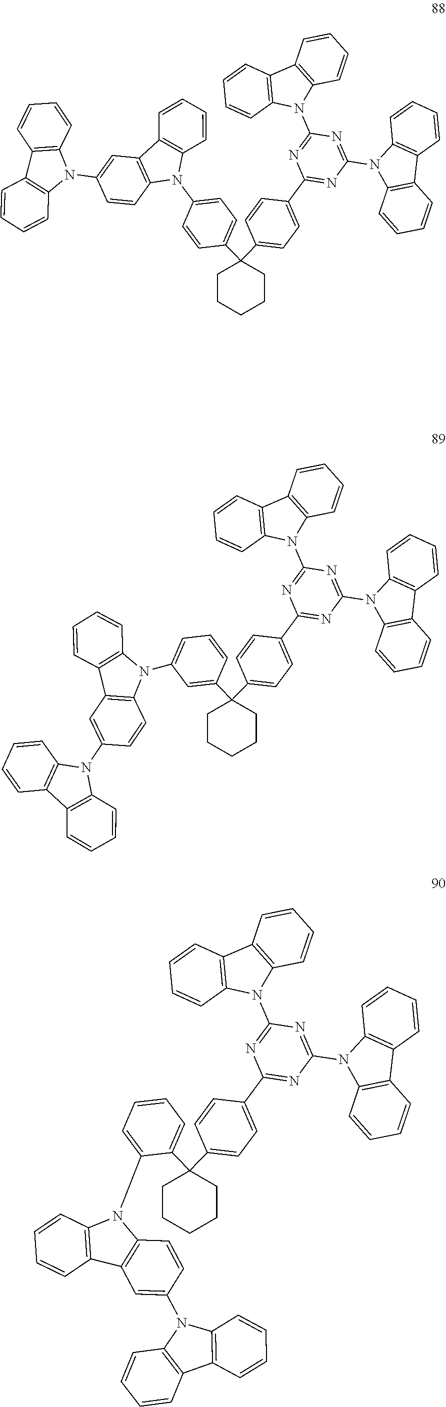 [SMILES: c1ccc2c(c1)c1ccccc1n2-c1ccc2c(c1)c1ccccc1n2-c1ccc(C2(c3ccc(-c4nc(-n5c6ccccc6c6ccccc65)nc(-n5c6ccccc6c6ccccc65)n4)cc3)CCCCC2)cc1 ; c1cc(-n2c3ccccc3c3cc(-n4c5ccccc5c5ccccc54)ccc32)cc(C2(c3ccc(-c4nc(-n5c6ccccc6c6ccccc65)nc(-n5c6ccccc6c6ccccc65)n4)cc3)CCCCC2)c1 ; c1ccc(C2(c3ccc(-c4nc(-n5c6ccccc6c6ccccc65)nc(-n5c6ccccc6c6ccccc65)n4)cc3)CCCCC2)c(-n2c3ccccc3c3cc(-n4c5ccccc5c5ccccc54)ccc32)c1]

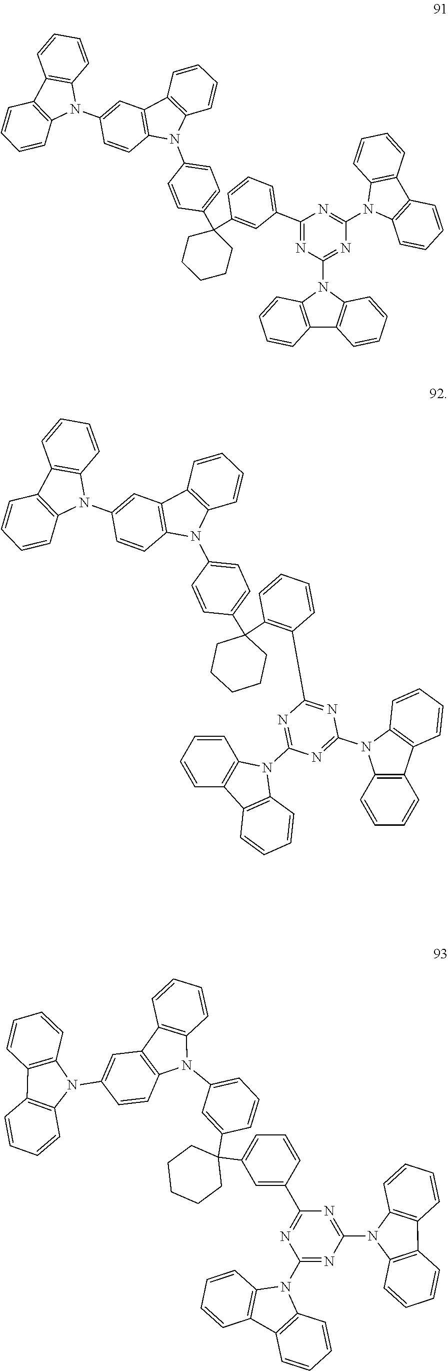 [SMILES: c1cc(-c2nc(-n3c4ccccc4c4ccccc43)nc(-n3c4ccccc4c4ccccc43)n2)cc(C2(c3ccc(-n4c5ccccc5c5cc(-n6c7ccccc7c7ccccc76)ccc54)cc3)CCCCC2)c1 ; c1ccc(C2(c3ccc(-n4c5ccccc5c5cc(-n6c7ccccc7c7ccccc76)ccc54)cc3)CCCCC2)c(-c2nc(-n3c4ccccc4c4ccccc43)nc(-n3c4ccccc4c4ccccc43)n2)c1 ; c1cc(-c2nc(-n3c4ccccc4c4ccccc43)nc(-n3c4ccccc4c4ccccc43)n2)cc(C2(c3cccc(-n4c5ccccc5c5cc(-n6c7ccccc7c7ccccc76)ccc54)c3)CCCCC2)c1]

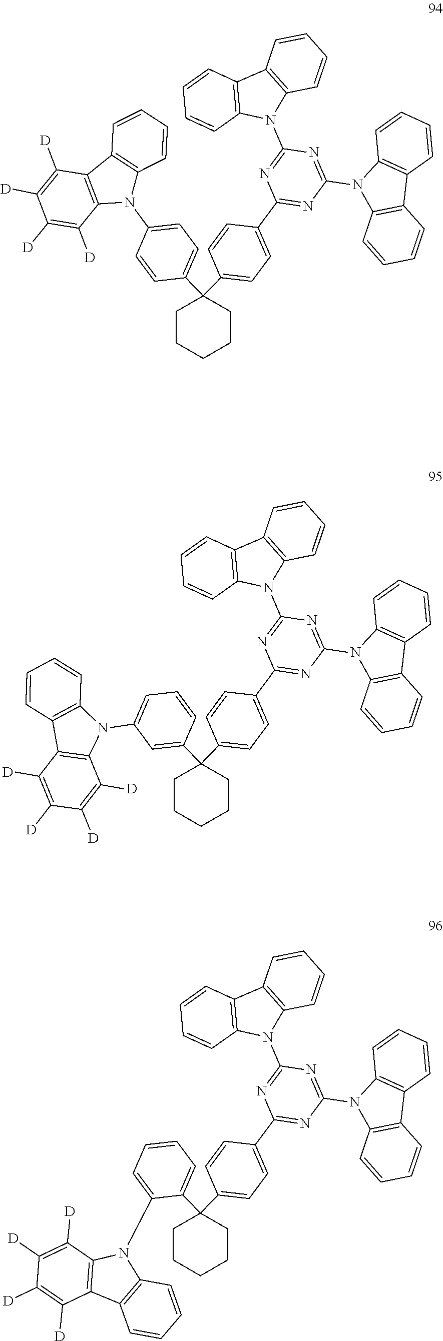 [SMILES: [2H]c1c([2H])c([2H])c2c(c1[2H])c1ccccc1n2-c1ccc(C2(c3ccc(-c4nc(-n5c6ccccc6c6ccccc65)nc(-n5c6ccccc6c6ccccc65)n4)cc3)CCCCC2)cc1 ; [2H]c1c([2H])c([2H])c2c(c1[2H])c1ccccc1n2-c1cccc(C2(c3ccc(-c4nc(-n5c6ccccc6c6ccccc65)nc(-n5c6ccccc6c6ccccc65)n4)cc3)CCCCC2)c1 ; [2H]c1c([2H])c([2H])c2c(c1[2H])c1ccccc1n2-c1ccccc1C1(c2ccc(-c3nc(-n4c5ccccc5c5ccccc54)nc(-n4c5ccccc5c5ccccc54)n3)cc2)CCCCC1]

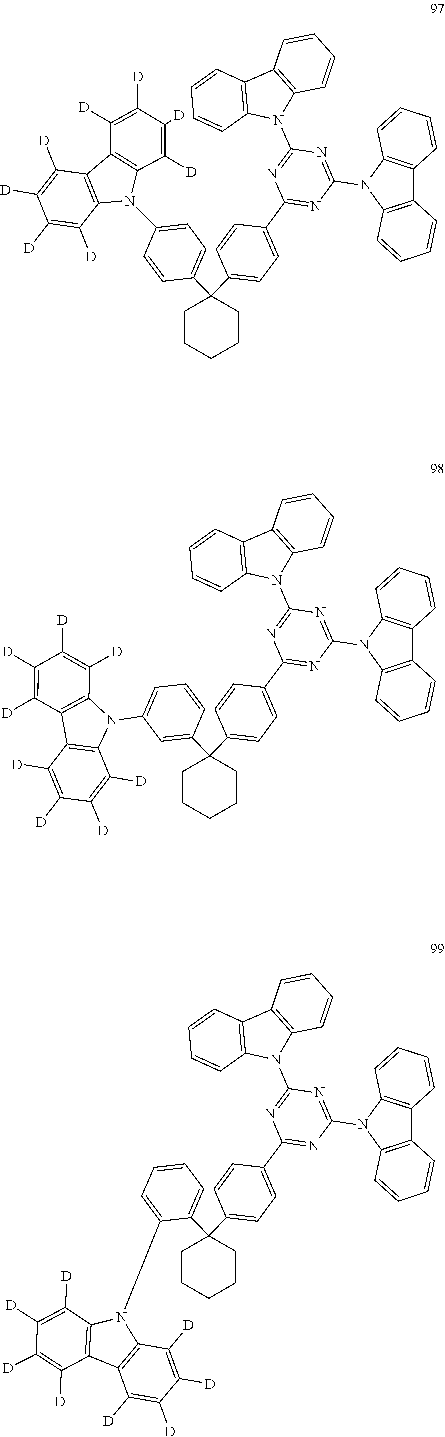 [SMILES: [2H]c1c([2H])c([2H])c2c(c1[2H])c1c([2H])c([2H])c([2H])c([2H])c1n2-c1ccc(C2(c3ccc(-c4nc(-n5c6ccccc6c6ccccc65)nc(-n5c6ccccc6c6ccccc65)n4)cc3)CCCCC2)cc1 ; [2H]c1c([2H])c([2H])c2c(c1[2H])c1c([2H])c([2H])c([2H])c([2H])c1n2-c1cccc(C2(c3ccc(-c4nc(-n5c6ccccc6c6ccccc65)nc(-n5c6ccccc6c6ccccc65)n4)cc3)CCCCC2)c1 ; [2H]c1c([2H])c([2H])c2c(c1[2H])c1c([2H])c([2H])c([2H])c([2H])c1n2-c1ccccc1C1(c2ccc(-c3nc(-n4c5ccccc5c5ccccc54)nc(-n4c5ccccc5c5ccccc54)n3)cc2)CCCCC1]

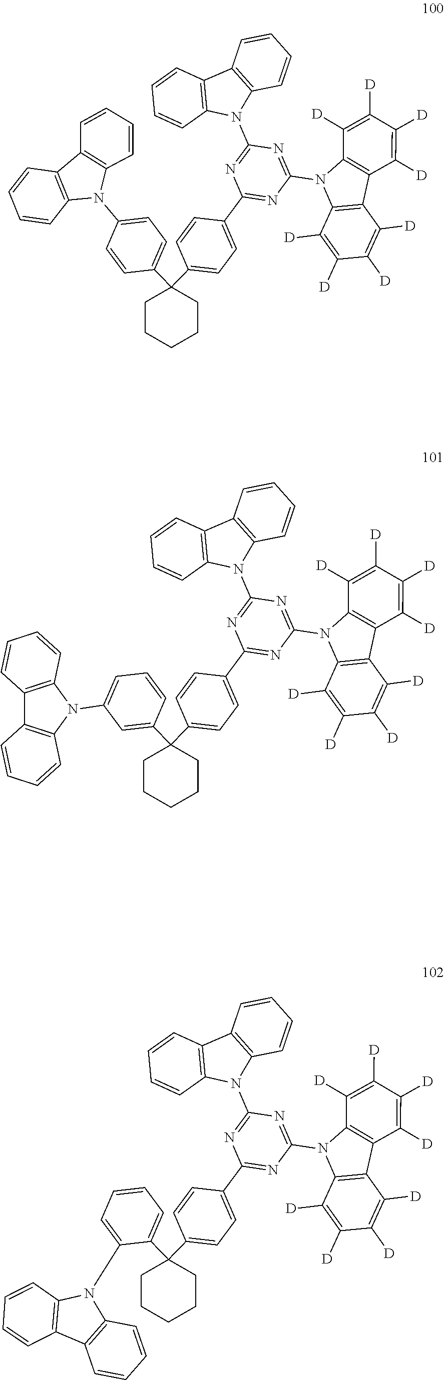 [SMILES: [2H]c1c([2H])c([2H])c2c(c1[2H])c1c([2H])c([2H])c([2H])c([2H])c1n2-c1nc(-c2ccc(C3(c4ccc(-n5c6ccccc6c6ccccc65)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4cccc(-n5c6ccccc6c6ccccc65)c4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccccc4-n4c5ccccc5c5ccccc54)CCCCC3)cc2)nc(-n2c3ccccc3c3ccccc32)n1]

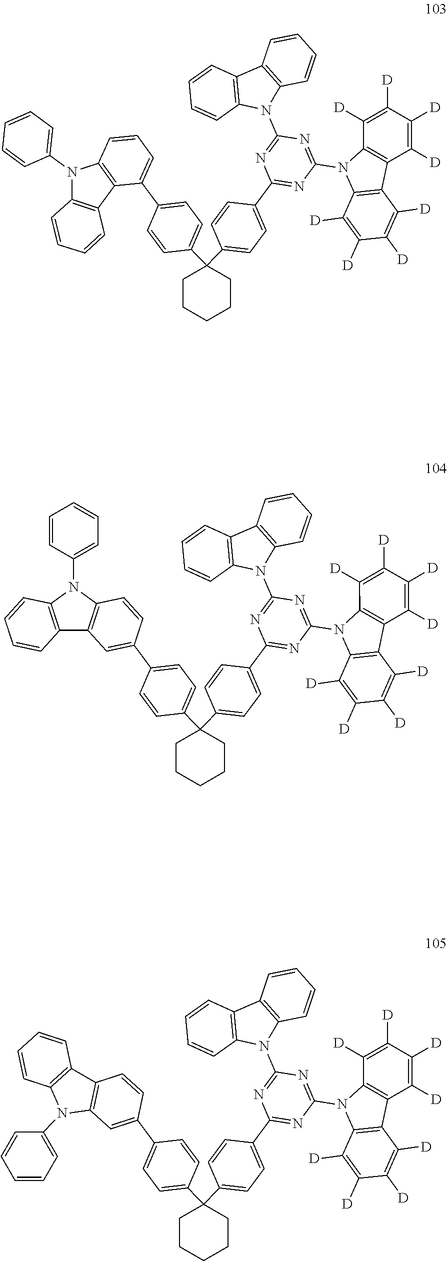 [SMILES: [2H]c1c([2H])c([2H])c2c(c1[2H])c1c([2H])c([2H])c([2H])c([2H])c1n2-c1nc(-c2ccc(C3(c4ccc(-c5cccc6c5c5ccccc5n6-c5ccccc5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6c(c5)c5ccccc5n6-c5ccccc5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6c7ccccc7n(-c7ccccc7)c6c5)cc4)CCCCC3)cc2)nc(-n2c3ccccc3c3ccccc32)n1]

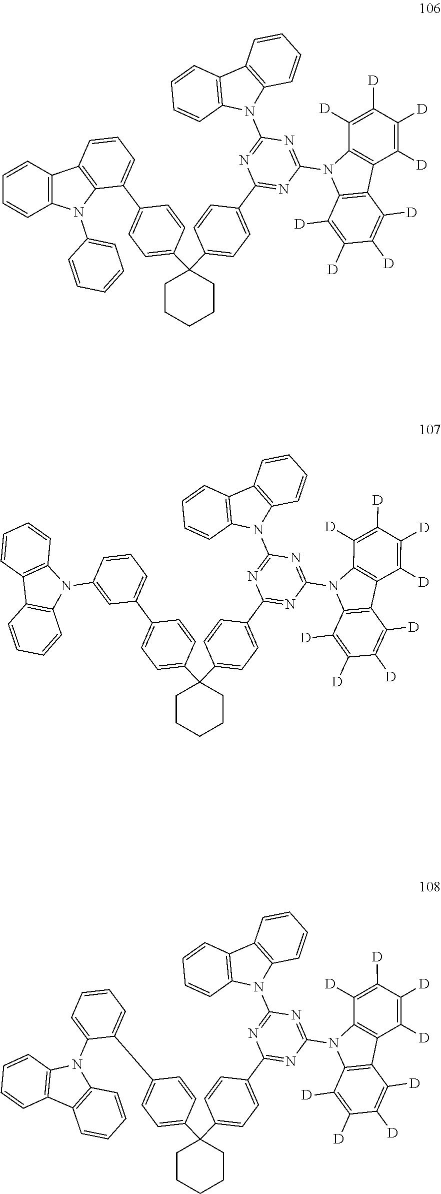 [SMILES: [2H]c1c([2H])c([2H])c2c(c1[2H])c1c([2H])c([2H])c([2H])c([2H])c1n2-c1nc(-c2ccc(C3(c4ccc(-c5cccc6c7ccccc7n(-c7ccccc7)c56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5cccc(-n6c7ccccc7c7ccccc76)c5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccccc5-n5c6ccccc6c6ccccc65)cc4)CCCCC3)cc2)nc(-n2c3ccccc3c3ccccc32)n1]

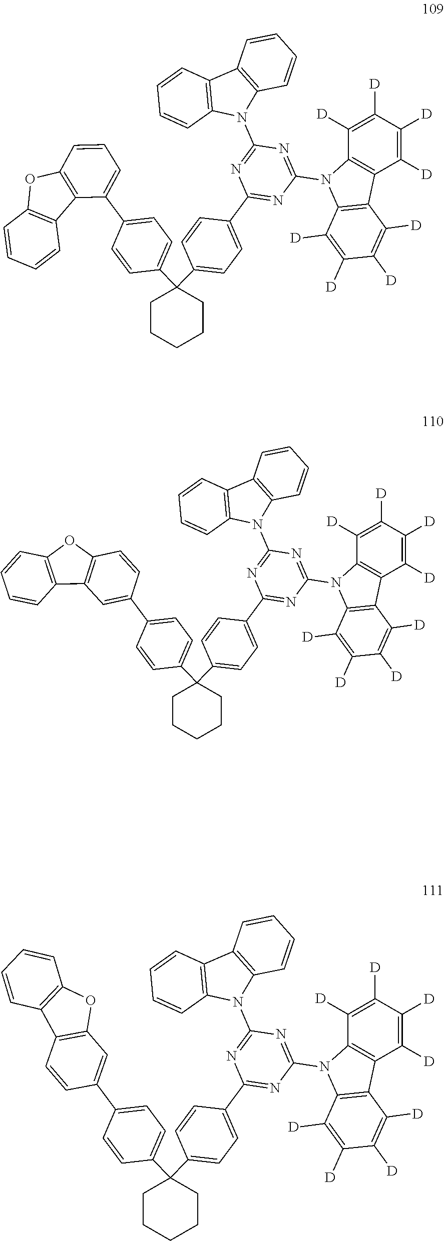 [SMILES: [2H]c1c([2H])c([2H])c2c(c1[2H])c1c([2H])c([2H])c([2H])c([2H])c1n2-c1nc(-c2ccc(C3(c4ccc(-c5cccc6oc7ccccc7c56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6oc7ccccc7c6c5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6c(c5)oc5ccccc56)cc4)CCCCC3)cc2)nc(-n2c3ccccc3c3ccccc32)n1]

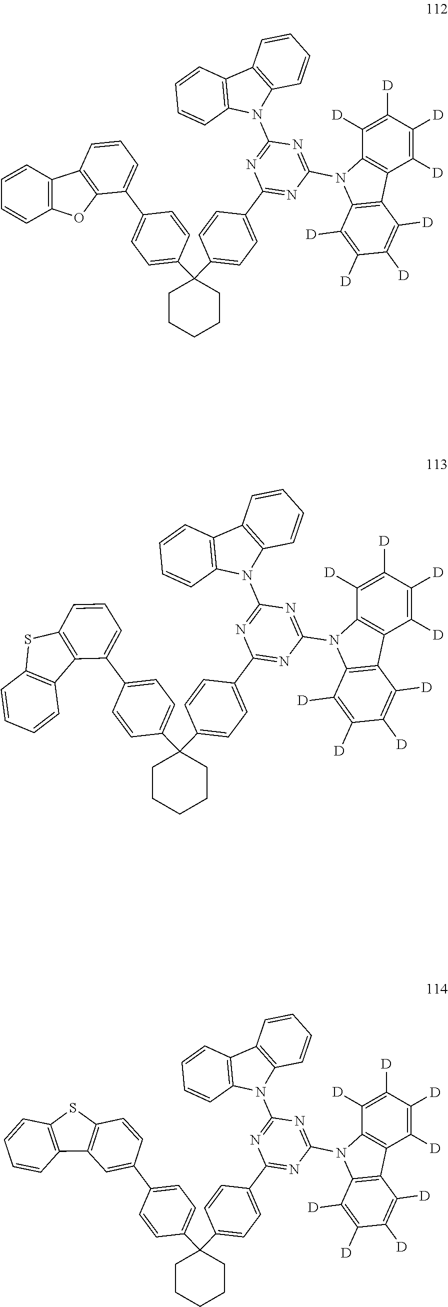 [SMILES: [2H]c1c([2H])c([2H])c2c(c1[2H])c1c([2H])c([2H])c([2H])c([2H])c1n2-c1nc(-c2ccc(C3(c4ccc(-c5cccc6c5oc5ccccc56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5cccc6sc7ccccc7c56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6sc7ccccc7c6c5)cc4)CCCCC3)cc2)nc(-n2c3ccccc3c3ccccc32)n1]

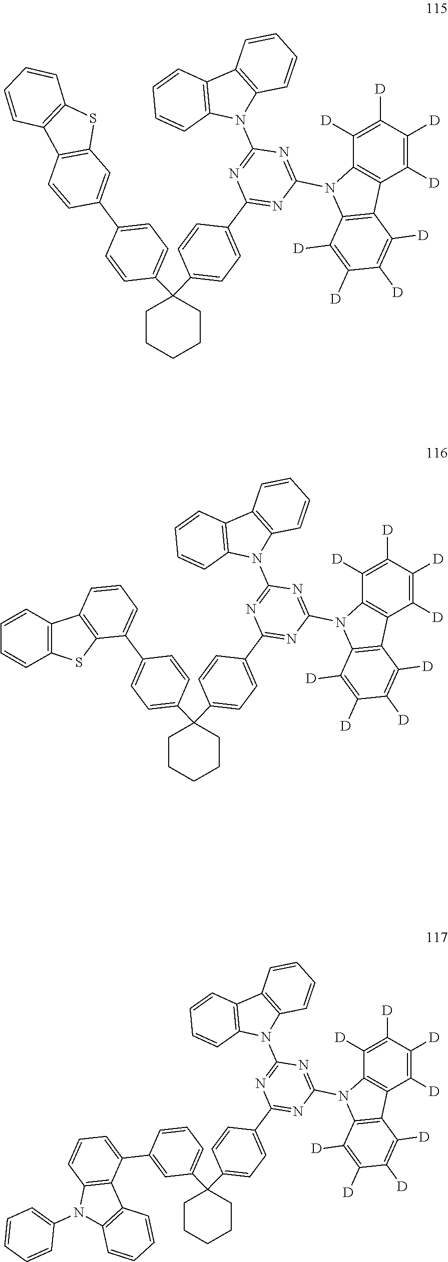 [SMILES: [2H]c1c([2H])c([2H])c2c(c1[2H])c1c([2H])c([2H])c([2H])c([2H])c1n2-c1nc(-c2ccc(C3(c4ccc(-c5ccc6c(c5)sc5ccccc56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5cccc6c5sc5ccccc56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4cccc(-c5cccc6c5c5ccccc5n6-c5ccccc5)c4)CCCCC3)cc2)nc(-n2c3ccccc3c3ccccc32)n1]

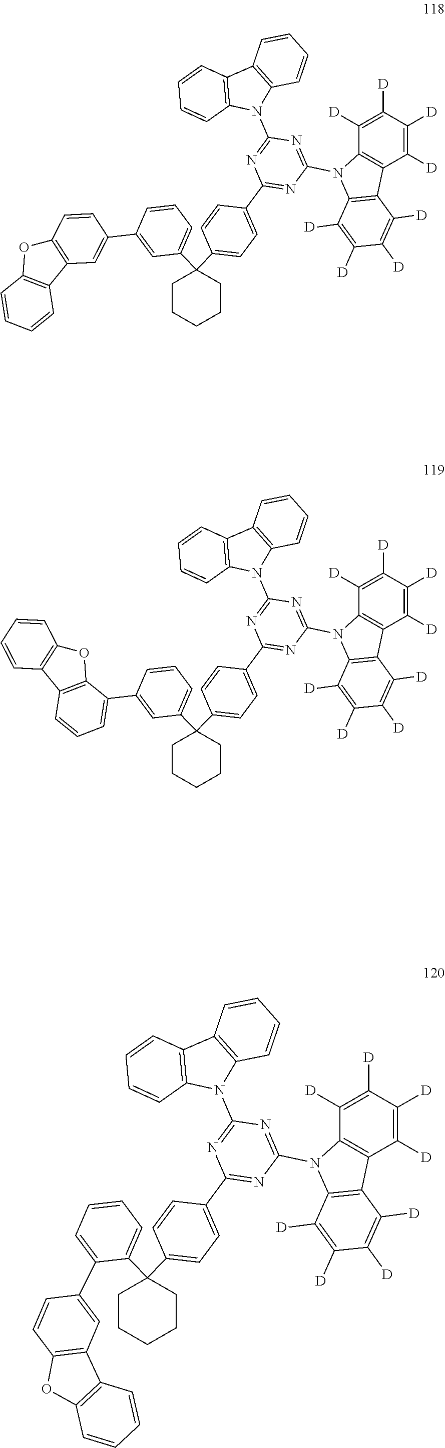 [SMILES: [2H]c1c([2H])c([2H])c2c(c1[2H])c1c([2H])c([2H])c([2H])c([2H])c1n2-c1nc(-c2ccc(C3(c4cccc(-c5ccc6oc7ccccc7c6c5)c4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4cccc(-c5cccc6c5oc5ccccc56)c4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccccc4-c4ccc5oc6ccccc6c5c4)CCCCC3)cc2)nc(-n2c3ccccc3c3ccccc32)n1]

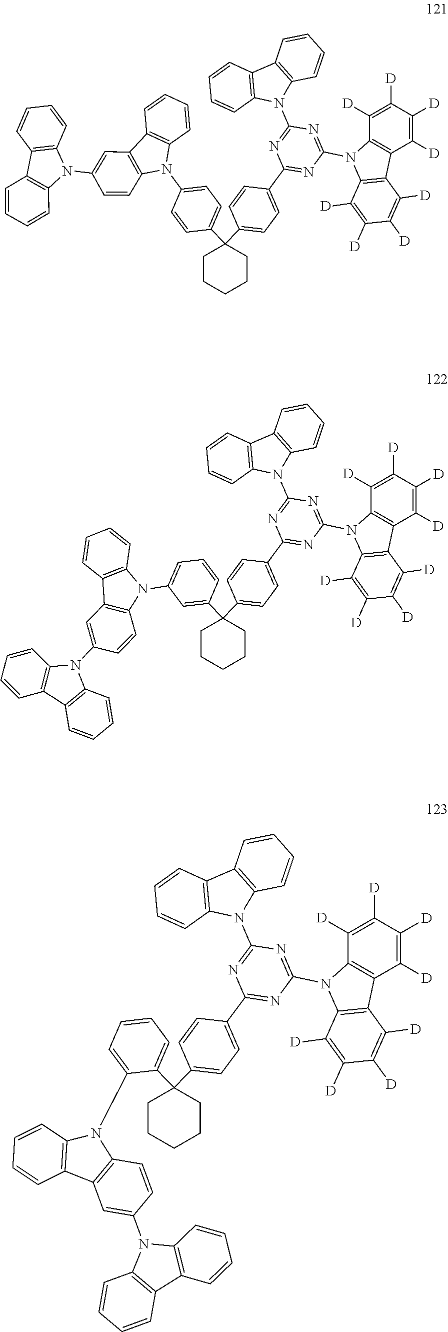

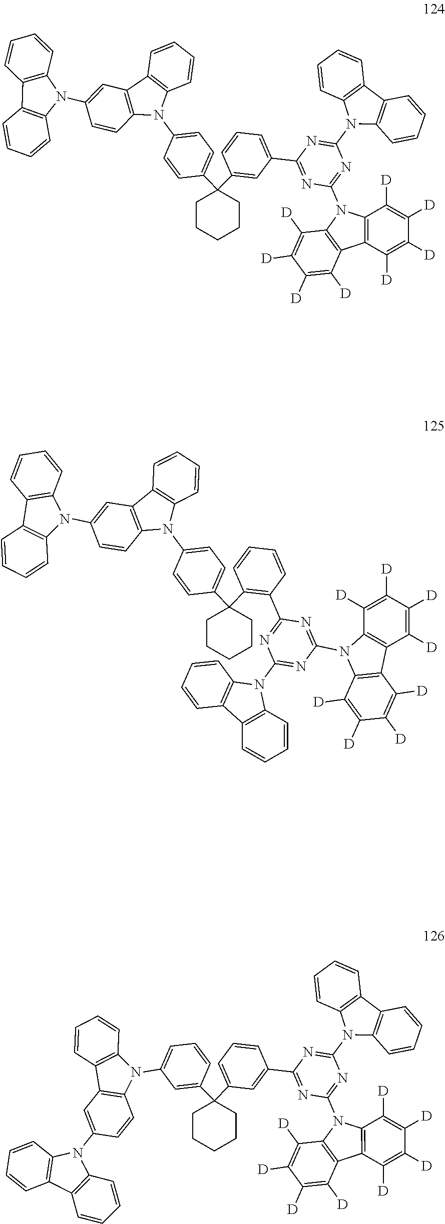

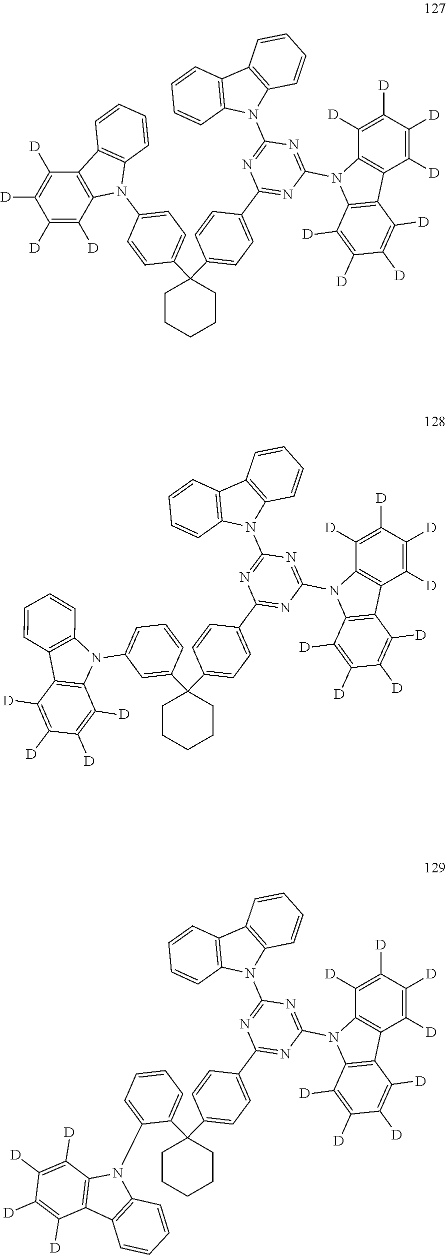

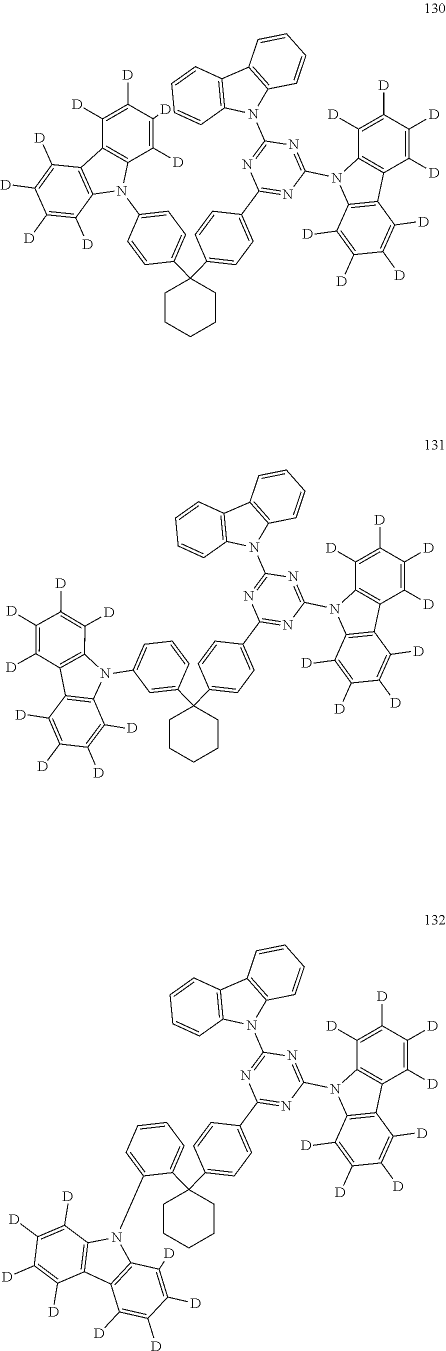

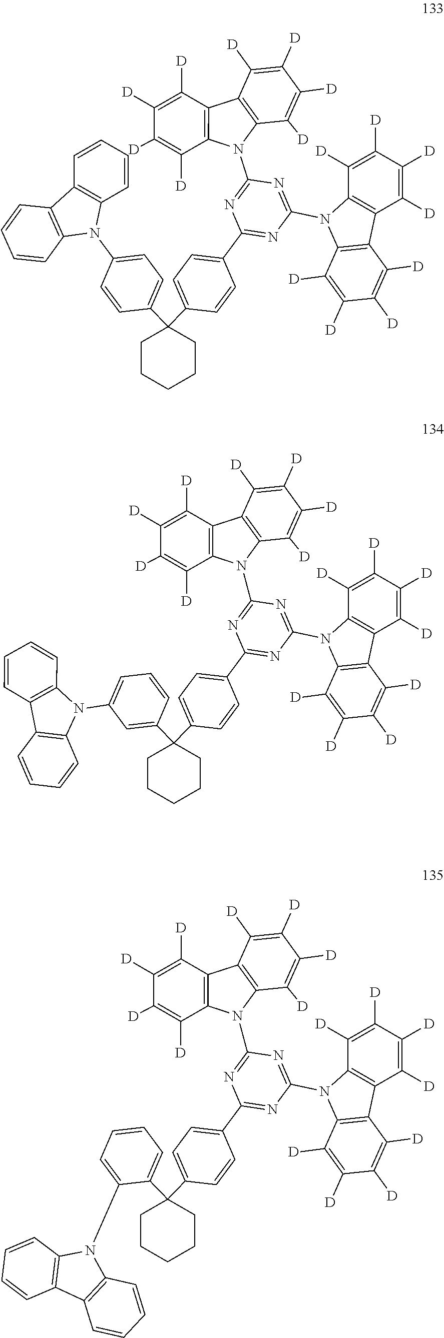

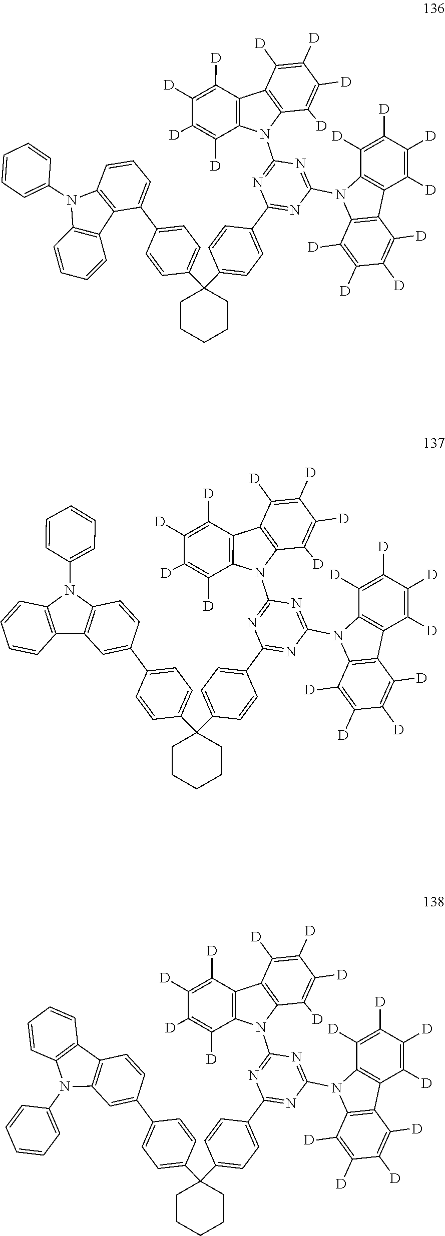

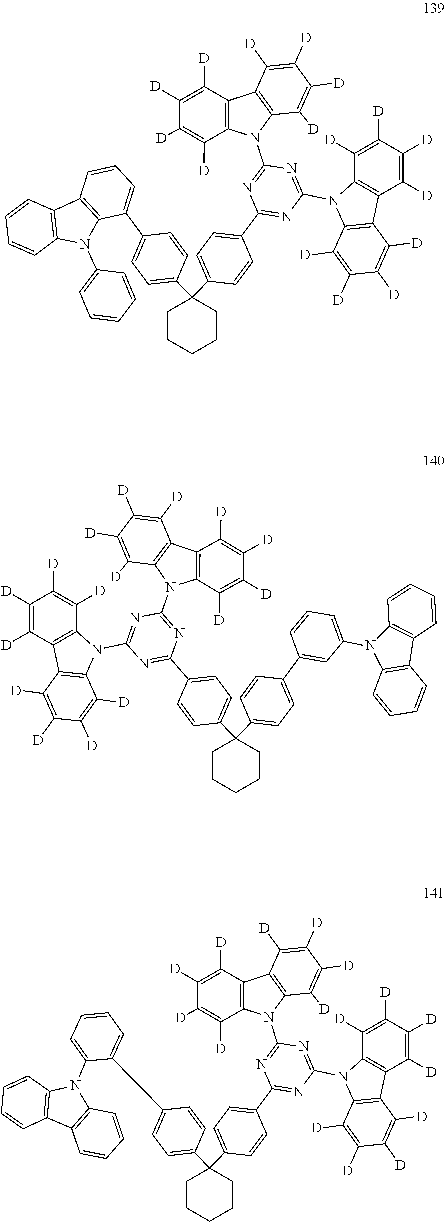

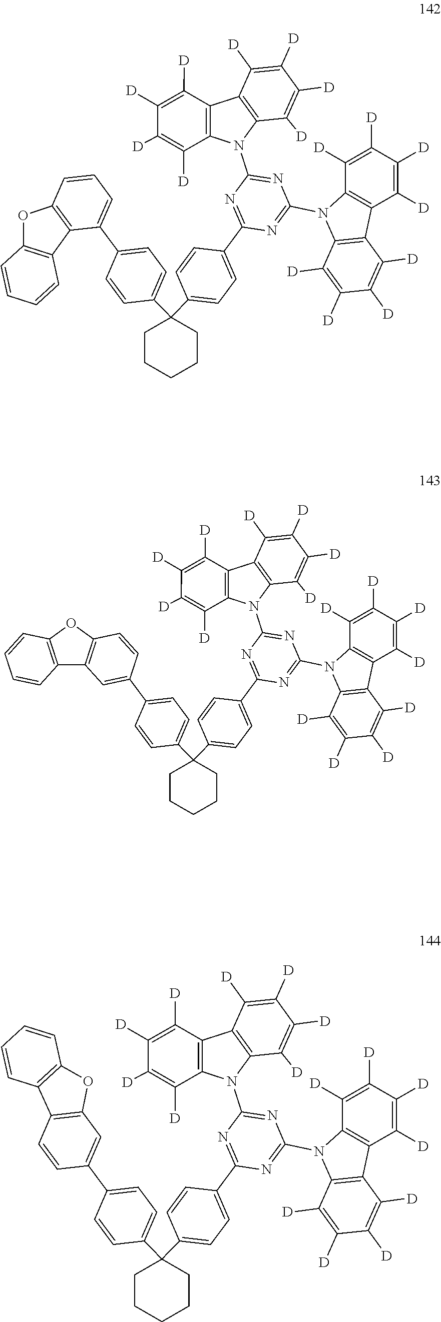

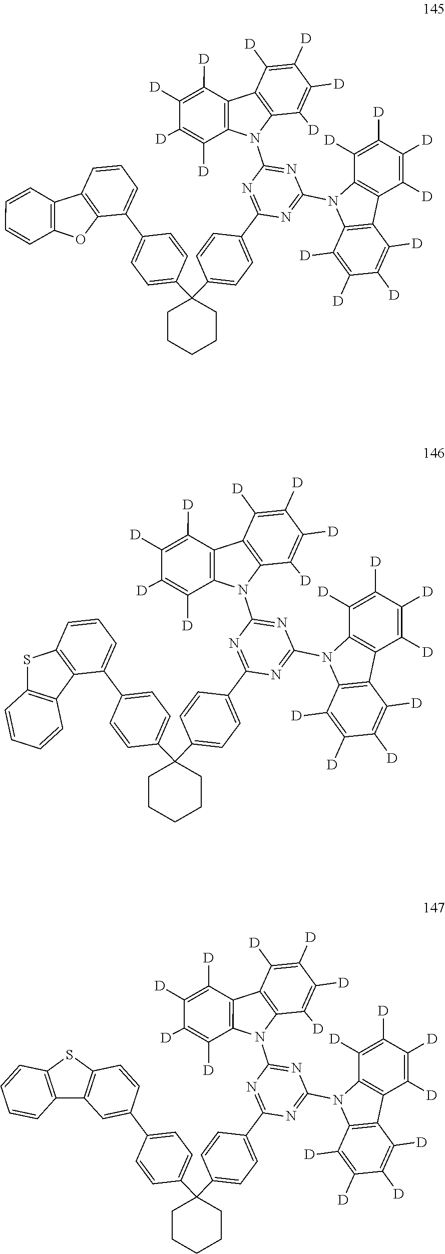

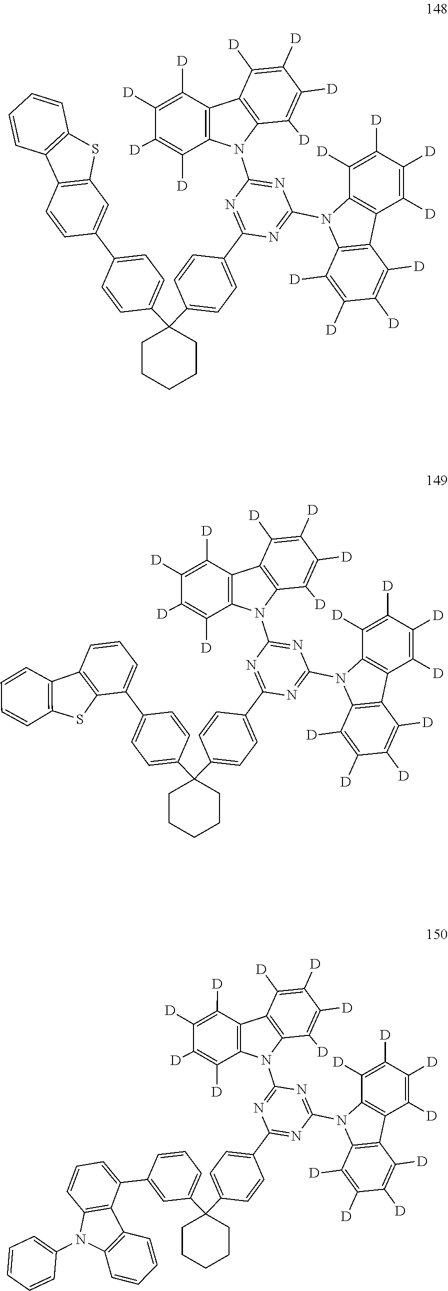

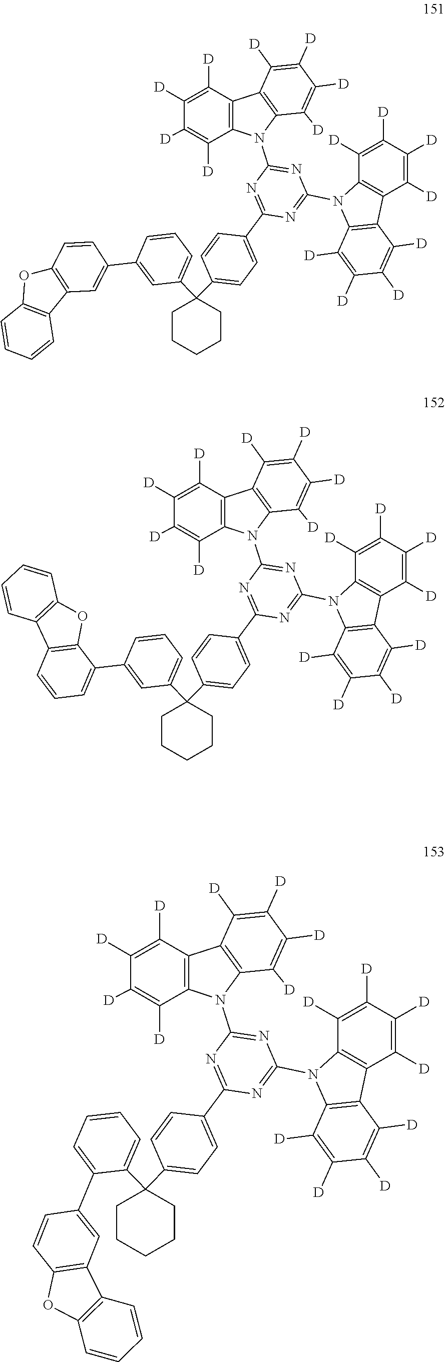

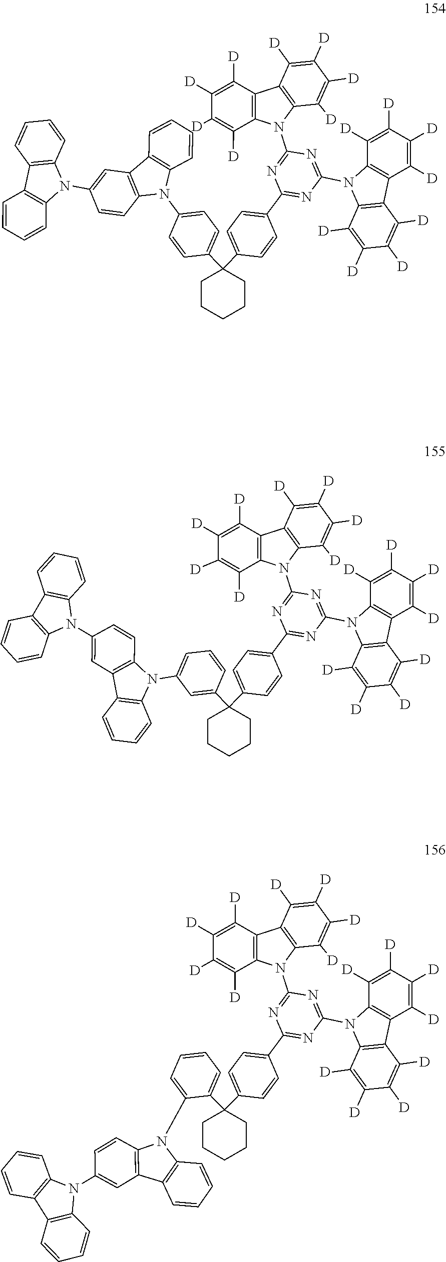

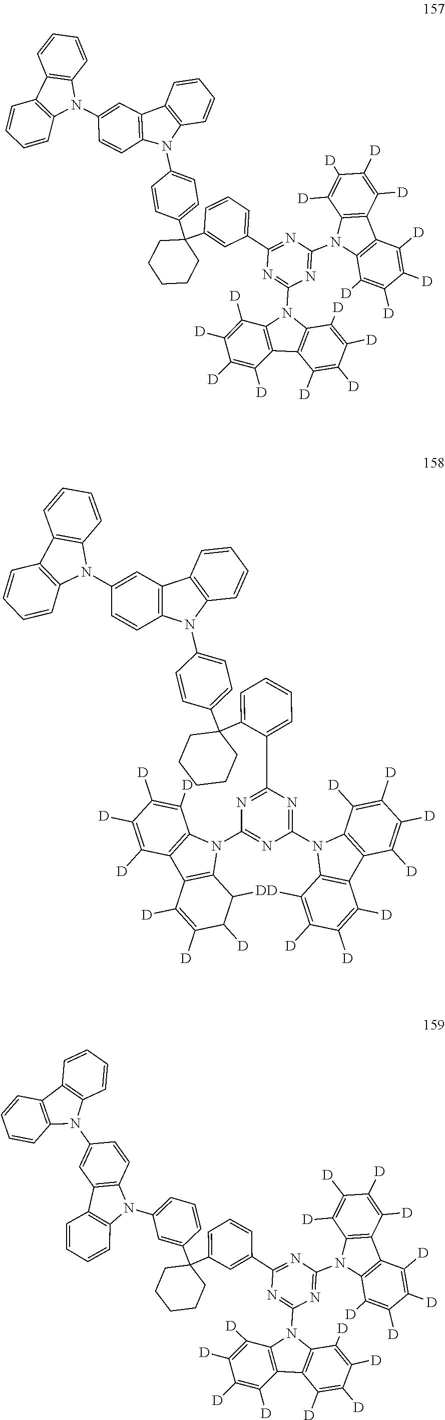

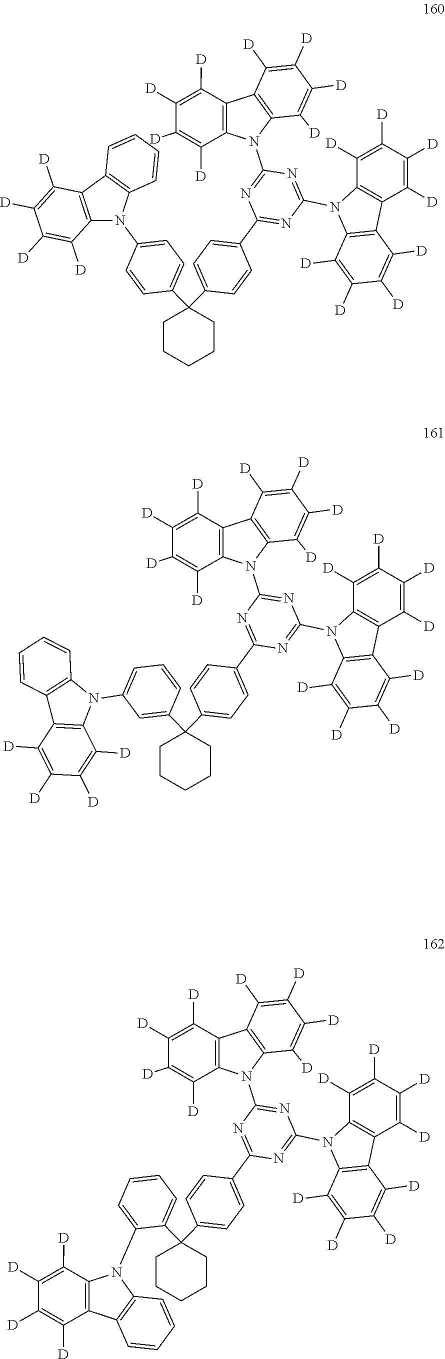

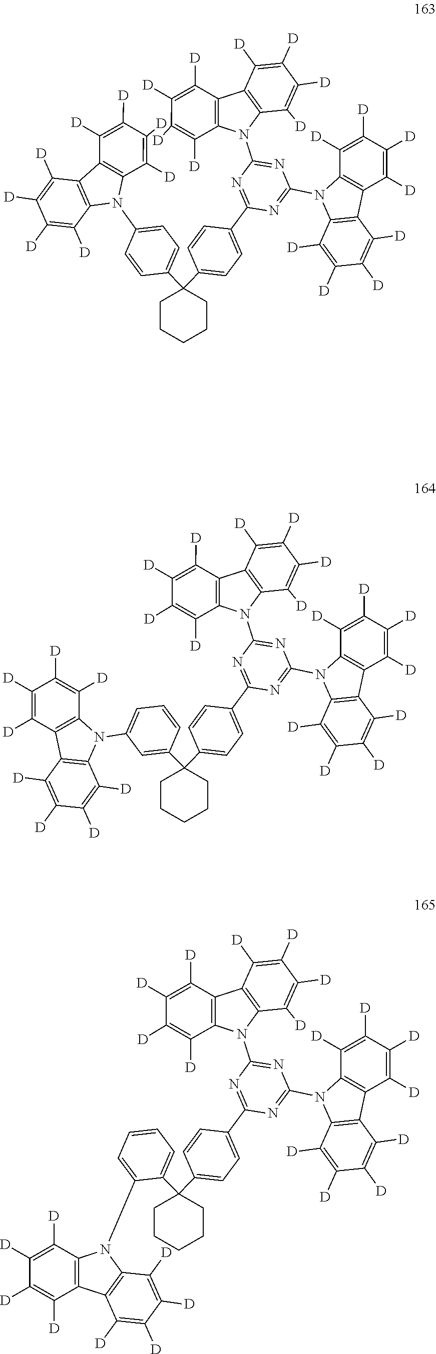

The heterocyclic compound represented by Formula 1 may have a non-conjugated cyclic linker as a core, and may include an acceptor including a heteroatom as a substituent of the core and a donor including a hetero atom. Therefore, the heterocyclic compound represented by Formula 1 may be a bipolar compound.

By including a non-conjugated cyclic linker as a core, charge transport properties may be improved. In addition, by including a bipolar structure, lifespan may be improved. Therefore, the heterocyclic compound represented by Formula 1 may have high thermal stability and hole stability.

By using the heterocyclic compound represented by Formula 1, the driving voltage, luminescence efficiency, and lifespan of the light-emitting device (for example, an organic light-emitting device) may be improved.

Synthesis methods of the heterocyclic compound represented by Formula 1 may be recognizable by one of ordinary skill in the art by referring to Synthesis Examples and/or Examples provided herein below.

At least one heterocyclic compound represented by Formula 1 may be used in a light-emitting device (for example, an organic light-emitting device). Therefore, provided is a light-emitting device including: a first electrode; a second electrode facing the first electrode; an interlayer between the first electrode and the second electrode and including an emission layer; and the heterocyclic compound represented by Formula 1 as described above.

In one or more embodiments, the first electrode may be an anode, the second electrode may be a cathode, the interlayer may further include a hole transport region between the first electrode and the emission layer and an electron transport region between the emission layer and the second electrode, the hole transport region may include a hole injection layer, a hole transport layer, an emission auxiliary layer, an electron blocking layer, or a combination thereof, and the electron transport region may include a hole blocking layer, an electron transport layer, an electron injection layer, an electron control layer, or a combination thereof.
the interlayer may further include a hole transport region between the first electrode and the emission layer and an electron transport region between the emission layer and the second electrode,
the hole transport region may include a hole injection layer, a hole transport layer, an emission auxiliary layer, an electron blocking layer, or a combination thereof, and
the electron transport region may include a buffer layer, a hole blocking layer, an electron control layer, an electron transport layer, an electron injection layer, or a combination thereof.

In one or more embodiments, the heterocyclic compound may be included between the first electrode and the second electrode of the light-emitting device. Accordingly, the heterocyclic compound may be included in the interlayer of the light-emitting device, for example, in the emission layer of the interlayer.

In some embodiments, the emission layer in the interlayer of the light-emitting device may include a dopant and a host, and the heterocyclic compound may be included in the host. For example, the heterocyclic compound may serve as a host. The emission layer may emit red light, green light, blue light, and/or white light. For example, the emission layer may emit blue light. The blue light may have a maximum emission wavelength (e.g., a peak emission wavelength) in a range of about 400 nm to about 490 nm, about 410 nm to about 490 nm, about 420 nm to about 480 nm, or about 430 nm to about 480 nm. In one or more embodiments, the dopant may be a phosphorescent dopant, a fluorescent dopant, a delayed fluorescence dopant, or a combination thereof.

In one or more embodiments, the light-emitting device may include a capping layer outside the first electrode or outside the second electrode.

In one or more embodiments, the light-emitting device may further include at least one selected from a first capping layer outside the first electrode and a second capping layer outside the second electrode, and at least one selected from the first capping layer and the second capping layer may include the heterocyclic compound represented by Formula 1. More details for the first capping layer and/or second capping layer are as described herein.

In one or more embodiments, the light-emitting device may include: a first capping layer including the first compound represented by Formula 1 outside the first electrode; a second capping layer including the first compound represented by Formula 1 outside the second electrode; or the first capping layer and the second capping layer.

The expression “(an interlayer and/or a capping layer) includes at least one heterocyclic compound” as used herein may include a case in which “(an interlayer and/or a capping layer) includes identical heterocyclic compounds represented by Formula 1” and a case in which “(an interlayer and/or a capping layer) includes two or more different heterocyclic compounds represented by Formula 1.”

For example, the interlayer and/or capping layer may include Compound 1 only as the heterocyclic compound. In this regard, Compound 1 may be present in the emission layer of the light-emitting device. In some embodiments, the interlayer may include, as the heterocyclic compound, Compound 1 and Compound 2. In this regard, Compound 1 and Compound 2 may be present in the same layer (for example, all of Compound 1 and Compound 2 may be present in the emission layer), or may be present in different layers (for example, Compound 1 may be present in the emission layer, and Compound 2 may be present in the electron transport region).

The term “interlayer” as used herein refers to a single layer and/or all of a plurality of layers between the first electrode and the second electrode of the light-emitting device.

Another aspect of embodiments provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In one or more embodiments, the electronic apparatus may further include a color filter, a color conversion layer, a touch screen layer, a polarizing layer, or a combination thereof. For more details on the electronic apparatus, related descriptions provided herein may be referred to.

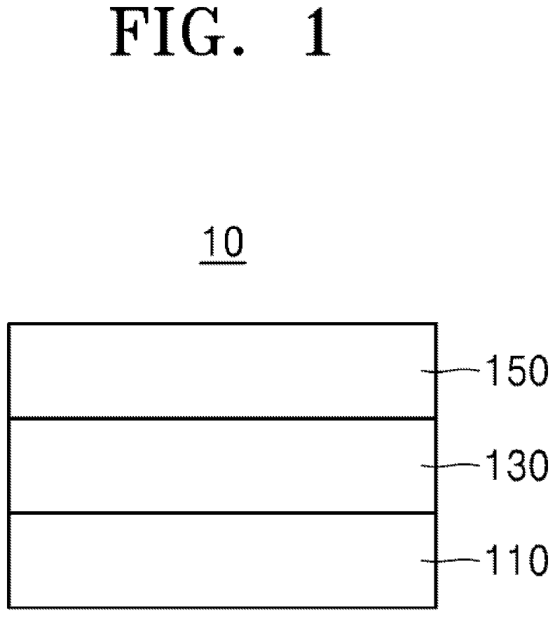

### DESCRIPTION OF FIG. 1

FIG. 1 is a schematic cross-sectional view of a light-emitting device 10 according to one or more embodiments. The light-emitting device 10 includes a first electrode 110, an interlayer 130, and a second electrode 150.

Hereinafter, the structure of the light-emitting device 10 according to one or more embodiments and a method of manufacturing the light-emitting device 10 will be described with reference to FIG. 1.

### First Electrode 110

In FIG. 1, a substrate may be additionally under the first electrode 110 and/or on the second electrode 150. As the substrate, a glass substrate and/or a plastic substrate may be used. In some embodiments, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or a combination thereof.

The first electrode 110 may be formed by, for example, depositing and/or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or a combination thereof. In some embodiments, when the first electrode 110 is a semi-transmissive electrode or a reflective electrode, a material for forming the first electrode 110 may include magnesium (Mg), silver (Ag), aluminum (AI), aluminum-lithium (Al—Li), calcium (Ca), magnesium-indium (Mg—In), magnesium-silver (Mg—Ag), or a combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be on the first electrode 110. The interlayer 130 may include an emission layer.

The interlayer 130 may further include a hole transport region between the first electrode 110 and the emission layer, and an electron transport region between the emission layer and the second electrode 150.

The interlayer 130 may further include, in addition to various suitable organic materials, a metal-containing compound such as an organometallic compound, an inorganic material such as quantum dots, and/or the like.

In some embodiments, the interlayer 130 may include, i) two or more emitting units sequentially stacked between the first electrode 110 and the second electrode 150, and ii) a charge generation layer between the two or more emitting units. When the interlayer 130 includes emitting units and a charge generation layer as described above, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, a hole transport layer, an emission auxiliary layer, an electron blocking layer, or a combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, the layers of each structure being stacked sequentially from the first electrode 110.

The hole transport region may include a compound represented by Formula 201, a compound represented by Formula 202, or a combination thereof:

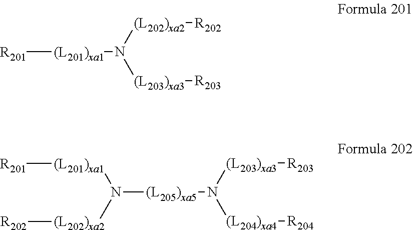

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one selected from groups represented by Formulae CY₂₀₁ to CY₂₁₇:

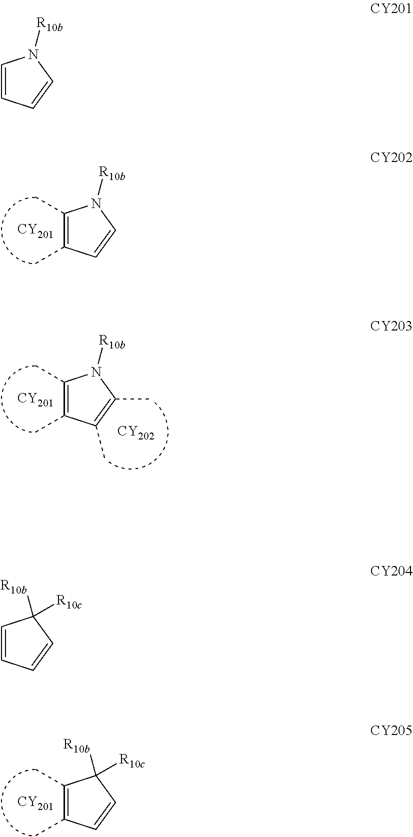

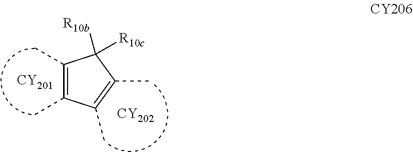

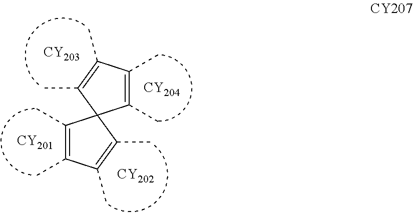

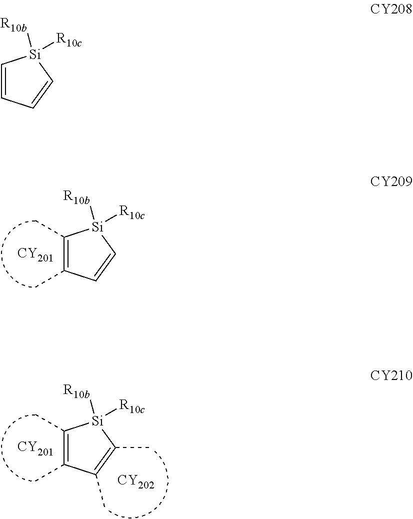

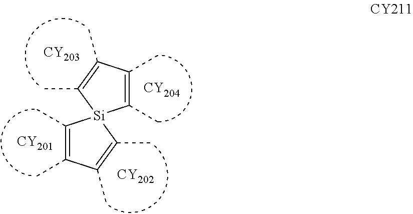

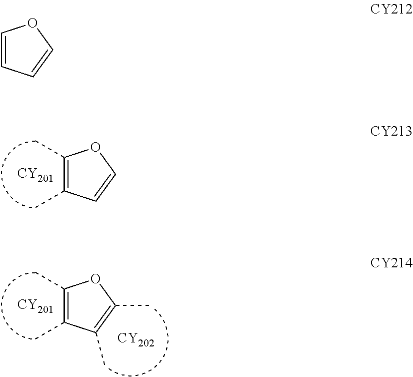

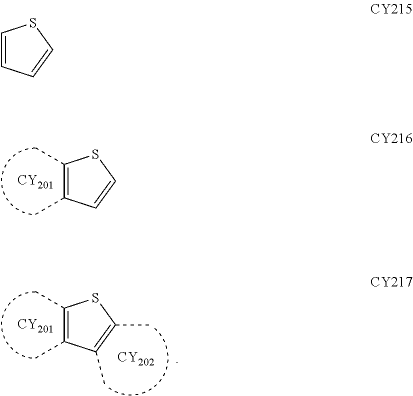

In Formulae CY₂₀₁ to CY₂₁₇, R_{10b} and R_{10c} may each be as described for R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY₂₀₁ to CY₂₁₇ may be unsubstituted or substituted with R₁₀ₐ as described above.

In one or more embodiments, ring CY₂₀₁ to ring CY₂₀₄ in Formulae CY₂₀₁ to CY₂₁₇ may each independently be a benzene group, a naphthalene group, a phenanthrene group, or an anthracene group.

In some embodiments, each of Formulae 201 and 202 may include at least one selected from groups represented by Formulae CY₂₀₁ to CY₂₀₃.

In some embodiments, Formula 201 may include at least one selected from the groups represented by Formulae CY₂₀₁ to CY₂₀₃ and at least one selected from the groups represented by Formulae CY₂₀₄ to CY₂₁₇.

In some embodiments, in Formula 201, xa1 may be 1, R₂₀₁ may be a group represented by one selected from Formulae CY₂₀₁ to CY₂₀₃, xa2 may be 0, and R₂₀₂ may be a group represented by one selected from Formulae CY₂₀₄ to CY₂₀₇.

In some embodiments, each of Formulae 201 and 202 may not include a group represented by one selected from Formulae CY201 to CY203.

In some embodiments, each of Formulae 201 and 202 may not include a group represented by one selected from Formulae CY201 to CY203, and may include at least one selected from the groups represented by Formulae CY204 to CY217.

In some embodiments, each of Formulae 201 and 202 may not include a group represented by one selected from Formulae CY201 to CY217.

In one or more embodiments, the hole transport region may include one selected from Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or a combination thereof:

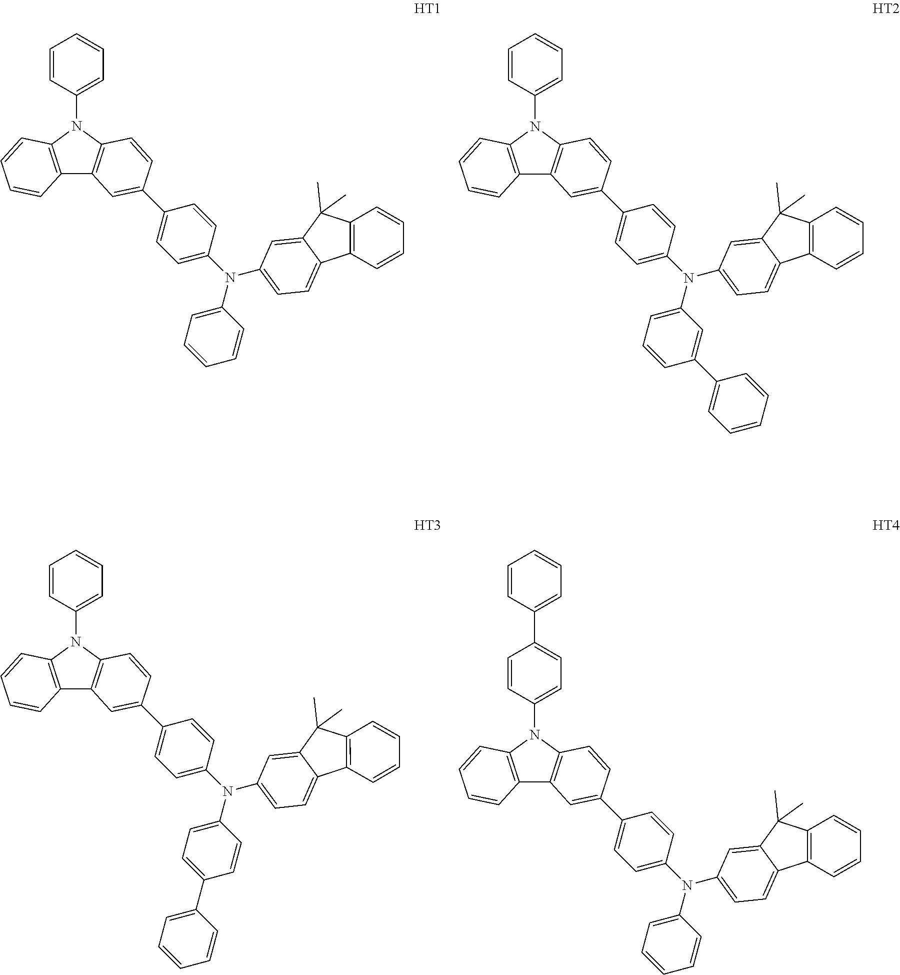 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

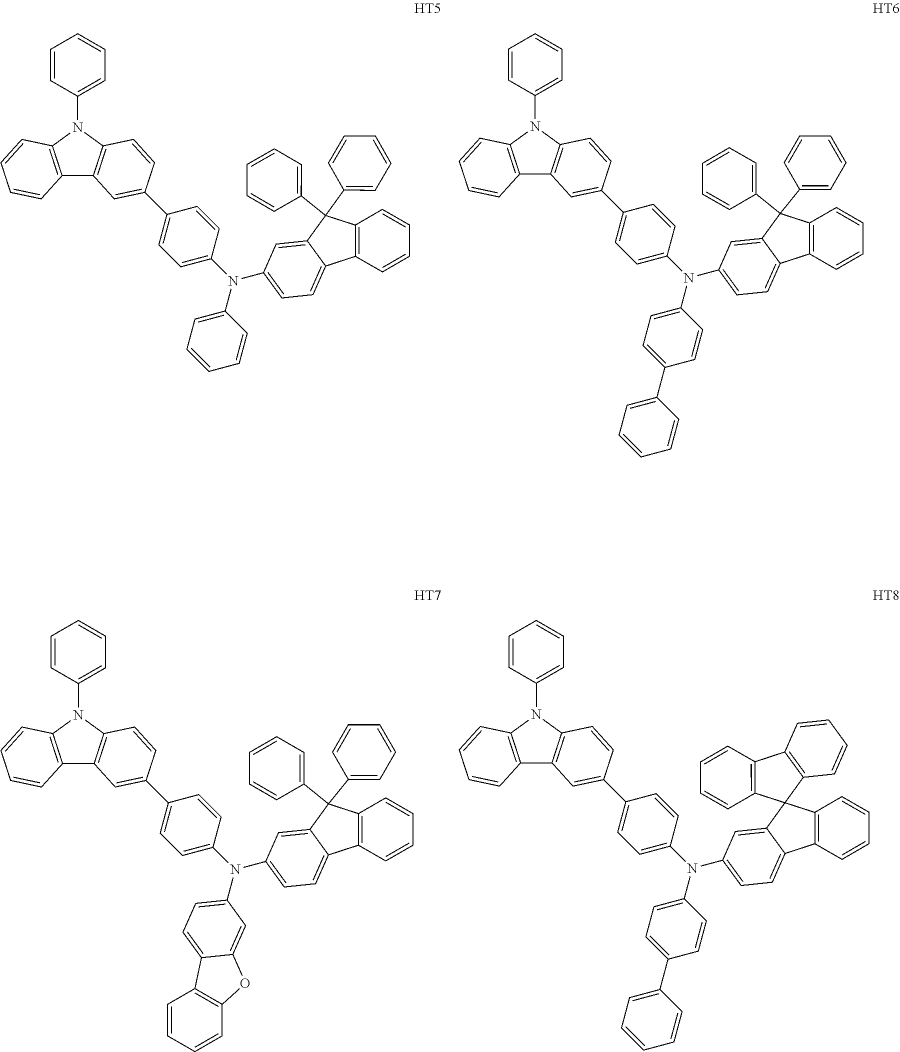 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

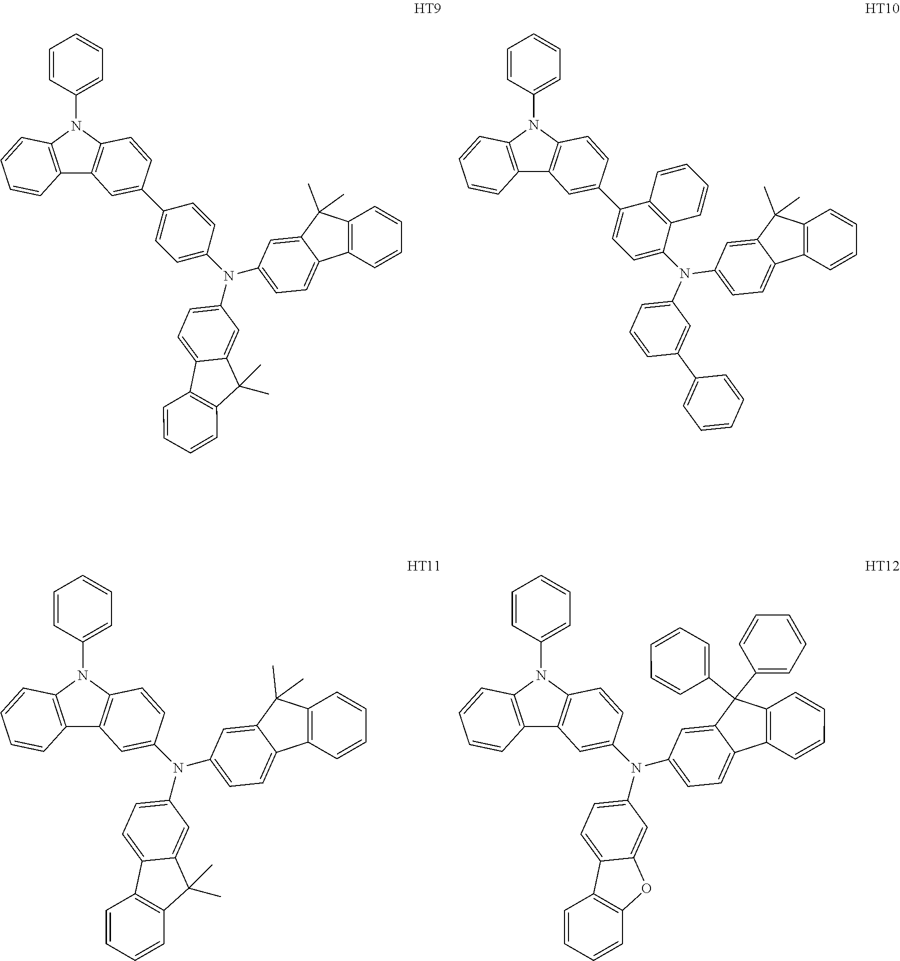 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

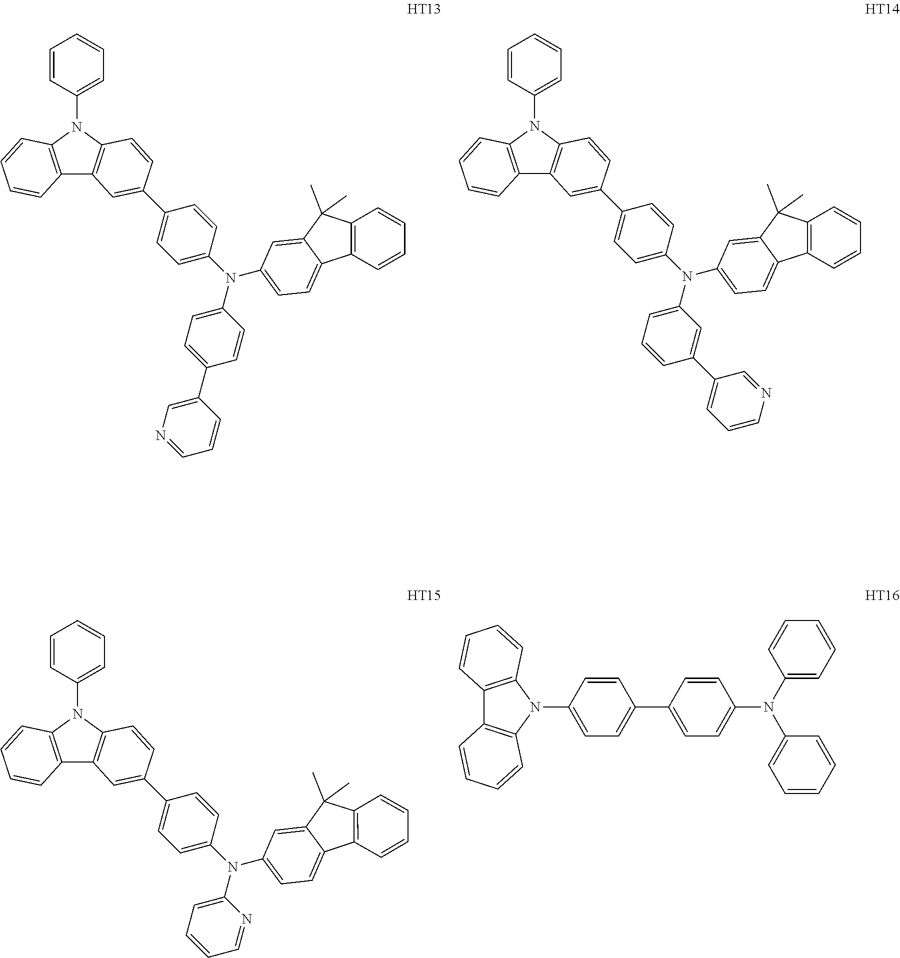 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

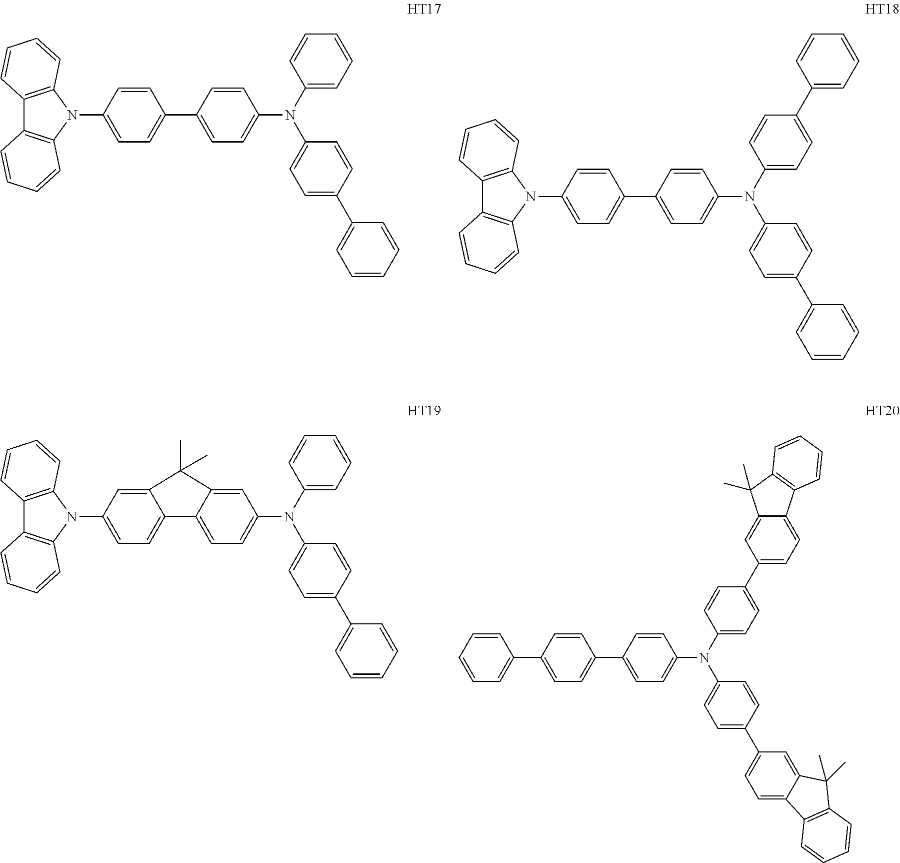 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

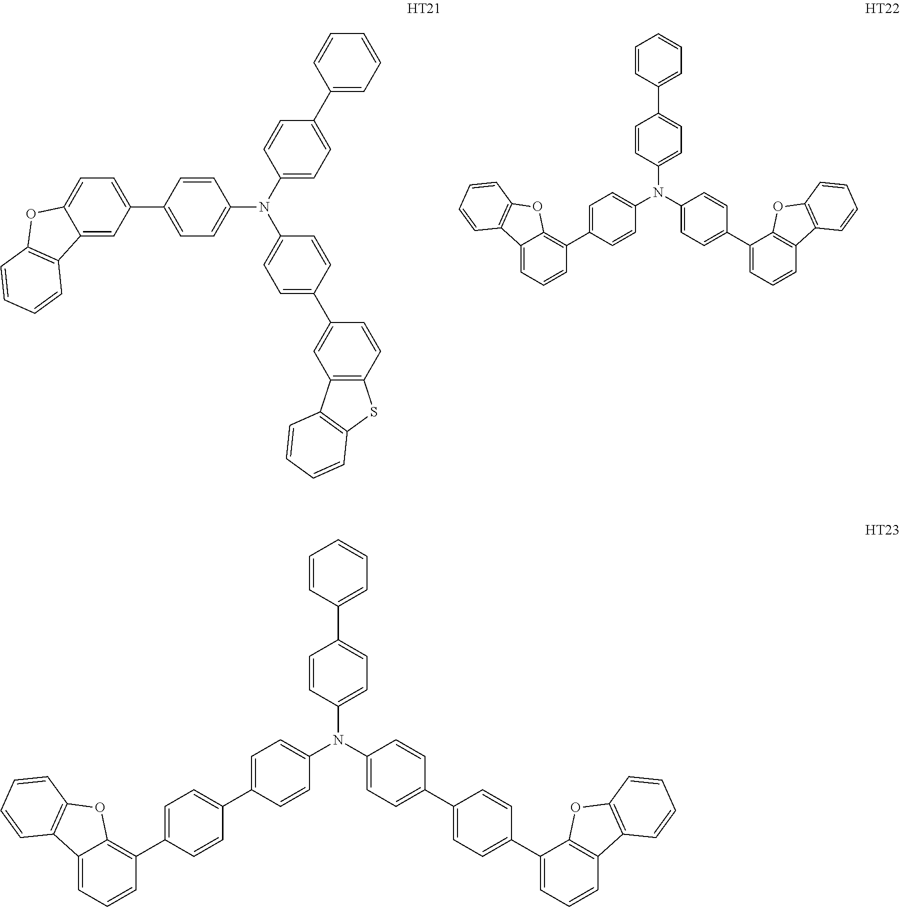 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

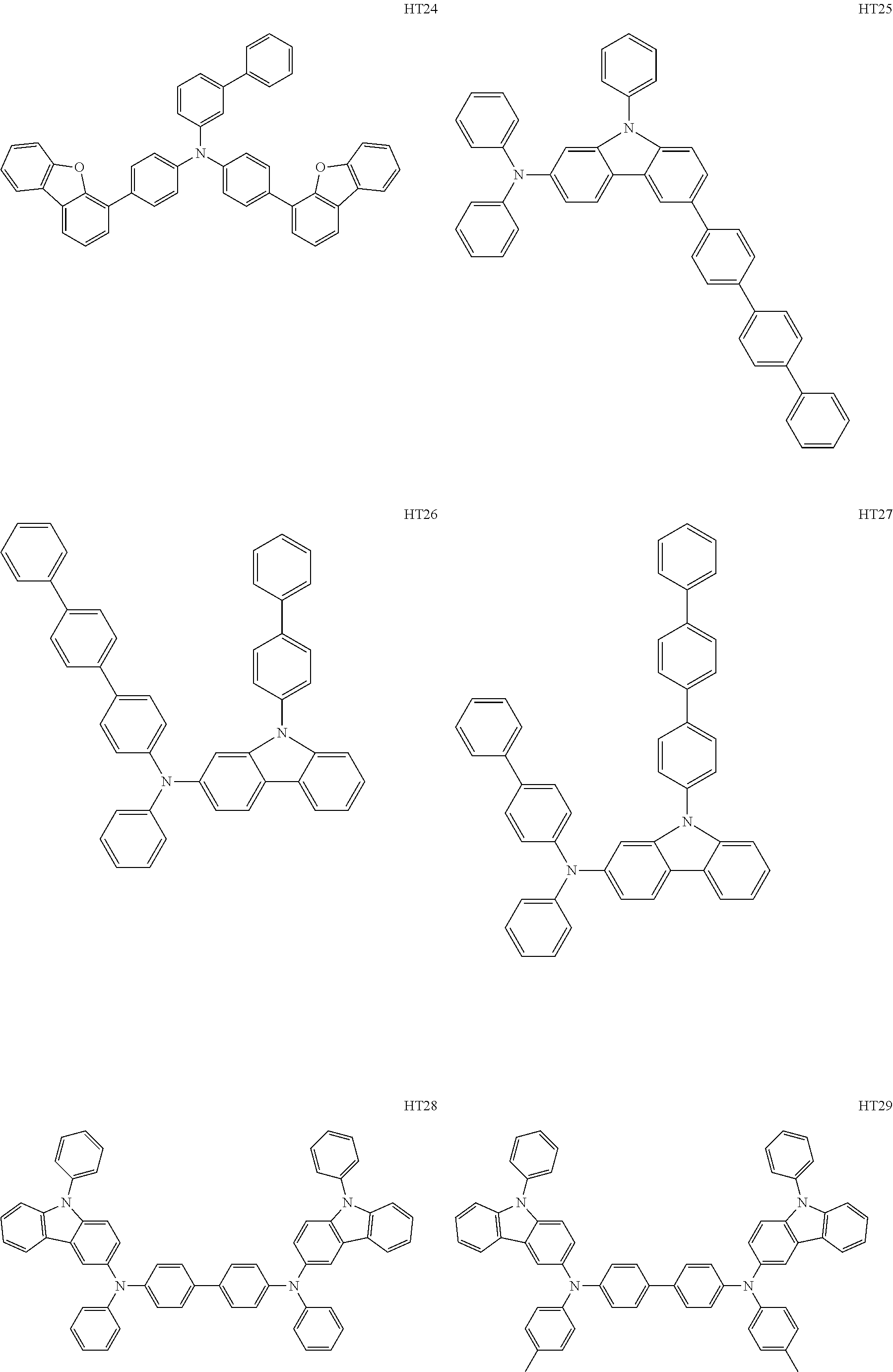

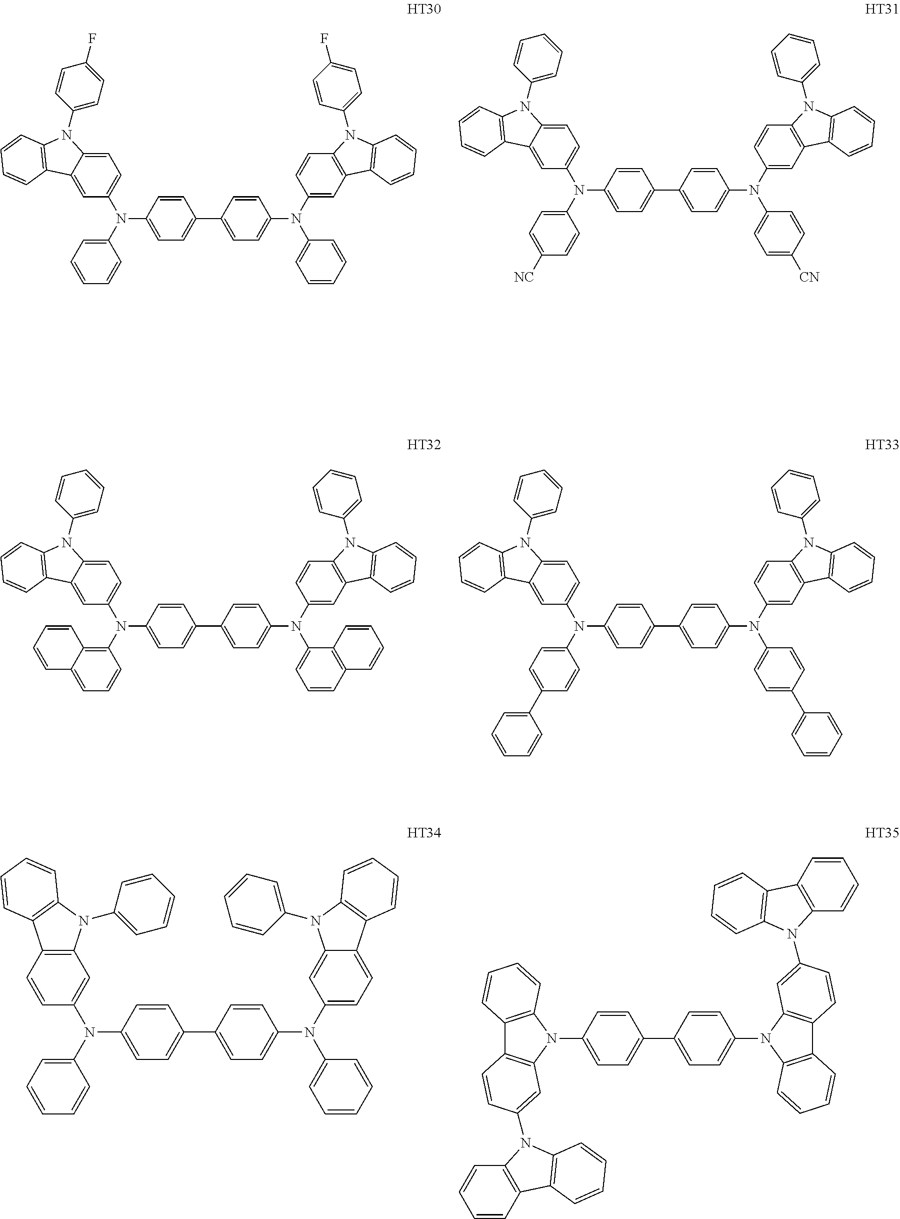

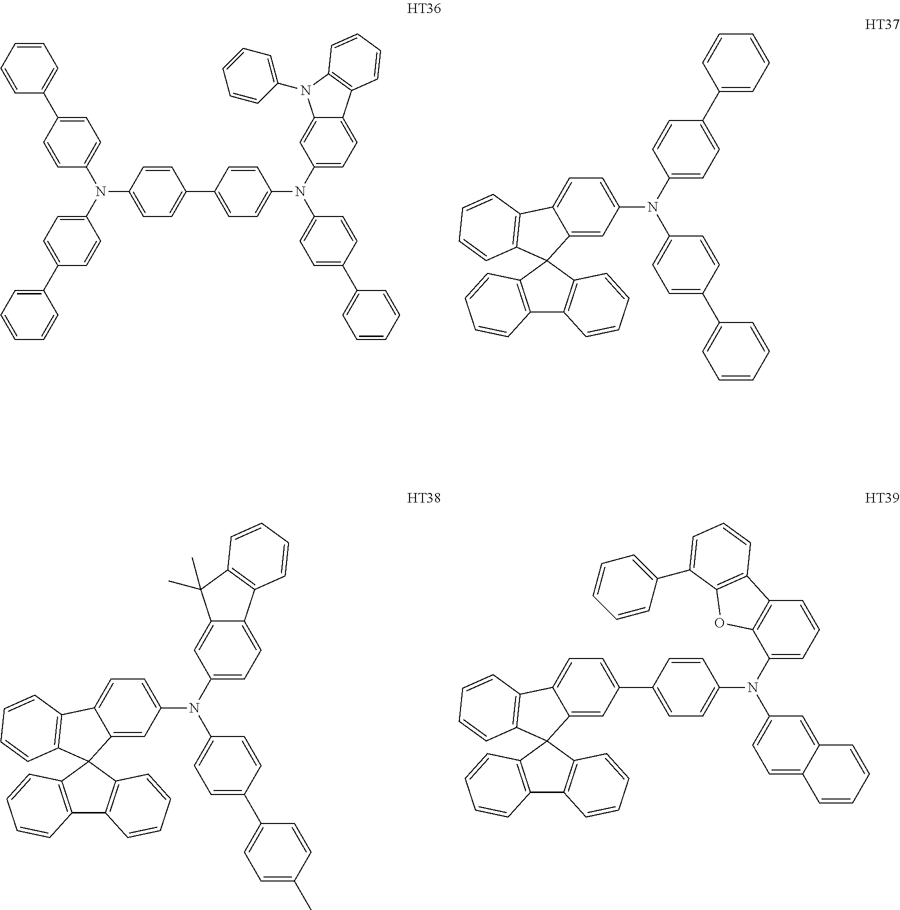 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

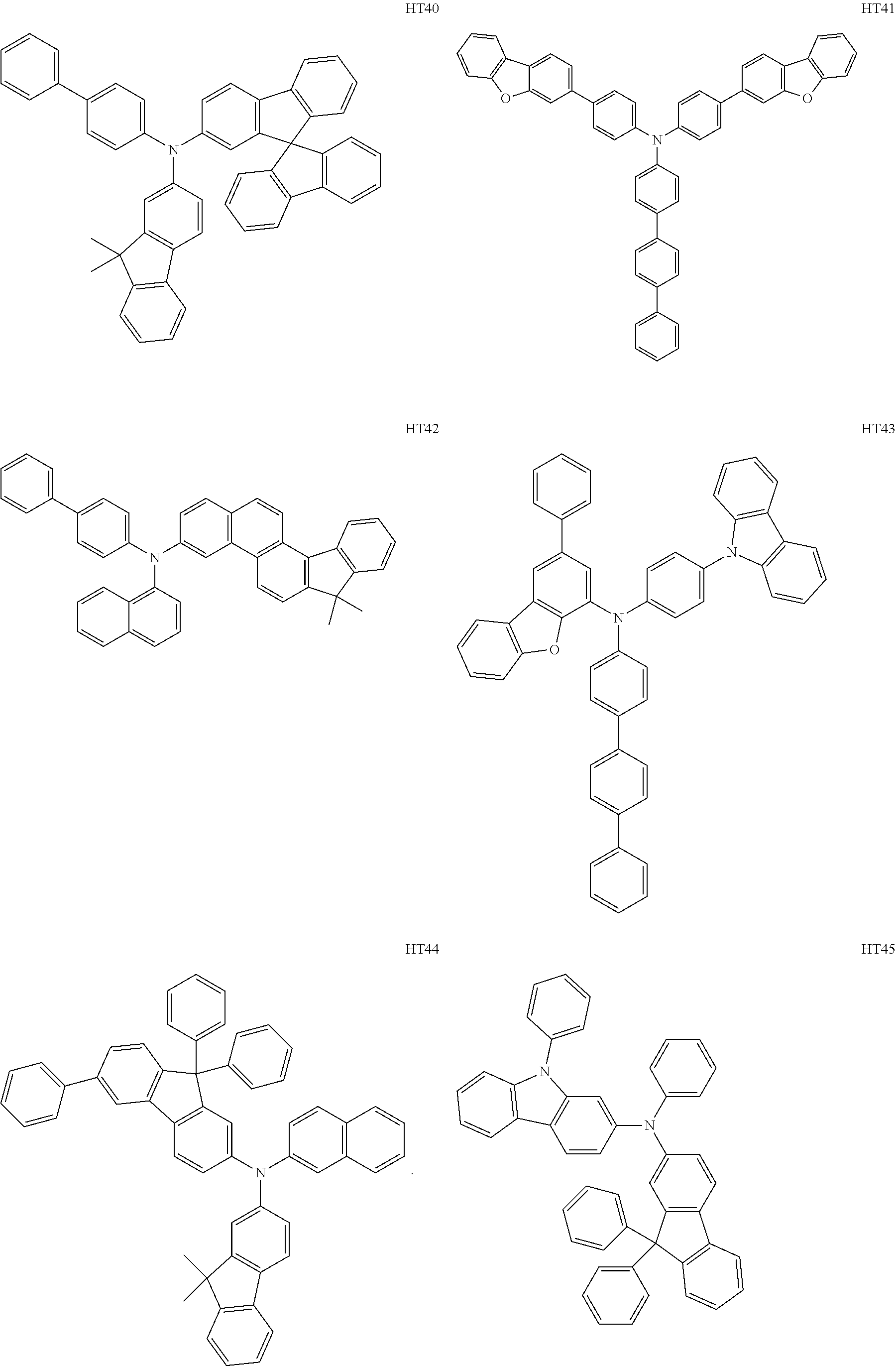

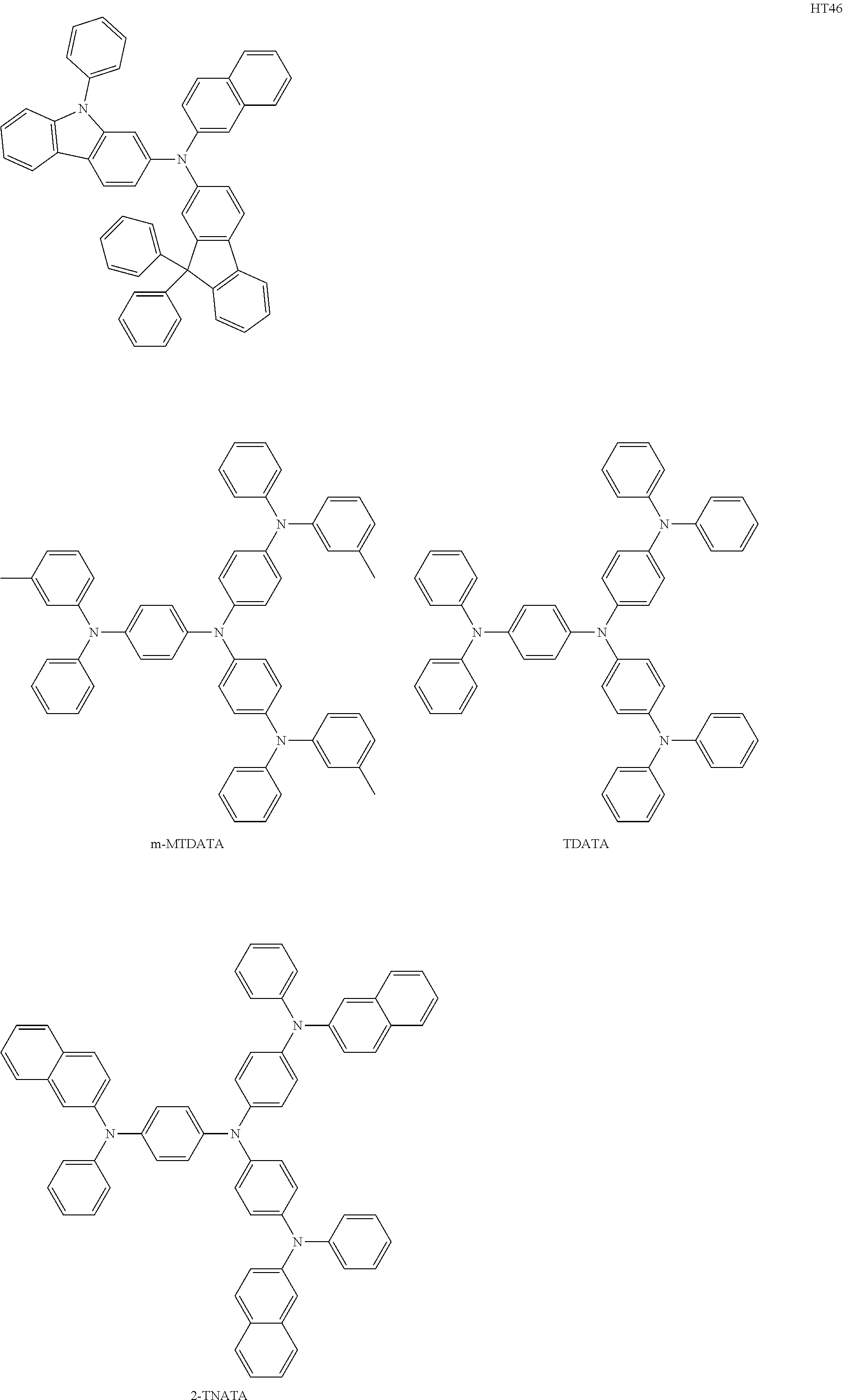

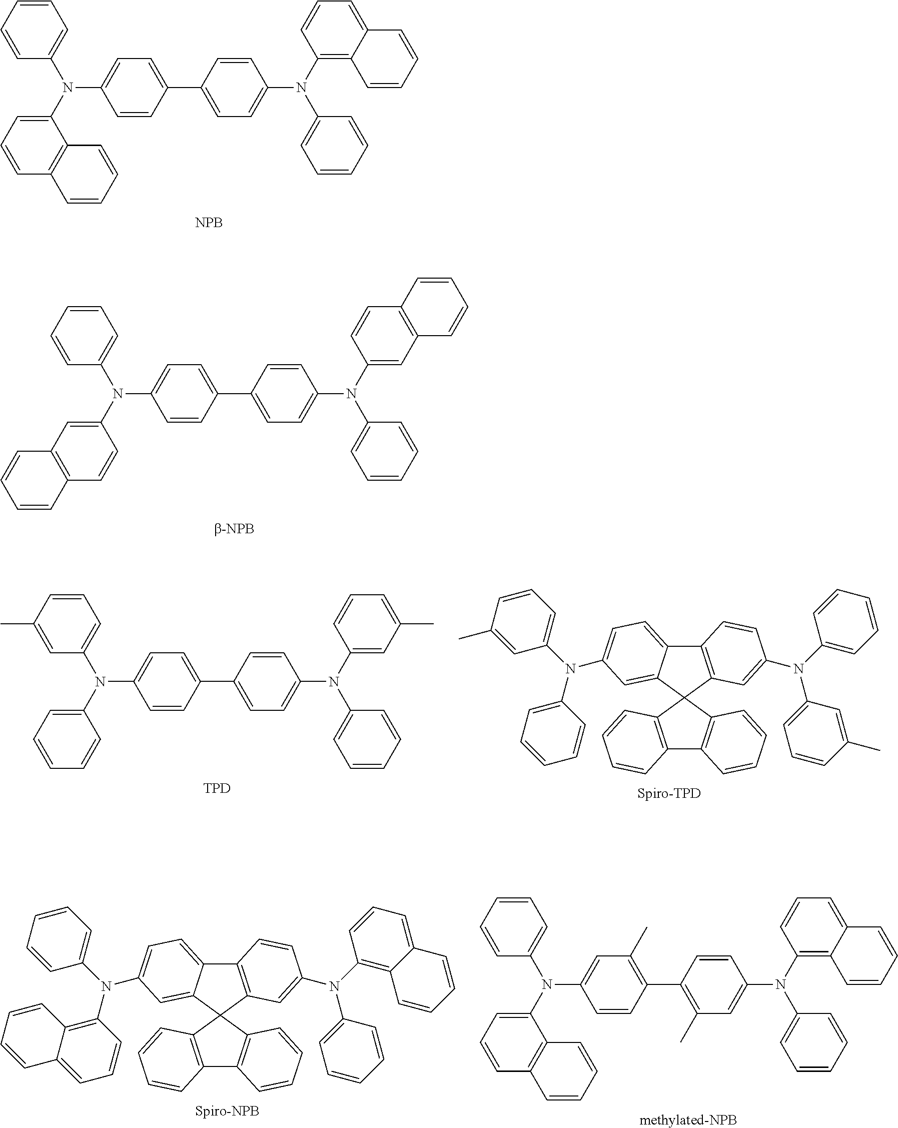

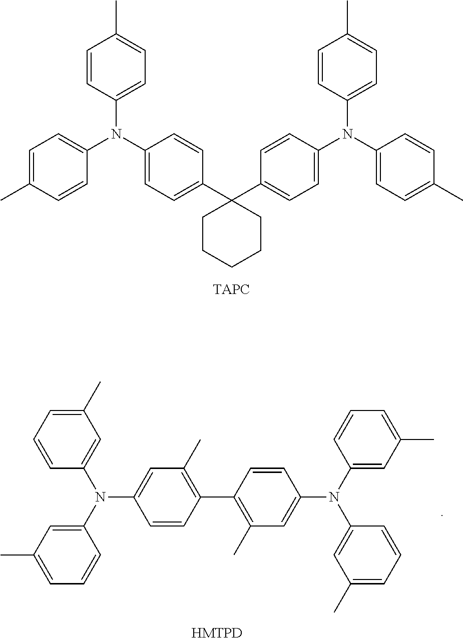

A thickness of the hole transport region may be in a range of about 50 Å to about 10,000 Å, for example, about 100 Å to about 4,000 Å. When the hole transport region includes a hole injection layer, a hole transport layer, or a combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, suitable or satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron blocking layer may block or reduce the leakage of electrons from an emission layer to a hole transport region. Materials that may be included in the hole transport region may be included in the emission auxiliary layer and the electron blocking layer.

p-dopant

The hole transport region may further include, in addition to these materials, a charge-generation material for the improvement of conductive properties (e.g., electrically conductive properties). The charge-generation material may be uniformly or non-uniformly dispersed in the hole transport region (for example, in the form of a single layer consisting of a charge-generation material).

The charge-generation material may be, for example, a p-dopant.

For example, the lowest unoccupied molecular orbital (LUMO) energy level of the p-dopant may be −3.5 eV or less.

In some embodiments, the p-dopant may include a quinone derivative, a cyano group-containing compound, a compound including element EL1 and element EL2, or a combination thereof.

Examples of the quinone derivative are TCNQ, F4-TCNQ, etc.

Examples of the cyano group-containing compound are HAT-CN and a compound represented by Formula 221 below.

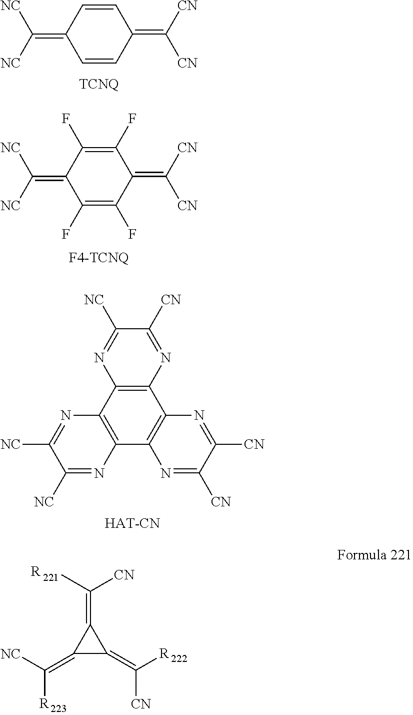

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or a combination thereof; or a combination thereof.

In the compound including element EL1 and element EL2, element EL1 may be metal, metalloid, or a combination thereof, and element EL2 may be non-metal, metalloid, or a combination thereof.

Examples of the metal are an alkali metal (for example, lithium (Li), sodium (Na), potassium (K), rubidium (Rb), cesium (Cs), etc.); alkaline earth metal (for example, beryllium (Be), magnesium (Mg), calcium (Ca), strontium (Sr), barium (Ba), etc.); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); post-transition metal (for example, zinc (Zn), indium (In), tin (Sn), etc.); and lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid are silicon (Si), antimony (Sb), and tellurium (Te).

Examples of the non-metal are oxygen (O) and halogen (for example, F, Cl, Br, I, etc.).

Examples of the compound including element EL1 and element EL2 are metal oxide, metal halide (for example, metal fluoride, metal chloride, metal bromide, and/or metal iodide), metalloid halide (for example, metalloid fluoride, metalloid chloride, metalloid bromide, and/or metalloid iodide), metal telluride, or a combination thereof.

Examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide are alkali metal halide, alkaline earth metal halide, transition metal halide, post-transition metal halide, and lanthanide metal halide.

Examples of the alkali metal halogen may include LiF, NaF, KF, RbF, CsF, LiCI, NaCl, KCl, RbCl, CsCl, LiBr, NaBr, KBr, RbBr, CsBr, LiI, NaI, KI, RbI, and CsI.

Examples of the alkaline earth metal halide are BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, Bel₂, Mgl₂, Cal₂, Srl₂, and Bal₂.

Examples of the transition metal halide are titanium halide (for example, TiF₄, TiCl₄, TiBr₄, Til₄, etc.), zirconium halide (for example, ZrF₄, ZrCl₄, ZrBr₄, Zrl₄, etc.), hafnium halide (for example, HfF₄, HfCl₄, HfBr₄, Hfl₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCI₃, WBr₃, WI₃, etc.), manganese halide (for example, MnF₂, MnCl₂, MnBr₂, MnI₂, etc.), technetium halide (for example, TcF₂, TcCl₂, TcBr₂, Tcl₂, etc.), rhenium halide (for example, ReF₂, ReCl₂, ReBr₂, Rel₂, etc.), iron halide (for example, FeF₂, FeCl₂, FeBr₂, Fel₂, etc.), ruthenium halide (for example, RuF₂, RuCl₂, RuBr₂, Rul₂, etc.), osmium halide (for example, OsF₂, OsCl₂, OsBr₂, OsI₂, etc.), cobalt halide (for example, CoF₂, COCl₂, CoBr₂, CoI₂, etc.), rhodium halide (for example, RhF₂, RhCl₂, RhBr₂, RhI₂, etc.), iridium halide (for example, IrF₂, IrI₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, Cul, etc.), silver halide (for example, AgF, AgCl, AgBr, AgI, etc.), and gold halide (for example, AuF, AuCl, AuBr, AuI, etc.).

Examples of the post-transition metal halide are zinc halide (for example, ZnF₂, ZnCl₂, ZnBr₂, ZnI₂, etc.), indium halide (for example, Ink₃, etc.), and tin halide (for example, SnI₂, etc.).

Examples of the lanthanide metal halide are YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, SmI₃, etc.

An example of the metalloid halide is antimony halide (for example, SbCl₅, etc.).

Examples of the metal telluride are alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In some embodiments, the emission layer may have a stacked structure of two or more layers of a red emission layer, a green emission layer, and a blue emission layer, in which the two or more layers contact each other or are separated from each other to emit white light. In some embodiments, the emission layer may include two or more materials of a red light-emitting material, a green light-emitting material, and a blue light-emitting material, in which the two or more materials are mixed together with each other in a single layer to emit white light.

The emission layer may include a host and a dopant. The dopant may include a phosphorescent dopant, a fluorescent dopant, or a combination thereof.

The amount of the dopant in the emission layer may be from about 0.01 part by weight to about 15 parts by weight based on 100 parts by weight of the host.

In some embodiments, the emission layer may include a quantum dot.

In some embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within these ranges, excellent light-emission characteristics may be obtained without a substantial increase in driving voltage.

### Host

In some embodiments, the host may include a compound represented by Formula 301 below:

[Ar₃₀₁]_{xb11}-[(L₃₀₁)_{xb1}-R₃₀₁]_{xb21} Formula 301

In Formula 301, Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and

Q₃₀₁ to Q₃₀₃ may each be as described for Q₁.

For example, when xb11 in Formula 301 is 2 or more, two or more of Ar₃₀₁ (s) may be linked to each other via a single bond.

In some embodiments, the host may include a compound represented by Formula 301-1, a compound represented by Formula 301-2, or a combination thereof:

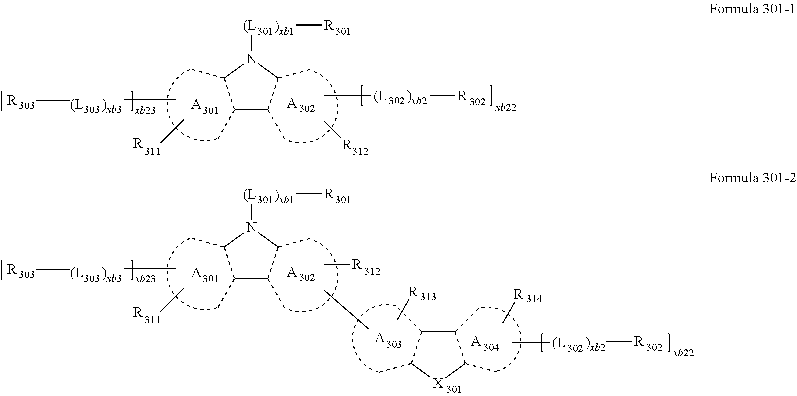

wherein, in Formula 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as described herein,
L₃₀₂ to L₃₀₄ may each independently be as described for L₃₀₁,
xb2 to xb4 may each independently be as described for xb1, and
R₃₀₂ to R₃₀₅ and R₃₁₁ to R₃₁₄ may each be as described for R₃₀₁.

In some embodiments, the host may include an alkali earth metal complex, a post-transition metal complex, or a combination thereof. For example, the host may include a Be complex (for example, Compound H55), an Mg complex, a Zn complex, or a combination thereof.

In one or more embodiments, the host may include: one selected from Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di-9-carbazolylbenzene (mCP); 1,3,5-tri(carbazol-9-yl)benzene (TCP); or a combination thereof:

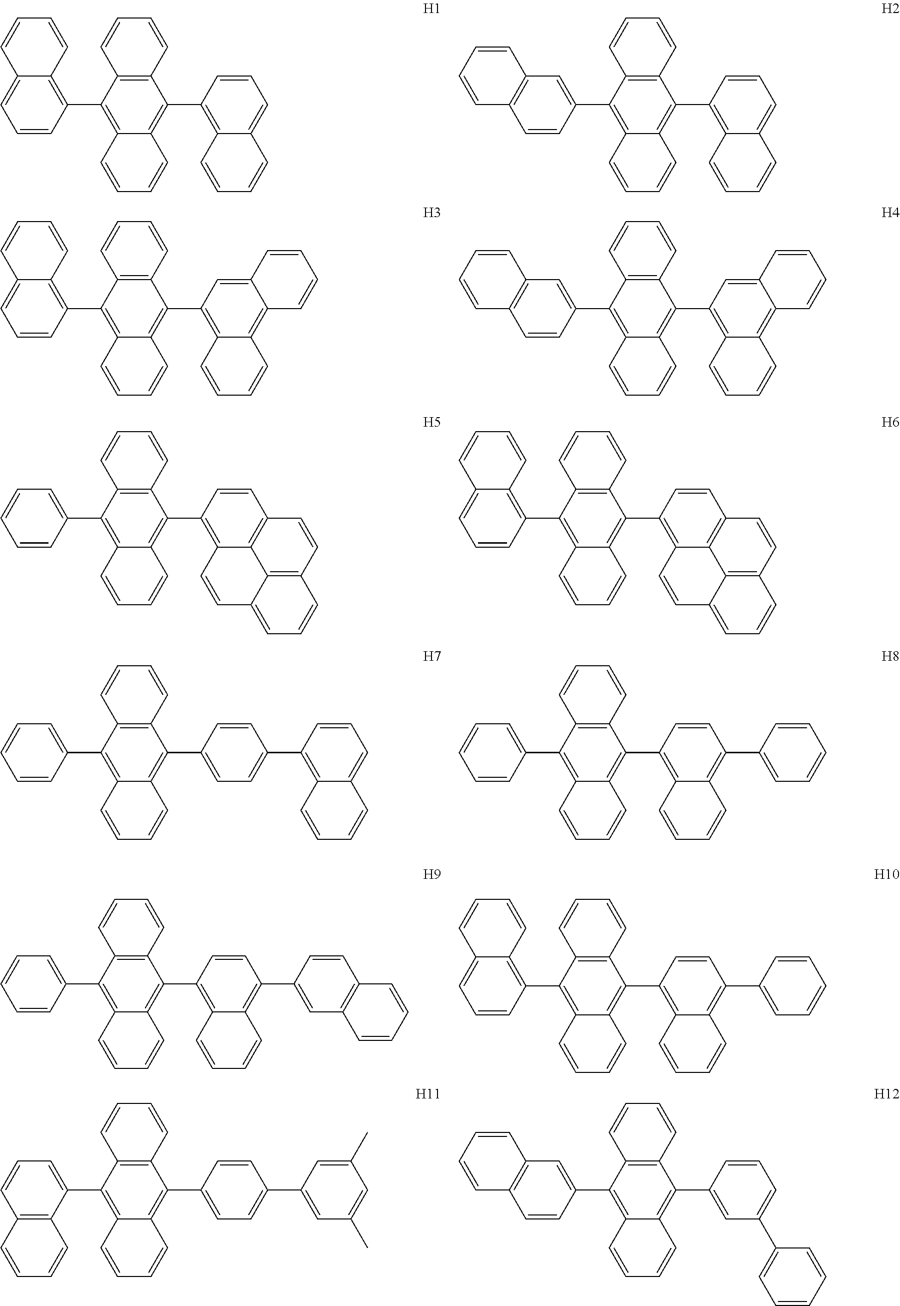

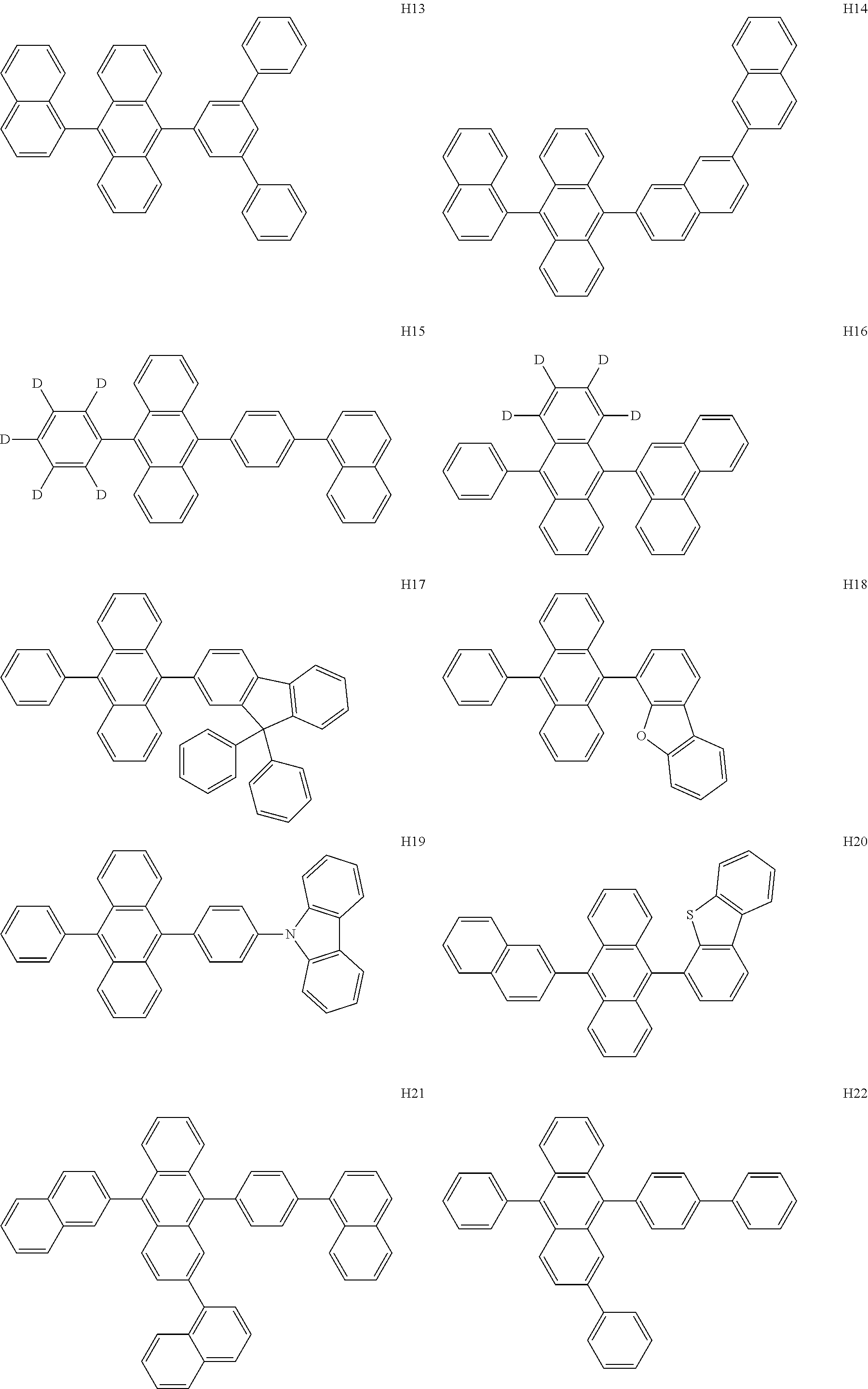

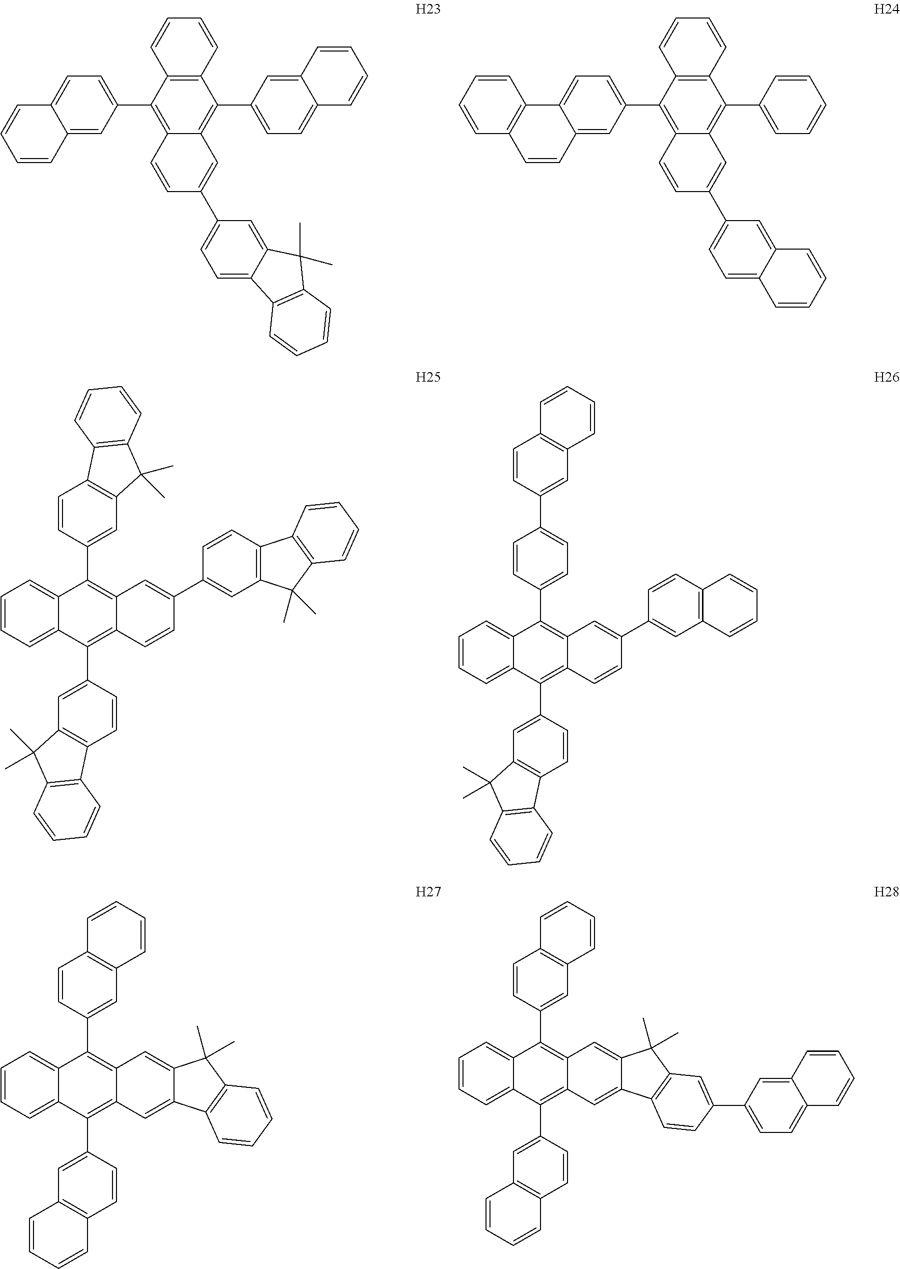

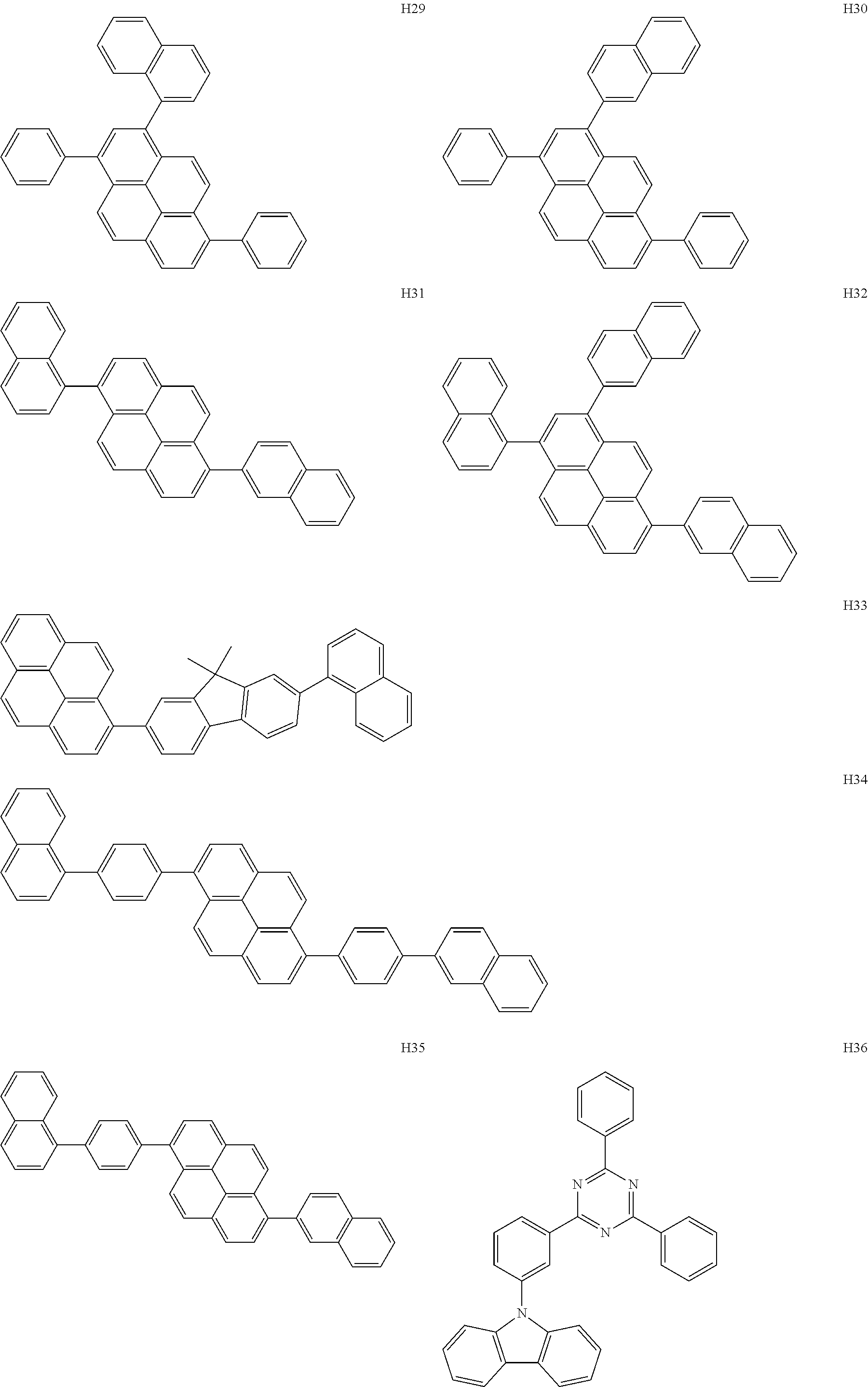

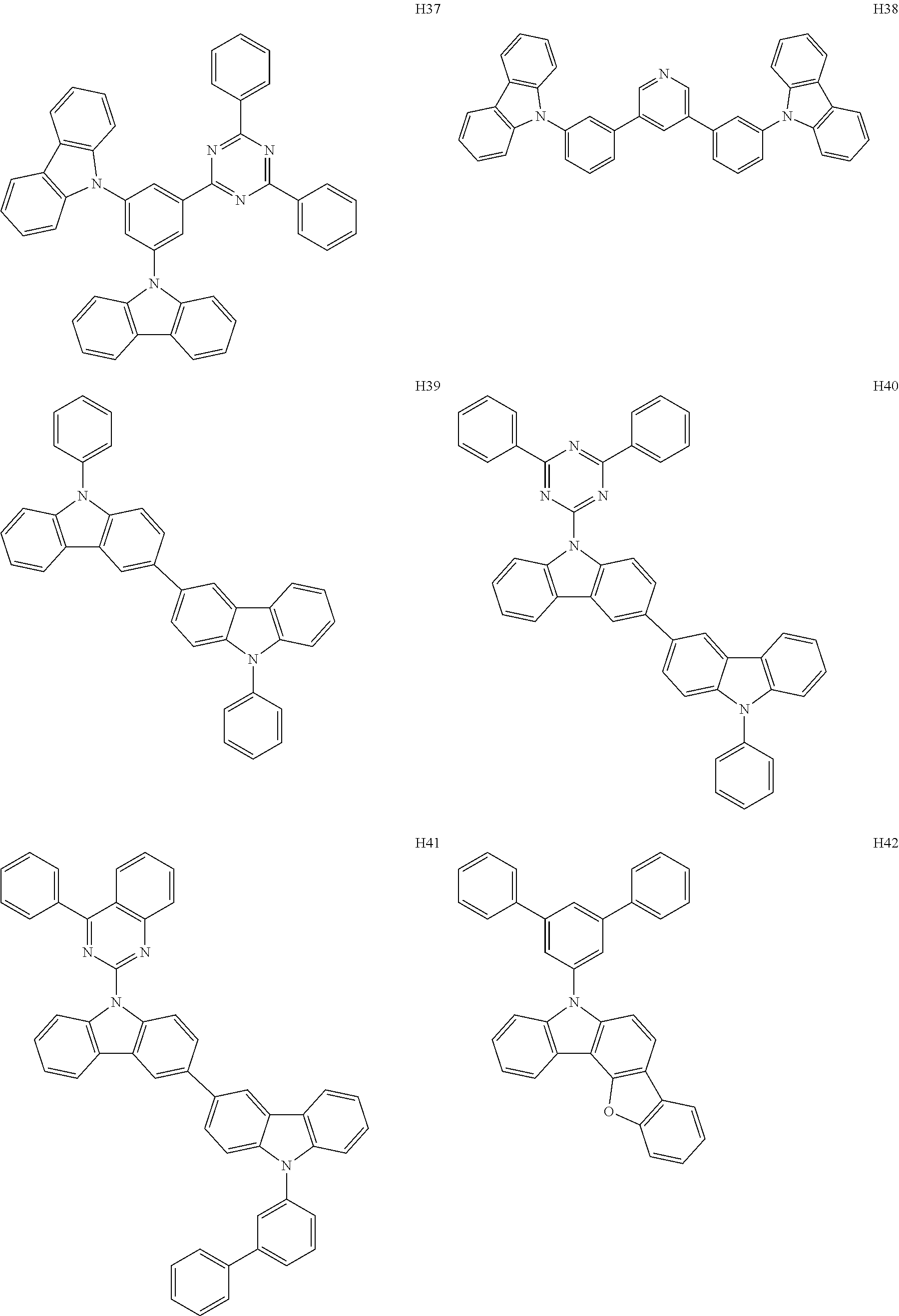

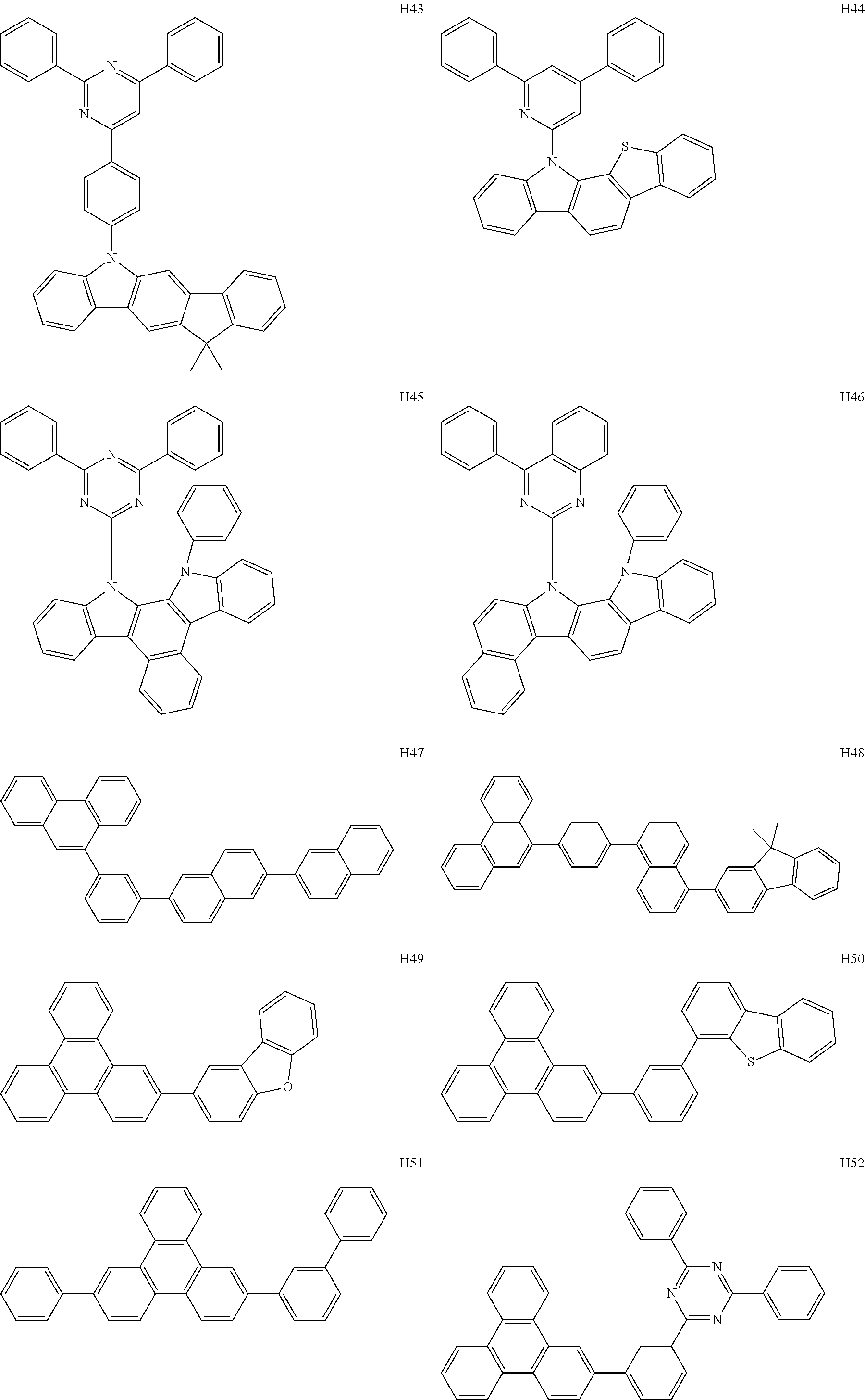

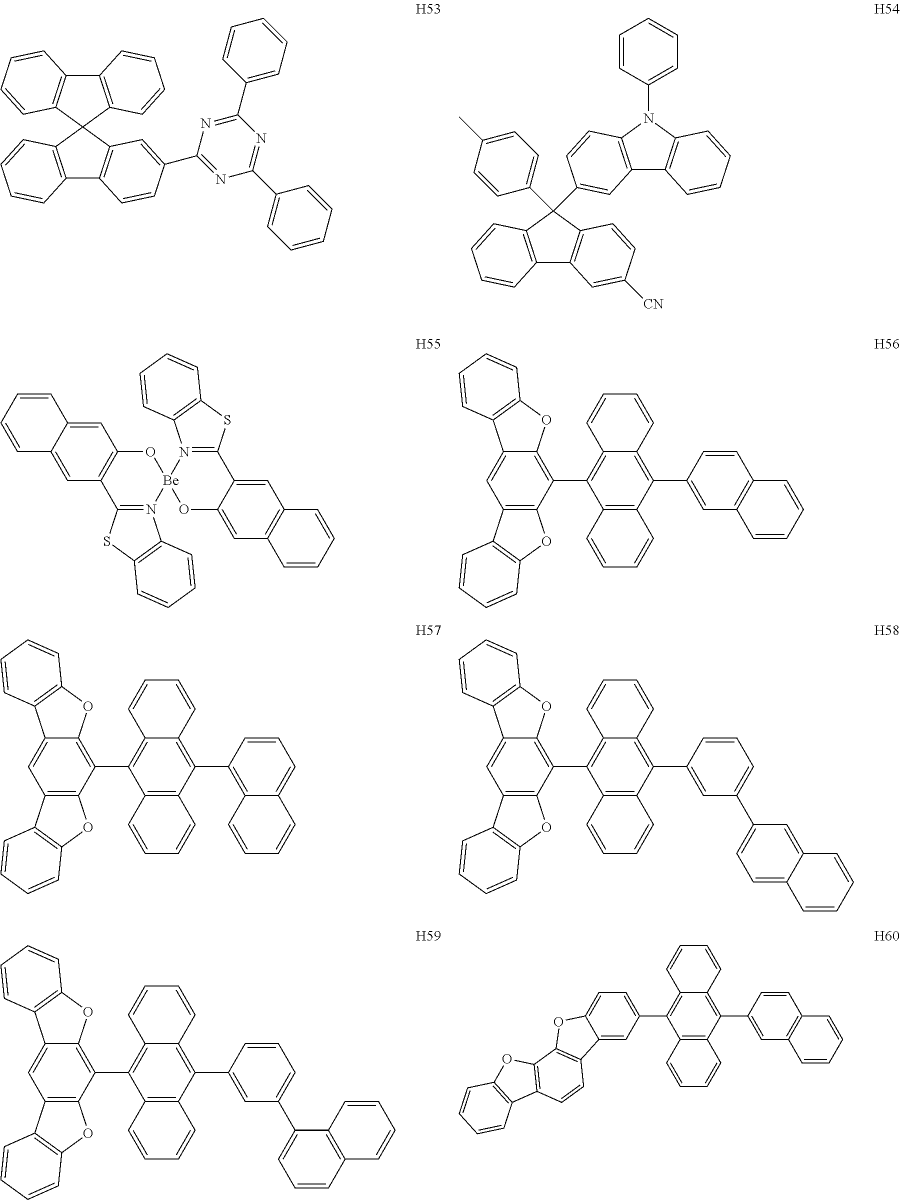

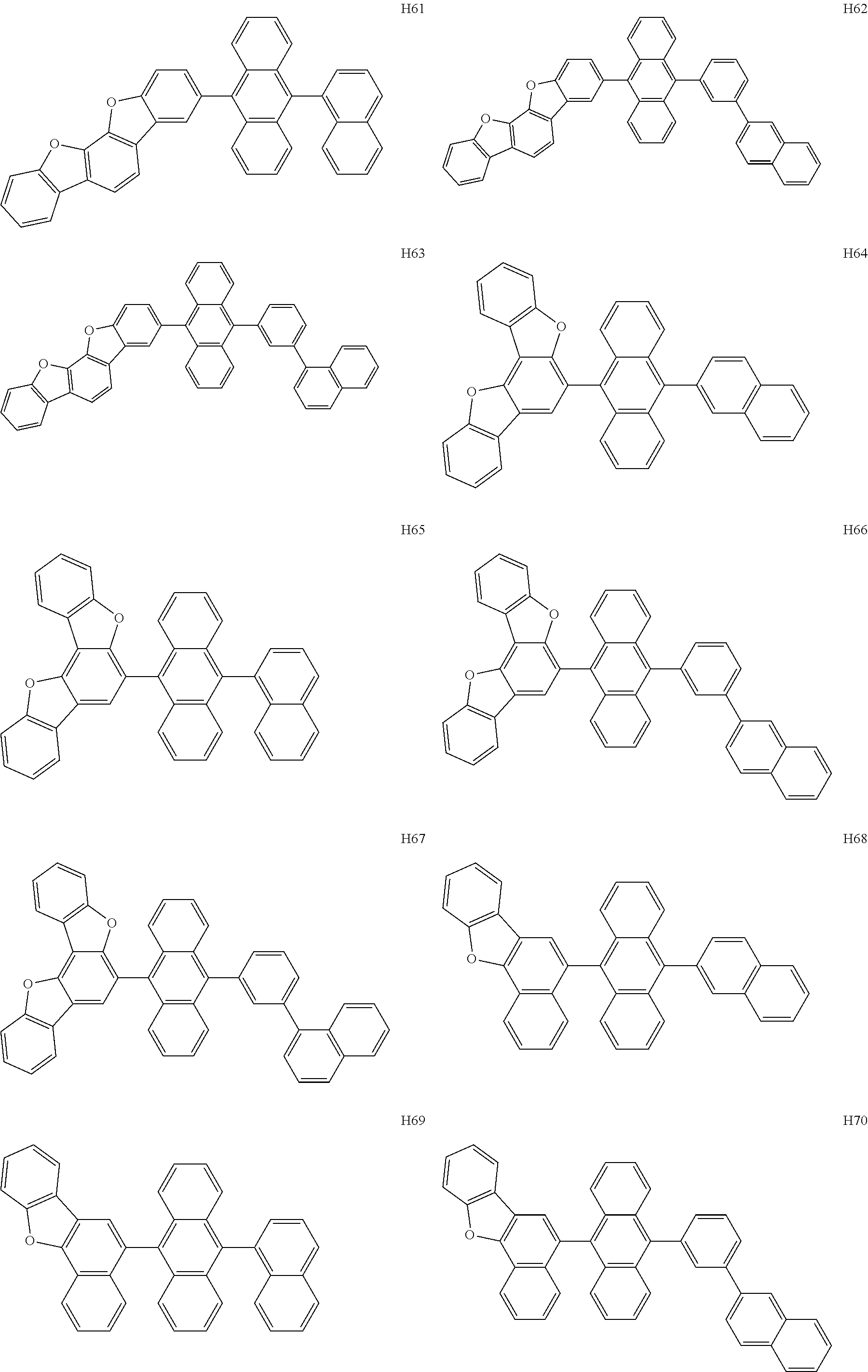

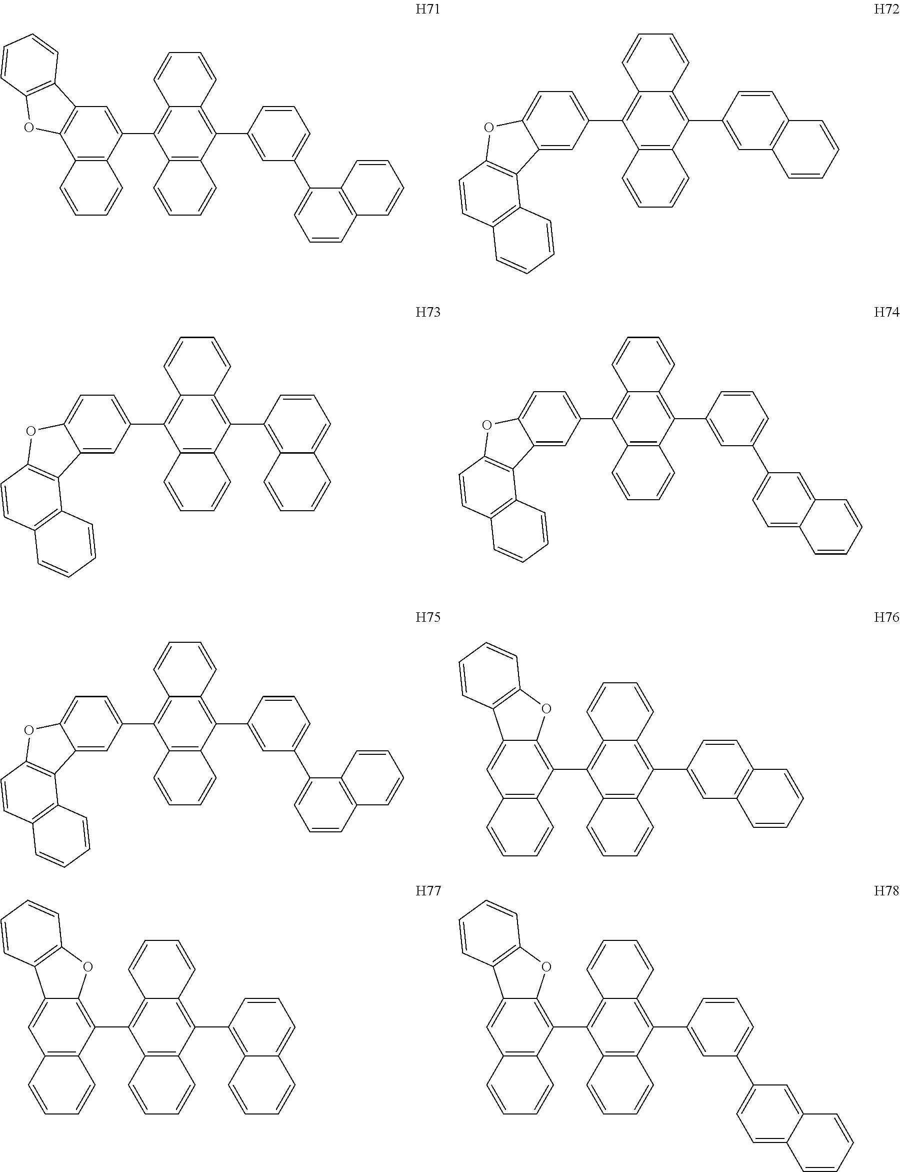

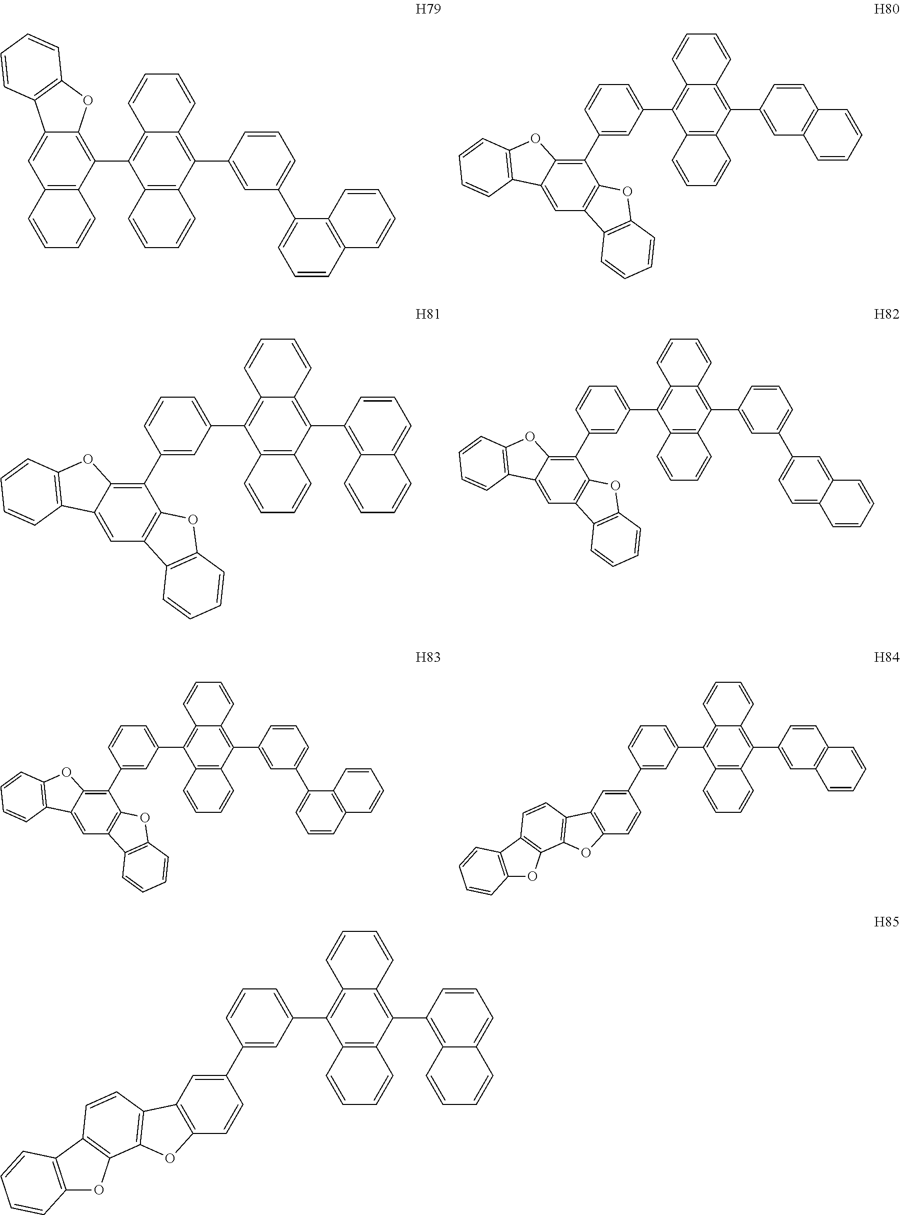

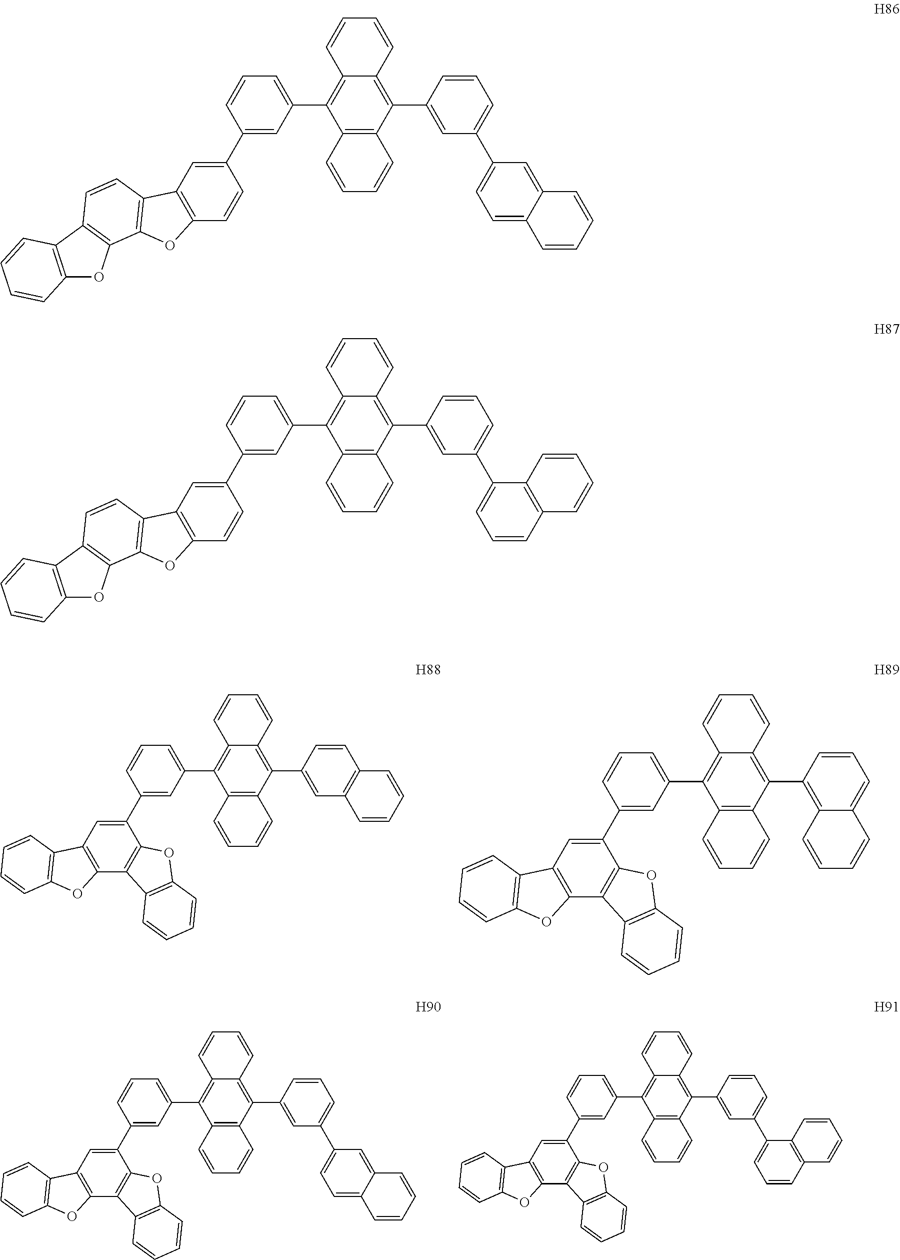

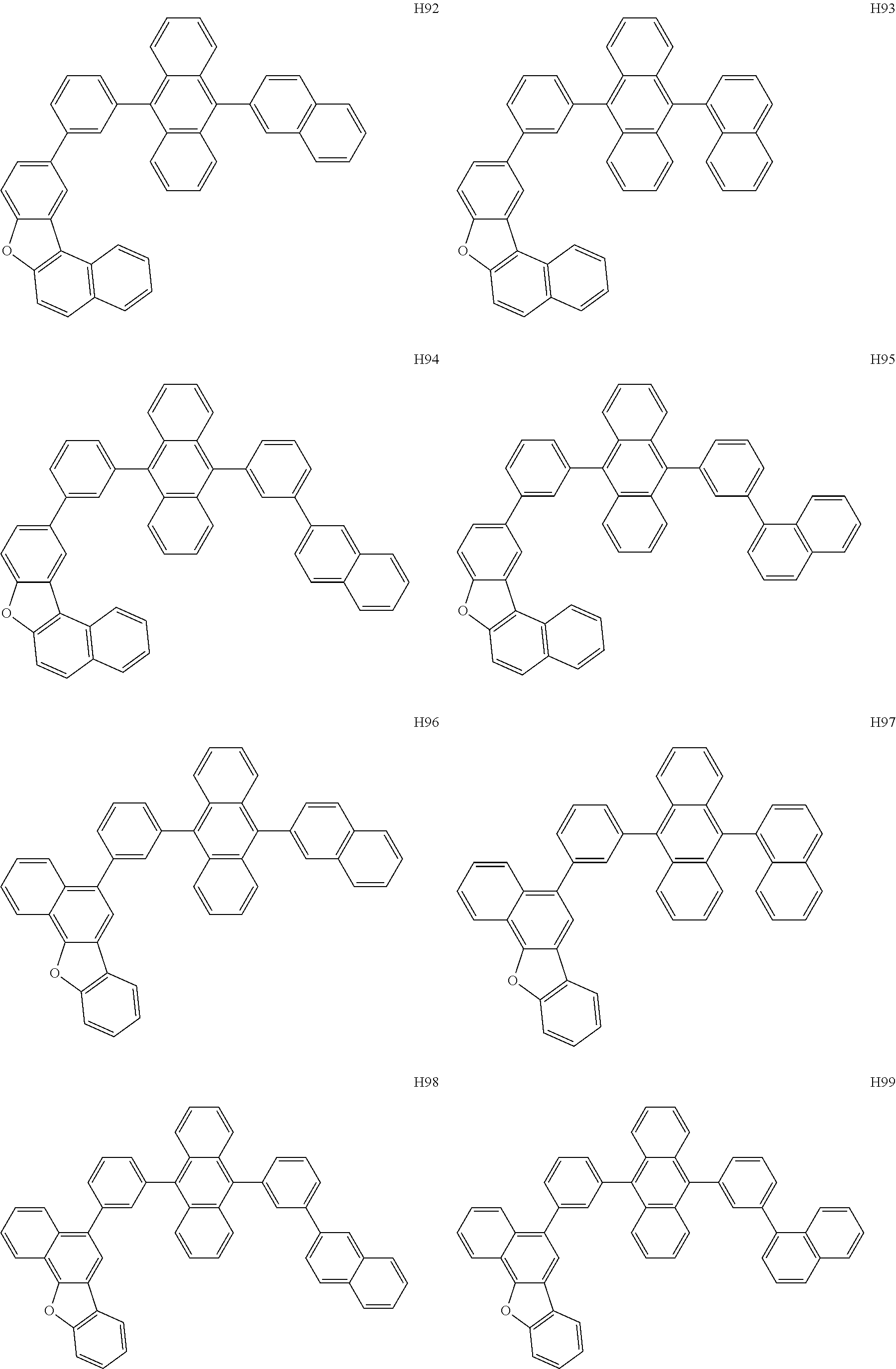

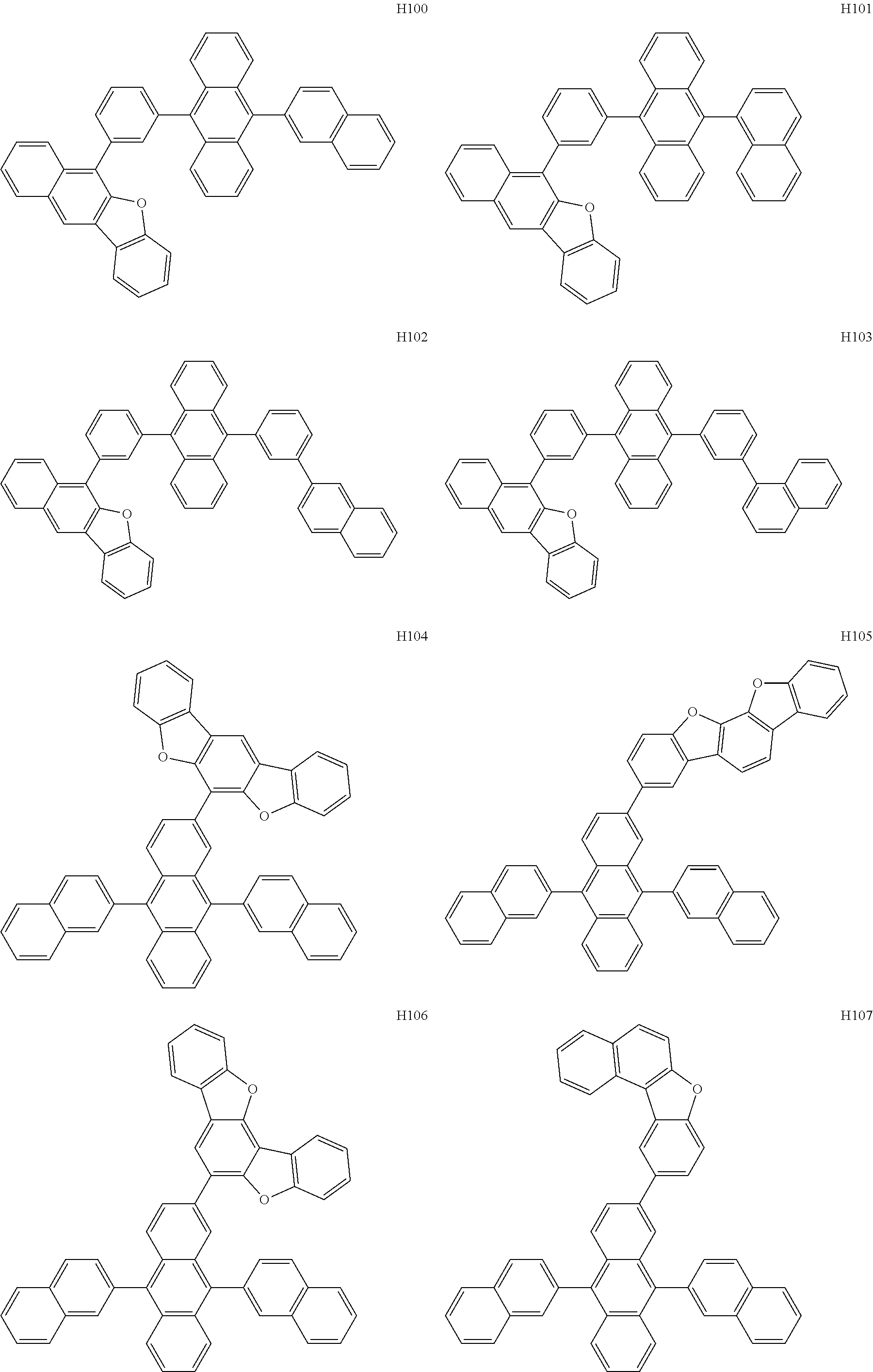

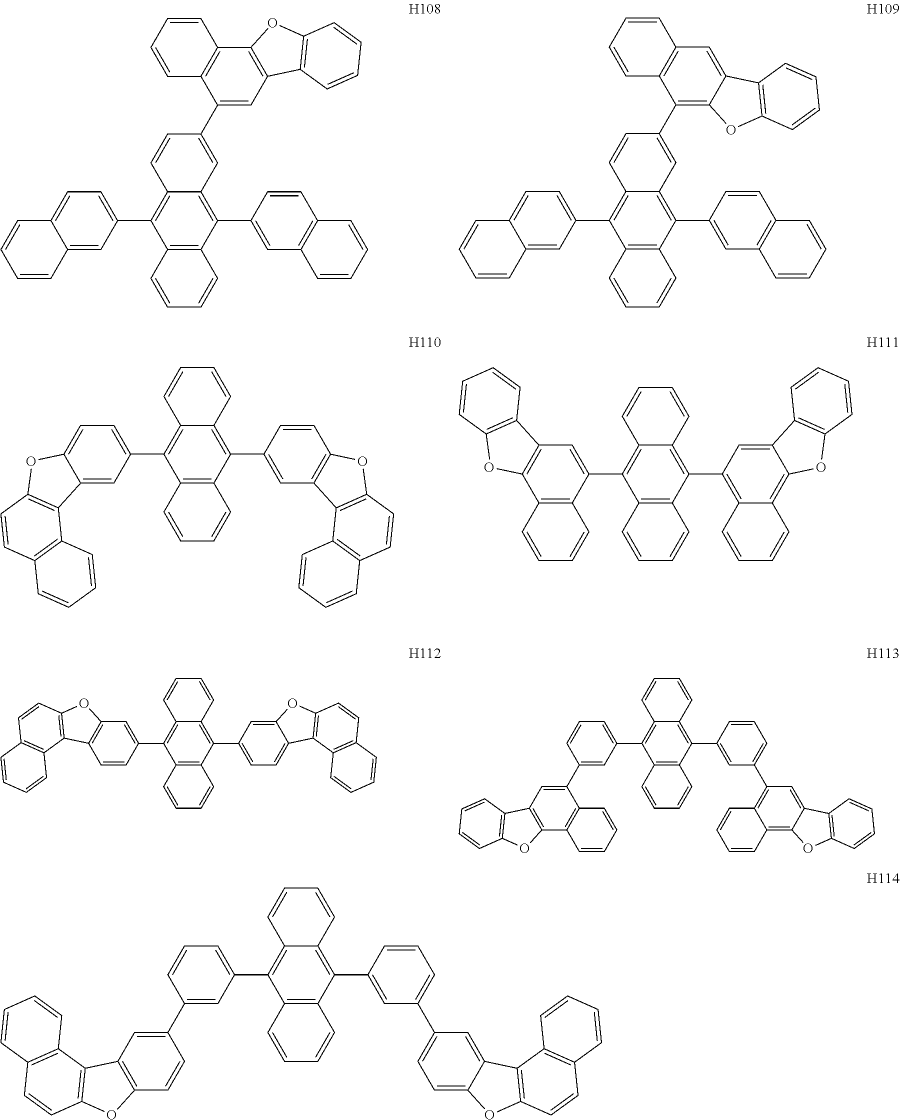

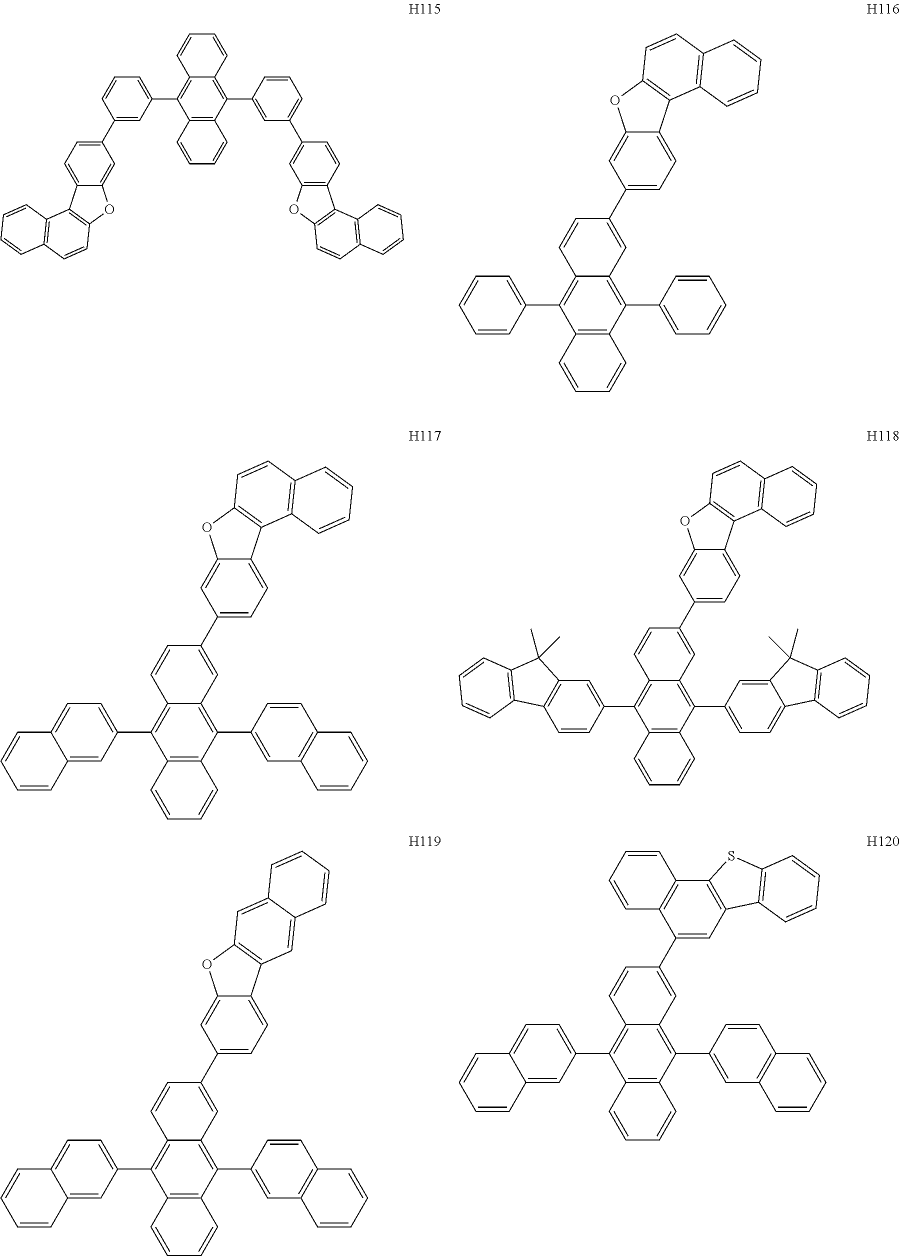

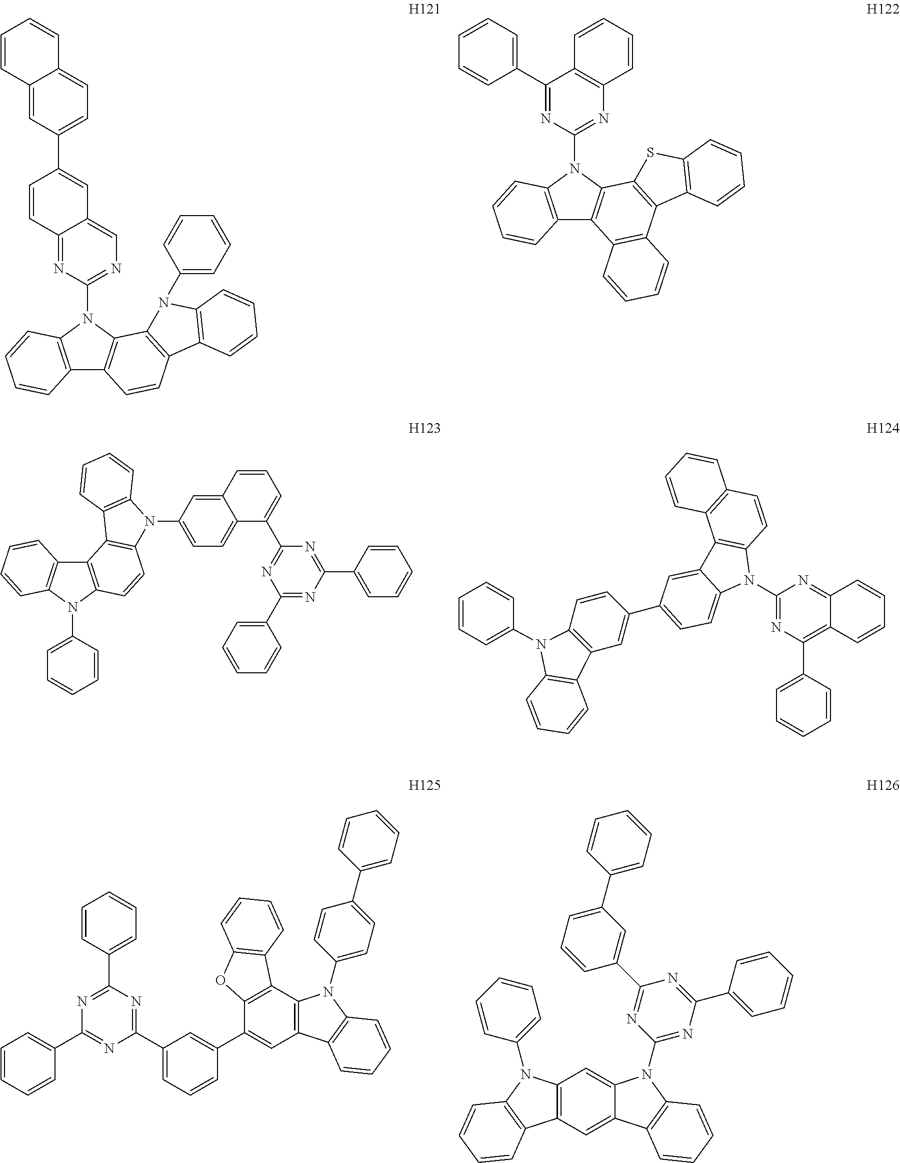

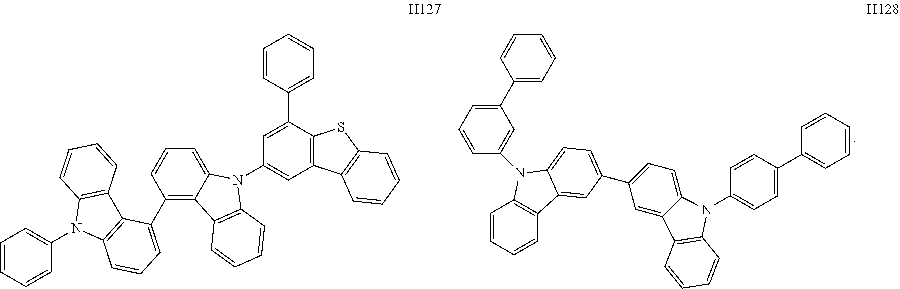 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may be the organometallic compound represented by Formula 1.

In some embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or a combination thereof.

The phosphorescent dopant may be electrically neutral.

For example, the phosphorescent dopant may include an organometallic compound represented by Formula 401:

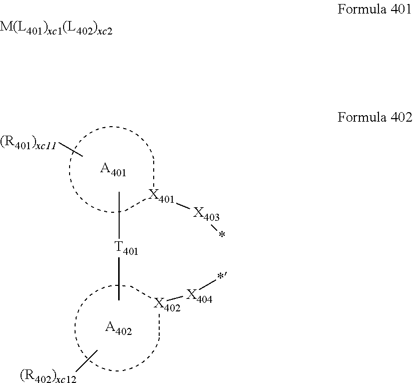

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₀₁ (s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C═*′,
X₄₀₃ and X₄₀₄ may each independently be a chemical bond (for example, a covalent bond or a coordination bond (which may also be referred to as a coordinate covalent bond or a dative bond)), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be as described for Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be as described for Q₁,
xc11 and xc12 may each independently be an integer from 0 to 10, and* and *′ in Formula 402 each indicates a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁ (s) among two or more of L₄₀₁ may optionally be bonded to each other via T₄₀₂, which is a linking group, and two ring A₄₀₂(s) among two or more of L₄₀₁ may optionally be bonded to each other via T₄₀₃, which is a linking group (see Compounds PD1 to PD4 and PD7). T₄₀₂ and T₄₀₃ may each be as described for T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or a combination thereof.

The phosphorescent dopant may include, for example, one selected from compounds PD1 to PD39, or a combination thereof:

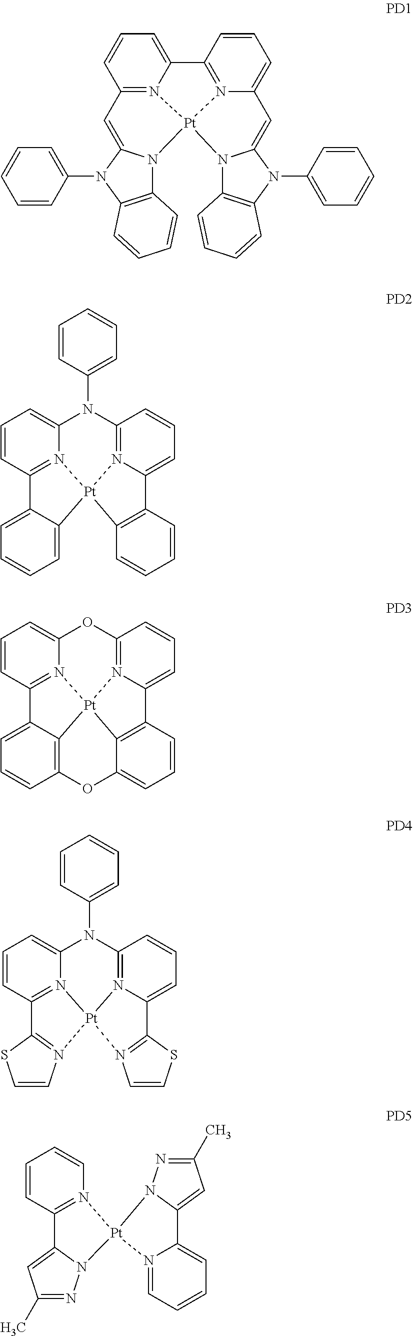

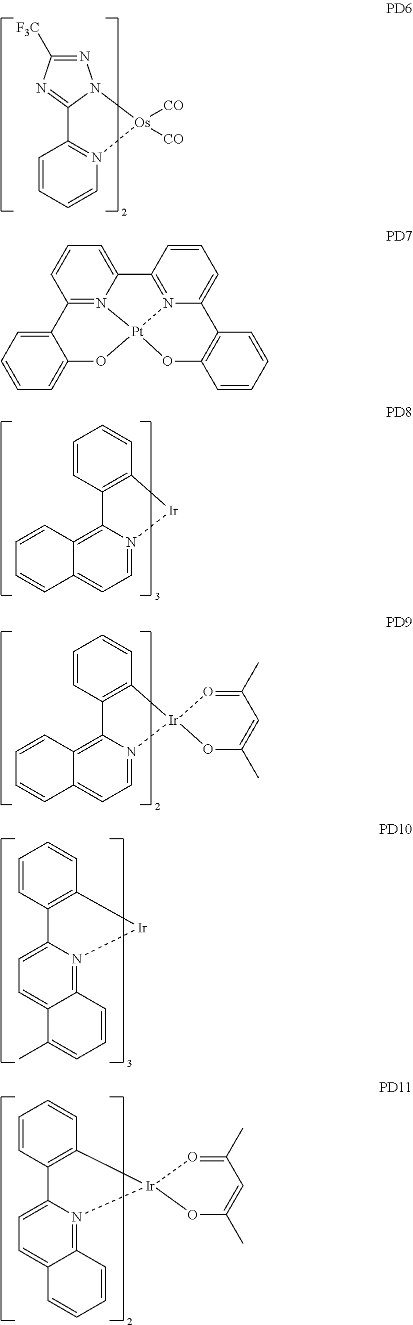

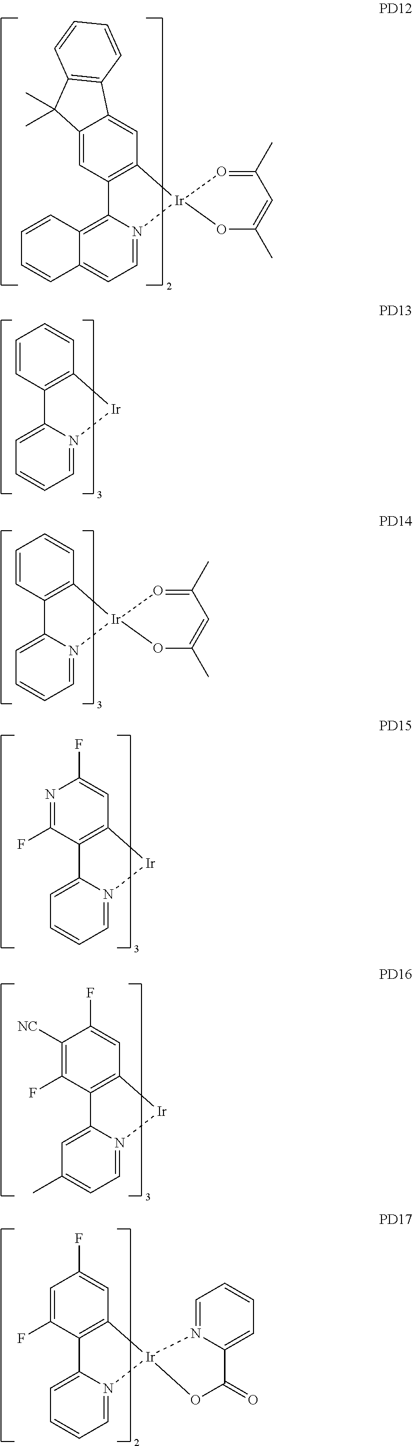

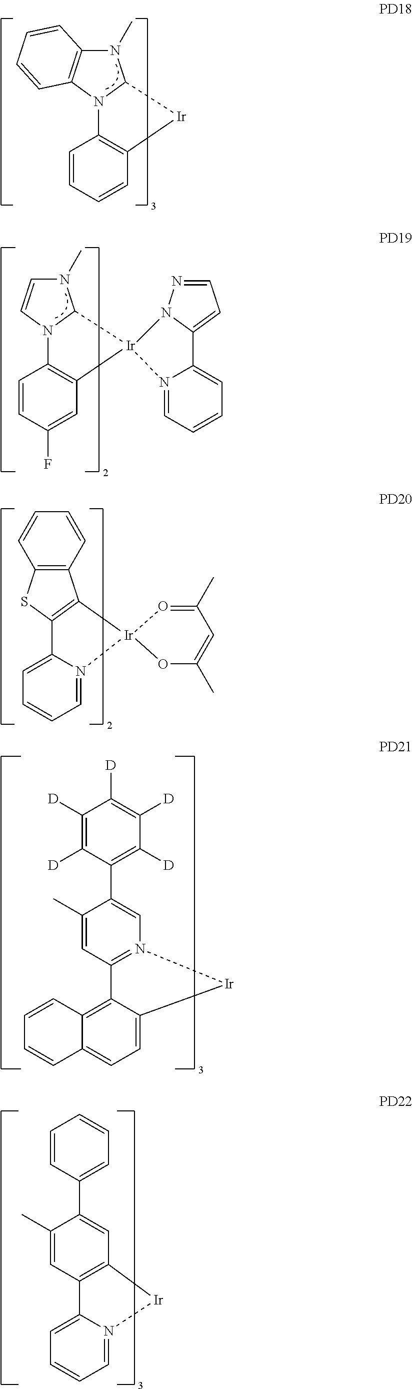

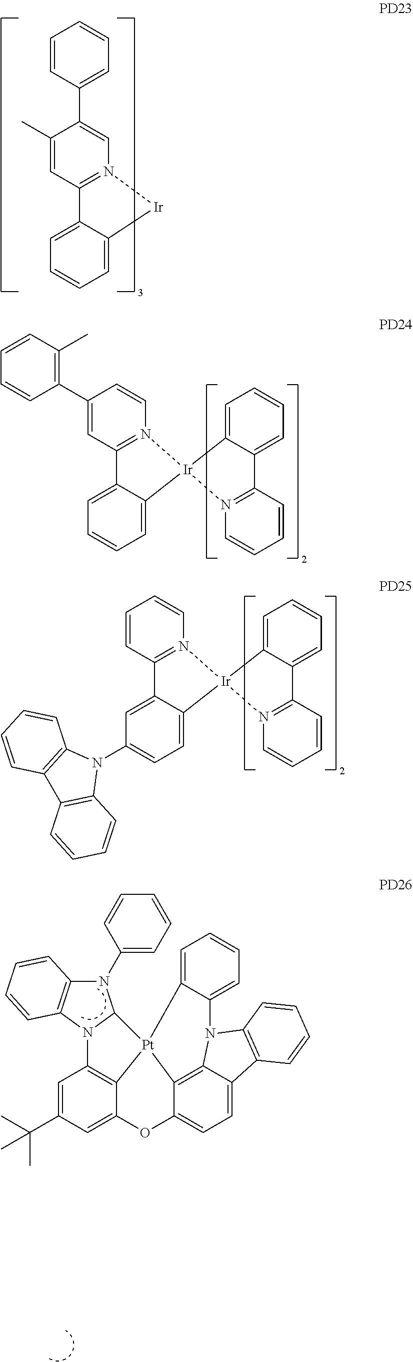

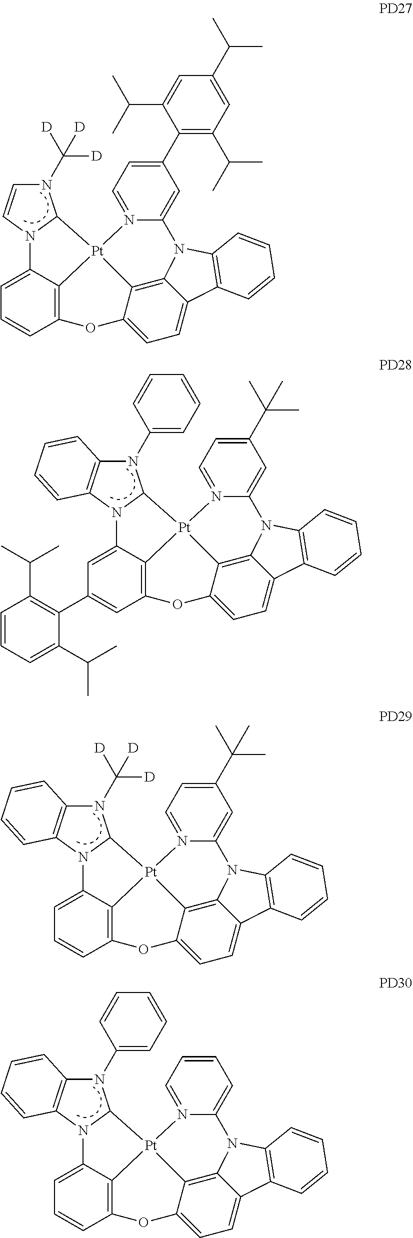

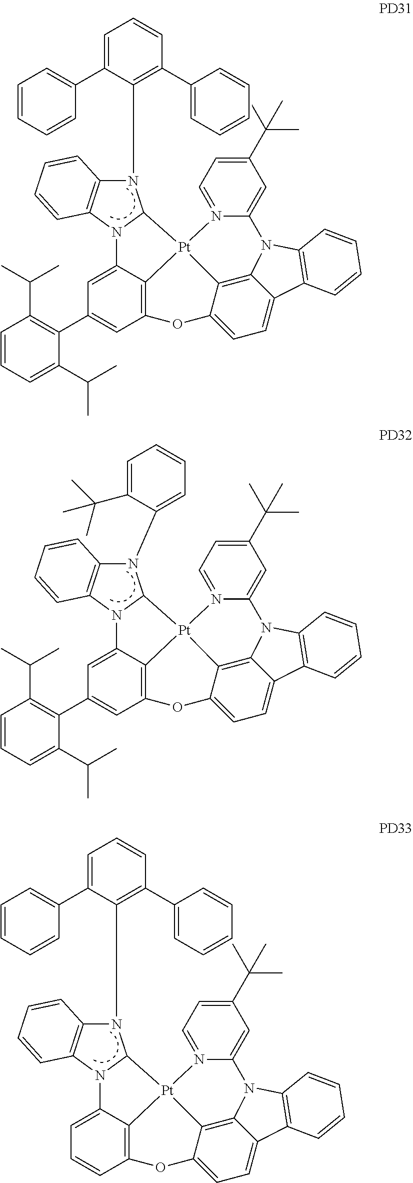

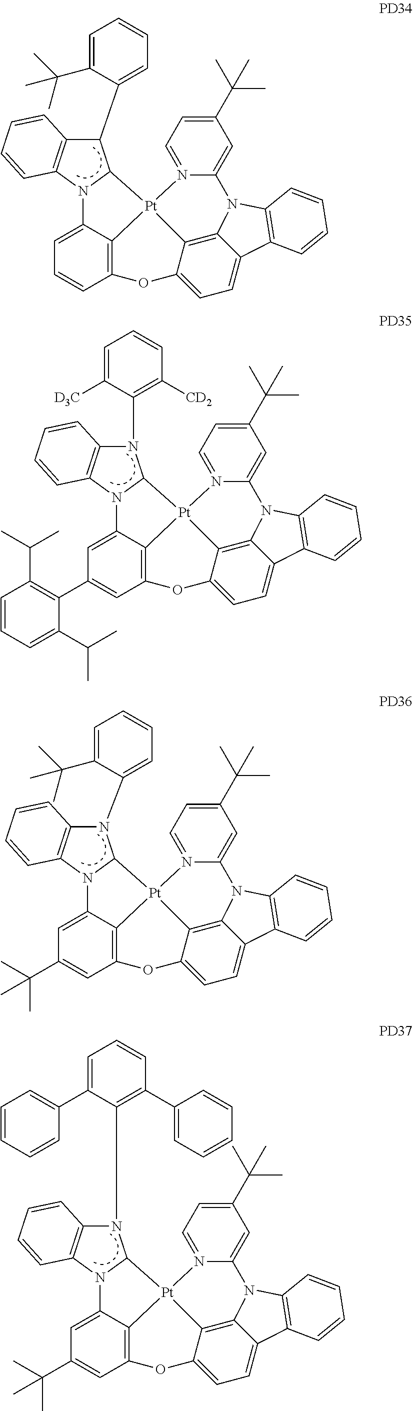

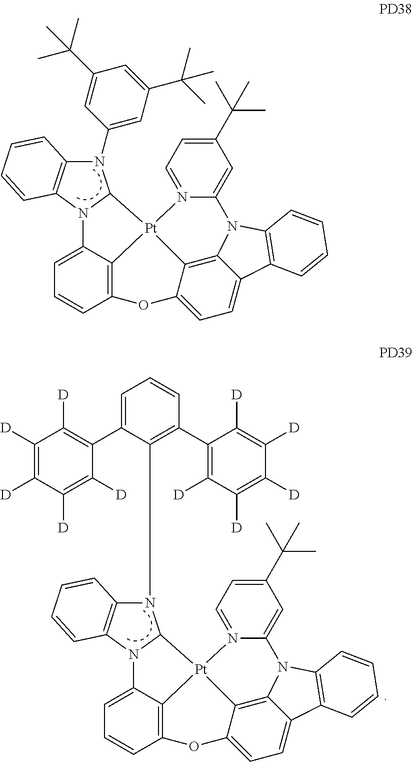

### Fluorescent Dopant

The fluorescent dopant may include an amine group-containing compound, a styryl group-containing compound, or a combination thereof.

For example, the fluorescent dopant may include a compound represented by Formula 501:

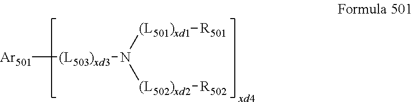

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In some embodiments, xd4 in Formula 501 may be 2.

In one or more embodiments, the fluorescent dopant may include: one selected from Compounds FD1 to FD37; DPVBi; DPAVBi; or a combination thereof:

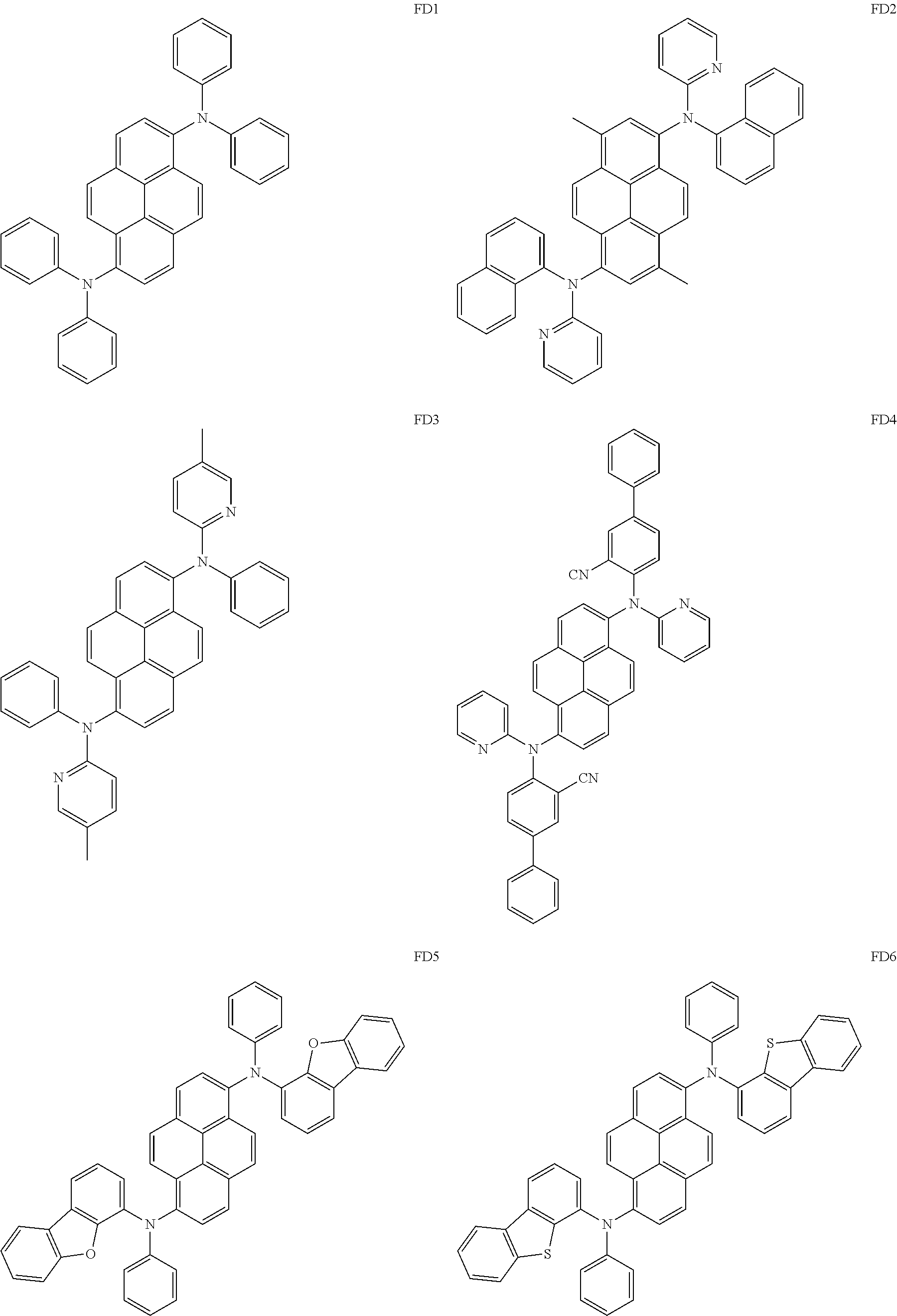

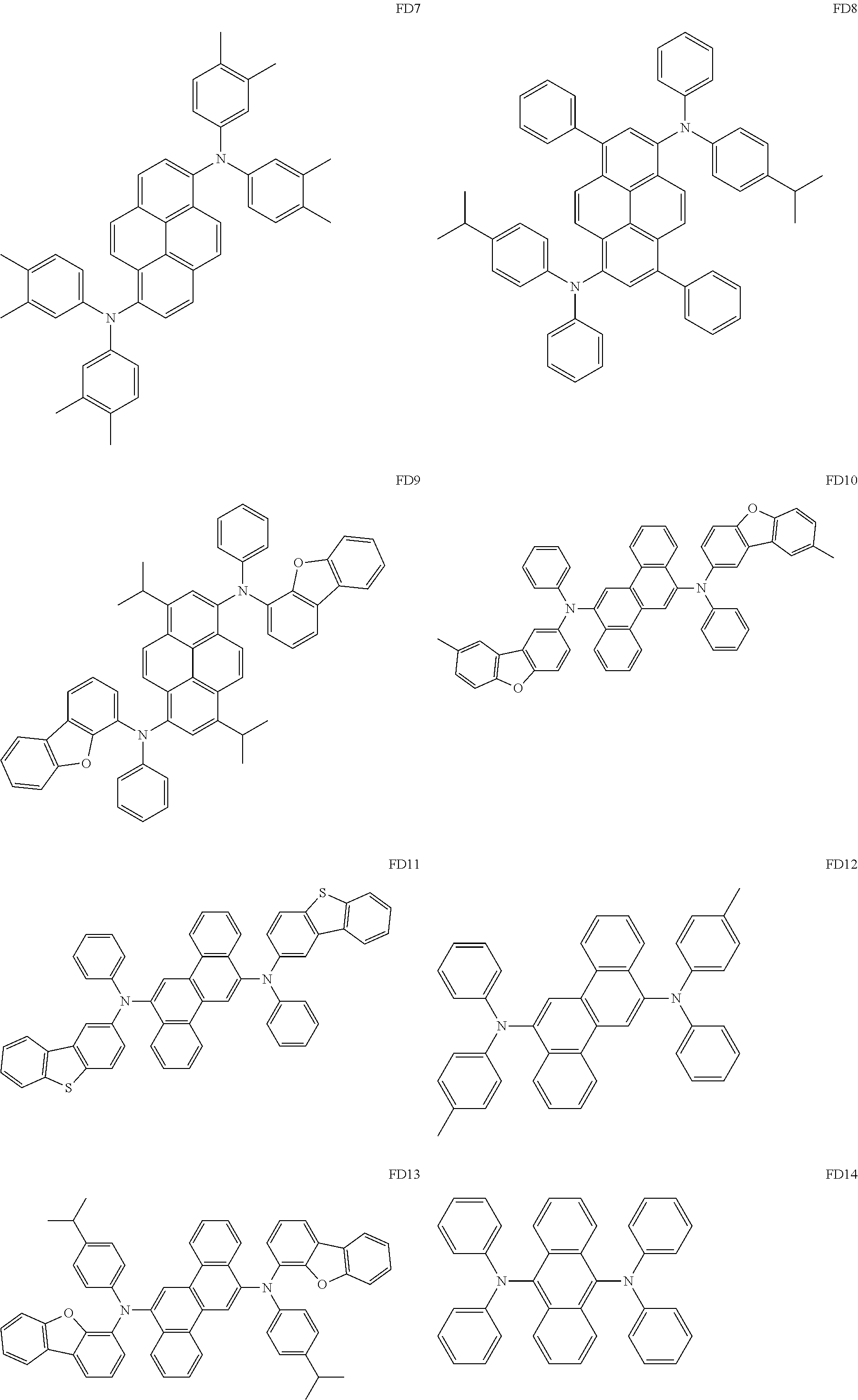

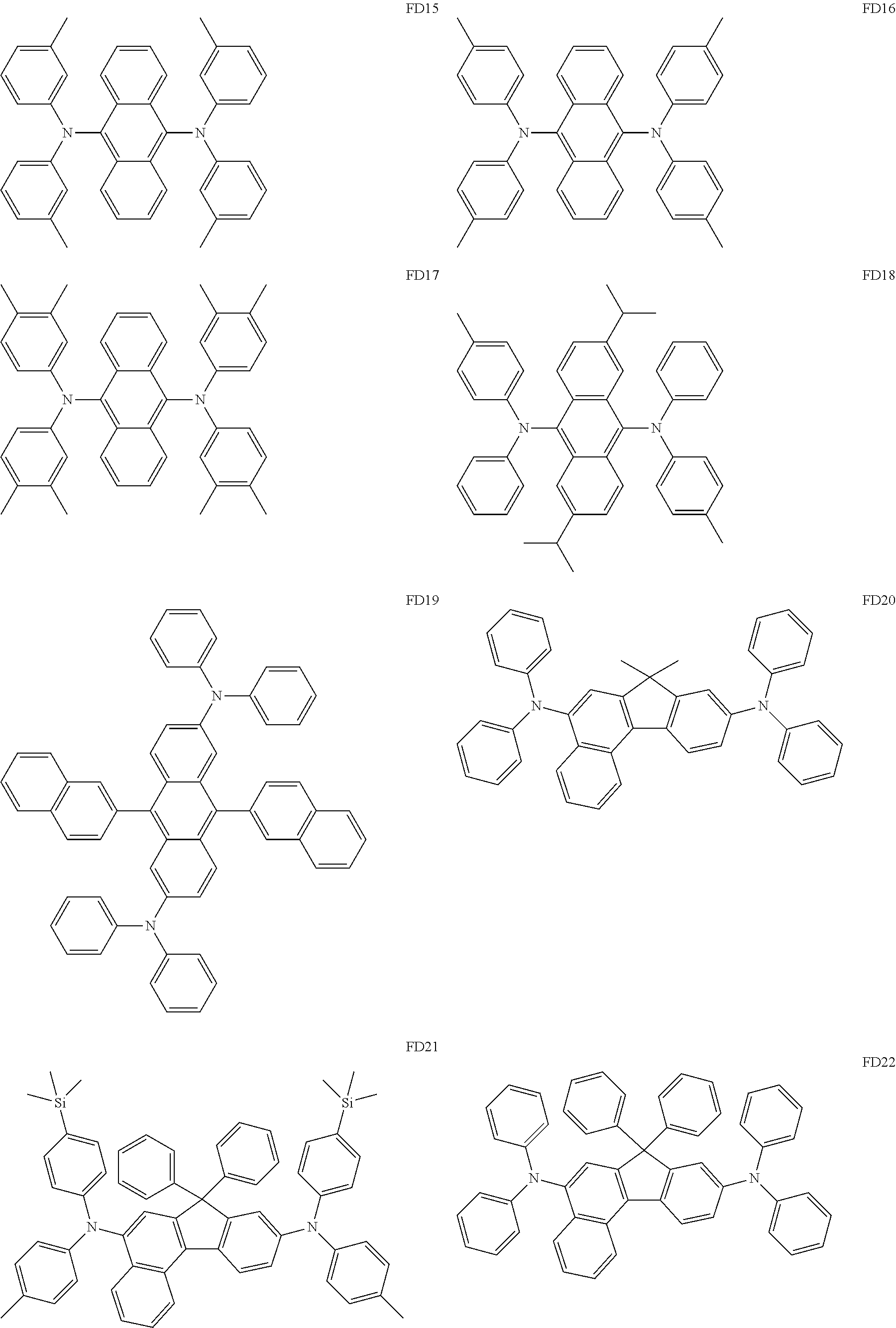

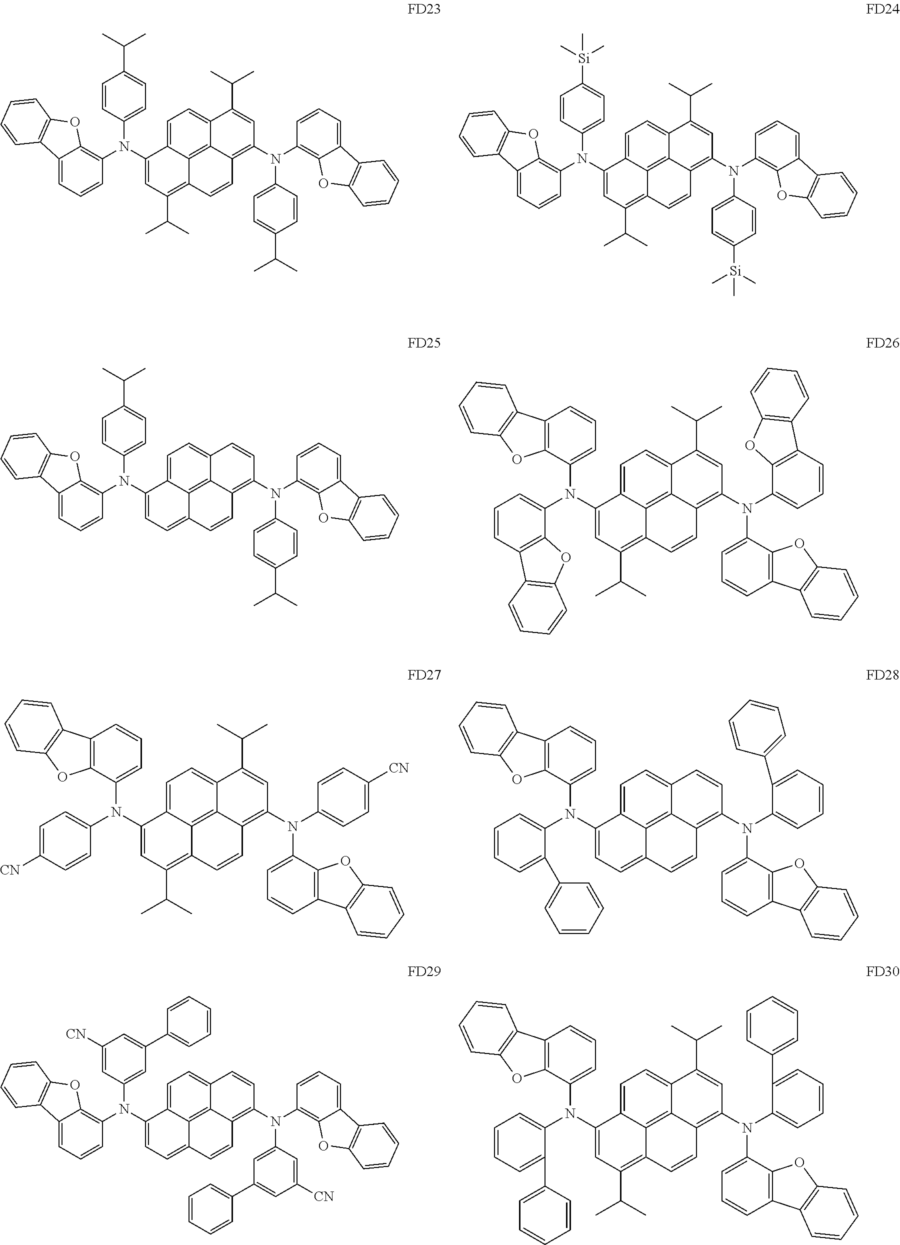

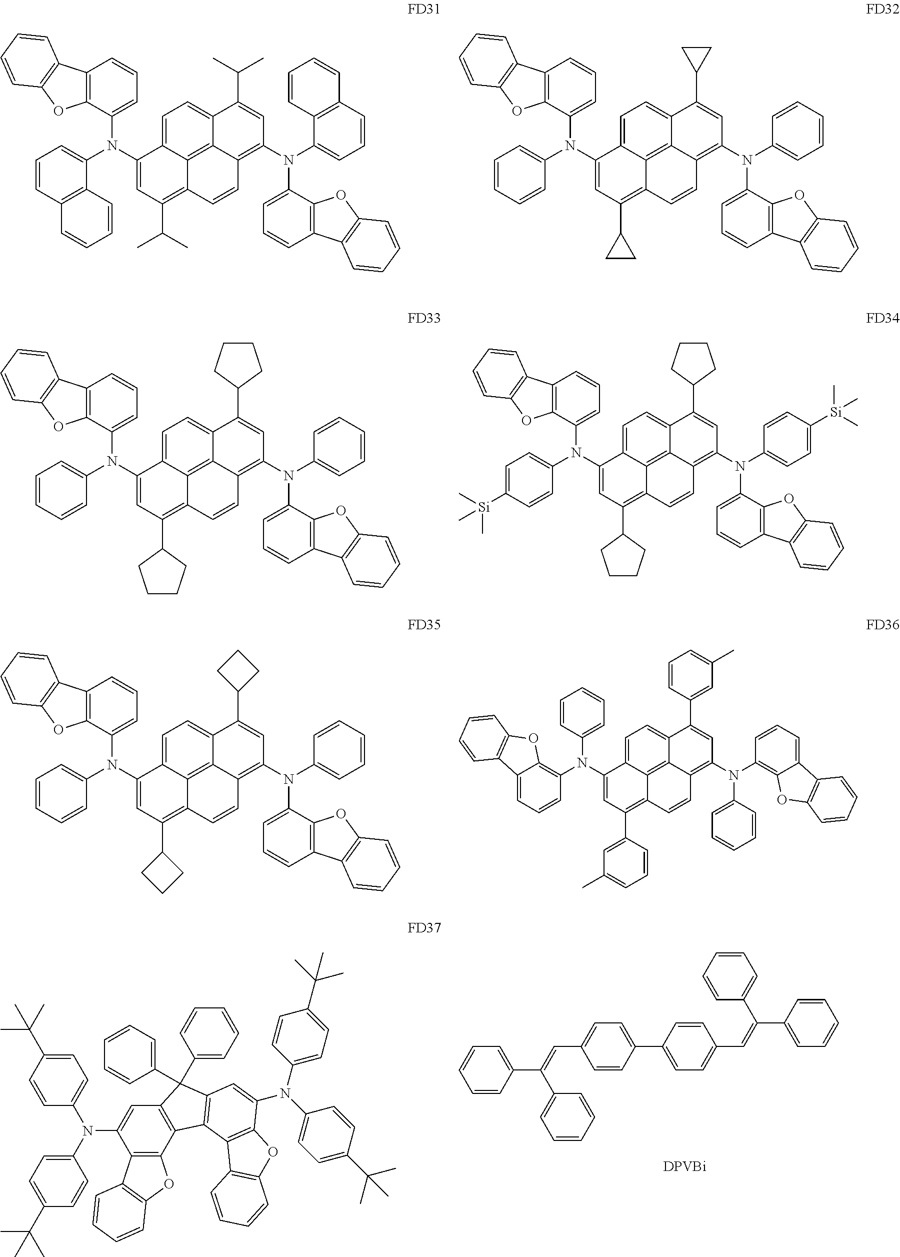

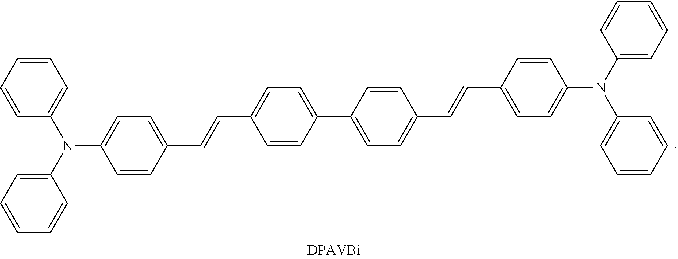 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Delayed Fluorescence Material

The emission layer may include a delayed fluorescence material.

In the present specification, the delayed fluorescence material may be selected from compounds capable of emitting delayed fluorescent light based on a delayed fluorescence emission mechanism.

The delayed fluorescence material included in the emission layer may act as a host or a dopant depending on the type (or kind) of other materials included in the emission layer.

In some embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed together while sharing boron (B).

Examples of the delayed fluorescence material may include at least one selected from Compounds DF1 to DF14:

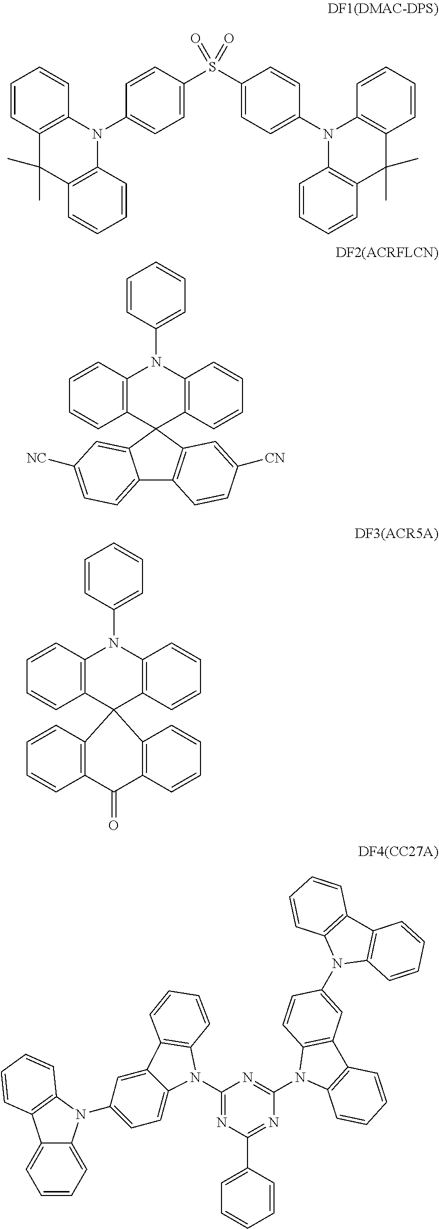

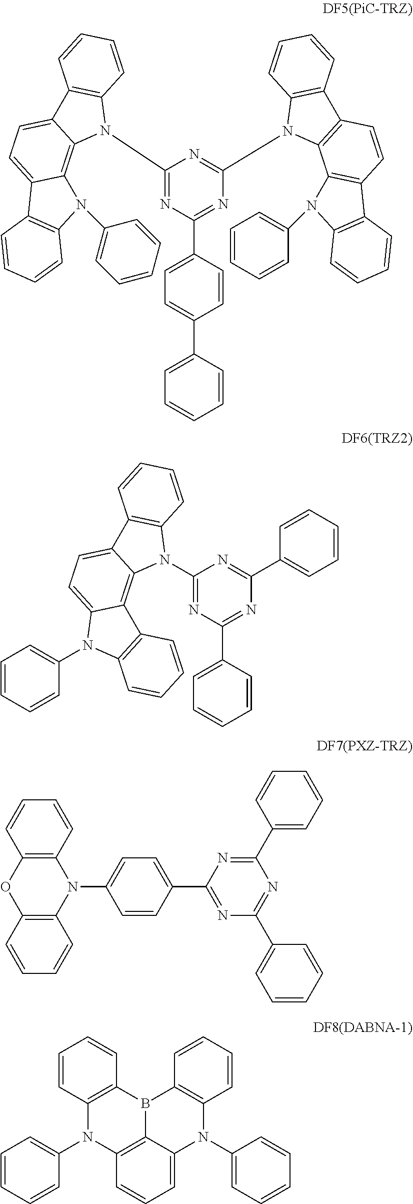 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1 ; c1ccc(N2c3ccccc3B3c4ccccc4N(c4ccccc4)c4cccc2c43)cc1]

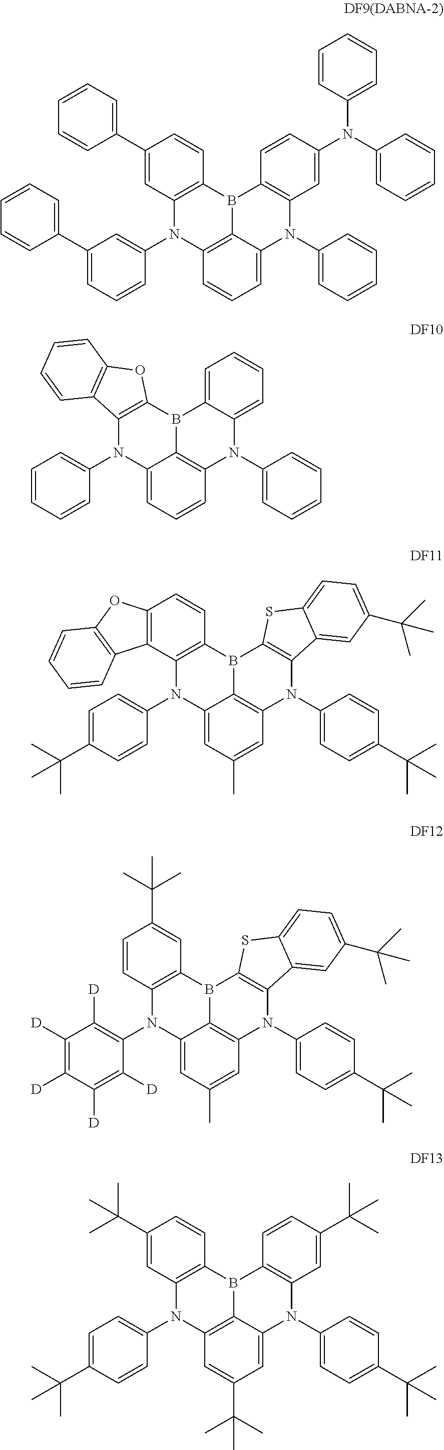

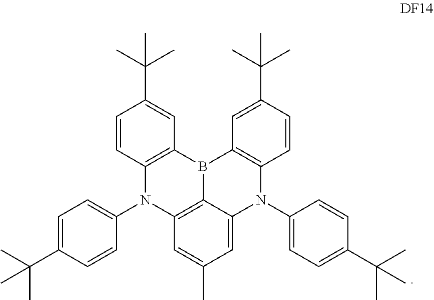 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer may include a quantum dot.

The term “quantum dot” as used herein refers to a crystal of a semiconductor compound, and may include any suitable material capable of emitting light of various suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any suitable process similar thereto.

The wet chemical process is a method including mixing a precursor material together with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE),

The quantum dot may include Group II-VI semiconductor compounds, Group Ill-V semiconductor compounds, Group III-VI semiconductor compounds, Group I-III-VI semiconductor compounds, Group IV-VI semiconductor compounds, a Group IV element or compound, or a combination thereof.

Examples of the Group II-VI semiconductor compound are a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or a combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or a combination thereof. In some embodiments, the Group III-V semiconductor compound may further include a Group II element. Examples of the Group III-V semiconductor compound further including a Group II element are InZnP, InGaZnP, InAlZnP, etc.

Examples of the Group III-VI semiconductor compound are: a binary compound, such as GaS, GaSe, Ga₂Se₃, GaTe, InS, InSe, In₂S₃, In₂Se₃, and/or InTe; a ternary compound, such as InGaS₃, and/or InGaSe₃; and a combination thereof.

Examples of the Group I-III-VI semiconductor compound are: a ternary compound, such as AgInS, AgInS₂, CuInS, CulnS₂, CuGaO₂, AgGaO₂, and/or AgAlO₂; or a combination thereof.

Examples of the Group IV-VI semiconductor compound are: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or a combination thereof.

The Group IV element or compound may include: a single element compound, such as Si or Ge; a binary compound, such as SiC and/or SiGe; or a combination thereof.

Each element included in a multi-element compound such as the binary compound, the ternary compound, and the quaternary compound may be present at a uniform concentration or non-uniform concentration in a particle.

In some embodiments, the quantum dot may have a single structure in which the concentration of each element in the quantum dot is uniform (e.g., substantially uniform), or a core-shell dual structure. For example, the material included in the core and the material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases along a direction toward the center of the core.

Examples of the shell of the quantum dot may be an oxide of metal, metalloid, and/or non-metal, a semiconductor compound, and a combination thereof. Examples of the oxide of metal, metalloid, and/or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃₀₄, and/or NiO; a ternary compound, such as MgAl₂O₄, CoFe₂O₄, NiFe₂O₄, and/or CoMn₂O₄; and a combination thereof. Examples of the semiconductor compound are, as described herein, a Group II-VI semiconductor compound; a Group III-V semiconductor compound; a Group III-VI semiconductor compound; a Group I-III-VI semiconductor compound; a Group IV-VI semiconductor compound; and a combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or a combination thereof.

A full width at half maximum (FWHM) of the emission wavelength spectrum of the quantum dot may be about 45 nm or less, for example, about 40 nm or less, for example, about 30 nm or less, and within these ranges, color purity and/or color reproducibility may be increased. In addition, because the light emitted through the quantum dot is emitted in all (e.g., substantially all) directions, the wide viewing angle may be improved.

In addition, the quantum dot may be in the form of a spherical particle, a pyramidal particle, a multi-arm particle, a cubic nanoparticle, a nanotube, a nanowire, a nanofiber, and/or a nanoplate particle.

Because the energy band gap may be adjusted by controlling the size of the quantum dot, light having various suitable wavelength bands may be obtained from the quantum dot emission layer. Accordingly, by using quantum dots of different sizes, a light-emitting device that emits light of various suitable wavelengths may be implemented. In some embodiments, the size of the quantum dot may be selected to emit red, green and/or blue light. In addition, the size of the quantum dot may be configured to emit white light by combination of light of various suitable colors.

### Electron Transport Region in Interlayer 130

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a buffer layer, a hole blocking layer, an electron control layer, an electron transport layer, an electron injection layer, or a combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, the constituting layers of each structure being sequentially stacked from an emission layer.

In one or more embodiments, the electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601 below:

[Ar₆₀₁]ₓₑ₁₁-[(L₆₀₁)ₓₑ₁-R₆₀₁]ₓₑ₂₁
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as described for Q₁,
xe21 may be 1, 2, 3, 4, or 5, and
at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In other embodiments, Ar₆₀₁ in Formula 601 may be an anthracene group unsubstituted or substituted with at least one R₁₀ₐ.

In other embodiments, the electron transport region may include a compound represented by Formula 601-1:

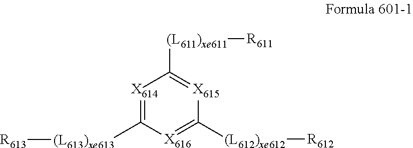

wherein, in Formula 601-1,
X₆₁₄ may be N or C(R₆₁₄), X₆₁₅ may be N or C(R₆₁₅), X₆₁₆ may be N or C(R₆₁₆), and at least one selected from X₆₁₄ to X₆₁₆ may be N,
L₆₁₁ to L₆₁₃ may each be as described for L₆₀₁,
xe611 to xe613 may each be as described for xe1,
R₆₁₁ to R₆₁₃ may each be as described for R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one selected from Compounds ET1 to ET45, 2,9-dimethyl-4,7-diphenyl-1,10-phenanthroline (BCP), 4,7-diphenyl-1,10-phenanthroline (Bphen), Alq₃, BAIq, TAZ, NTAZ, TSPO1 or a combination thereof:

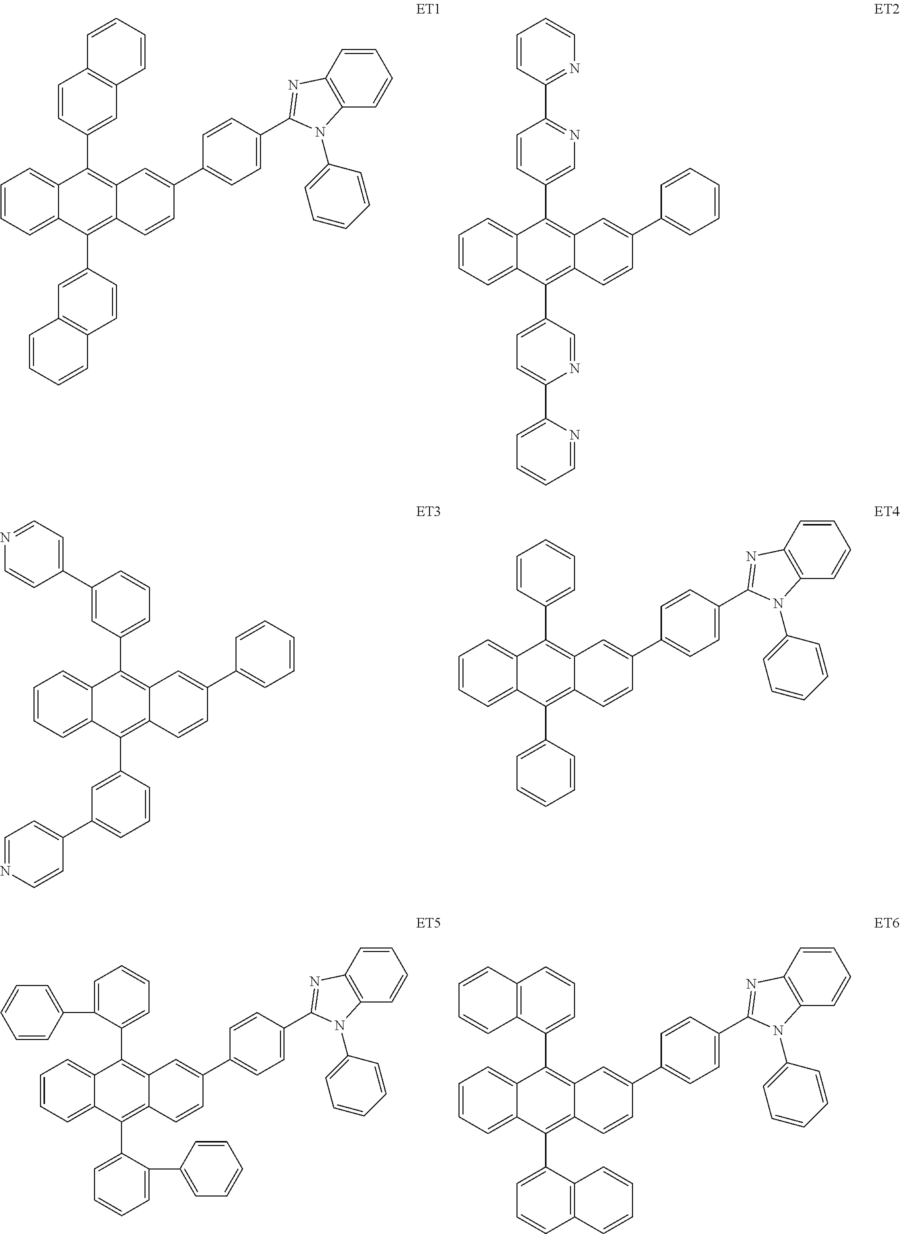

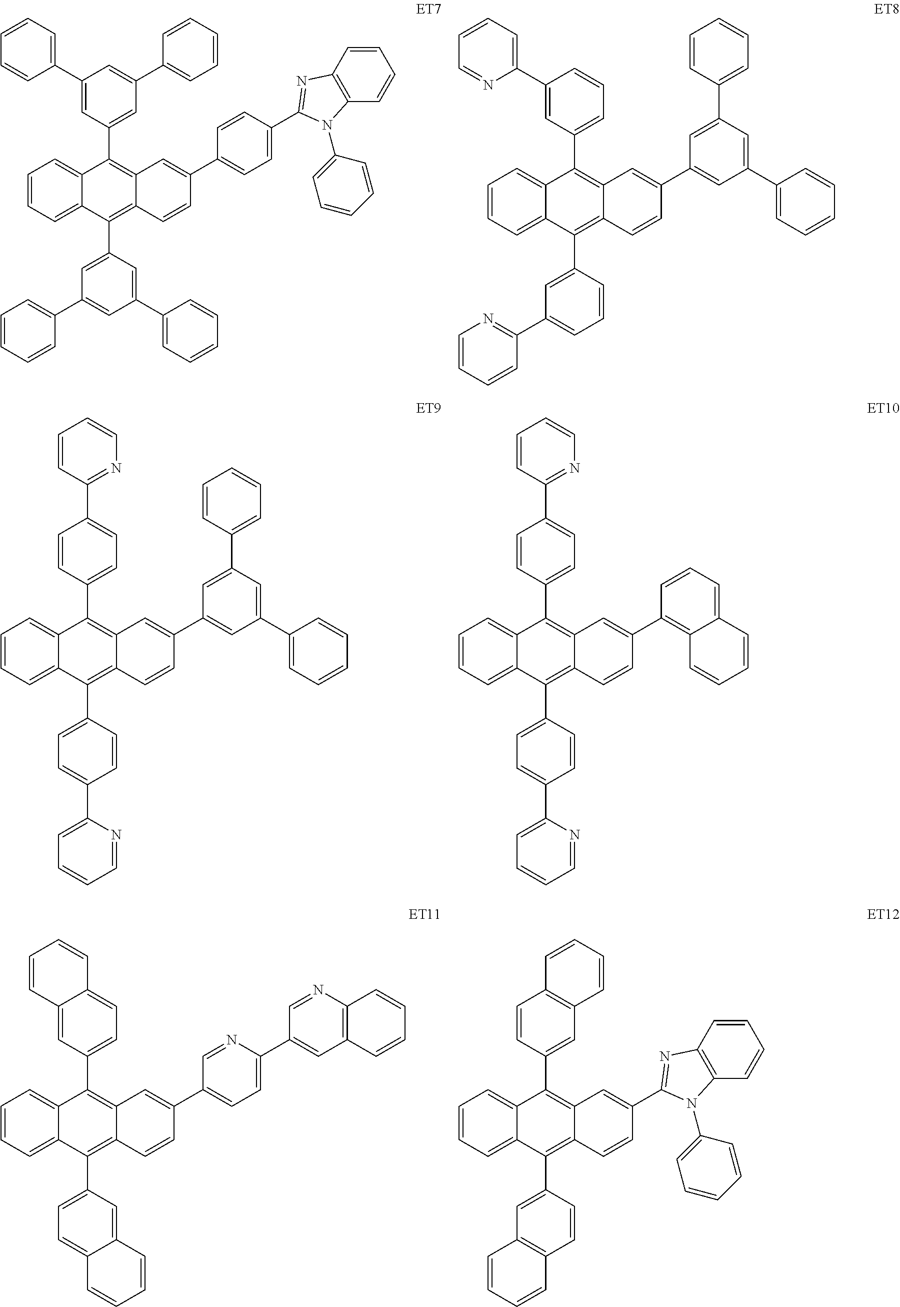

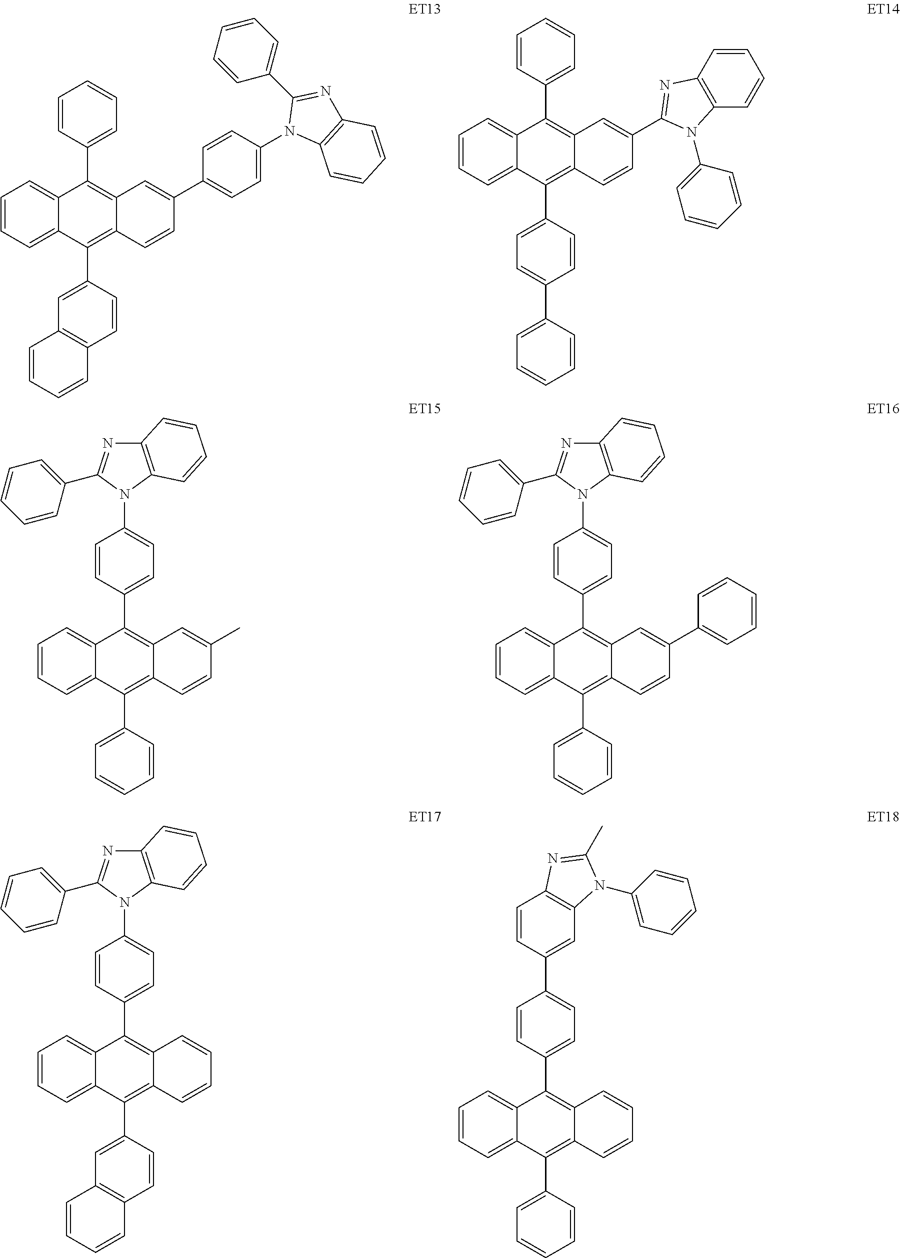

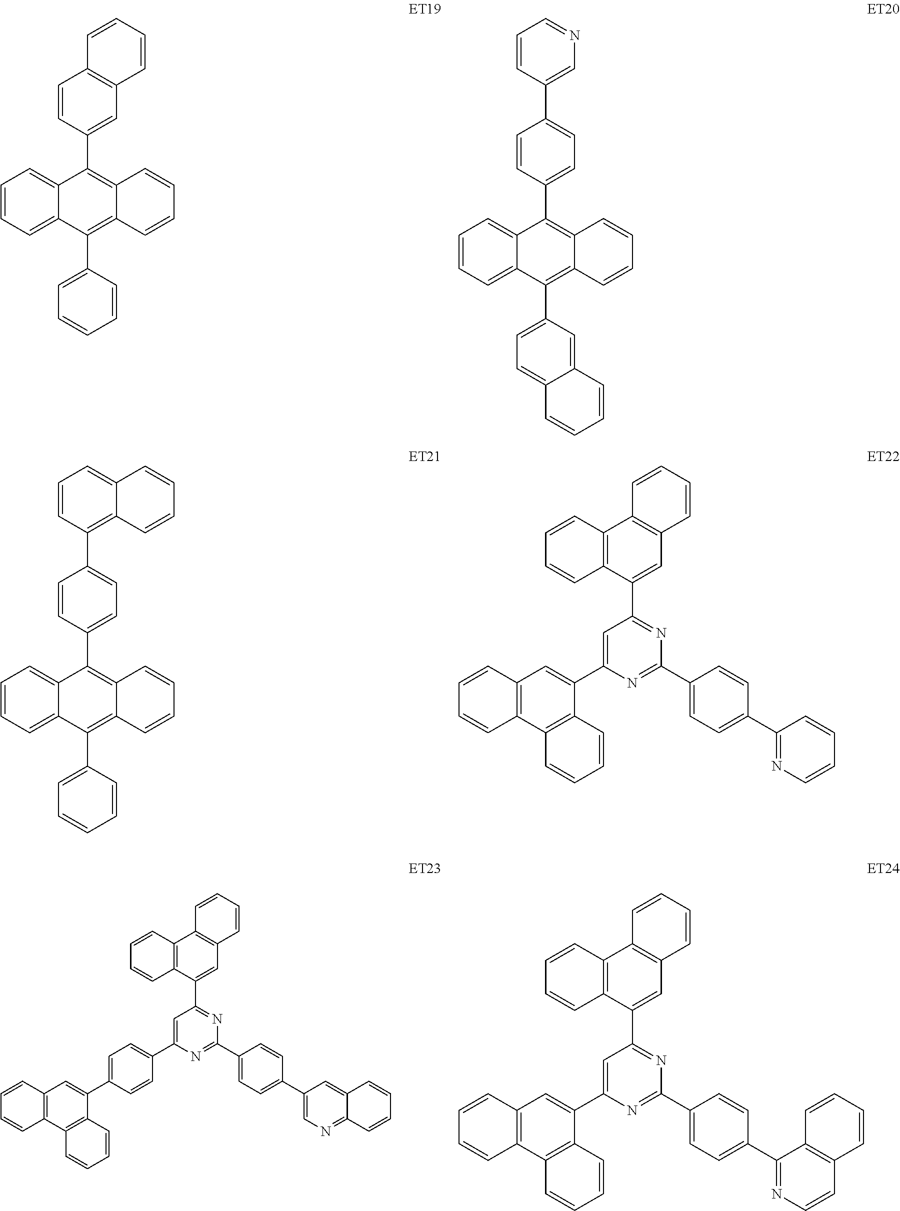

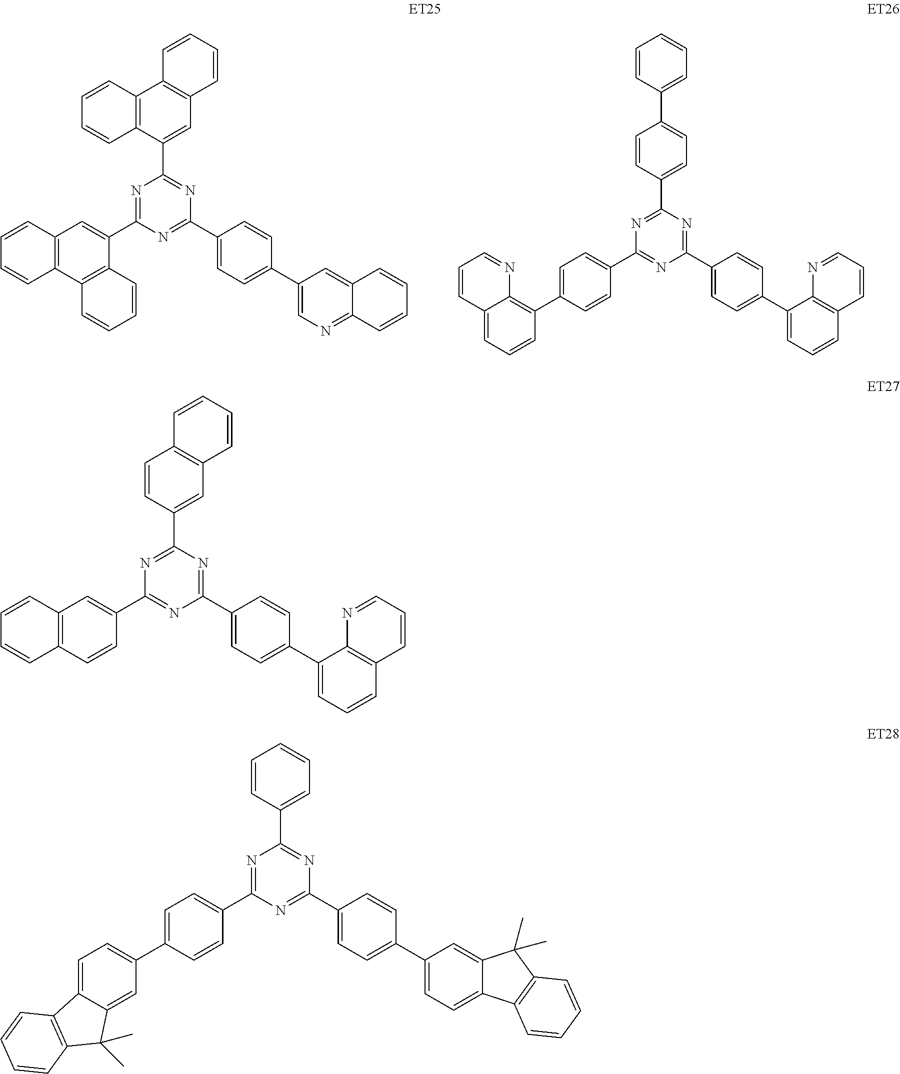 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

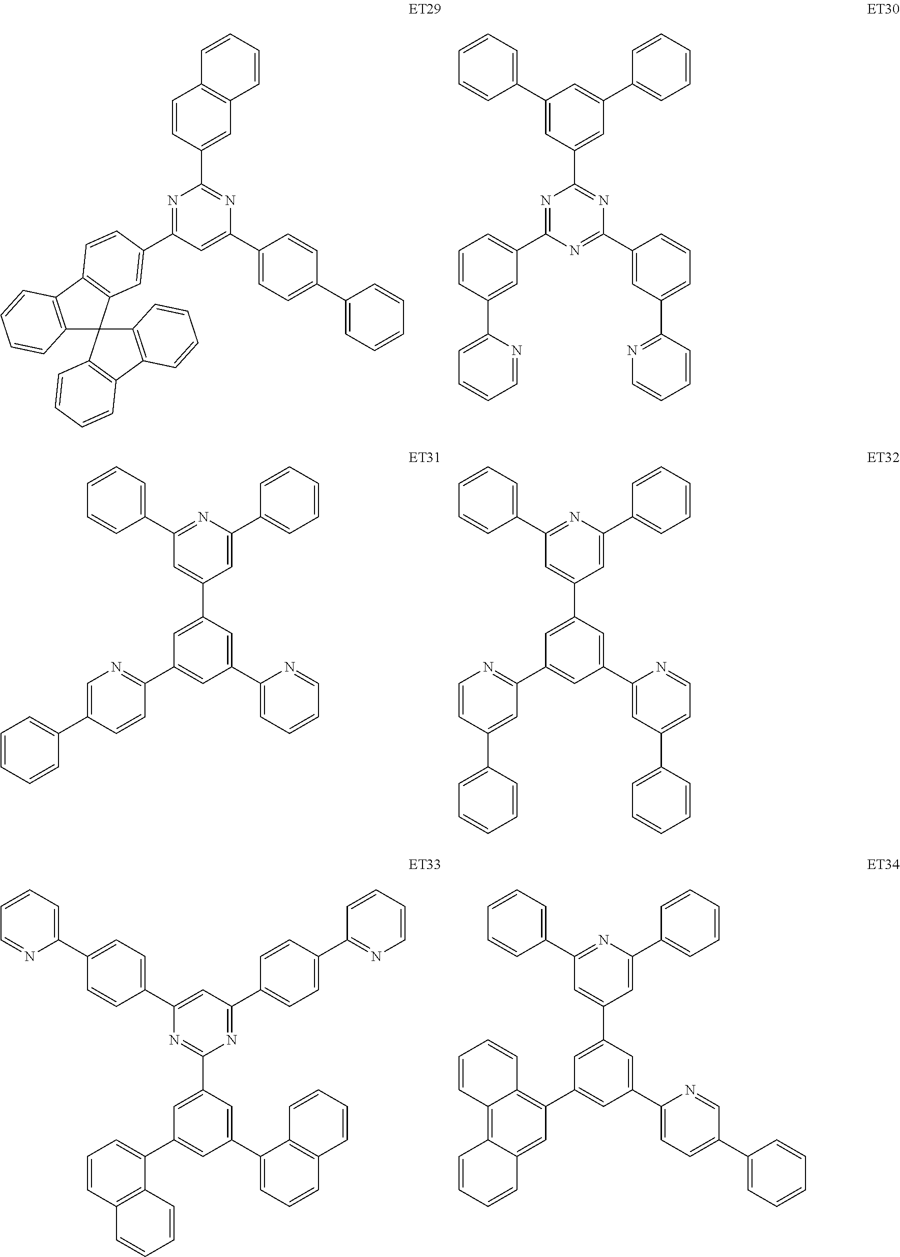

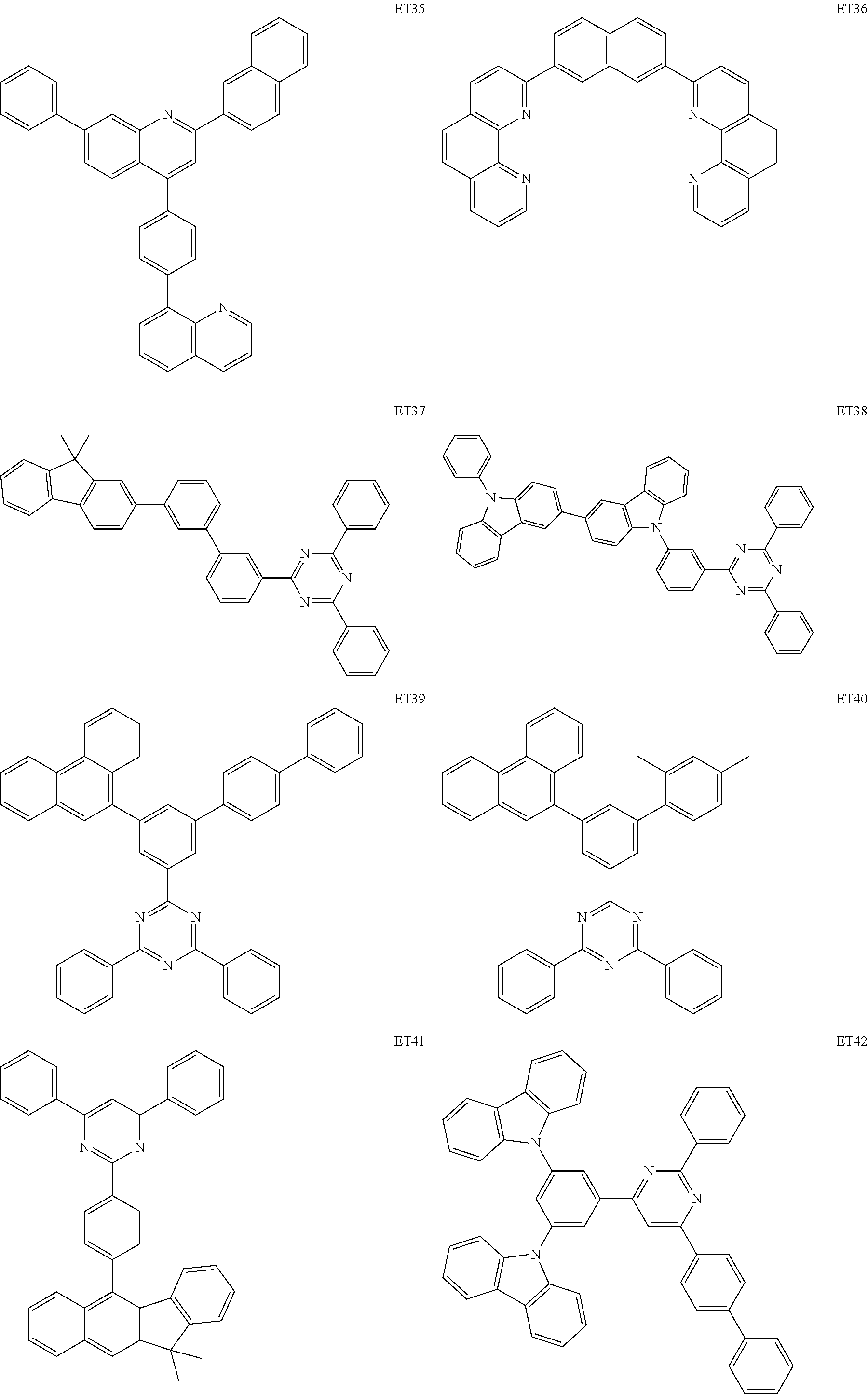

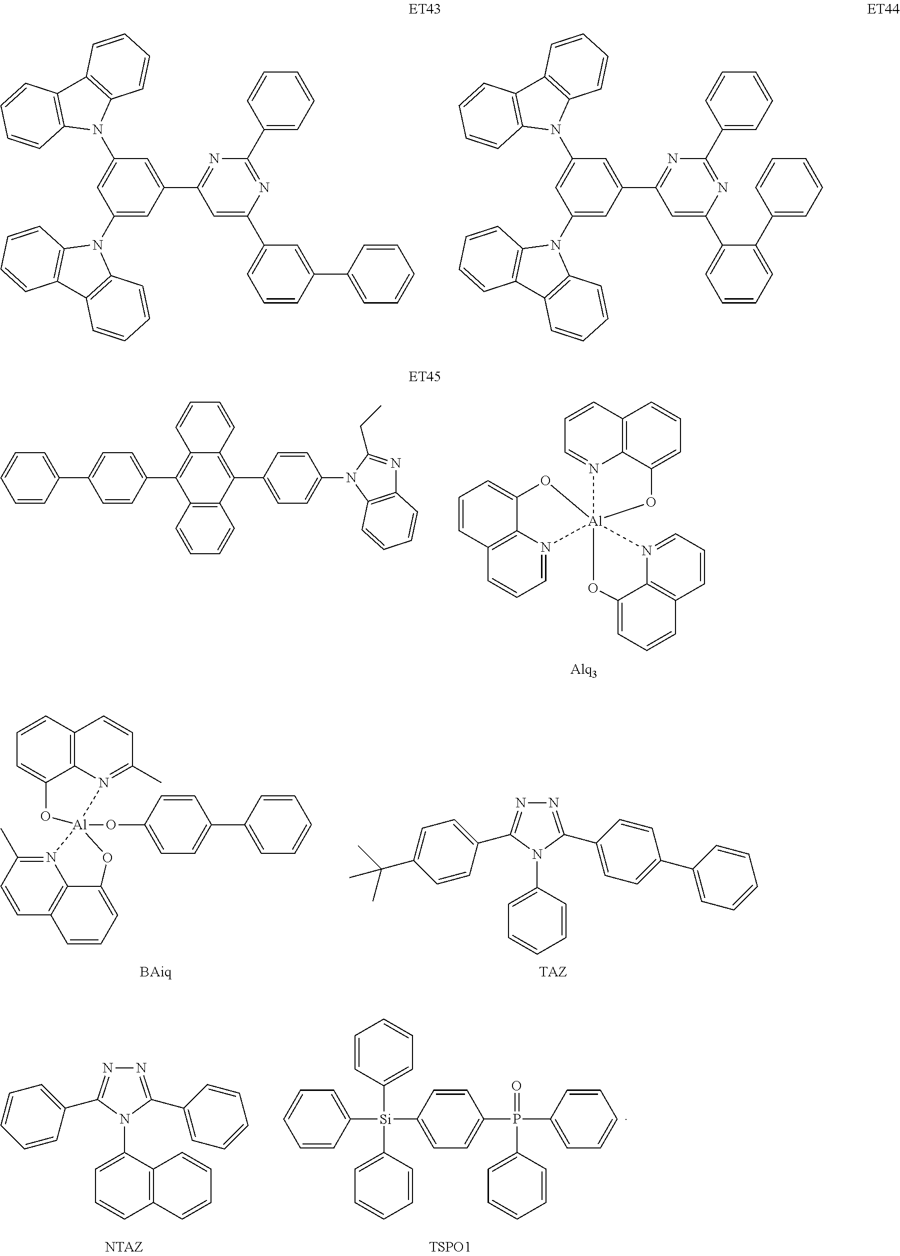

A thickness of the electron transport region may be from about 100 Å to about 5,000 Å, for example, about 160 Å to about 4,000 Å. When the electron transport region includes a buffer layer, a hole blocking layer, an electron control layer, an electron transport layer, or a combination thereof, the thickness of the buffer layer, the hole blocking layer, or the electron control layer may each independently be from about 20 Å to about 1000 Å, for example, about 30 Å to about 300 Å, and the thickness of the electron transport layer may be from about 100 Å to about 1000 Å, for example, about 150 Å to about 500 Å. When the thickness of the buffer layer, the hole blocking layer, the electron control layer, the electron transport layer, and/or the electron transport region are within these ranges, suitable or satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or a combination thereof. The metal ion of an alkali metal complex may be a Li ion, a Na ion, a K ion, a Rb ion, and/or a Cs ion, and the metal ion of an alkaline earth metal complex may be a Be ion, a Mg ion, a Ca ion, a Sr ion, and/or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or a combination thereof.

For example, the metal-containing material may include a Li complex. The Li complex may include, for example, Compound ET-D1 (LiQ) and/or ET-D2:

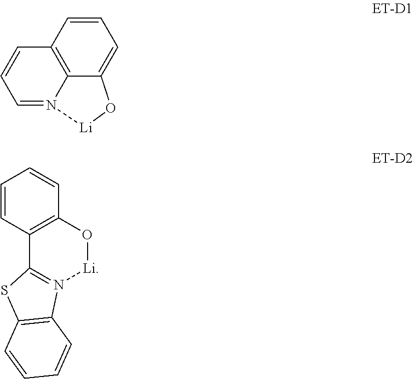

The electron transport region may include an electron injection layer that facilitates the injection of electrons from the second electrode 150. The electron injection layer may directly contact the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, alkaline earth metal, a rare earth metal, an alkali metal-containing compound, alkaline earth metal-containing compound, a rare earth metal-containing compound, an alkali metal complex, an alkaline earth metal complex, a rare earth metal complex, or a combination thereof.

The alkali metal may include Li, Na, K, Rb, Cs, or a combination thereof. The alkaline earth metal may include Mg, Ca, Sr, Ba, or a combination thereof. The rare earth metal may include Sc, Y, Ce, Tb, Yb, Gd, or a combination thereof.

The alkali metal-containing compound, the alkaline earth metal-containing compound, and the rare earth metal-containing compound may be oxides, halides (for example, fluorides, chlorides, bromides, and/or iodides), and/or tellurides of the alkali metal, the alkaline earth metal, and the rare earth metal, or a combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or a combination thereof. The alkaline earth metal-containing compound may include an alkaline earth metal compound, such as BaO, SrO, CaO, BaxSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or a combination thereof. In some embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one selected from ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or a combination thereof.

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof, as described above. In some embodiments, the electron injection layer may further include an organic material (for example, a compound represented by Formula 601).

In some embodiments, the electron injection layer may consist of: i) an alkali metal-containing compound (for example, an alkali metal halide); or ii) a) an alkali metal-containing compound (for example, an alkali metal halide), and b) an alkali metal, an alkaline earth metal, a rare earth metal, or a combination thereof. For example, the electron injection layer may be a KI:Yb co-deposited layer, an RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or a combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges described above, suitable or satisfactory electron injection characteristics may be obtained without a substantial increase in driving voltage.

### Second Electrode 150

The second electrode 150 may be on the interlayer 130 having a structure as described above. The second electrode 150 may be a cathode, which is an electron injection electrode, and as the material for the second electrode 150, a metal, an alloy, an electrically conductive compound, or a combination thereof, each having a low-work function, may be used.

The second electrode 150 may include lithium (Li), silver (Ag), magnesium (Mg), aluminum (AI), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be outside the first electrode 110, and/or a second capping layer may be outside the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in an emission layer of the interlayer 130 of the light-emitting device 10 may be extracted toward the outside through the first electrode 110 which is a semi-transmissive electrode or a transmissive electrode, and the first capping layer. Light generated in an emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include carbocyclic compounds, heterocyclic compounds, amine group-containing compounds, porphine derivatives, phthalocyanine derivatives, a naphthalocyanine derivative, alkali metal complexes, alkaline earth metal complexes, or a combination thereof. Optionally, the carbocyclic compound, the heterocyclic compound, and the amine group-containing compound may be substituted with a substituent including O, N, S, Se, Si, F, Cl, Br, I, or a combination thereof.

In some embodiments, at least one selected from the first capping layer and the second capping layer may each independently include an amine group-containing compound.

For example, at least one selected from the first capping layer and the second capping layer may each independently include a compound represented by Formula 201, a compound represented by Formula 202, or a combination thereof.

In some embodiments, at least one selected from the first capping layer and the second capping layer may each independently include one selected from Compounds HT28 to HT33, one selected from Compounds CP1 to CP6, β-NPB, or a combination thereof:

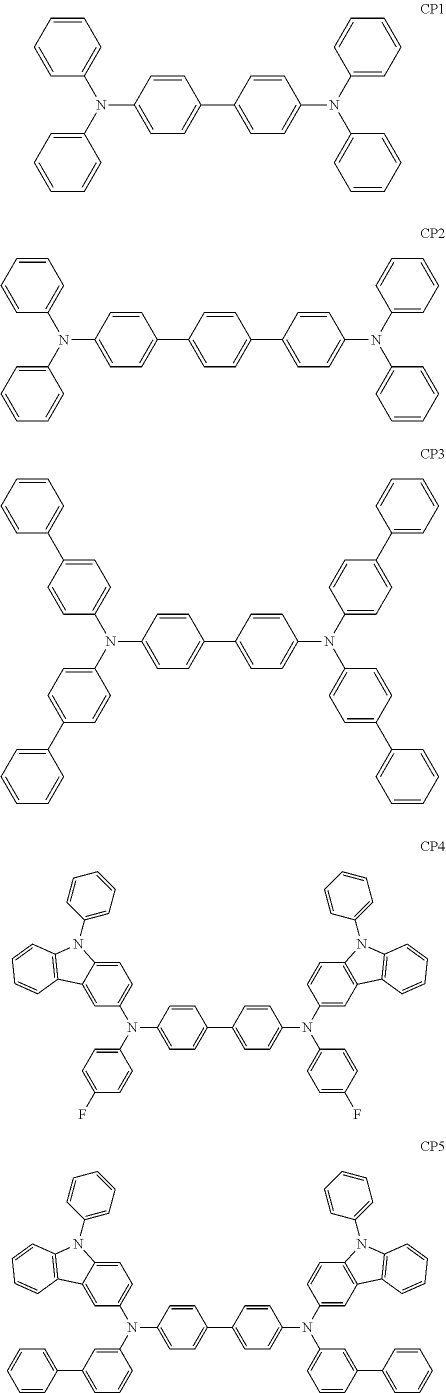 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

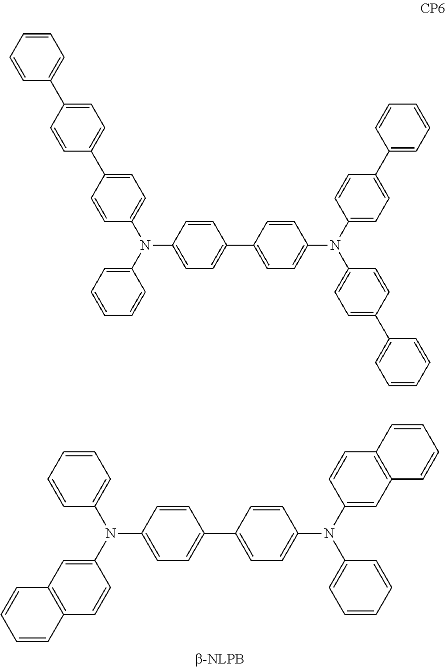 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The organometallic compound represented by Formula 1 may be included in various suitable films. According to one or more embodiments, a film including an organometallic compound represented by Formula 1 may be provided. The film may be, for example, an optical member (e.g., a light control means) (for example, a color filter, a color conversion member, a capping layer, a light extraction efficiency enhancement layer, a selective light absorbing layer, a polarizing layer, a quantum dot-containing layer, and/or like), a light-blocking member (for example, a light reflective layer, a light absorbing layer, and/or the like), and/or a protective member (for example, an insulating layer, a dielectric layer, and/or the like).

### Electronic Apparatus

The light-emitting device may be included in various suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be in at least one direction in which light emitted from the light-emitting device travels. For example, the light emitted from the light-emitting device may be blue light or white light. For more details of the light-emitting device, related description provided above may be referred to. In some embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The plurality of color filter areas (or the plurality of color conversion areas) may include a first area that emits a first color light, a second area that emits a second color light, and/or a third area that emits a third color light, wherein the first color light, the second color light, and/or the third color light may have different maximum emission wavelengths (e.g., different peak emission wavelengths) from one another. For example, the first color light may be red light, the second color light may be green light, and the third color light may be blue light. For example, the plurality of color filter areas (or the plurality of color conversion areas) may include quantum dots. In one or more embodiments, the first area may include a red quantum dot, the second area may include a green quantum dot, and the third area may not include a quantum dot. For more details of the quantum dot, related descriptions provided herein may be referred to. The first area, the second area, and/or the third area may each include a scatterer (e.g., a light scatterer).

For example, the light-emitting device may emit a first light, the first area may absorb the first light to emit a first-first color light, the second area may absorb the first light to emit a second-first color light, and the third area may absorb the first light to emit a third-first color light. In this regard, the first-first color light, the second-first color light, and the third-first color light may have different maximum emission wavelengths (e.g., different peak emission wavelengths). In one or more embodiments, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein any one selected from the source electrode and the drain electrode may be electrically connected to any one selected from the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, or the like.

The electronic apparatus may further include a sealing portion for sealing the light-emitting device. The sealing portion may be between the color filter and/or the color conversion layer and the light-emitting device. The sealing portion allows light from the light-emitting device to be extracted to the outside, and concurrently (e.g., simultaneously) prevents or reduces ambient air and/or moisture from penetrating into the light-emitting device. The sealing portion may be a sealing substrate including a transparent glass substrate and/or a plastic substrate. The sealing portion may be a thin-film encapsulation layer including at least one layer of an interlayer and/or an inorganic layer. When the sealing portion is a thin film encapsulation layer, the electronic apparatus may be flexible.

Various suitable functional layers may be additionally on the sealing portion, in addition to the color filter and/or the color conversion layer, according to the use of the electronic apparatus. Examples of the functional layers may include a touch screen layer, a polarizing layer, and the like. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

The electronic apparatus may be applied to various suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (for example, meters for a vehicle, an aircraft, and/or a vessel), projectors, and/or the like.

### Electronic Device

The light-emitting device may be included in various suitable electronic devices.

For example, the electronic device including the light-emitting device may be a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including a plurality of displays tiled together, a theater or stadium screen, a phototherapy device, and/or a signboard.

The light-emitting device may have excellent effects in terms of luminescence efficiency and long lifespan, and thus the electronic device including the light-emitting device may have characteristics such as high luminance, high resolution, and low power consumption.

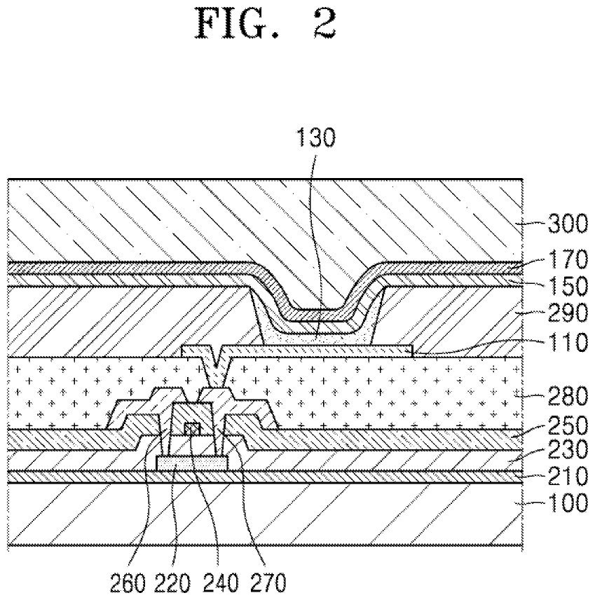

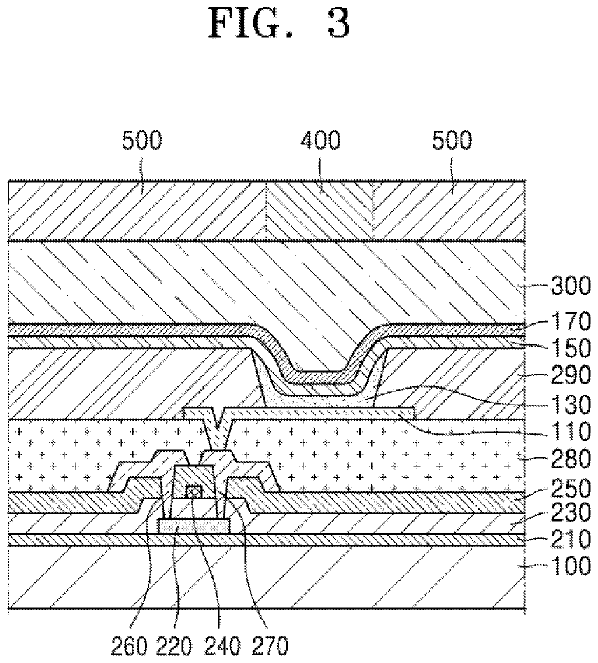

### Description of FIGS. 2 and 3

FIG. 2 is a cross-sectional view showing a light-emitting apparatus according to one or more embodiments.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100 and may provide a flat surface on the substrate 100.

A TFT may be on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270, to insulate from one another.

The source electrode 260 and the drain electrode 270 may be on the interlayer insulating film 250. The interlayer insulating film 250 and the gate insulating film 230 may expose the source region and the drain region of the activation layer 220, and the source electrode 260 and the drain electrode 270 may be in contact with the exposed portions of the source region and the drain region of the activation layer 220.

The TFT is electrically connected to a light-emitting device to drive the light-emitting device, and is covered and protected by a passivation layer 280. The passivation layer 280 may include an inorganic insulating film, an organic insulating film, or a combination thereof. A light-emitting device is provided on the passivation layer 280. The light-emitting device may include a first electrode 110, an interlayer 130, and a second electrode 150.

The first electrode 110 may be on the passivation layer 280. The passivation layer 280 may expose a portion of the drain electrode 270, not fully covering the drain electrode 270, and the first electrode 110 may be connected to the exposed portion of the drain electrode 270.

A pixel defining layer 290 including an insulating material may be on the first electrode 110. The pixel defining layer 290 may expose a certain region of the first electrode 110, and an interlayer 130 may be formed in the exposed region of the first electrode 110. The pixel defining layer 290 may be a polyimide and/or polyacrylic organic film. In some embodiments, at least some layers of the interlayer 130 may extend beyond the upper portion of the pixel defining layer 290 to be in the form of a common layer.

The second electrode 150 may be on the interlayer 130, and a capping layer 170 may be additionally formed on the second electrode 150. The capping layer 170 may be formed to cover the second electrode 150.

The encapsulation portion 300 may be on the capping layer 170. The encapsulation portion 300 may be on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or a combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or a combination thereof; or a combination of the inorganic films and the organic films.

FIG. 3 shows a cross-sectional view showing a light-emitting apparatus according to one or more embodiments.

The light-emitting apparatus of FIG. 3 is the same as the light-emitting apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In one or more embodiments, the light-emitting device included in the light-emitting apparatus of FIG. 3 may be a tandem light-emitting device.

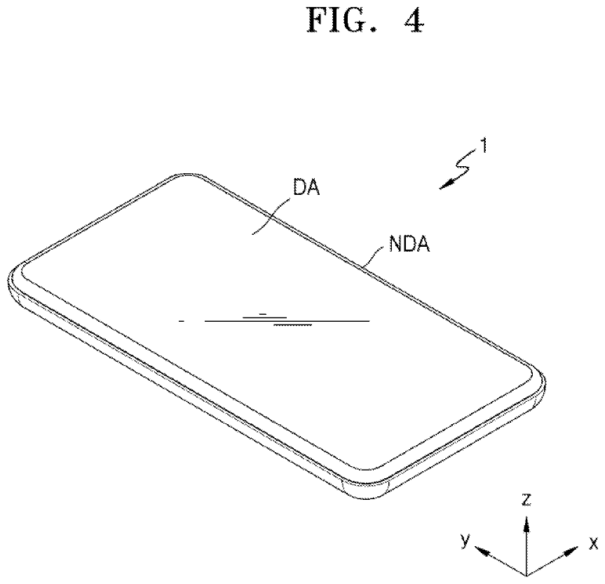

### Description of FIG. 4

FIG. 4 is a schematic perspective view of an electronic device 1 including a light-emitting device according to one or more embodiments. The electronic device 1 may be, as a device apparatus, that displays a moving image and/or still image, a portable electronic device, such as a mobile phone, a smart phone, a tablet personal computer (PC), a mobile communication terminal, an electronic notebook, an electronic book, a portable multimedia player (PMP), a navigation, and/or a ultra mobile PC (UMPC) as well as various suitable products, such as a television, a laptop, a monitor, a billboards and/or an Internet of things (IOT) device. The electronic device 1 may be such a product above or a part thereof. In addition, the electronic device 1 may be a wearable device, such as a smart watch, a watch phone, a glasses-type display, and/or a head mounted display (HMD), or a part of the wearable device. However, embodiments of the disclosure are not limited thereto. For example, the electronic device 1 may be a center information display (CID) on an instrument panel and a center fascia and/or dashboard of a vehicle, a room mirror display instead of a side mirror of a vehicle, an entertainment display for the rear seat of a car and/or a display placed on the back of the front seat, head up display (HUD) installed in front of a vehicle and/or projected on a front window glass, and/or a computer generated hologram augmented reality head up display (CGH AR HUD). FIG. 4 illustrates a case in which the electronic device 1 is a smartphone for convenience of explanation.

The electronic device 1 may include a display area DA and a non-display area NDA outside the display area DA. A display device may implement an image through an array of a plurality of pixels that are two-dimensionally arranged in the display area DA.

The non-display area NDA is an area that does not (or is not designed to) display an image, and may entirely surround the display area DA. On the non-display area NDA, a driver for providing electrical signals or power to display devices arranged on the display area DA may be arranged. On the non-display area NDA, a pad, which is an area to which an electronic element or a printing circuit board may be electrically connected, may be arranged.

In the electronic device 1, a length in the x-axis direction and a length in the y-axis direction may be different from each other. For example, as shown in FIG. 4, the length in the x-axis direction may be shorter than the length in the y-axis direction. In some embodiments, the length in the x-axis direction may be the same as the length in the y-axis direction. In some embodiments, the length in the x-axis direction may be longer than the length in the y-axis direction.

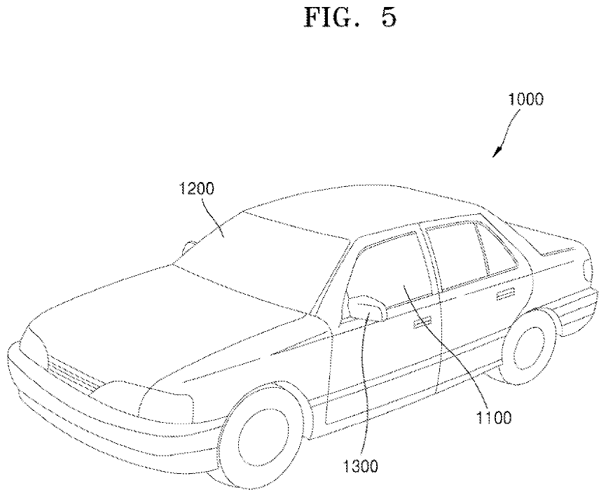

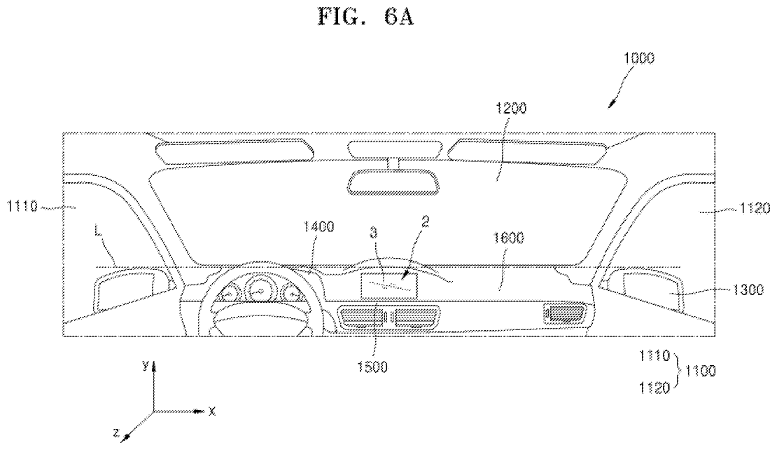

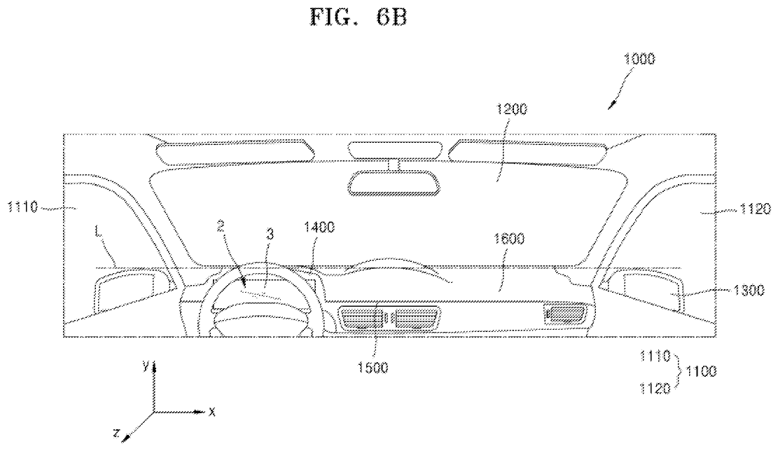

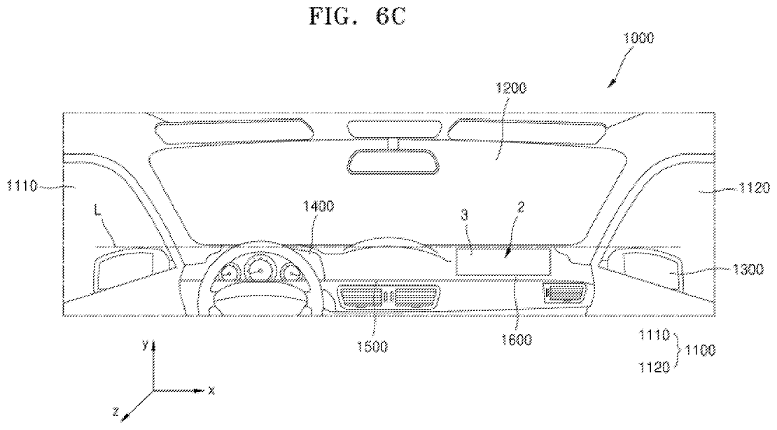

### Descriptions of FIGS. 5 and 6A to 6C

FIG. 5 is a diagram illustrating the exterior of a vehicle 1000 as an electronic device including a light-emitting device according to one or more embodiments. FIGS. 6A to 6C are each a schematic view illustrating the interior of the vehicle 1000 according to one or more embodiments.

Referring to FIGS. 5, 6A, 6B, and 6C, the vehicle 1000 may refer to various suitable apparatuses for moving a subject object to be transported, such as a human, an object, and/or an animal, from a departure point to a destination. The vehicle 1000 may include a vehicle traveling on a road and/or track, a vessel moving over a sea and/or river, an airplane flying in the sky using the action of air, and/or the like.

The vehicle 1000 may travel on a road and/or a track. The vehicle 1000 may move in a set or predetermined direction according to the rotation of at least one wheel. For example, the vehicle 1000 may include a three-wheeled or four-wheeled vehicle, a construction machine, a two-wheeled vehicle, a prime mover device, a bicycle, and/or a train running on a track.

The vehicle 1000 may include a body having an interior and an exterior, and a chassis in which mechanical apparatuses useful or necessary for driving are installed as other parts except for the body. The exterior of the vehicle body may include a front panel, a bonnet, a roof panel, a rear panel, a trunk, a filler provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a filler arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in the x-direction or the −x-direction. For example, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, an hodometer, an automatic shift selector indicator lamp, a door open warning lamp, an engine oil warning lamp, and/or a low fuel warning light.

The center fascia 1500 may include a control panel including a plurality of buttons for adjusting an audio device, an air conditioning device, and/or a heater of a seat. The center fascia 1500 may be arranged on one side of the cluster 1400.

A passenger seat dashboard 1600 may be spaced apart from the cluster 1400 with the center fascia 1500 arranged therebetween. In one or more embodiments, the cluster 1400 may be arranged to correspond to a driver seat, and the passenger seat dashboard 1600 may correspond to a passenger seat. In one or more embodiments, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In one or more embodiments, the display device 2 may be arranged between the side window glasses 1100 facing each other. The display device 2 may be arranged on at least one selected from the cluster 1400, the center fascia 1500, and the passenger seat dashboard 1600.

The display device 2 may include an organic light-emitting display device, an inorganic electroluminescent (EL) display device, a quantum dot display device, and/or the like. Hereinafter, as the display device 2 according to one or more embodiments, an organic light-emitting display device display including the light-emitting device according to the disclosure will be described as an example, but various suitable types (or kinds) of display devices as described above may be used in embodiments of the disclosure.

Referring to FIG. 6A, the display device 2 may be arranged on the center fascia 1500. In one or more embodiments, the display device 2 may display navigation information. In one or more embodiments, the display device 2 may display audio, video, and/or information regarding vehicle settings.

Referring to FIG. 6B, the display device 2 may be arranged on the cluster 1400. When the display device 2 is arranged on the cluster 1400, the cluster 1400 may display driving information and/or the like through the display device 2. In one or more embodiments, the cluster 1400 may be implemented digitally. The digital cluster 1400 may display vehicle information and driving information as images. For example, a needle and/or a gauge of a tachometer and/or various suitable warning light icons may be displayed by a digital signal.

Referring to FIG. 6C, the display device 2 may be arranged on the dashboard 1600 of the passenger seat. The display device 2 may be embedded in the passenger seat dashboard 1600 or arranged on the passenger seat dashboard 1600. In one or more embodiments, the display device 2 arranged on the dashboard 1600 for the passenger seat may display an image related to information displayed on the cluster 1400 and/or information displayed on the center fascia 1500. In some embodiments, the display device 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10−3 torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group has 3 to 61 ring-forming atoms.

The “cyclic group” as used herein may include the C₃-C₆₀ carbocyclic group, and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

For example,
the C₃-C₆₀ carbocyclic group may be i) a group T₁ or ii) a condensed cyclic group in which two or more groups T₁ are condensed together with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed together with each other, or iii) a condensed cyclic group in which at least one group T2 and at least one group T1 are condensed together with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed together with each other, iii) a group T3, iv) a condensed cyclic group in which two or more groups T3 are condensed together with each other, or v) a condensed cyclic group in which at least one group T3 and at least one group T1 are condensed together with each other (for example, the C₃-C₆₀ carbocyclic group, a ¹H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
the π electron-deficient nitrogen-containing C₁-C₆₀ cyclic group may be i) a group T4, ii) a condensed cyclic group in which two or more groups T4 are condensed together with each other, iii) a condensed cyclic group in which at least one group T4 and at least one group T1 are condensed together with each other, iv) a condensed cyclic group in which at least one group T4 and at least one group T3 are condensed together with each other, or v) a condensed cyclic group in which at least one group T4, at least one group T1, and at least one group T3 are condensed together with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the group T3 may be a furan group, a thiophene group, a 1H-pyrrole group, a silole group, or a borole group, and
the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group, a monovalent group, or a polyvalent group (for example, a divalent group, a trivalent group, a tetravalent group, etc.) according to the structure of a formula for which the corresponding term is used. In one or more embodiments, “a benzene group” may be a benzo group, a phenyl group, a phenylene group, or the like, which may be easily understand by one of ordinary skill in the art according to the structure of a formula including the “benzene group.”

In some embodiments, examples of the monovalent C₃-C₆₀ carbocyclic group and the monovalent C₁-C₆₀ heterocyclic group may include a C₃-C₁ cycloalkyl group, a C₁-C₁₀ heterocycloalkyl group, a C₃-C₁ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the divalent C₁-C₆₀ heterocyclic group may include a C₃-C₁ cycloalkylene group, a C₁-C₁₀ heterocycloalkylene group, a C₃-C₁ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof are an ethenyl group, a propenyl group, and a butenyl group. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group, a propynyl group, etc. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by -OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁ cycloalkylene group” as used herein refers to a divalent group having substantially the same structure as the C₃-C₁a cycloalkyl group.

The term “C₁-C₁₀ heterocycloalkyl group” as used herein refers to a monovalent cyclic group of 1 to 10 carbon atoms, further including, in addition to carbon atoms, at least one heteroatom, as ring-forming atoms, and examples are a 1,2,3,4-oxatriazolidinyl group, a tetrahydrofuranyl group, and a tetrahydrothiophenyl group. The term “C₁-C₁₀ heterocycloalkylene group” as used herein refers to a divalent group having substantially the same structure as the C₁-C₁₀ heterocycloalkyl group.

The term C₃-C₁a cycloalkenyl group used herein refers to a monovalent cyclic group that has three to ten carbon atoms and at least one carbon-carbon double bond in the ring thereof and no aromaticity (e.g., is not aromatic), and examples thereof are a cyclopentenyl group, a cyclohexenyl group, and a cycloheptenyl group. The term “C₃-C₁a cycloalkenylene group” as used herein refers to a divalent group having substantially the same structure as the C₃-C₁a cycloalkenyl group.

The term “C₁-C₁₀ heterocycloalkenyl group” as used herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed together with each other.

The term “C₁-C₆₀ heteroaryl group” as used herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as used herein refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed together with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure (e.g., is not aromatic when considered as a whole). Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having substantially the same structure as the monovalent non-aromatic condensed polycyclic group described above.

The term “monovalent non-aromatic condensed heteropolycyclic group” as used herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure (e.g., is not aromatic when considered as a whole). Examples of the monovalent non-aromatic condensed heteropolycyclic group include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as used herein refers to a divalent group having substantially the same structure as the monovalent non-aromatic condensed heteropolycyclic group described above.

The term “C₆-C₆₀ aryloxy group” as used herein indicates -OA₁₀₂ (wherein A₁₀₂ is a C₆-C₆₀ aryl group), and the term “C₆-C₆₀ arylthio group” as used herein indicates -SA₁₀₃ (wherein A₁₀₃ is a C₆-C₆₀ aryl group).

The term “C₇-C₆₀ arylalkyl group” used herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroarylalkyl group” used herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as used herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂ (Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂ (Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂ (Q₃₁), or —P(═O)(Q₃₁)(Q₃₂).

Q₁ to Q₃, Q₁₁ to Q₁₃, Q₂₁ to Q₂₃ and Q₃₁ to Q₃₃ used herein may each independently be: hydrogen; deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

The term “heteroatom” as used herein refers to any atom other than a carbon atom. Examples of the heteroatom are O, S, N, P, Si, B, Ge, Se, and a combinations thereof.

The term “third-row transition metal” used herein includes hafnium (Hf), tantalum (Ta), tungsten (W), rhenium (Re), osmium (Os), iridium (Ir), platinum (Pt), gold (Au), and the like.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.
* and *′ as used herein, unless defined otherwise, each refer to a binding site to a neighboring atom in a corresponding formula or moiety.

In the present specification, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-emitting devices according to embodiments will be described in more detail with reference to the following synthesis examples and examples. The wording “B was used instead of A” used in describing Synthesis Examples refers to that an identical molar equivalent of B was used in place of A.

### EXAMPLES

### Synthesis Example 1 (Synthesis of Compound 1)

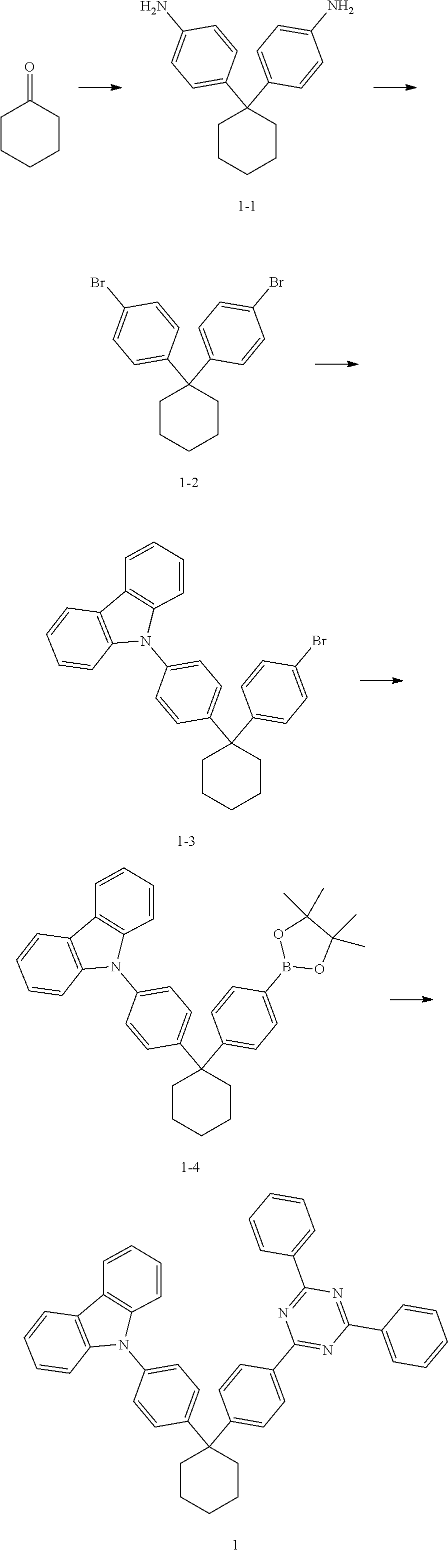 [SMILES: O=C1CCCCC1 ; Nc1ccc(C2(c3ccc(N)cc3)CCCCC2)cc1 ; Brc1ccc(C2(c3ccc(Br)cc3)CCCCC2)cc1 ; Brc1ccc(C2(c3ccc(-n4c5ccccc5c5ccccc54)cc3)CCCCC2)cc1 ; CC1(C)OB(c2ccc(C3(c4ccc(-n5c6ccccc6c6ccccc65)cc4)CCCCC3)cc2)OC1(C)C ; c1ccc(-c2nc(-c3ccccc3)nc(-c3ccc(C4(c5ccc(-n6c7ccccc7c7ccccc76)cc5)CCCCC4)cc3)n2)cc1]

### Synthesis of Intermediate 1-1

In a nitrogen atmosphere, cyclohexanone (1 eq), aniline (6 eq), and HCl (6 eq) were stirred at a temperature of 160° C. for 48 hours. After cooling the obtained solution, an aqueous sodium bicarbonate solution was slowly added dropwise thereto at 0° C. until the pH reached 7. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 1-1 (yield of 55%).

### Synthesis of Intermediate 1-2

In a nitrogen atmosphere, Intermediate 1-1 (1 eq) and copper(I) bromide (3 eq) were dissolved in ethyl alcohol and hydrobromic acid (1 eq) was slowly added dropwise thereto at a temperature of 0° C. Then, sodium nitrite (10 eq) dissolved in pure water was slowly added thereto at a temperature of 0° C. Then the resultant mixture was stirred at room temperature for 15 hours. The obtained solution was neutralized by adding sodium thiosulfate aqueous solution thereto. Then, an organic layer obtained by washing the obtained solution three times with ethylacetate and water was dried under reduced pressure after magnesium sulfate was added thereto. Subsequently, the separation-purification process was performed by column chromatography to obtain Intermediate 1-2 (yield of 38%).

### Synthesis of Intermediate 1-3

In a nitrogen atmosphere, Intermediate 1-2 (1 eq), 9H-carbazole (1 eq), tris(dibenzylideneacetone)dipalladium(0) (0.05 eq), tri-tert-butylphosphine (0.1 eq), and sodium tert-butoxide (2 eq) were dissolved in toluene, and then stirred at 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 1-3 (yield of 83%).

### Synthesis of Intermediate 1-4

In a nitrogen atmosphere, Intermediate 1-3 (1 eq), bis(pinacolato)diboron (1.3 eq), dichlorobis(triphenylphosphine)palladium (II) (0.05 eq), and potassium acetate (2.5 eq) were dissolved in toluene and stirred at a temperature of 110° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 1-4 (yield of 82%).

### Synthesis of Compound 1

In a nitrogen atmosphere, Intermediate 1-4 (1 eq), 2-chloro-4,6-diphenyl-1,3,5-triazine (1 eq), Tetrakis(triphenylphosphine)palladium (0) (0.05 eq), and potassium carbonate (2.5 eq) were dissolved in a solution including tetrahydrofuran and H₂O mixed together at a ratio of 2:1 and then stirred at a temperature of 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Compound 1 (yield of 75%).

### Synthesis Example 2 (Synthesis of Compound 22)

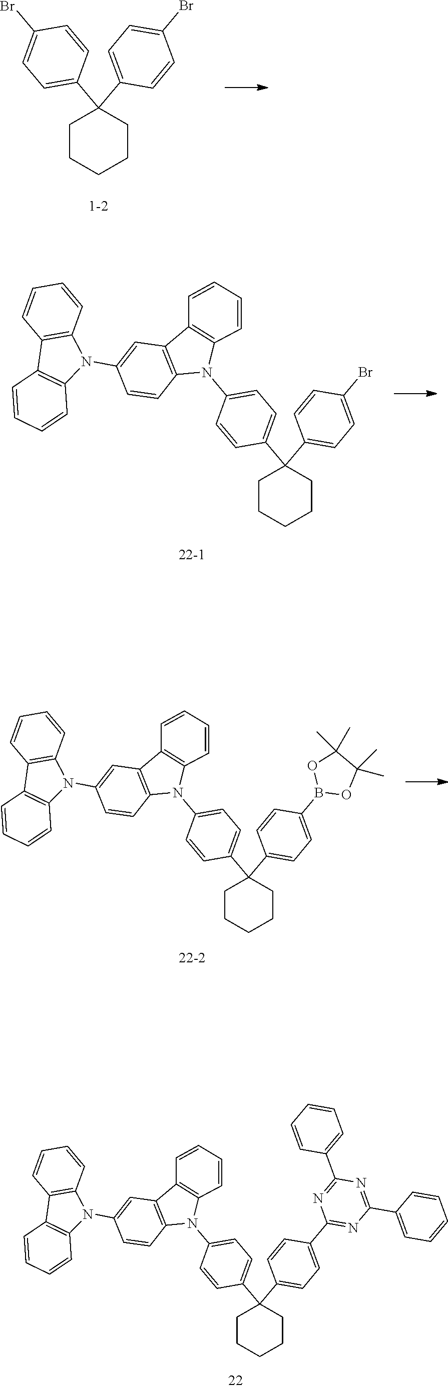 [SMILES: Brc1ccc(C2(c3ccc(Br)cc3)CCCCC2)cc1 ; Brc1ccc(C2(c3ccc(-n4c5ccccc5c5cc(-n6c7ccccc7c7ccccc76)ccc54)cc3)CCCCC2)cc1 ; CC1(C)OB(c2ccc(C3(c4ccc(-n5c6ccccc6c6cc(-n7c8ccccc8c8ccccc87)ccc65)cc4)CCCCC3)cc2)OC1(C)C ; c1ccc(-c2nc(-c3ccccc3)nc(-c3ccc(C4(c5ccc(-n6c7ccccc7c7cc(-n8c9ccccc9c9ccccc98)ccc76)cc5)CCCCC4)cc3)n2)cc1]

### Synthesis of Intermediate 22-1

In a nitrogen atmosphere, Intermediate 1-2 (1 eq), 9H-3,9′-bicarbazole (1 eq), tris(dibenzylideneacetone)dipalladium(0) (0.05 eq), tri-tert-butylphosphine (0.1 eq), and sodium tert-butoxide (2 eq) were dissolved in toluene, and then stirred at 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 22-1 (yield of 69%).

### Synthesis of Intermediate 22-2

In a nitrogen atmosphere, Intermediate 22-1 (1 eq), bis(pinacolato)diboron (1.3 eq), dichlorobis(triphenylphosphine)palladium(II) (0.05 eq), and potassium acetate (2.5 eq) were dissolved in toluene and stirred at a temperature of 110° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 22-2 (yield of 67%).

### Synthesis of Compound 22

In a nitrogen atmosphere, Intermediate 22-2 (1 eq), 2-chloro-4,6-diphenyl-1,3,5-triazine (1 eq), tetrakis(triphenylphosphine)palladium (0) (0.05 eq), and potassium carbonate (2.5 eq) were dissolved in a solution including tetrahydrofuran and H₂O mixed together at a ratio of 2:1 and then stirred at a temperature of 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Compound 22 (yield of 73%).

### Synthesis Example 3 (synthesis of Compound 46)

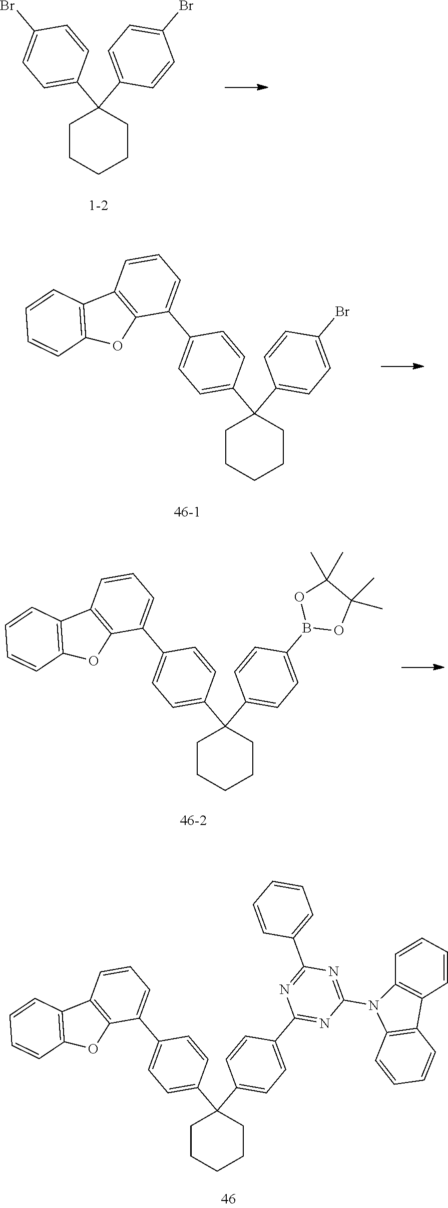 [SMILES: Brc1ccc(C2(c3ccc(Br)cc3)CCCCC2)cc1 ; Brc1ccc(C2(c3ccc(-c4cccc5c4oc4ccccc45)cc3)CCCCC2)cc1 ; CC1(C)OB(c2ccc(C3(c4ccc(-c5cccc6c5oc5ccccc56)cc4)CCCCC3)cc2)OC1(C)C ; c1ccc(-c2nc(-c3ccc(C4(c5ccc(-c6cccc7c6oc6ccccc67)cc5)CCCCC4)cc3)nc(-n3c4ccccc4c4ccccc43)n2)cc1]

### Synthesis of Intermediate 46-1

In a nitrogen atmosphere, Intermediate 1-2 (1 eq), 2-(dibenzo[b,d]furan-4-yl)-4,4,5,5-tetramethyl-1,3,2-dioxaborolane (1 eq), tetrakis(triphenylphosphine)palladium(0) (0.05 eq), and potassium carbonate (2.5 eq) were dissolved in a solution including tetrahydrofuran and H₂O mixed together at a ratio of 2:1 and then stirred at a temperature of 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 46-1 (yield of 71%).

### Synthesis of Intermediate 46-2

In a nitrogen atmosphere, Intermediate 46-1 (1 eq), bis(pinacolato)diboron (1.3 eq), dichlorobis(triphenylphosphine)palladium(II) (0.05 eq), and potassium acetate (2.5 eq) were dissolved in toluene and stirred at a temperature of 110° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Intermediate 46-2 (yield of 71%).

### Synthesis of Compound 46

In a nitrogen atmosphere, Intermediate 46-2 (1 eq), 9-(4-chloro-6-phenyl-1,3,5-triazin-2-yl)-9H-carbazole (1 eq), tetrakis(triphenylphosphine)palladium(0) (0.05 eq), and potassium carbonate (2.5 eq) were dissolved in a solution including tetrahydrofuran and H₂O mixed together at a ratio of 2:1 and stirred at a temperature of 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Compound 46 (yield of 67%).

### Synthesis Example 4 (Synthesis of Compound 67)

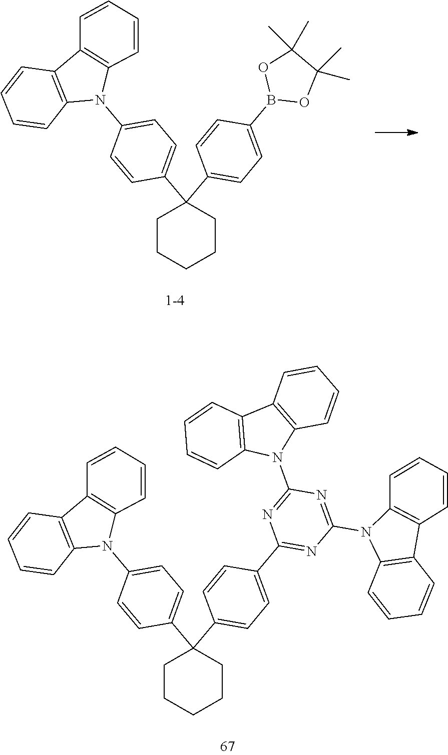 [SMILES: CC1(C)OB(c2ccc(C3(c4ccc(-n5c6ccccc6c6ccccc65)cc4)CCCCC3)cc2)OC1(C)C ; c1ccc2c(c1)c1ccccc1n2-c1ccc(C2(c3ccc(-c4nc(-n5c6ccccc6c6ccccc65)nc(-n5c6ccccc6c6ccccc65)n4)cc3)CCCCC2)cc1]

### Synthesis of Compound 67

In a nitrogen atmosphere, Intermediate 1-4 (1 eq), 9,9′-(6-chloro-1,3,5-triazine-2,4-diyl)bis(9H-carbazole) (1 eq), tetrakis(triphenylphosphine)palladium(0) (0.05 eq), and potassium carbonate (2.5 eq) were dissolved in a solution including tetrahydrofuran and H₂O mixed together at a ratio of 2:1 and stirred at a temperature of 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Compound 67 (yield of 61%).

### Synthesis Example 5: (synthesis of Compound 121)

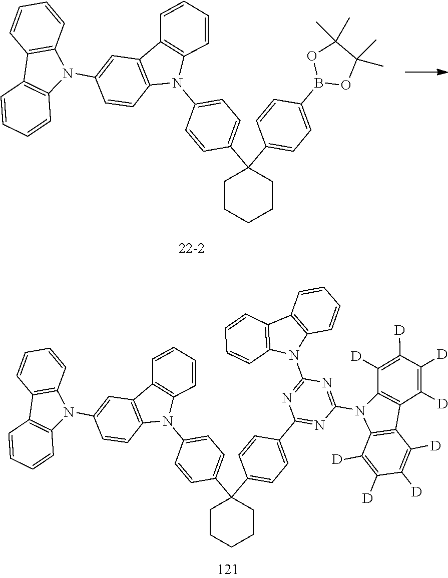 [SMILES: CC1(C)OB(c2ccc(C3(c4ccc(-n5c6ccccc6c6cc(-n7c8ccccc8c8ccccc87)ccc65)cc4)CCCCC3)cc2)OC1(C)C ; [2H]c1c([2H])c([2H])c2c(c1[2H])c1c([2H])c([2H])c([2H])c([2H])c1n2-c1nc(-c2ccc(C3(c4ccc(-n5c6ccccc6c6cc(-n7c8ccccc8c8ccccc87)ccc65)cc4)CCCCC3)cc2)nc(-n2c3ccccc3c3ccccc32)n1]

### Synthesis of Compound 121

In a nitrogen atmosphere, Intermediate 22-2 (1 eq), 9-(4-(9H-carbazol-9-yl)-6-chloro-1,3,5-triazin-2-yl)-9H-carbazole-1,2,3,4,5,6,7,8-d8 (1 eq), tetrakis(triphenylphosphine)palladium(0) (0.05 eq), and potassium carbonate (2.5 eq) were dissolved in a solution including tetrahydrofuran and H₂O mixed together at a ratio of 2:1 at a temperature of 80° C. for 15 hours. Then, an organic layer obtained by washing the obtained solution three times with ethylacetate and salt water was dried under reduced pressure after magnesium sulfate was added thereto. Subsequently, the separation-purification process was performed by column chromatography to obtain Compound 121 (yield of 57%).

### Synthesis Example 6 (synthesis of Compound 163)

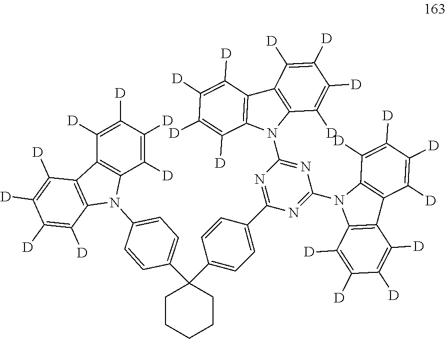 [SMILES: [2H]c1c([2H])c([2H])c2c(c1[2H])c1c([2H])c([2H])c([2H])c([2H])c1n2-c1ccc(C2(c3ccc(-c4nc(-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)cc3)CCCCC2)cc1]

Compound 163 (yield of 53%) was made by a reaction in substantially the same manner as in synthesizing Intermediate 1-3 except that 9H-carbazole-1,2,3,4,5,6,7,8-d8 was used as a starting material instead of 9H-carbazole, and a reaction in substantially the same manner as in synthesizing Intermediate 1 except that 9,9′-(5-chloro-1,3-phenylene)bis(9H-carbazole-1,2,3,4,5,6,7,8-d8) was used as a starting material instead of 2-chloro-4,6-diphenyl-1,3,5-triazine.

¹H nuclear magnetic resonance (NMR) spectroscopy and fast atom bombardment mass spectrometry (MS/FAB) of the compounds synthesized according to Synthesis Examples 1 to 5 are shown in Table 1. Synthesis methods of other compounds in addition to the compounds synthesized in Synthesis Examples 1 to 5 may be easily recognized by those skilled in the art by referring to the synthesis paths and source materials.

TABLE 1

| | | MS/FAB | |
|---|---|---|---|
| Compound | ¹H-NMR (CDCl₃, 500 MHz) | calc. | found |
| 1 | δ = 8.58-8.53(m, 3H), 8.38-8.33(m, 4H), | 632.81 | 632.77 |
| | 8.19(d, 1H), 7.94(d, 1H), 7.59-7.50(m, 8H), | | |
| | 7.38-7.15(m, 9H), 2.21-1.89(m, 4H), 1.55- | | |
| | 1.43(m, 6H) | | |
| 22 | δ = 8.56-8.53(m, 4H), 8.37-8.35(m, 4H), | 797.35 | 797.31 |
| | 8.20(d, 1H), 7.95-7.93(m, 2H), 7.71-7.67(m, | | |
| | 2H), 7.58-7.50(m, 8H), 7.38-7.17(m, 12H), | | |
| | 2.16-1.90(m, 4H), 1.54-1.44(m, 6H) | | |
| 46 | δ = 8.57-8.53(m, 3H), 8.37-8.34(m, 2H), | 722.89 | 722.83 |
| | 8.19(d, 1H), 8.03-7.98(m, 4H), 7.59-7.51(m, | | |
| | 7H), 7.39-7.31(m, 9H), 7.20-7.16(m, 2H), | | |
| | 2.18-1.90(m, 4H), 1.53-1.43(m, 6H) | | |
| 67 | δ = 8.57-8.52(m, 5H), 8.19(d, 3H), 7.93(d, | 811.01 | 810.97 |
| | 3H), 7.59-7.50(m, 6H), 7.38-7.15(m, 15H), | | |
| | 2.16-1.90(m, 4H), 1.53-1.43(m, 6H) | | |
| 121 | δ = 8.56-8.52(m, 5H), 8.18(d, 2H), 7.94(d, | 983.46 | 983.38 |
| | 3H), 7.73-7.67(m, 2H), 7.57-7.49(m, 4H), | | |
| | 7.38-7.27(m, 15H), 2.16-1.91(m, 4H), 1.53- | | |
| | 1.43(m, 6H) | | |
| 163 | δ = 8.56(d, 2H), 7.37-7.28(m, 6H), 2.15- | 834.50 | 834.47 |
| | 1.90(m, 4H), 1.53-1.43(m, 6H) | | |

### Example 1

As an anode, a 15 Ω/cm² (1,200 Å) ITO glass substrate (product of Corning Inc.) was cut to a size of 50 mm×50 mm×0.7 mm, sonicated using isopropyl alcohol and pure water each for 5 minutes, and then washed by irradiation of ultraviolet rays and exposure of ozone thereto for 30 minutes. Then, the glass substrate was mounted on a vacuum deposition apparatus.

NPD was vacuum-deposited on the substrate to form a hole injection layer having a thickness of 300 Å, and then, mCP as a hole transport compound was vacuum-deposited thereon to form a hole transport layer having a thickness of 200 Å.

Compound 1 and Ir(pmp)₃ were co-deposited at a weight ratio of 92:8 on the hole transport layer to form an emission layer having a thickness of 250 Å.

TAZ was deposited on the emission layer to form an electron transport layer having a thickness of 200 Å, LiF was deposited on the electron transport layer to form an electron injection layer having a thickness of 10 Å, and an Al electrode was vacuum-deposited to a thickness of 100 Å to form a LiF/AI electrode, thereby completing the manufacture of an organic electroluminescent device.

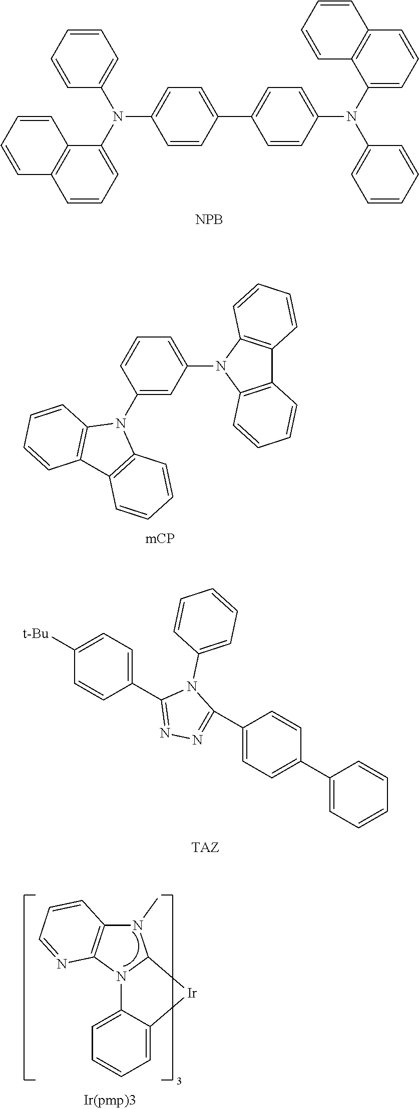

### Examples 2 to 6 and Comparative Examples 1 to 4

Organic electroluminescent devices were manufactured in substantially the same manner as in Example 1, except that, in forming an emission layer, the compounds shown in Table 2 were each used instead of Compound 1.

### Evaluation Example 1

The characteristics of the organic electroluminescent devices according to Examples and Comparative Examples were evaluated by measuring the driving voltage at the current density of 10 mA/cm², current density, and maximum quantum efficiency thereof. The driving voltage and current density of the organic electroluminescent devices were measured using a source meter (Keithley Instrument Inc., 2400 series), and the maximum quantum efficiency was measured using the external quantum efficiency measurement apparatus C9920-2-12 of Hamamatsu Photonics Inc. In evaluating the maximum quantum efficiency, the luminance/current density was measured using a luminance meter that was calibrated for wavelength sensitivity, and the maximum quantum efficiency was converted by assuming an angular luminance distribution (Lambertian) which introduced a perfect reflecting diffuser. Table 2 shows the evaluation results of the characteristics of the organic electroluminescent devices.

TABLE 2

| | | | Maximum | Half | |
|---|---|---|---|---|---|
| | | Driving | quantum | lifespan | Emis- |
| | Luminescent | voltage | efficiency | (hr @ 10 | sion |
| | material | (V) | (%) | mA/cm²) | color |
| Example 1 | Compound 1 | 3.3 | 15.7 | 73 | Blue |
| Example 2 | Compound 22 | 3.6 | 17.7 | 81 | Blue |
| Example 3 | Compound 46 | 3.4 | 17.2 | 69 | Blue |
| Example 4 | Compound 67 | 3.6 | 18.1 | 85 | Blue |
| Example 5 | Compound 121 | 3.4 | 18.8 | 93 | Blue |
| Example 6 | Compound 163 | 3.3 | 18.5 | 97 | Blue |
| Comparative | R01 | 3.9 | 12.1 | 61 | Blue |
| Example 1 | | | | | |
| Comparative | R02 | 5.9 | 8.7 | 40 | Blue |
| Example 2 | | | | | |
| Comparative | R03 | 4.3 | 13.9 | 57 | Blue |
| Example 3 | | | | | |
| Comparative | R04 | 4.1 | 13.7 | 49 | Blue |
| Example 4 | | | | | |

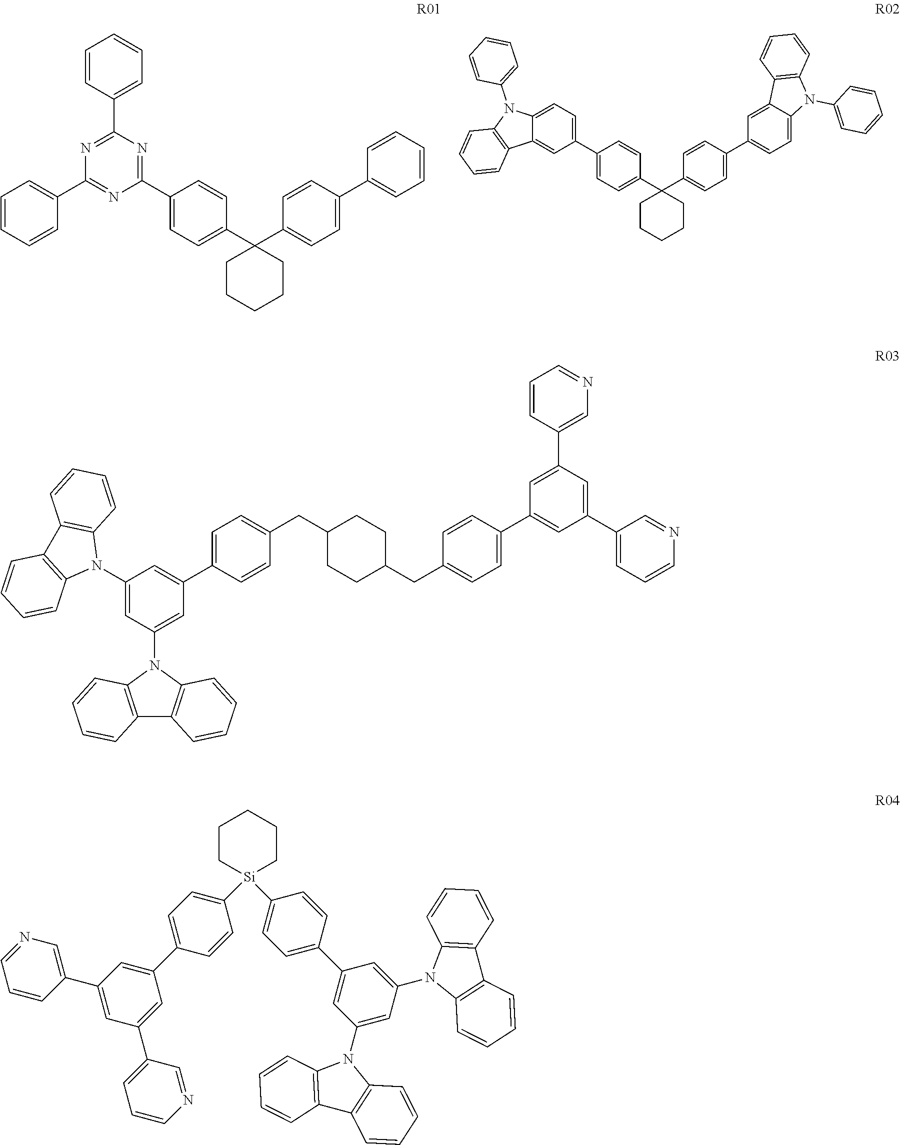

Referring to Table 2, the organic light-emitting devices according to Examples 1 to 6 were found to have lower driving voltage, higher efficiency, and longer lifespan than those of the organic light-emitting devices according to Comparative Examples 1 to 4.

As described above, according to the one or more embodiments, inclusion of a heterocyclic compound represented by Formula 1 may enable the manufacture of a light-emitting device having a low driving voltage and high efficiency and a high-quality electronic apparatus including the light-emitting device.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims, and equivalents thereof.

What is claimed is:

1. A light-emitting device comprising:
a first electrode;
a second electrode facing the first electrode;
an interlayer between the first electrode and the second electrode and comprising an emission layer; and
a heterocyclic compound represented by Formula 1: 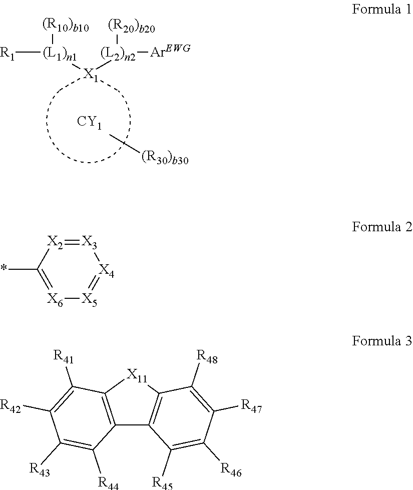
wherein, in Formulae 1 to 3,
X₁ is an sp3 hybridized carbon atom,
CY₁ is a C₁-C₆₀ carbocyclic group or a C₁-C₆₀ heterocyclic group,
Ar^{EWG} is a group represented by Formula 2, X₂ is N or C(R₂₎, X₃ is N or C(R₃), X₄ is N, C(R₄), or C(Ar₄), X₅ is N, C(R₅), or C(Ar₅), and X₆ is N or C(R₆),
at least one selected from X₂ to X₆ is N,
R₁ to R₆ are each independently a group represented by Formula 3, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂₎, —B(Q₁)(Q₂₎, —C(═O)(Q₁), —S(═O)₂ (Q₁), or —P(═O)(Q₁)(Q₂₎,
at least one selected from R₁ to R₆ is Ar^{EDG},
Ar^{EDG} is a group represented by Formula 3,
L₁ and L₂ are each independently a C₃-C₆₀ carbocyclic group or a C₁-C₆₀ heterocyclic group, n1 and n2 are each independently an integer from 1 to 5, i) when n1 is an integer of 2 or more, a plurality of L₁(s) are identical to or different from each other, and ii) when n2 is an integer of 2 or more, a plurality of L₂ (s) are identical to or different from each other,
R₁₀, R₂₀, and R₃₀ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂₎, —B(Q₁)(Q₂₎, —C(═O)(Q₁), —S(═O)₂ (Q₁), or —P(═O)(Q₁)(Q₂₎,
b10, b20, and b30 are each independently 0, 1, 2, 3, 4, 5, 6, 7, 8, 9, or 10,
X₁₁ is O, S, Se, N(R₄₉), or P(R₄₉),
R₄₁ to R₄₉ are independently a binding site to a neighboring atom, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂₎, —B(Q₁)(Q₂₎, —C(═O)(Q₁), —S(═O)₂ (Q₁), or —P(═O)(Q₁)(Q₂₎, any one selected from R₄₁ to R₄₉ is a binding site to a neighboring atom,
R₁₀ₐ is,
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂ (Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂ (Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

2. The light-emitting device of claim 1, wherein:
the first electrode is an anode,
the second electrode is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or a combination thereof, and
the electron transport region comprises a hole blocking layer, an electron transport layer, an electron injection layer, an electron control layer, or a combination thereof.

3. The light-emitting device of claim 1, wherein the emission layer comprises the heterocyclic compound represented by Formula 1.

4. The light-emitting device of claim 1, wherein:
the emission layer comprises a host and a dopant, and
the host comprises the heterocyclic compound represented by Formula 1.

5. The light-emitting device of claim 4, wherein the dopant comprises a phosphorescent dopant, a fluorescent dopant, a delayed fluorescence dopant, or a combination thereof.

6. An electronic apparatus comprising the light-emitting device of claim 1.

7. The electronic apparatus of claim 6, further comprising a thin-film transistor, wherein:
the thin-film transistor comprises a source electrode and a drain electrode, and
the first electrode of the light-emitting device is electrically connected to at least one selected from the source electrode and the drain electrode of the thin-film transistor.

8. The electronic apparatus of claim 6, further comprising a color filter, a color conversion layer, a touch screen layer, a polarizing layer, or a combination thereof.

9. A heterocyclic compound represented by Formula 1: 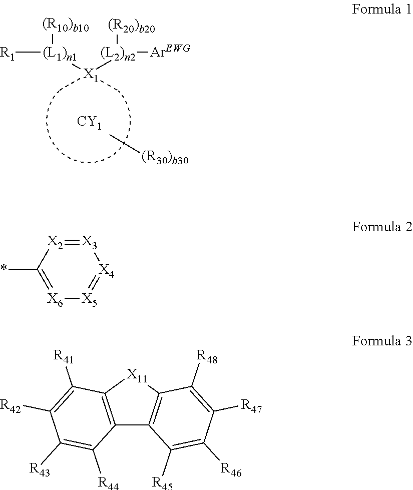
wherein, in Formulae 1 to 3,
X₁ is an sp3 hybridized carbon atom,
CY₁ is a C₁-C₆₀ carbocyclic group or a C₁-C₆₀ heterocyclic group,
Ar^{EWG} is a group represented by Formula 2, X₂ is N or C(R₂₎, X₃ is N or C(R₃), X₄ is N, C(R₄), or C(Ar₄), X₅ is N, C(R₅), or C(Ar₅), and X₆ is N or C(R₆),
at least one selected from X₂ to X₆ is N,
R₁ to R₆ are each independently a group represented by Formula 3, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂₎, —B(Q₁)(Q₂₎, —C(═O)(Q₁), —S(═O)₂ (Q₁), or —P(═O)(Q₁)(Q₂₎,
at least one selected from R₁ to R₆ is Ar^{EDG},
Ar^{EDG} is a group represented by Formula 3,
L₁ and L₂ are each independently a C₃-C₆₀ carbocyclic group or a C₁-C₆₀ heterocyclic group, n1 and n2 are each independently an integer from 1 to 5, i) when n1 is an integer of 2 or more, a plurality of L₁(s) are identical to or different from each other, and ii) when n2 is an integer of 2 or more, a plurality of L₂ (s) are identical to or different from each other,
R₁₀, R₂₀, and R₃₀ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂₎, —B(Q₁)(Q₂₎, —C(═O)(Q₁), —S(═O)₂ (Q₁), or —P(═O)(Q₁)(Q₂₎,
b10, b20, and b30 are each independently 0, 1, 2, 3, 4, 5, 6, 7, 8, 9, or 10,
X₁₁ is O, S, Se, N(R₄₉), or P(R₄₉),
R₄₁ to R₄₉ are each independently a binding site to a neighboring atom, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂₎, —B(Q₁)(Q₂₎, —C(═O)(Q₁), —S(═O)₂ (Q₁), or —P(═O)(Q₁)(Q₂₎,
any one selected from R₄₁ to R₄₉ is a binding site to a neighboring atom, R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂ (Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂ (Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂ (Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

10. The heterocyclic compound of claim 9, wherein L₁ and L₂ are each independently a benzene group, a naphthalene group, or a phenanthrene group.

11. The heterocyclic compound of claim 9, wherein Formula 1 is represented by one selected from Formulae 1 Å to Formula 11: 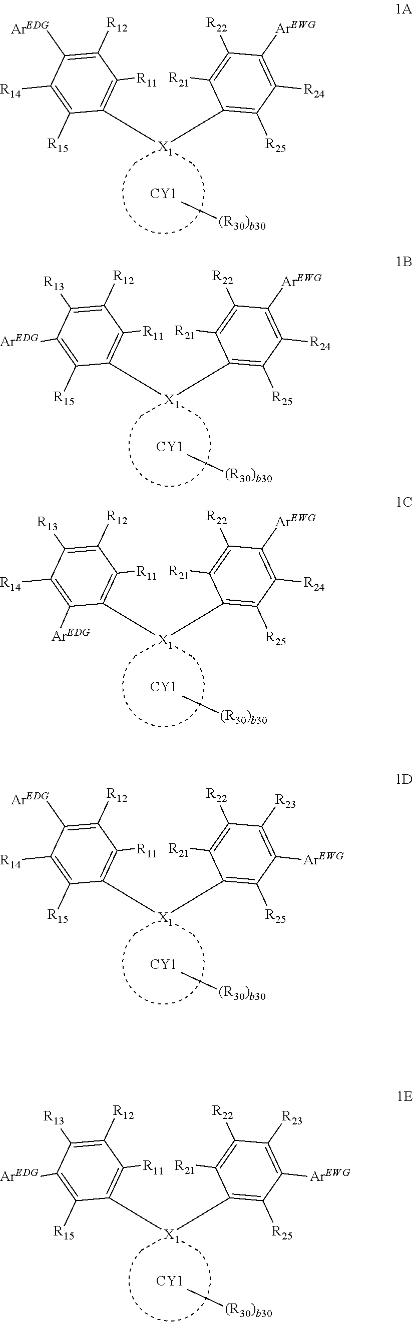 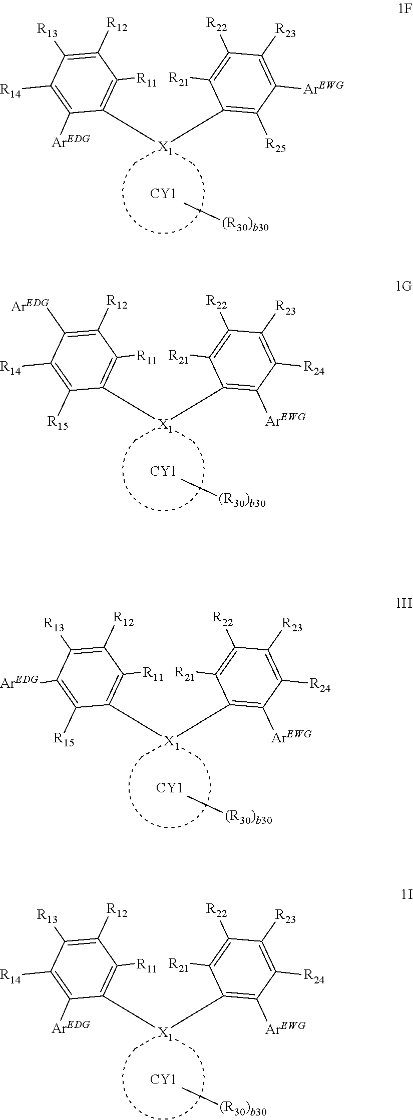
wherein, in Formulae 1A to 11,
X₁, CY₁, R₃₀, b30, Ar^{EDG}, and Ar^{EWG} are as defined in claim 9,
R₁₁ to R₁₅ are each as defined for R₁₀ in claim 9, and
R₂₁ to R₂₅ are each as defined for R₂₀ in claim 9.

12. The heterocyclic compound of claim 9, wherein CY₁ is represented by Formula 3 Å or 3B: 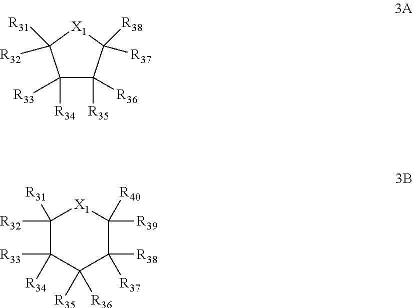
wherein, in Formulae 3A and 3B,
X₁ is as described in claim 9, and
R₃₁ to R₄₀ are each independently as defined for R₃₀ in claim 9.

13. The heterocyclic compound of claim 9, wherein R₁ is Ar^{EDG}.

14. The heterocyclic compound of claim 9, wherein Ar^{EDG} is a group represented by one selected from Formulae 3A to 3E: 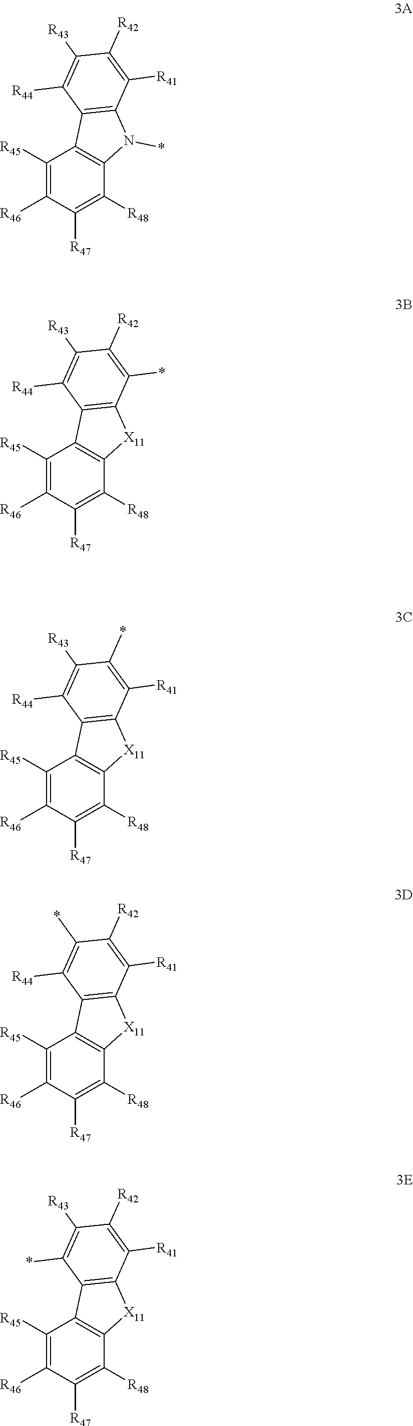
wherein, in Formulae 3A to 3E,
X₁₁ and R₄₁ to R₄₈ are as defined in claim 9, and
* indicates a binding site to Formula 1.

15. The heterocyclic compound of claim 9, wherein X₁₁ is O, S, or N(R₁).

16. The heterocyclic compound of claim 9, wherein:
R₄₁ to R₄₈ are each independently a binding site to a neighboring atom, hydrogen, deuterium, a C₁-C₂₀ alkyl group unsubstituted or substituted with at least one R₁₀ₐ, a C₂-C₂₀ alkenyl group unsubstituted or substituted with at least one R₁₀ₐ, a C₂-C₂₀ alkynyl group unsubstituted or substituted with at least one R₁₀ₐ, a hydroxyl group, a C₁-C₆₀ alkoxy group unsubstituted or substituted with at least one R₁₀ₐ, —N(Q₁)(Q₂₎, a C₃-C₂₀ cycloalkyl group unsubstituted or substituted with at least one R₁₀ₐ, a C₃-C₂₀ cycloalkenyl group unsubstituted or substituted with at least one R₁₀ₐ, a C₃-C₂₀ cycloalkynyl group unsubstituted or substituted with at least one R₁₀ₐ, a C₆-C₂₀ aryl group unsubstituted or substituted with at least one R₁₀ₐ, or a C₁-C₂₀ heteroaryl group unsubstituted or substituted with at least one R₁₀ₐ, and
R₁₀ₐ, Q₁, and Q₂ are as defined in claim 9.

17. The heterocyclic compound of claim 9, wherein Ar^{EWG} is represented by one selected from Formulae 2A to 2G: 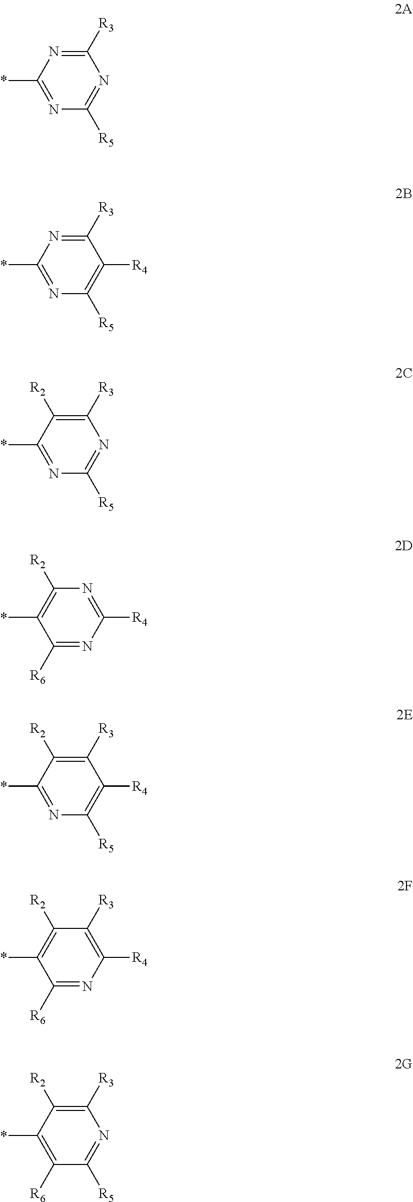
wherein, in Formulae 2A to 2G, R₂ to R₆ are as defined in claim 9.

18. The heterocyclic compound of claim 9, wherein:
R₂ to R₆ are each independently:
Ar^{EDG}; or
a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, or a chrysenyl group, each unsubstituted or substituted with at least one R₁₀ₐ, and
R₁₀ₐ is as defined in claim 9.

19. The heterocyclic compound of claim 9, wherein R₂ to R₆ are each independently a phenyl group unsubstituted or substituted with deuterium or a carbazolyl group unsubstituted or substituted with deuterium.

20. The heterocyclic compound of claim 9, wherein the heterocyclic compound is one selected from Compounds 1 to 165: 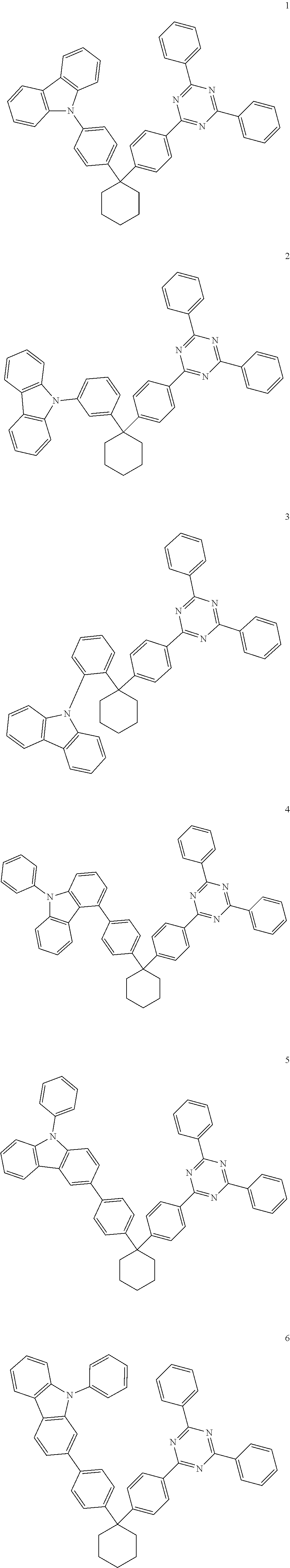 [chemical structure] 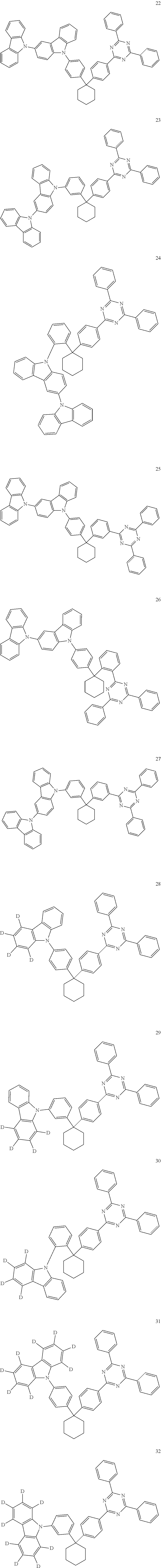 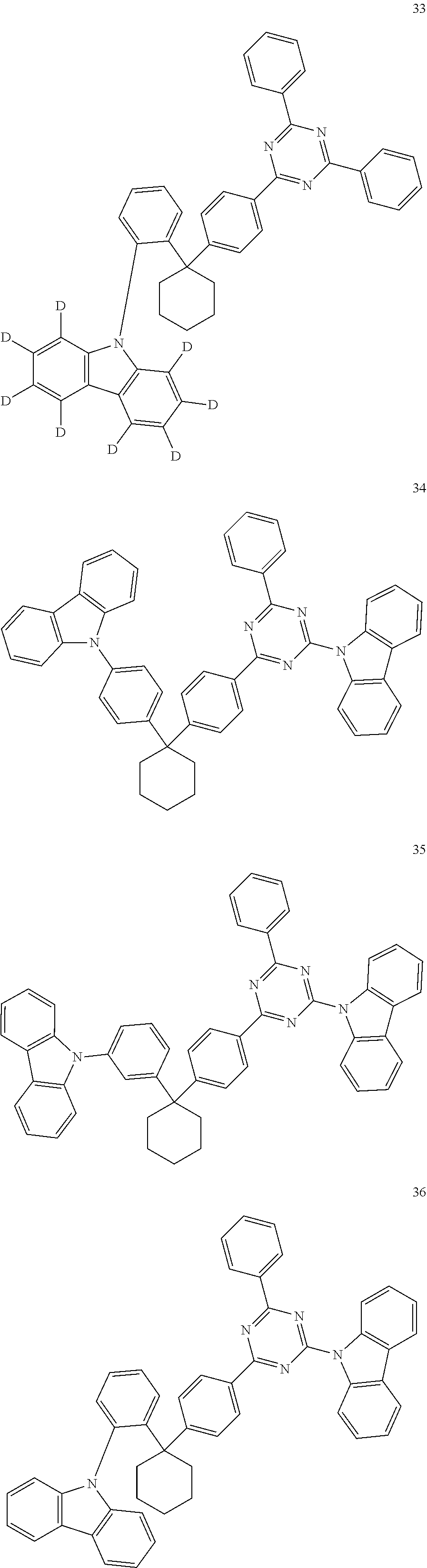 [SMILES: [2H]c1c([2H])c([2H])c2c(c1[2H])c1c([2H])c([2H])c([2H])c([2H])c1n2-c1ccccc1C1(c2ccc(-c3nc(-c4ccccc4)nc(-c4ccccc4)n3)cc2)CCCCC1 ; c1ccc(-c2nc(-c3ccc(C4(c5ccc(-n6c7ccccc7c7ccccc76)cc5)CCCCC4)cc3)nc(-n3c4ccccc4c4ccccc43)n2)cc1 ; c1ccc(-c2nc(-c3ccc(C4(c5cccc(-n6c7ccccc7c7ccccc76)c5)CCCCC4)cc3)nc(-n3c4ccccc4c4ccccc43)n2)cc1 ; c1ccc(-c2nc(-c3ccc(C4(c5ccccc5-n5c6ccccc6c6ccccc65)CCCCC4)cc3)nc(-n3c4ccccc4c4ccccc43)n2)cc1] 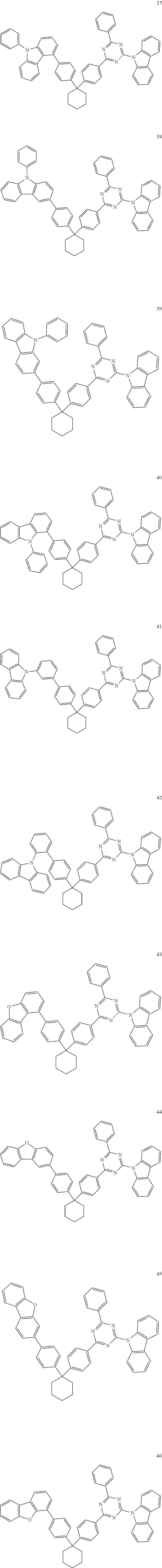 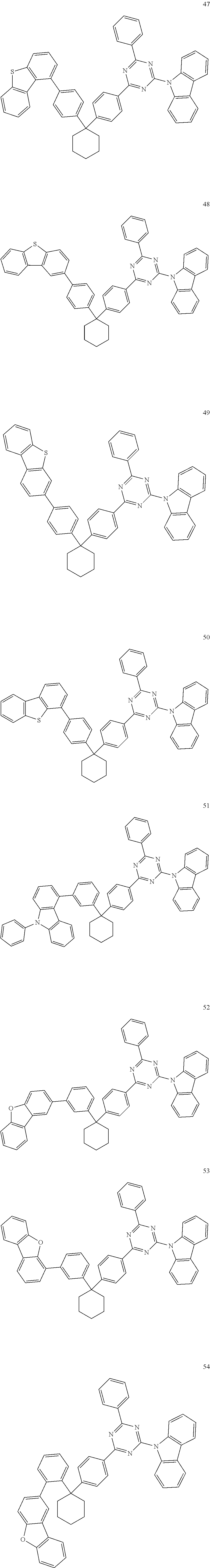 [chemical structure] 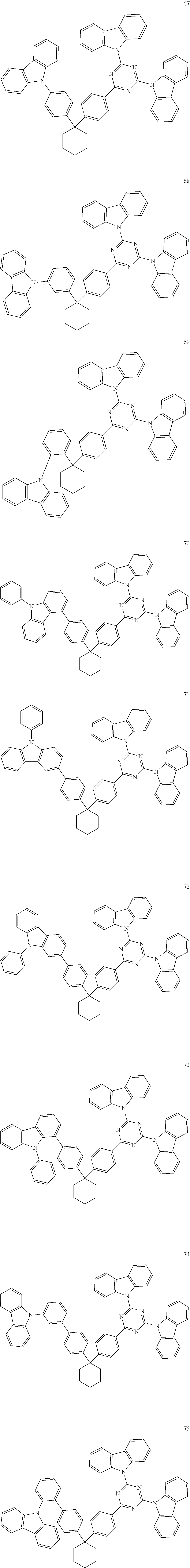 [chemical structure] 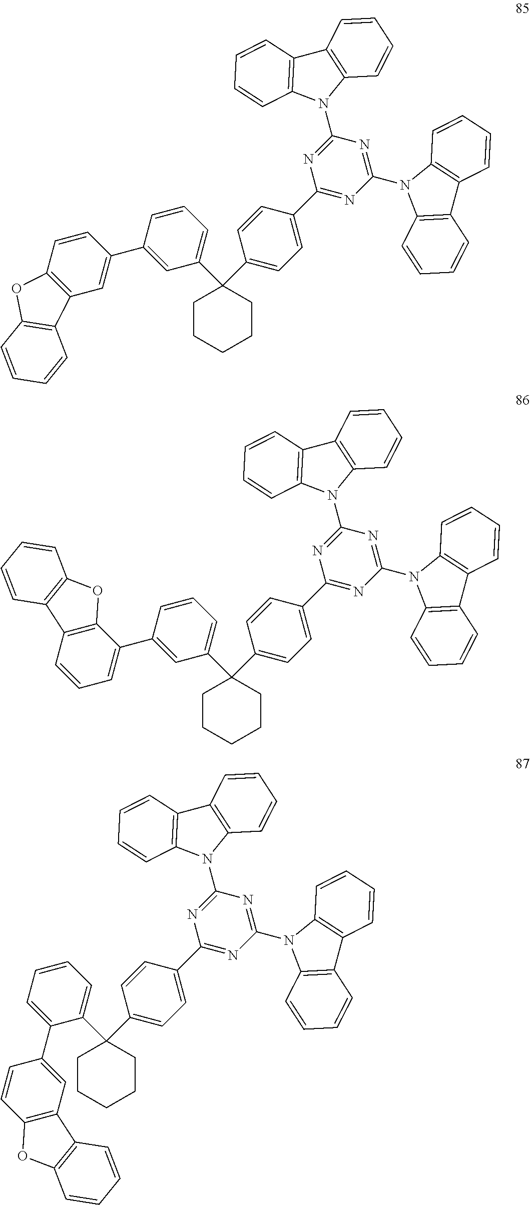 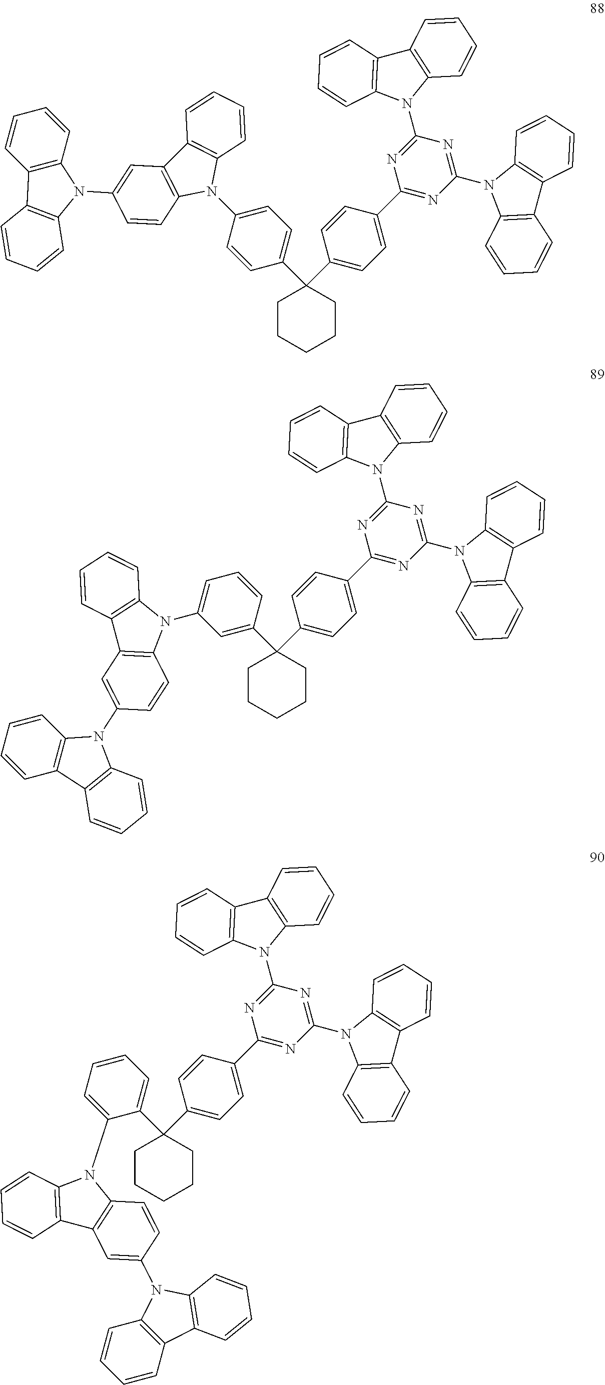 [SMILES: c1ccc2c(c1)c1ccccc1n2-c1ccc2c(c1)c1ccccc1n2-c1ccc(C2(c3ccc(-c4nc(-n5c6ccccc6c6ccccc65)nc(-n5c6ccccc6c6ccccc65)n4)cc3)CCCCC2)cc1 ; c1cc(-n2c3ccccc3c3cc(-n4c5ccccc5c5ccccc54)ccc32)cc(C2(c3ccc(-c4nc(-n5c6ccccc6c6ccccc65)nc(-n5c6ccccc6c6ccccc65)n4)cc3)CCCCC2)c1 ; c1ccc(C2(c3ccc(-c4nc(-n5c6ccccc6c6ccccc65)nc(-n5c6ccccc6c6ccccc65)n4)cc3)CCCCC2)c(-n2c3ccccc3c3cc(-n4c5ccccc5c5ccccc54)ccc32)c1] 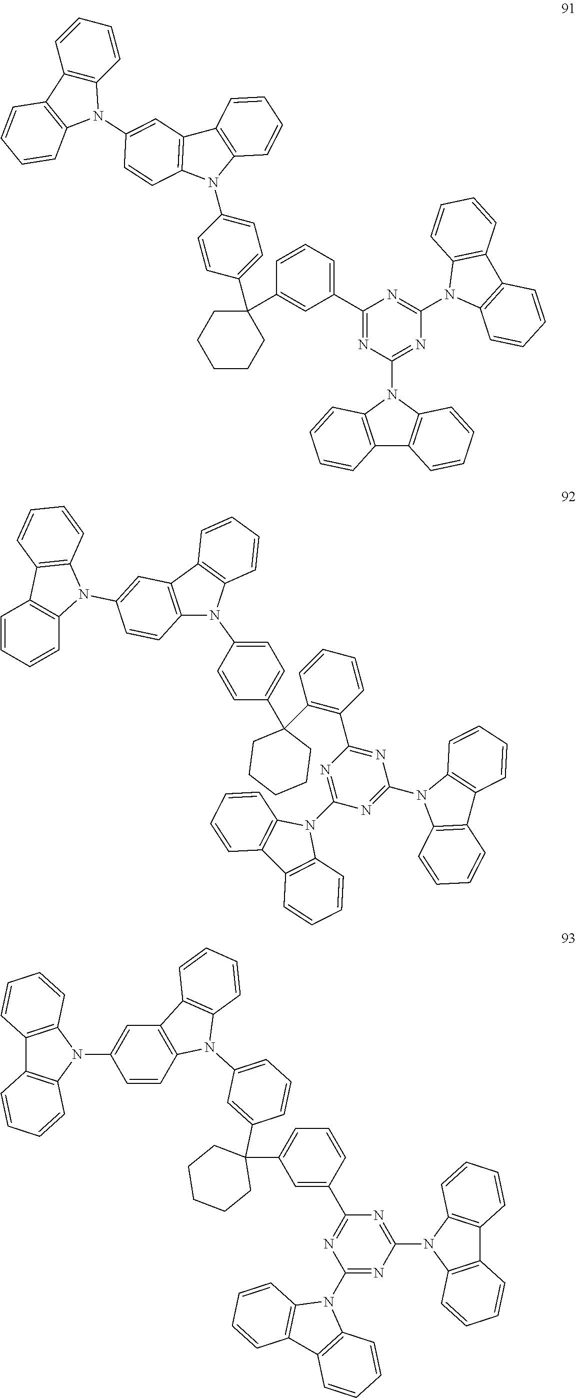 [SMILES: c1cc(-c2nc(-n3c4ccccc4c4ccccc43)nc(-n3c4ccccc4c4ccccc43)n2)cc(C2(c3ccc(-n4c5ccccc5c5cc(-n6c7ccccc7c7ccccc76)ccc54)cc3)CCCCC2)c1 ; c1ccc(C2(c3ccc(-n4c5ccccc5c5cc(-n6c7ccccc7c7ccccc76)ccc54)cc3)CCCCC2)c(-c2nc(-n3c4ccccc4c4ccccc43)nc(-n3c4ccccc4c4ccccc43)n2)c1 ; c1cc(-c2nc(-n3c4ccccc4c4ccccc43)nc(-n3c4ccccc4c4ccccc43)n2)cc(C2(c3cccc(-n4c5ccccc5c5cc(-n6c7ccccc7c7ccccc76)ccc54)c3)CCCCC2)c1] 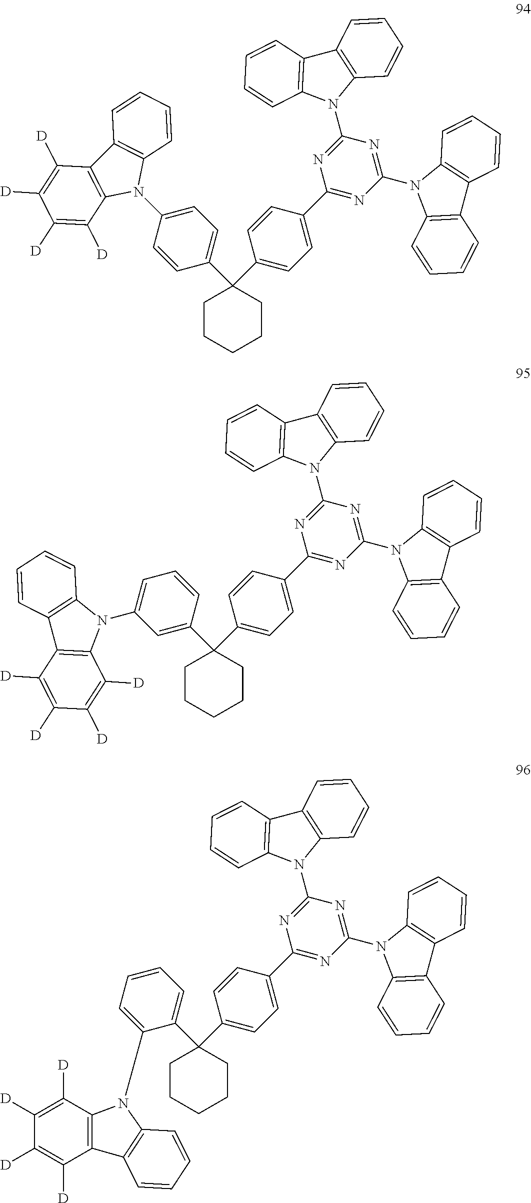 [SMILES: [2H]c1c([2H])c([2H])c2c(c1[2H])c1ccccc1n2-c1ccc(C2(c3ccc(-c4nc(-n5c6ccccc6c6ccccc65)nc(-n5c6ccccc6c6ccccc65)n4)cc3)CCCCC2)cc1 ; [2H]c1c([2H])c([2H])c2c(c1[2H])c1ccccc1n2-c1cccc(C2(c3ccc(-c4nc(-n5c6ccccc6c6ccccc65)nc(-n5c6ccccc6c6ccccc65)n4)cc3)CCCCC2)c1 ; [2H]c1c([2H])c([2H])c2c(c1[2H])c1ccccc1n2-c1ccccc1C1(c2ccc(-c3nc(-n4c5ccccc5c5ccccc54)nc(-n4c5ccccc5c5ccccc54)n3)cc2)CCCCC1] 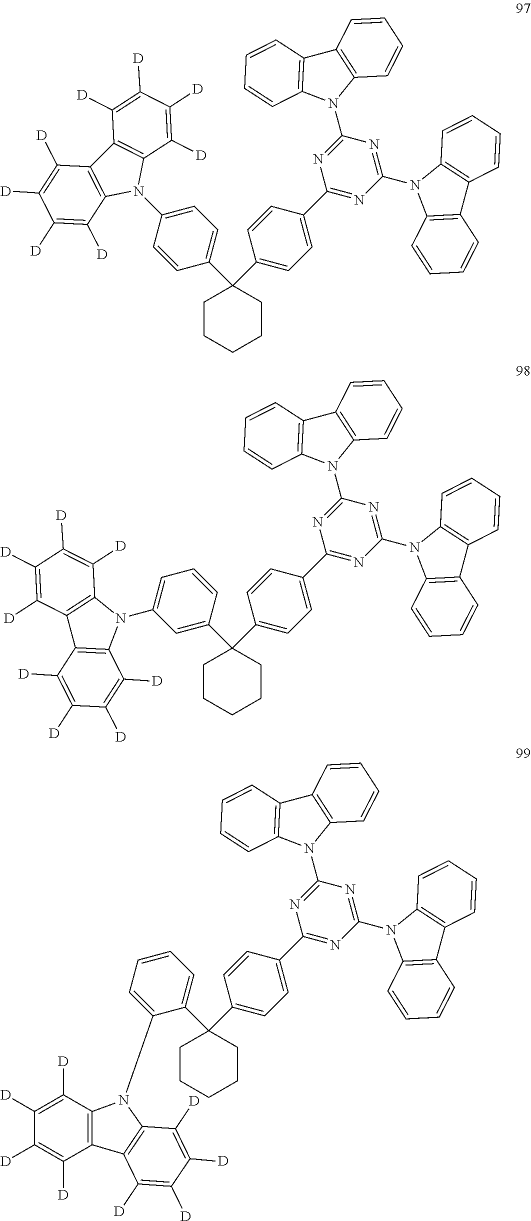 [SMILES: [2H]c1c([2H])c([2H])c2c(c1[2H])c1c([2H])c([2H])c([2H])c([2H])c1n2-c1ccc(C2(c3ccc(-c4nc(-n5c6ccccc6c6ccccc65)nc(-n5c6ccccc6c6ccccc65)n4)cc3)CCCCC2)cc1 ; [2H]c1c([2H])c([2H])c2c(c1[2H])c1c([2H])c([2H])c([2H])c([2H])c1n2-c1cccc(C2(c3ccc(-c4nc(-n5c6ccccc6c6ccccc65)nc(-n5c6ccccc6c6ccccc65)n4)cc3)CCCCC2)c1 ; [2H]c1c([2H])c([2H])c2c(c1[2H])c1c([2H])c([2H])c([2H])c([2H])c1n2-c1ccccc1C1(c2ccc(-c3nc(-n4c5ccccc5c5ccccc54)nc(-n4c5ccccc5c5ccccc54)n3)cc2)CCCCC1] 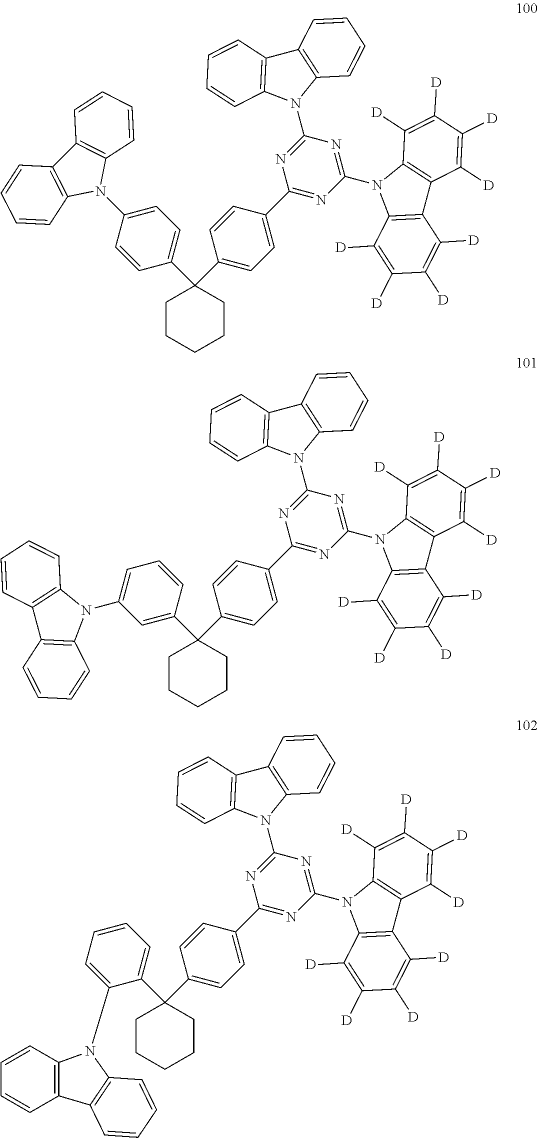 [SMILES: [2H]c1c([2H])c([2H])c2c(c1[2H])c1c([2H])c([2H])c([2H])c([2H])c1n2-c1nc(-c2ccc(C3(c4ccc(-n5c6ccccc6c6ccccc65)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4cccc(-n5c6ccccc6c6ccccc65)c4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccccc4-n4c5ccccc5c5ccccc54)CCCCC3)cc2)nc(-n2c3ccccc3c3ccccc32)n1] 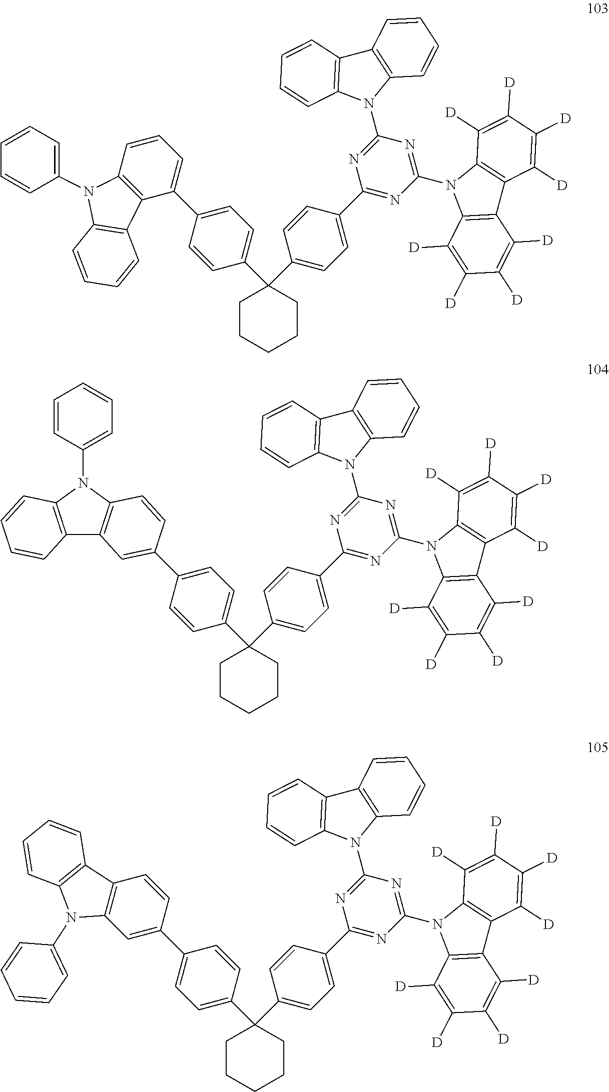 [SMILES: [2H]c1c([2H])c([2H])c2c(c1[2H])c1c([2H])c([2H])c([2H])c([2H])c1n2-c1nc(-c2ccc(C3(c4ccc(-c5cccc6c5c5ccccc5n6-c5ccccc5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6c(c5)c5ccccc5n6-c5ccccc5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6c7ccccc7n(-c7ccccc7)c6c5)cc4)CCCCC3)cc2)nc(-n2c3ccccc3c3ccccc32)n1] 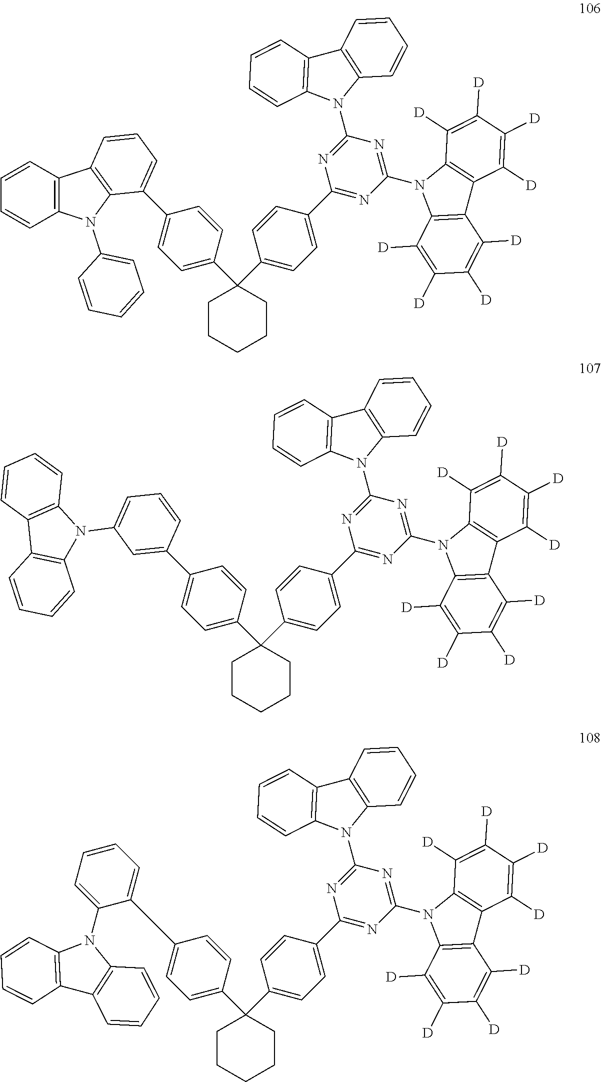 [SMILES: [2H]c1c([2H])c([2H])c2c(c1[2H])c1c([2H])c([2H])c([2H])c([2H])c1n2-c1nc(-c2ccc(C3(c4ccc(-c5cccc6c7ccccc7n(-c7ccccc7)c56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5cccc(-n6c7ccccc7c7ccccc76)c5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccccc5-n5c6ccccc6c6ccccc65)cc4)CCCCC3)cc2)nc(-n2c3ccccc3c3ccccc32)n1] 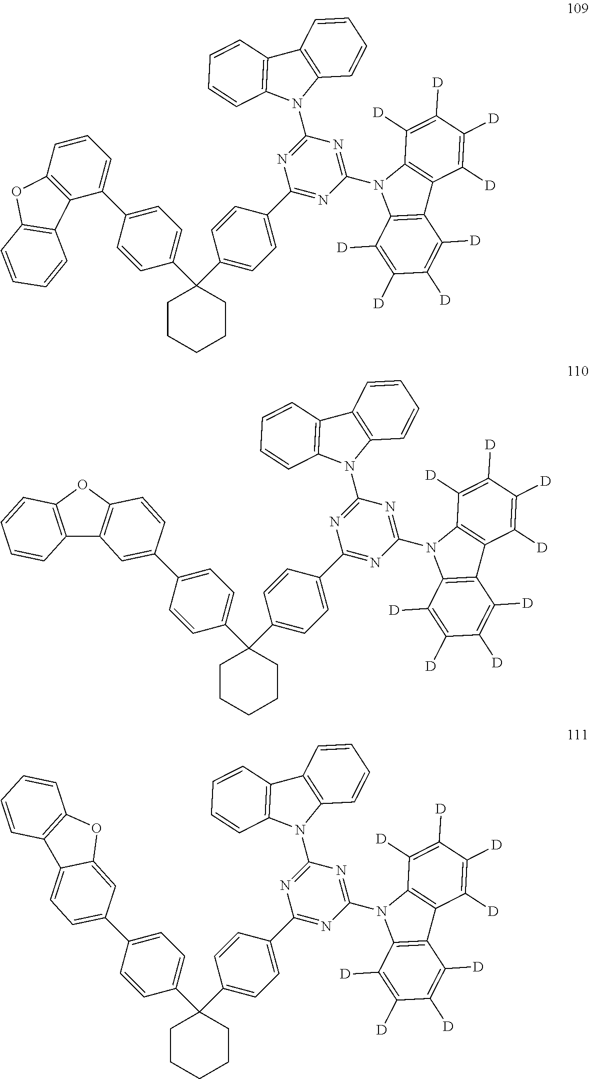 [SMILES: [2H]c1c([2H])c([2H])c2c(c1[2H])c1c([2H])c([2H])c([2H])c([2H])c1n2-c1nc(-c2ccc(C3(c4ccc(-c5cccc6oc7ccccc7c56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6oc7ccccc7c6c5)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6c(c5)oc5ccccc56)cc4)CCCCC3)cc2)nc(-n2c3ccccc3c3ccccc32)n1] 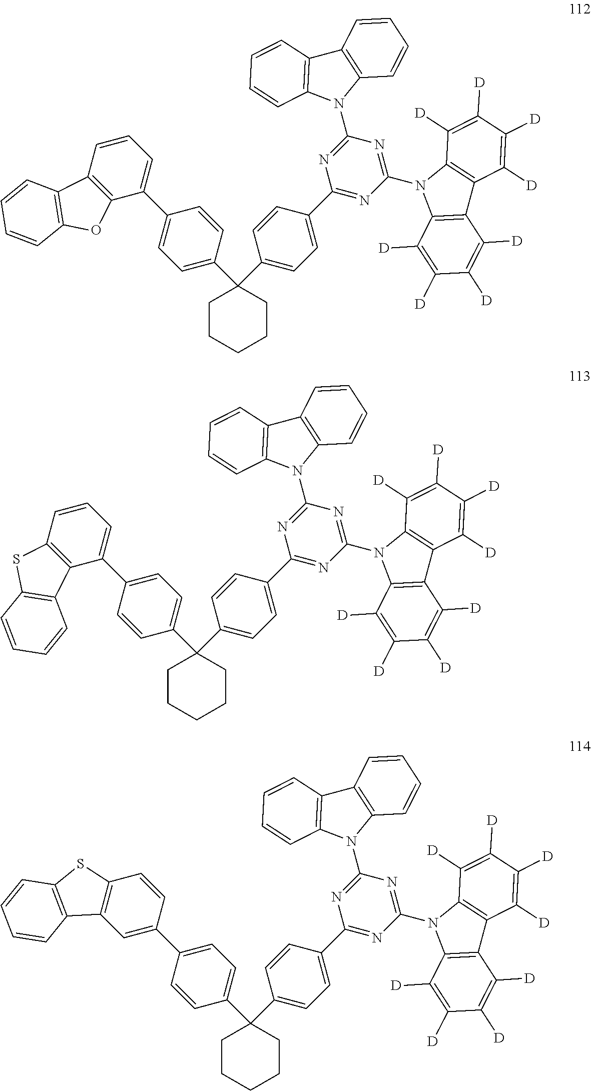 [SMILES: [2H]c1c([2H])c([2H])c2c(c1[2H])c1c([2H])c([2H])c([2H])c([2H])c1n2-c1nc(-c2ccc(C3(c4ccc(-c5cccc6c5oc5ccccc56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5cccc6sc7ccccc7c56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5ccc6sc7ccccc7c6c5)cc4)CCCCC3)cc2)nc(-n2c3ccccc3c3ccccc32)n1] 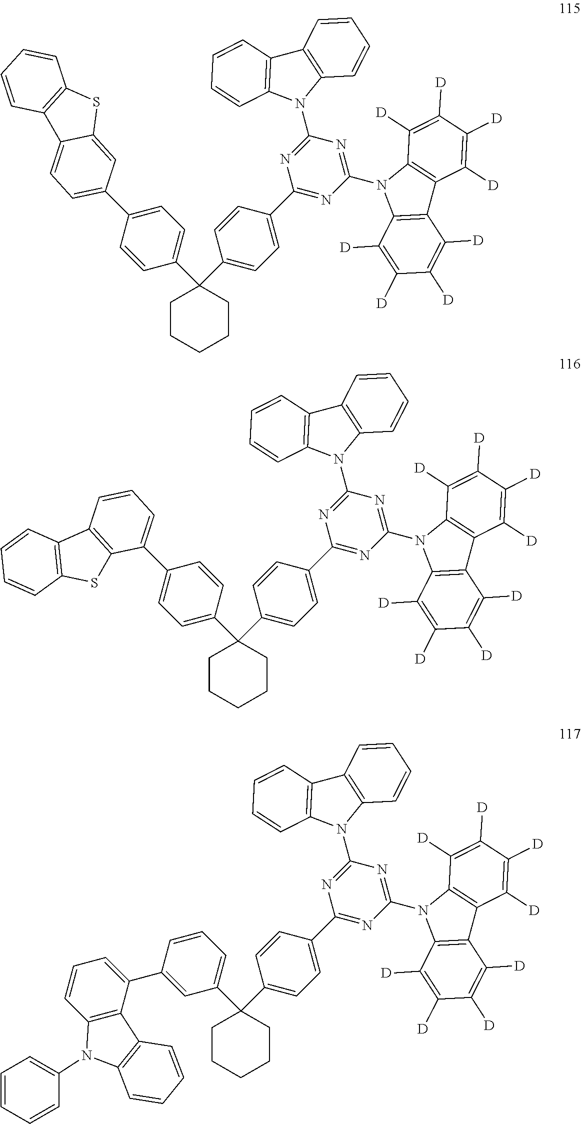 [SMILES: [2H]c1c([2H])c([2H])c2c(c1[2H])c1c([2H])c([2H])c([2H])c([2H])c1n2-c1nc(-c2ccc(C3(c4ccc(-c5ccc6c(c5)sc5ccccc56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4ccc(-c5cccc6c5sc5ccccc56)cc4)CCCCC3)cc2)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccc(C3(c4cccc(-c5cccc6c5c5ccccc5n6-c5ccccc5)c4)CCCCC3)cc2)nc(-n2c3ccccc3c3ccccc32)n1] 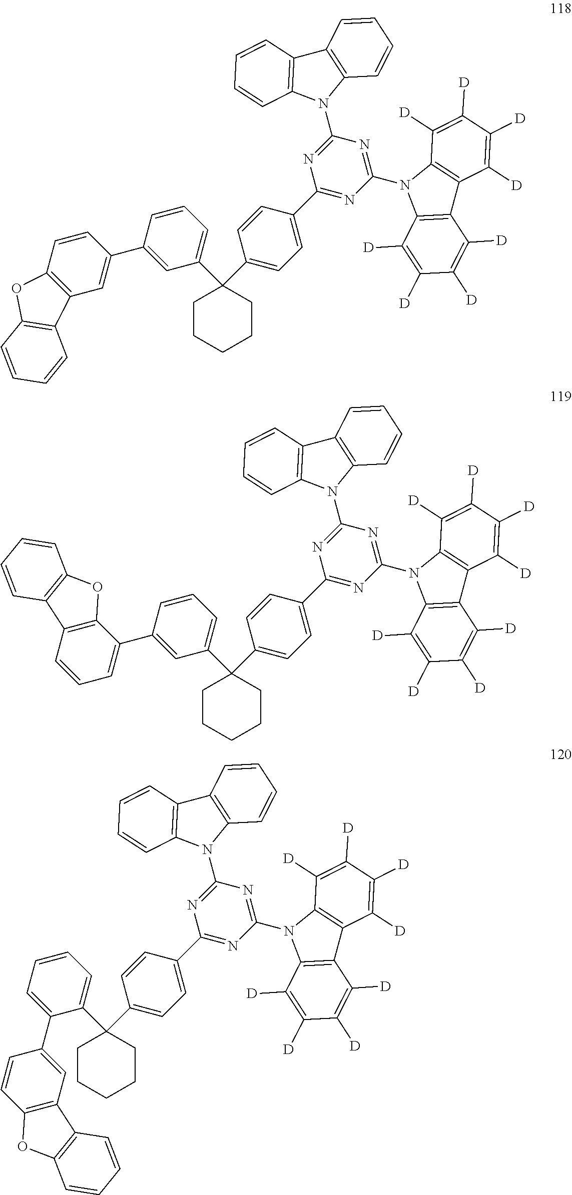 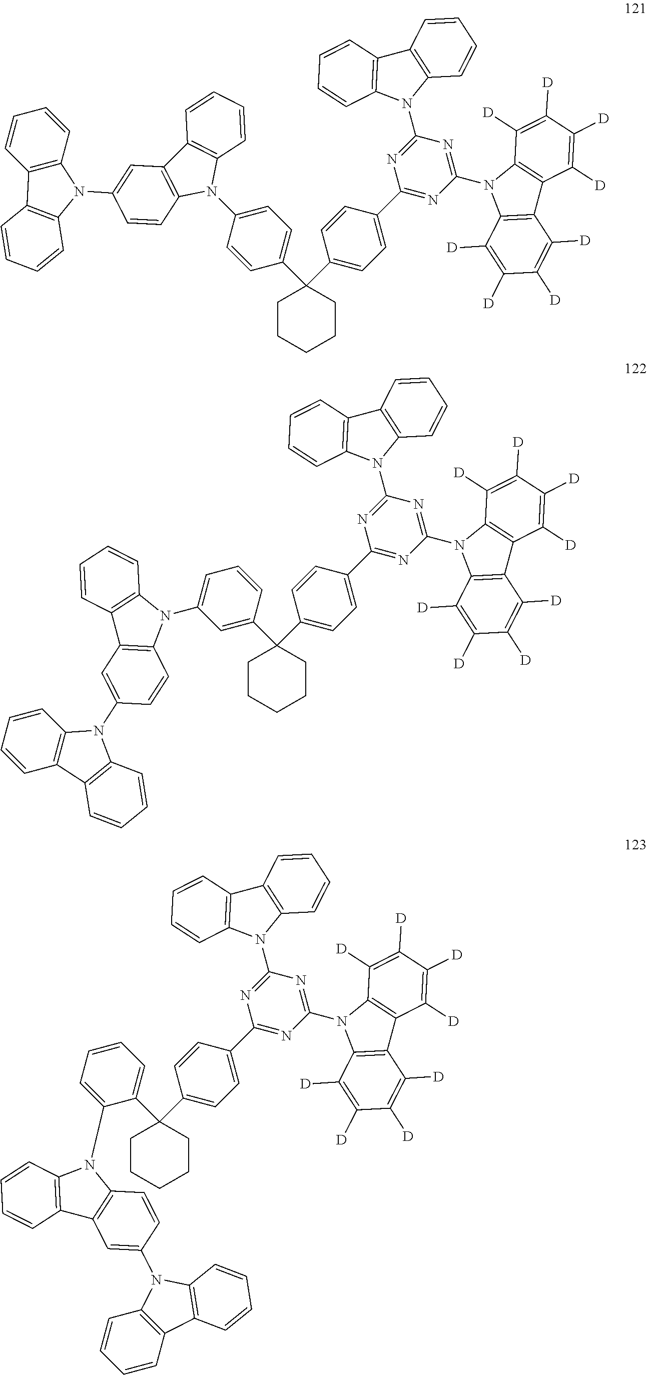 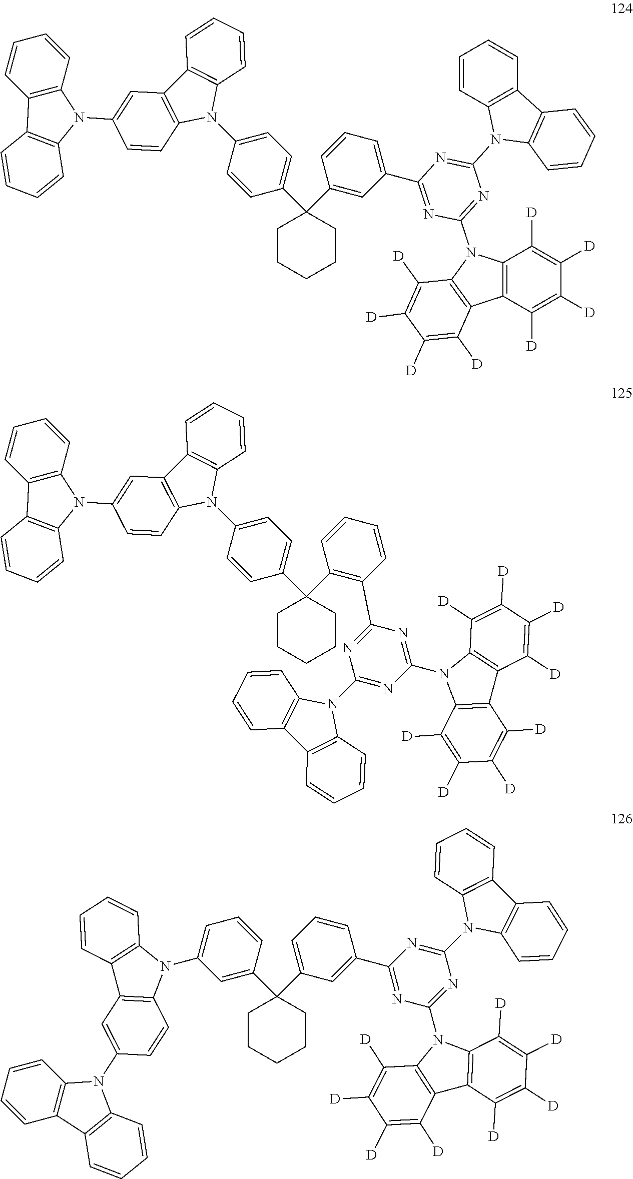 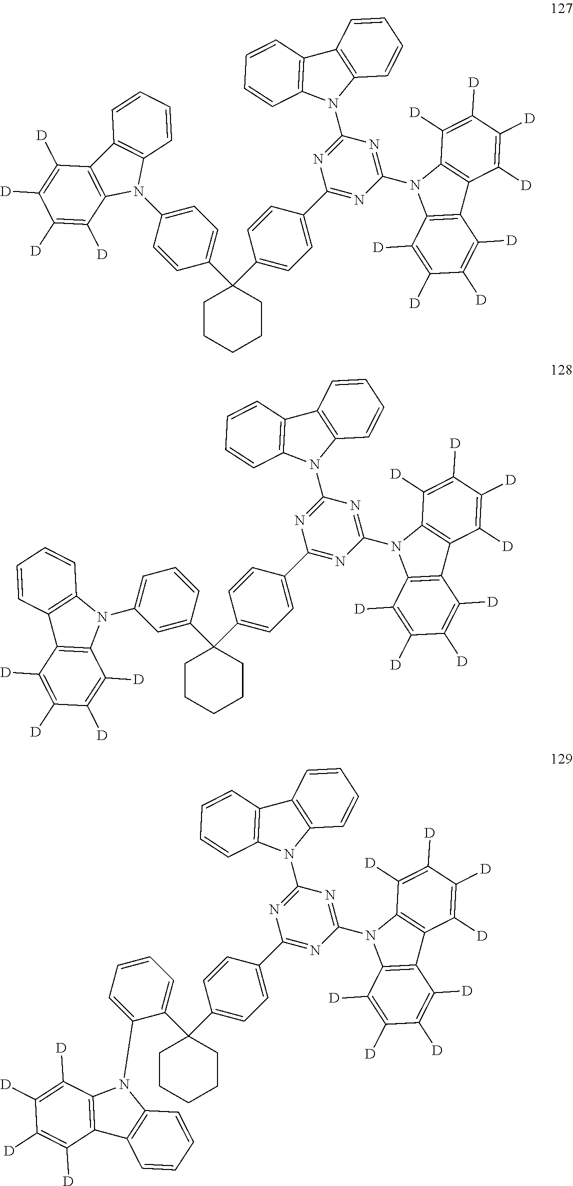 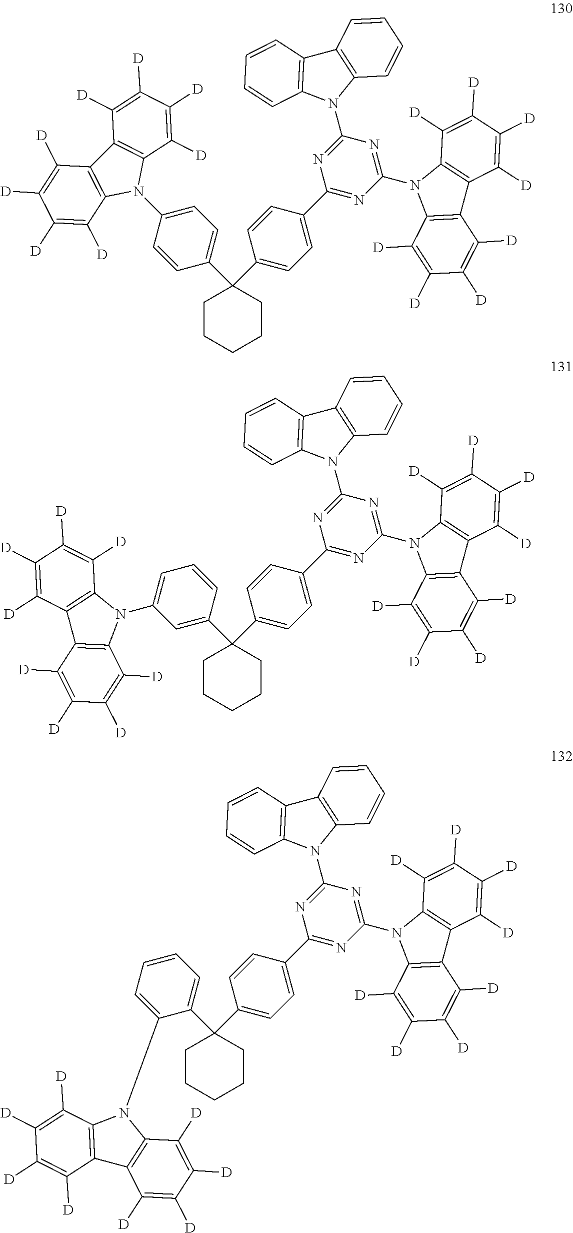 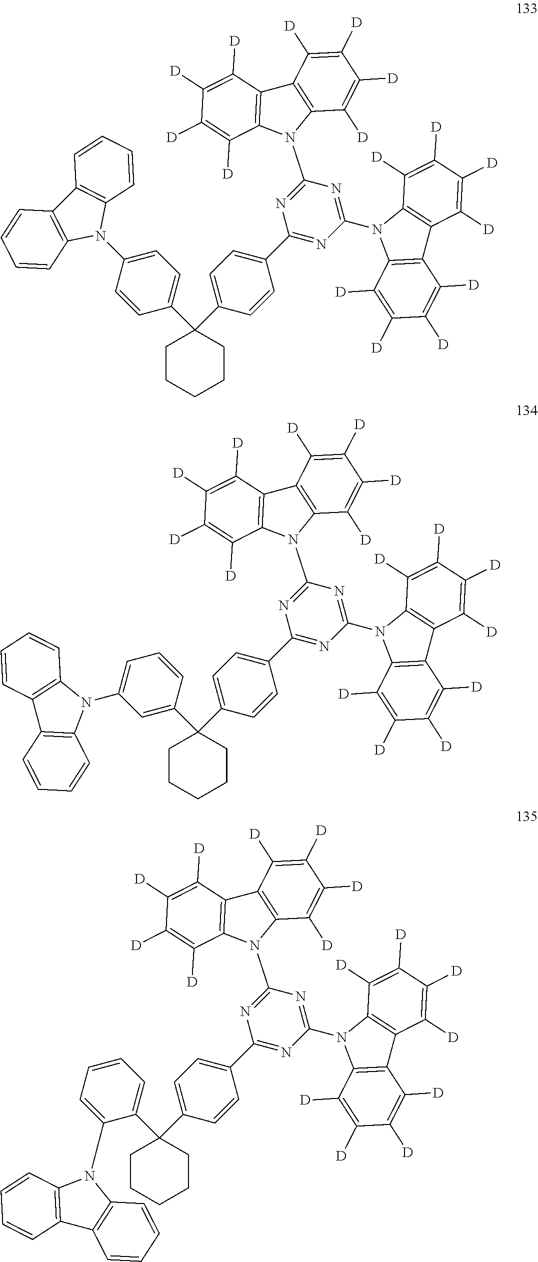 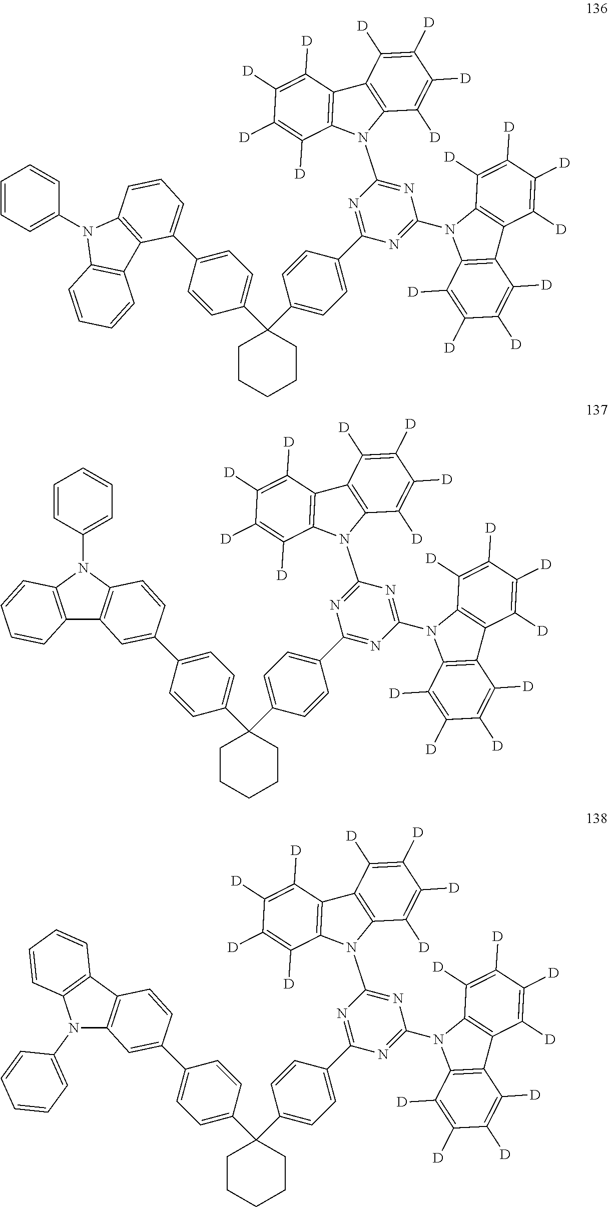 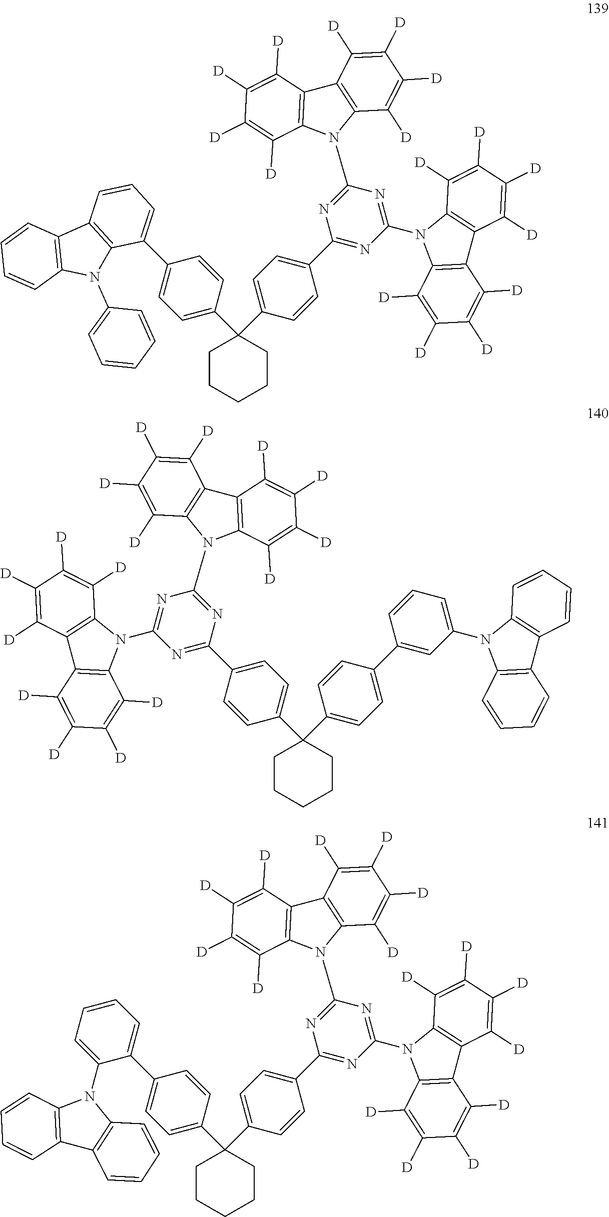 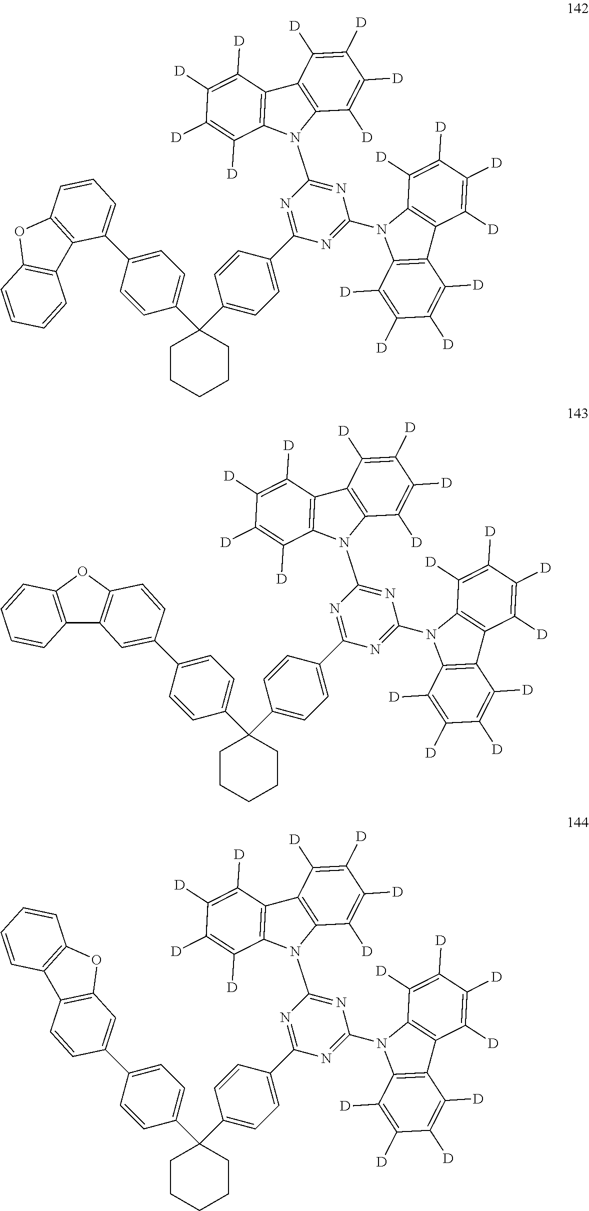 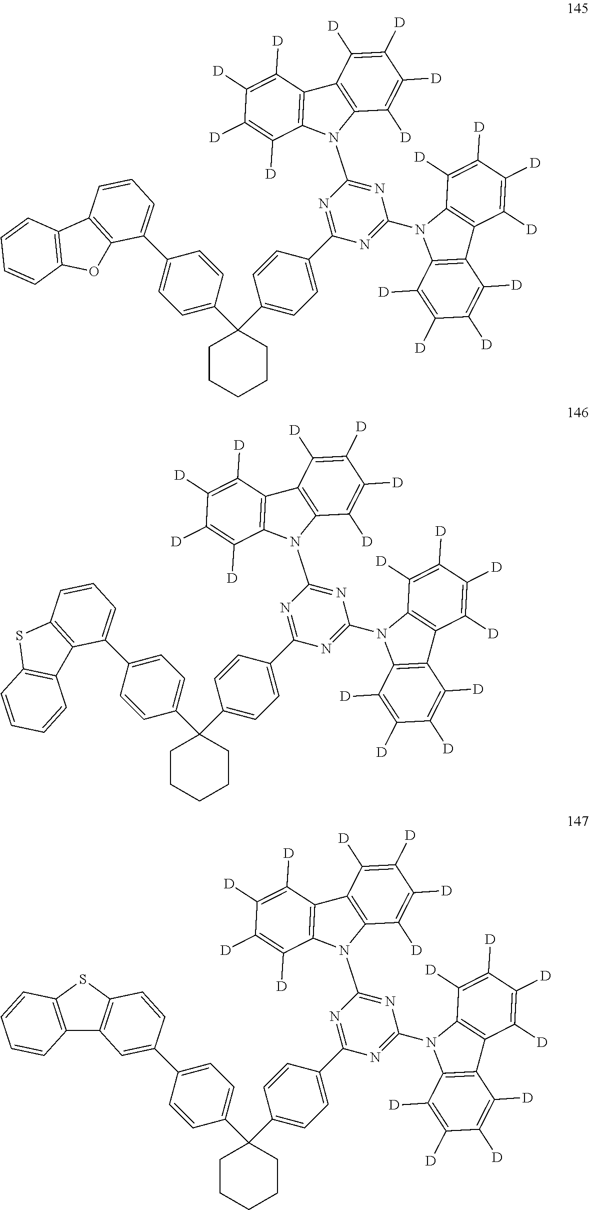 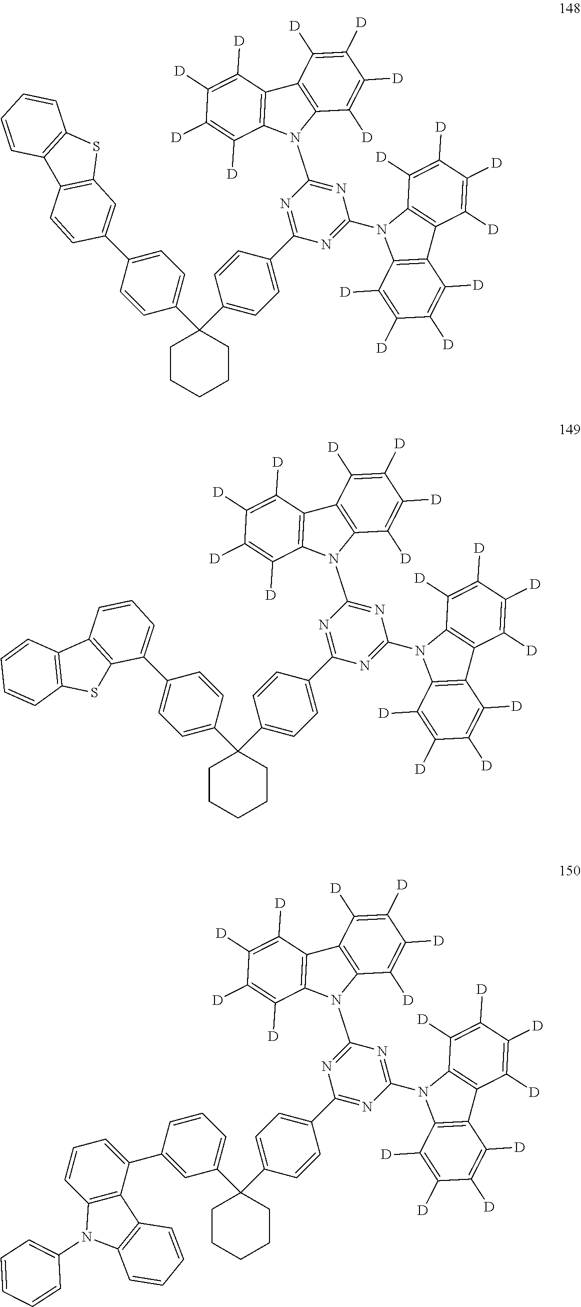 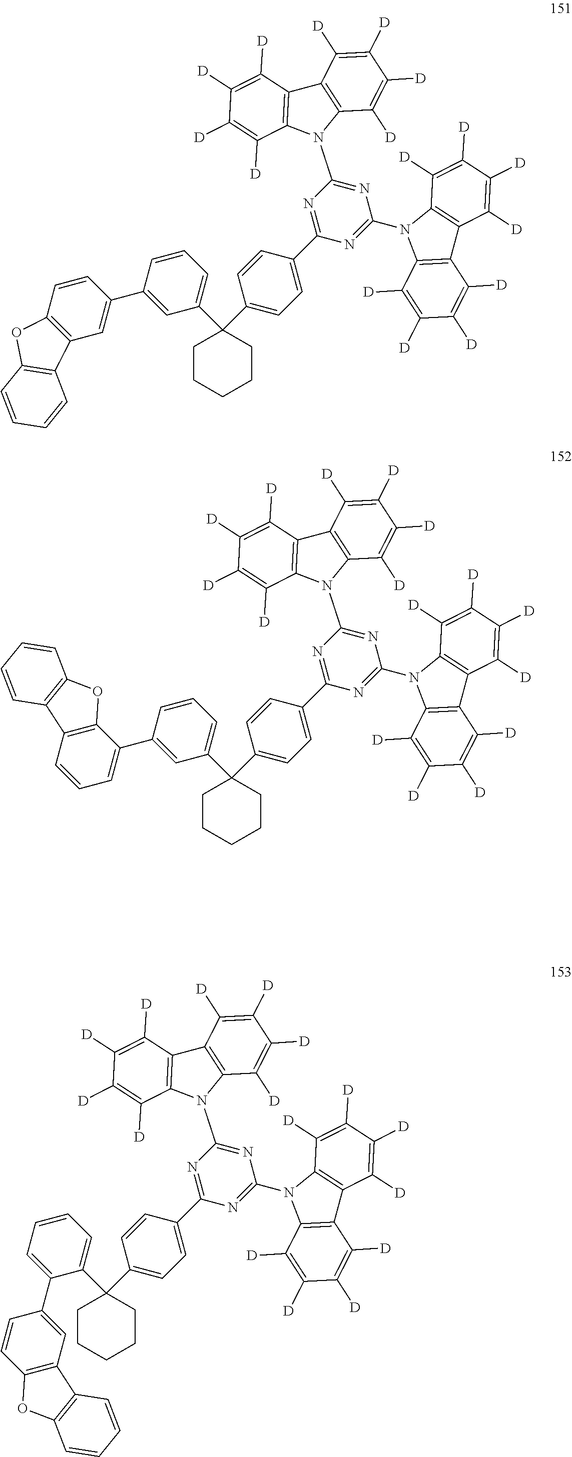 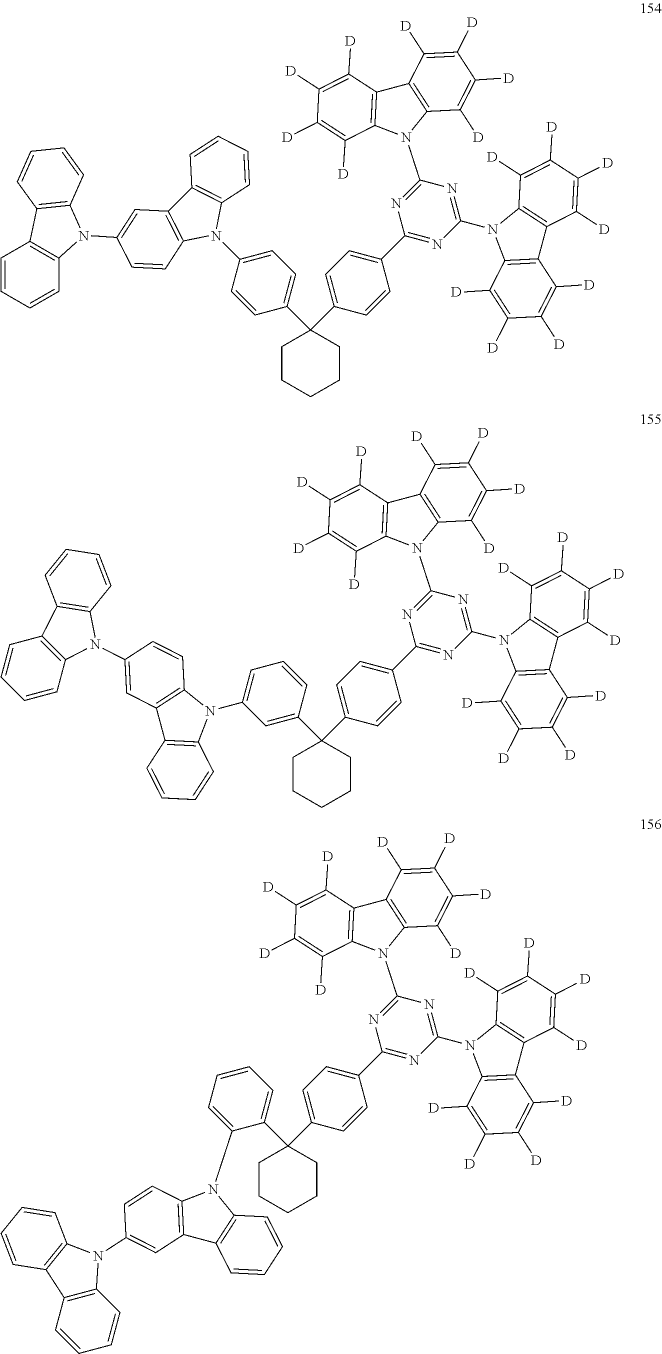 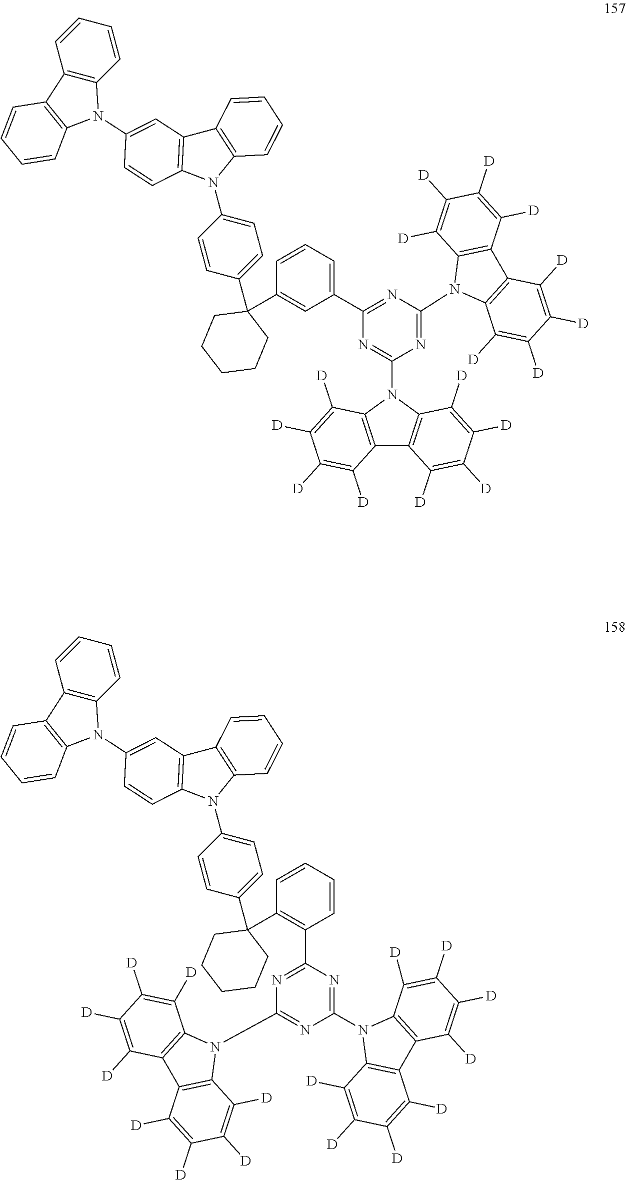 [SMILES: [2H]c1c([2H])c([2H])c2c(c1[2H])c1c([2H])c([2H])c([2H])c([2H])c1n2-c1nc(-c2cccc(C3(c4ccc(-n5c6ccccc6c6cc(-n7c8ccccc8c8ccccc87)ccc65)cc4)CCCCC3)c2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2C2(c3ccc(-n4c5ccccc5c5cc(-n6c7ccccc7c7ccccc76)ccc54)cc3)CCCCC2)nc(-n2c3c([2H])c([2H])c([2H])c([2H])c3c3c([2H])c([2H])c([2H])c([2H])c32)n1] 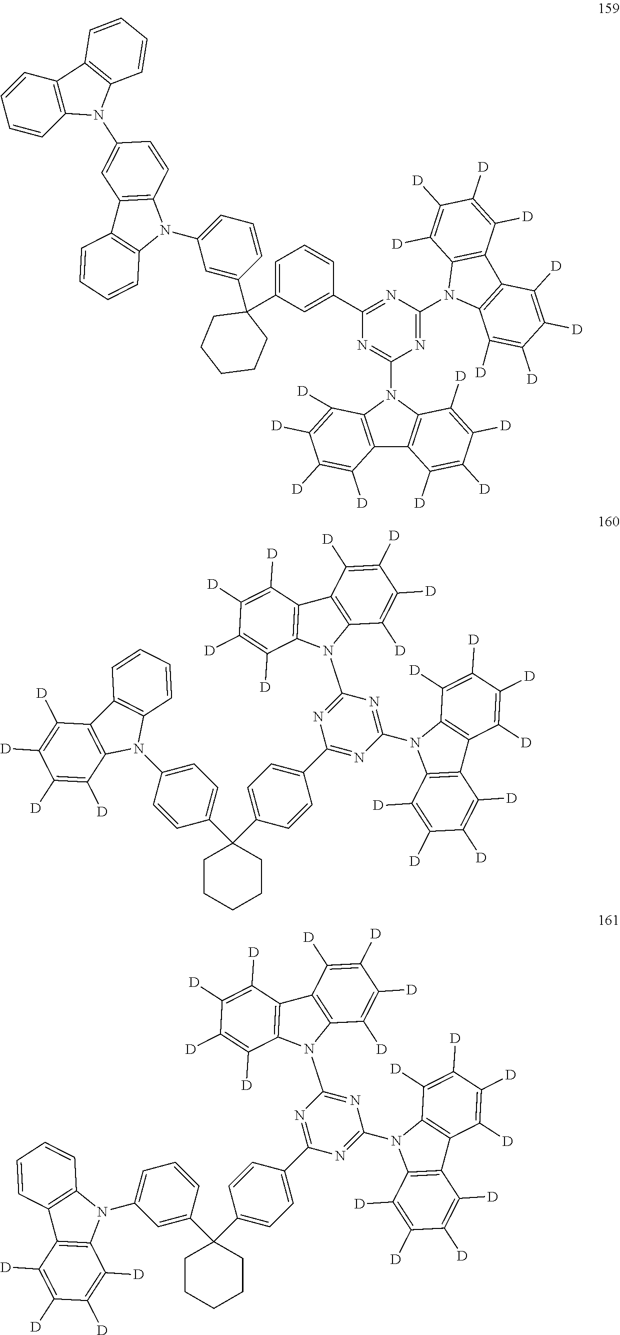 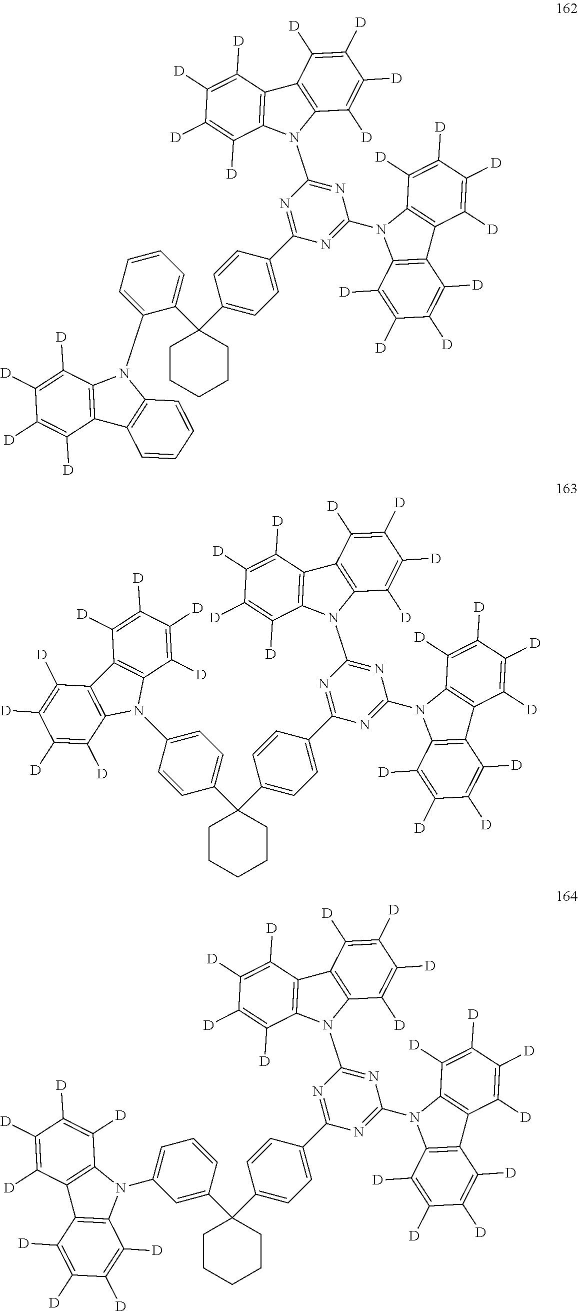 [chemical structure]
